SEQUENCE LISTING (15 sequences)

SEQ ID NO: 1 - RNA, 1632 nt, synthetic construct
  misc_feature 1..1632: note = Synthetic polynucleotide
  variation 1527: note = May or may not be present
  variation 1627: note = May or may not be present
  source 1..1632: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatgcagcgcgtgaacatgatcatggcagaatcaccaggcctcatcaccatctgccttttaggatatctactcagtgctgaatgtacagtttttcttgatcatgaaaacgccaacaaaattctgaggcggagaaggaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgaggctgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaacgggtggcatccctgtgacccctccccagtgcctctcctggccctggaagttgccactccagtgcccaccagccttgtcctaataaaattaagttgcatcaaagct

SEQ ID NO: 2 - RNA, 1485 nt, synthetic construct
  misc_feature 1..1485: note = Synthetic polynucleotide
  variation 1380: note = May or may not be present
  variation 1480: note = May or may not be present
  source 1..1485: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatgagcagcaagggcagcgtggtgctggcctacagcggcggcctggacaccagctgcatcctggtgtggctgaaggagcagggctacgacgtgatcgcctacctggccaacatcggccagaaggaggacttcgaggaggcccgcaagaaggccctgaagctgggcgccaagaaggtgttcatcgaggacgtgagccgcgagttcgtggaggagttcatctggcccgccatccagagcagcgccctgtacgaggaccgctacctgctgggcaccagcctggcccgcccctgcatcgcccgcaagcaggtggagatcgcccagcgcgagggcgccaagtacgtgagccacggcgccaccggcaagggcaacgaccaggtgcgcttcgagctgagctgctacagcctggccccccagatcaaggtgatcgccccctggcgcatgcccgagttctacaaccgcttcaagggccgcaacgacctgatggagtacgccaagcagcacggcatccccatccccgtgacccccaagaacccctggagcatggacgagaacctgatgcacatcagctacgaggccggcatcctggagaaccccaagaaccaggccccccccggcctgtacaccaagacccaggaccccgccaaggcccccaacacccccgacatcctggagatcgagttcaagaagggcgtgcccgtgaaggtgaccaacgtgaaggacggcaccacccaccagaccagcctggagctgttcatgtacctgaacgaggtggccggcaagcacggcgtgggccgcatcgacatcgtggagaaccgcttcatcggcatgaagagccgcggcatctacgagacccccgccggcaccatcctgtaccacgcccacctggacatcgaggccttcaccatggaccgcgaggtgcgcaagatcaagcagggcctgggcctgaagttcgccgagctggtgtacaccggcttctggcacagccccgagtgcgagttcgtgcgccactgcatcgccaagagccaggagcgcgtggagggcaaggtgcaggtgagcgtgctgaagggccaggtgtacatcctgggccgcgagagccccctgagcctgtacaacgaggagctggtgagcatgaacgtgcagggcgactacgagcccaccgacgccaccggcttcatcaacatcaacagcctgcgcctgaaggagtaccaccgcctgcagagcaaggtgaccgccaagtgacgggtggcatccctgtgacccctccccagtgcctctcctggccctggaagttgccactccagtgcccaccagccttgtcctaataaaattaagttgcatcaaagct

SEQ ID NO: 3 - RNA, 1899 nt, synthetic construct
  misc_feature 1..1899: note = Synthetic polynucleotide
  variation 1794: note = May or may not be present
  variation 1894: note = May or may not be present
  source 1..1899: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaacgggtggcatccctgtgacccctccccagtgcctctcctggccctggaagttgccactccagtgcccaccagccttgtcctaataaaattaagttgcatcaaagct

SEQ ID NO: 4 - RNA, 1131 nt, synthetic construct
  misc_feature 1..1131: note = Synthetic polynucleotide
  variation 1026: note = May or may not be present
  variation 1126: note = May or may not be present
  source 1..1131: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatggccatgagcagcggaggcagcggcggaggagtgcccgagcaggaggacagcgtgctgttcaggagaggcaccggccagagcgatgacagcgatatctgggacgataccgctctgatcaaggcctacgacaaggccgtggccagcttcaagcacgccctgaaaaacggcgacatctgcgagaccagcggcaagcccaagacaacccccaagagaaagcccgccaagaagaataagagccagaaaaagaacaccgccgccagcctgcagcagtggaaggtgggcgacaagtgcagcgccatctggagcgaggacggctgcatctaccccgccaccatcgccagcatcgacttcaagagagagacctgcgtggtcgtgtacaccggctacggcaacagagaggagcagaacctgagcgacctgctgagccccatttgtgaggtggccaataacatcgaacagaacgcccaggagaacgagaatgaaagccaggtgagcaccgacgagagcgagaacagcagatctcctggcaacaagagcgacaacatcaagcctaagtctgccccttggaacagcttcctgccccctcctccacccatgcccggacccagactgggacccggaaaacctggcctgaagttcaacggaccacctccccctccacctcctcccccacctcatctcctgagctgctggctgccacccttccccagcggaccccctatcatcccaccaccccctcccatctgccccgacagcctggacgacgccgatgccctgggcagcatgctgatcagctggtacatgagcggctaccacacaggatactacatgggcttcagacagaaccagaaggagggcagatgctcccactccctgaactgacgggtggcatccctgtgacccctccccagtgcctctcctggccctggaagttgccactccagtgcccaccagccttgtcctaataaaattaagttgcatcaaagct

SEQ ID NO: 5 - RNA, 140 nt, synthetic construct
  misc_feature 1..140: note = Synthetic polynucleotide
  source 1..140: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacg

SEQ ID NO: 6 - RNA, 105 nt, synthetic construct
  misc_feature 1..105: note = Synthetic polynucleotide
  source 1..105: mol_type = other RNA
cgggtggcatccctgtgacccctccccagtgcctctcctggccctggaagttgccactccagtgcccaccagccttgtcctaataaaattaagttgcatcaagct

SEQ ID NO: 7 - RNA, 105 nt, synthetic construct
  misc_feature 1..105: note = Synthetic polynucleotide
  source 1..105: mol_type = other RNA
gggtggcatccctgtgacccctccccagtgcctctcctggccctggaagttgccactccagtgcccaccagccttgtcctaataaaattaagttgcatcaaagct

SEQ ID NO: 8 - RNA, 4719 nt, synthetic construct
  misc_feature 1..4719: note = Synthetic polynucleotide
  variation 4614: note = May or may not be present
  variation 4714: note = May or may not be present
  source 1..4719: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatgcagcggtccccgctcgaaaaggccagtgtcgtgtccaaactcttcttctcatggactcggcctatccttagaaaggggtatcggcagaggcttgagttgtctgacatctaccagatcccctcggtagattcggcggataacctctcggagaagctcgaacgggaatgggaccgcgaactcgcgtctaagaaaaacccgaagctcatcaacgcactgagaaggtgcttcttctggcggttcatgttctacggtatcttcttgtatctcggggaggtcacaaaagcagtccaacccctgttgttgggtcgcattatcgcctcgtacgaccccgataacaaagaagaacggagcatcgcgatctacctcgggatcggactgtgtttgcttttcatcgtcagaacacttttgttgcatccagcaatcttcggcctccatcacatcggtatgcagatgcgaatcgctatgtttagcttgatctacaaaaagacactgaaactctcgtcgcgggtgttggataagatttccatcggtcagttggtgtccctgcttagtaataacctcaacaaattcgatgagggactggcgctggcacatttcgtgtggattgccccgttgcaagtcgcccttttgatgggccttatttgggagctgttgcaggcatctgccttttgtggcctgggatttctgattgtgttggcattgtttcaggctgggcttgggcggatgatgatgaagtatcgcgaccagagagcgggtaaaatctcggaaagactcgtcatcacttcggaaatgatcgaaaacatccagtcggtcaaagcctattgctgggaagaagctatggagaagatgattgaaaacctccgccaaactgagctgaaactgacccgcaaggcggcgtatgtccggtatttcaattcgtcagcgttcttcttttccgggttcttcgttgtctttctctcggttttgccttatgccttgattaaggggattatcctccgcaagattttcaccacgatttcgttctgcattgtattgcgcatggcagtgacacggcaatttccgtgggccgtgcagacatggtatgactcgcttggagcgatcaacaaaatccaagacttcttgcaaaagcaagagtacaagaccctggagtacaatcttactactacggaggtagtaatggagaatgtgacggctttttgggaagagggttttggagaactgtttgagaaagcaaagcagaataacaacaaccgcaagacctcaaatggggacgattccctgtttttctcgaacttctccctgctcggaacacccgtgttgaaggacatcaatttcaagattgagaggggacagcttctcgcggtagcgggaagcactggtgcgggaaaaactagcctcttgatggtgattatgggggagcttgagcccagcgaggggaagattaaacactccgggcgtatctcattctgtagccagttttcatggatcatgcccggaaccattaaagagaacatcattttcggagtatcctatgatgagtaccgatacagatcggtcattaaggcgtgccagttggaagaggacatttctaagttcgccgagaaggataacatcgtcttgggagaagggggtattacattgtcgggagggcagcgagcgcggatcagcctcgcgagagcggtatacaaagatgcagatttgtatctgcttgattcaccgtttggatacctcgacgtattgacagaaaaagaaatcttcgagtcgtgcgtgtgtaaacttatggctaataagacgagaatcctggtgacatcaaaaatggaacaccttaagaaggcggacaagatcctgatcctccacgaaggatcgtcctacttttacggcactttctcagagttgcaaaacttgcagccggacttctcaagcaaactcatggggtgtgactcattcgaccagttcagcgcggaacggcggaactcgatcttgacggaaacgctgcaccgattctcgcttgagggtgatgccccggtatcgtggaccgagacaaagaagcagtcgtttaagcagacaggagaatttggtgagaaaagaaagaacagtatcttgaatcctattaactcaattcgcaagttctcaatcgtccagaaaactccactgcagatgaatggaattgaagaggattcggacgaacccctggagcgcaggcttagcctcgtgccggattcagagcaaggggaggccattcttccccggatttcggtgatttcaaccggacctacacttcaggcgaggcgaaggcaatccgtgctcaacctcatgacgcattcggtaaaccaggggcaaaacattcaccgcaaaacgacggcctcaacgagaaaagtgtcacttgcaccccaggcgaatttgactgaactcgacatctacagccgtaggctttcgcaagaaaccggacttgagatcagcgaagaaatcaatgaagaagatttgaaagagtgtttctttgatgacatggaatcaatcccagcggtgacaacgtggaacacatacttgcgttacatcacggtgcacaagtccttgattttcgtcctcatctggtgtctcgtgatctttctcgctgaggtcgcagcgtcacttgtggtcctctggctgcttggtaatacgcccttgcaagacaaaggcaattctacacactcaagaaacaattcctatgccgtgattatcacttctacaagctcgtattacgtgttttacatctacgtaggagtggccgacactctgctcgcgatgggtttcttccgaggactcccactcgttcacacgcttatcactgtctccaagattctccaccataagatgcttcatagcgtactgcaggctcccatgtccaccttgaatacgctcaaggcgggaggtattttgaatcgcttctcaaaagatattgcaattttggatgaccttctgcccctgacgatcttcgacttcatccagttgttgctgatcgtgattggggctattgcagtagtcgctgtcctccagccttacatttttgtcgcgaccgttccggtgatcgtggcgtttatcatgctgcgggcctatttcttgcagacgtcacagcagcttaagcaactggagtctgaagggaggtcgcctatctttacgcatcttgtgaccagtttgaagggattgtggacgttgcgcgcctttggcaggcagccctactttgaaacactgttccacaaagcgctgaatctccatacggcaaattggtttttgtatttgagtaccctccgatggtttcagatgcgcattgagatgatttttgtgatcttctttatcgcggtgacttttatctccatcttgaccacgggagagggcgagggacgggtcggtattatcctgacactcgccatgaacattatgagcactttgcagtgggcagtgaacagctcgattgatgtggatagcctgatgaggtccgtttcgagggtctttaagttcatcgacatgccgacggagggaaagcccacaaaaagtacgaaaccctataagaatgggcaattgagtaaggtaatgatcatcgagaacagtcacgtgaagaaggatgacatctggcctagcgggggtcagatgaccgtgaaggacctgacggcaaaatacaccgagggagggaacgcaatccttgaaaacatctcgttcagcattagccccggtcagcgtgtggggttgctcgggaggaccgggtcaggaaaatcgacgttgctgtcggccttcttgagacttctgaatacagagggtgagatccagatcgacggcgtttcgtgggatagcatcaccttgcagcagtggcggaaagcgtttggagtaatcccccaaaaggtctttatctttagcggaaccttccgaaagaatctcgatccttatgaacagtggtcagatcaagagatttggaaagtcgcggacgaggttggccttcggagtgtaatcgagcagtttccgggaaaactcgactttgtccttgtagatgggggatgcgtcctgtcgcatgggcacaagcagctcatgtgcctggcgcgatccgtcctctctaaagcgaaaattcttctcttggatgaaccttcggcccatctggacccggtaacgtatcagatcatcagaaggacacttaagcaggcgtttgccgactgcacggtgattctctgtgagcatcgtatcgaggccatgctcgaatgccagcaatttcttgtcatcgaagagaataaggtccgccagtacgactccatccagaagctgcttaatgagagatcattgttccggcaggcgatttcaccatccgatagggtgaaactttttccacacagaaattcgtcgaagtgcaagtccaaaccgcagatcgcggccttgaaagaagagactgaagaagaagttcaagacacgcgtcttcaccatcaccatcaccatcaccatcaccattaacgggtggcatccctgtgacccctccccagtgcctctcctggccctggaagttgccactccagtgcccaccagccttgtcctaataaaattaagttgcatcaaagct

SEQ ID NO: 9 - RNA, 4689 nt, synthetic construct
  misc_feature 1..4689: note = Synthetic polynucleotide
  variation 4584: note = May or may not be present
  variation 4684: note = May or may not be present
  source 1..4689: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatgcagcggtccccgctcgaaaaggccagtgtcgtgtccaaactcttcttctcatggactcggcctatccttagaaaggggtatcggcagaggcttgagttgtctgacatctaccagatcccctcggtagattcggcggataacctctcggagaagctcgaacgggaatgggaccgcgaactcgcgtctaagaaaaacccgaagctcatcaacgcactgagaaggtgcttcttctggcggttcatgttctacggtatcttcttgtatctcggggaggtcacaaaagcagtccaacccctgttgttgggtcgcattatcgcctcgtacgaccccgataacaaagaagaacggagcatcgcgatctacctcgggatcggactgtgtttgcttttcatcgtcagaacacttttgttgcatccagcaatcttcggcctccatcacatcggtatgcagatgcgaatcgctatgtttagcttgatctacaaaaagacactgaaactctcgtcgcgggtgttggataagatttccatcggtcagttggtgtccctgcttagtaataacctcaacaaattcgatgagggactggcgctggcacatttcgtgtggattgccccgttgcaagtcgcccttttgatgggccttatttgggagctgttgcaggcatctgccttttgtggcctgggatttctgattgtgttggcattgtttcaggctgggcttgggcggatgatgatgaagtatcgcgaccagagagcgggtaaaatctcggaaagactcgtcatcacttcggaaatgatcgaaaacatccagtcggtcaaagcctattgctgggaagaagctatggagaagatgattgaaaacctccgccaaactgagctgaaactgacccgcaaggcggcgtatgtccggtatttcaattcgtcagcgttcttcttttccgggttcttcgttgtctttctctcggttttgccttatgccttgattaaggggattatcctccgcaagattttcaccacgatttcgttctgcattgtattgcgcatggcagtgacacggcaatttccgtgggccgtgcagacatggtatgactcgcttggagcgatcaacaaaatccaagacttcttgcaaaagcaagagtacaagaccctggagtacaatcttactactacggaggtagtaatggagaatgtgacggctttttgggaagagggttttggagaactgtttgagaaagcaaagcagaataacaacaaccgcaagacctcaaatggggacgattccctgtttttctcgaacttctccctgctcggaacacccgtgttgaaggacatcaatttcaagattgagaggggacagcttctcgcggtagcgggaagcactggtgcgggaaaaactagcctcttgatggtgattatgggggagcttgagcccagcgaggggaagattaaacactccgggcgtatctcattctgtagccagttttcatggatcatgcccggaaccattaaagagaacatcattttcggagtatcctatgatgagtaccgatacagatcggtcattaaggcgtgccagttggaagaggacatttctaagttcgccgagaaggataacatcgtcttgggagaagggggtattacattgtcgggagggcagcgagcgcggatcagcctcgcgagagcggtatacaaagatgcagatttgtatctgcttgattcaccgtttggatacctcgacgtattgacagaaaaagaaatcttcgagtcgtgcgtgtgtaaacttatggctaataagacgagaatcctggtgacatcaaaaatggaacaccttaagaaggcggacaagatcctgatcctccacgaaggatcgtcctacttttacggcactttctcagagttgcaaaacttgcagccggacttctcaagcaaactcatggggtgtgactcattcgaccagttcagcgcggaacggcggaactcgatcttgacggaaacgctgcaccgattctcgcttgagggtgatgccccggtatcgtggaccgagacaaagaagcagtcgtttaagcagacaggagaatttggtgagaaaagaaagaacagtatcttgaatcctattaactcaattcgcaagttctcaatcgtccagaaaactccactgcagatgaatggaattgaagaggattcggacgaacccctggagcgcaggcttagcctcgtgccggattcagagcaaggggaggccattcttccccggatttcggtgatttcaaccggacctacacttcaggcgaggcgaaggcaatccgtgctcaacctcatgacgcattcggtaaaccaggggcaaaacattcaccgcaaaacgacggcctcaacgagaaaagtgtcacttgcaccccaggcgaatttgactgaactcgacatctacagccgtaggctttcgcaagaaaccggacttgagatcagcgaagaaatcaatgaagaagatttgaaagagtgtttctttgatgacatggaatcaatcccagcggtgacaacgtggaacacatacttgcgttacatcacggtgcacaagtccttgattttcgtcctcatctggtgtctcgtgatctttctcgctgaggtcgcagcgtcacttgtggtcctctggctgcttggtaatacgcccttgcaagacaaaggcaattctacacactcaagaaacaattcctatgccgtgattatcacttctacaagctcgtattacgtgttttacatctacgtaggagtggccgacactctgctcgcgatgggtttcttccgaggactcccactcgttcacacgcttatcactgtctccaagattctccaccataagatgcttcatagcgtactgcaggctcccatgtccaccttgaatacgctcaaggcgggaggtattttgaatcgcttctcaaaagatattgcaattttggatgaccttctgcccctgacgatcttcgacttcatccagttgttgctgatcgtgattggggctattgcagtagtcgctgtcctccagccttacatttttgtcgcgaccgttccggtgatcgtggcgtttatcatgctgcgggcctatttcttgcagacgtcacagcagcttaagcaactggagtctgaagggaggtcgcctatctttacgcatcttgtgaccagtttgaagggattgtggacgttgcgcgcctttggcaggcagccctactttgaaacactgttccacaaagcgctgaatctccatacggcaaattggtttttgtatttgagtaccctccgatggtttcagatgcgcattgagatgatttttgtgatcttctttatcgcggtgacttttatctccatcttgaccacgggagagggcgagggacgggtcggtattatcctgacactcgccatgaacattatgagcactttgcagtgggcagtgaacagctcgattgatgtggatagcctgatgaggtccgtttcgagggtctttaagttcatcgacatgccgacggagggaaagcccacaaaaagtacgaaaccctataagaatgggcaattgagtaaggtaatgatcatcgagaacagtcacgtgaagaaggatgacatctggcctagcgggggtcagatgaccgtgaaggacctgacggcaaaatacaccgagggagggaacgcaatccttgaaaacatctcgttcagcattagccccggtcagcgtgtggggttgctcgggaggaccgggtcaggaaaatcgacgttgctgtcggccttcttgagacttctgaatacagagggtgagatccagatcgacggcgtttcgtgggatagcatcaccttgcagcagtggcggaaagcgtttggagtaatcccccaaaaggtctttatctttagcggaaccttccgaaagaatctcgatccttatgaacagtggtcagatcaagagatttggaaagtcgcggacgaggttggccttcggagtgtaatcgagcagtttccgggaaaactcgactttgtccttgtagatgggggatgcgtcctgtcgcatgggcacaagcagctcatgtgcctggcgcgatccgtcctctctaaagcgaaaattcttctcttggatgaaccttcggcccatctggacccggtaacgtatcagatcatcagaaggacacttaagcaggcgtttgccgactgcacggtgattctctgtgagcatcgtatcgaggccatgctcgaatgccagcaatttcttgtcatcgaagagaataaggtccgccagtacgactccatccagaagctgcttaatgagagatcattgttccggcaggcgatttcaccatccgatagggtgaaactttttccacacagaaattcgtcgaagtgcaagtccaaaccgcagatcgcggccttgaaagaagagactgaagaagaagttcaagacacgcgtctttaacgggtggcatccctgtgacccctccccagtgcctctcctggccctggaagttgccactccagtgcccaccagccttgtcctaataaaattaagttgcatcaaagct

SEQ ID NO: 10 - RNA, 4542 nt, synthetic construct
  misc_feature 1..4542: note = Synthetic polynucleotide
  source 1..4542: mol_type = other RNA
atggccactggatcaagaacctcactgctgctcgcttttggactgctttgcctgccctggttgcaagaaggatcggctttcccgaccatcccactctccatgcagcggtccccgctcgaaaaggccagtgtcgtgtccaaactcttcttctcatggactcggcctatccttagaaaggggtatcggcagaggcttgagttgtctgacatctaccagatcccctcggtagattcggcggataacctctcggagaagctcgaacgggaatgggaccgcgaactcgcgtctaagaaaaacccgaagctcatcaacgcactgagaaggtgcttcttctggcggttcatgttctacggtatcttcttgtatctcggggaggtcacaaaagcagtccaacccctgttgttgggtcgcattatcgcctcgtacgaccccgataacaaagaagaacggagcatcgcgatctacctcgggatcggactgtgtttgcttttcatcgtcagaacacttttgttgcatccagcaatcttcggcctccatcacatcggtatgcagatgcgaatcgctatgtttagcttgatctacaaaaagacactgaaactctcgtcgcgggtgttggataagatttccatcggtcagttggtgtccctgcttagtaataacctcaacaaattcgatgagggactggcgctggcacatttcgtgtggattgccccgttgcaagtcgcccttttgatgggccttatttgggagctgttgcaggcatctgccttttgtggcctgggatttctgattgtgttggcattgtttcaggctgggcttgggcggatgatgatgaagtatcgcgaccagagagcgggtaaaatctcggaaagactcgtcatcacttcggaaatgatcgaaaacatccagtcggtcaaagcctattgctgggaagaagctatggagaagatgattgaaaacctccgccaaactgagctgaaactgacccgcaaggcggcgtatgtccggtatttcaattcgtcagcgttcttcttttccgggttcttcgttgtctttctctcggttttgccttatgccttgattaaggggattatcctccgcaagattttcaccacgatttcgttctgcattgtattgcgcatggcagtgacacggcaatttccgtgggccgtgcagacatggtatgactcgcttggagcgatcaacaaaatccaagacttcttgcaaaagcaagagtacaagaccctggagtacaatcttactactacggaggtagtaatggagaatgtgacggctttttgggaagagggttttggagaactgtttgagaaagcaaagcagaataacaacaaccgcaagacctcaaatggggacgattccctgtttttctcgaacttctccctgctcggaacacccgtgttgaaggacatcaatttcaagattgagaggggacagcttctcgcggtagcgggaagcactggtgcgggaaaaactagcctcttgatggtgattatgggggagcttgagcccagcgaggggaagattaaacactccgggcgtatctcattctgtagccagttttcatggatcatgcccggaaccattaaagagaacatcattttcggagtatcctatgatgagtaccgatacagatcggtcattaaggcgtgccagttggaagaggacatttctaagttcgccgagaaggataacatcgtcttgggagaagggggtattacattgtcgggagggcagcgagcgcggatcagcctcgcgagagcggtatacaaagatgcagatttgtatctgcttgattcaccgtttggatacctcgacgtattgacagaaaaagaaatcttcgagtcgtgcgtgtgtaaacttatggctaataagacgagaatcctggtgacatcaaaaatggaacaccttaagaaggcggacaagatcctgatcctccacgaaggatcgtcctacttttacggcactttctcagagttgcaaaacttgcagccggacttctcaagcaaactcatggggtgtgactcattcgaccagttcagcgcggaacggcggaactcgatcttgacggaaacgctgcaccgattctcgcttgagggtgatgccccggtatcgtggaccgagacaaagaagcagtcgtttaagcagacaggagaatttggtgagaaaagaaagaacagtatcttgaatcctattaactcaattcgcaagttctcaatcgtccagaaaactccactgcagatgaatggaattgaagaggattcggacgaacccctggagcgcaggcttagcctcgtgccggattcagagcaaggggaggccattcttccccggatttcggtgatttcaaccggacctacacttcaggcgaggcgaaggcaatccgtgctcaacctcatgacgcattcggtaaaccaggggcaaaacattcaccgcaaaacgacggcctcaacgagaaaagtgtcacttgcaccccaggcgaatttgactgaactcgacatctacagccgtaggctttcgcaagaaaccggacttgagatcagcgaagaaatcaatgaagaagatttgaaagagtgtttctttgatgacatggaatcaatcccagcggtgacaacgtggaacacatacttgcgttacatcacggtgcacaagtccttgattttcgtcctcatctggtgtctcgtgatctttctcgctgaggtcgcagcgtcacttgtggtcctctggctgcttggtaatacgcccttgcaagacaaaggcaattctacacactcaagaaacaattcctatgccgtgattatcacttctacaagctcgtattacgtgttttacatctacgtaggagtggccgacactctgctcgcgatgggtttcttccgaggactcccactcgttcacacgcttatcactgtctccaagattctccaccataagatgcttcatagcgtactgcaggctcccatgtccaccttgaatacgctcaaggcgggaggtattttgaatcgcttctcaaaagatattgcaattttggatgaccttctgcccctgacgatcttcgacttcatccagttgttgctgatcgtgattggggctattgcagtagtcgctgtcctccagccttacatttttgtcgcgaccgttccggtgatcgtggcgtttatcatgctgcgggcctatttcttgcagacgtcacagcagcttaagcaactggagtctgaagggaggtcgcctatctttacgcatcttgtgaccagtttgaagggattgtggacgttgcgcgcctttggcaggcagccctactttgaaacactgttccacaaagcgctgaatctccatacggcaaattggtttttgtatttgagtaccctccgatggtttcagatgcgcattgagatgatttttgtgatcttctttatcgcggtgacttttatctccatcttgaccacgggagagggcgagggacgggtcggtattatcctgacactcgccatgaacattatgagcactttgcagtgggcagtgaacagctcgattgatgtggatagcctgatgaggtccgtttcgagggtctttaagttcatcgacatgccgacggagggaaagcccacaaaaagtacgaaaccctataagaatgggcaattgagtaaggtaatgatcatcgagaacagtcacgtgaagaaggatgacatctggcctagcgggggtcagatgaccgtgaaggacctgacggcaaaatacaccgagggagggaacgcaatccttgaaaacatctcgttcagcattagccccggtcagcgtgtggggttgctcgggaggaccgggtcaggaaaatcgacgttgctgtcggccttcttgagacttctgaatacagagggtgagatccagatcgacggcgtttcgtgggatagcatcaccttgcagcagtggcggaaagcgtttggagtaatcccccaaaaggtctttatctttagcggaaccttccgaaagaatctcgatccttatgaacagtggtcagatcaagagatttggaaagtcgcggacgaggttggccttcggagtgtaatcgagcagtttccgggaaaactcgactttgtccttgtagatgggggatgcgtcctgtcgcatgggcacaagcagctcatgtgcctggcgcgatccgtcctctctaaagcgaaaattcttctcttggatgaaccttcggcccatctggacccggtaacgtatcagatcatcagaaggacacttaagcaggcgtttgccgactgcacggtgattctctgtgagcatcgtatcgaggccatgctcgaatgccagcaatttcttgtcatcgaagagaataaggtccgccagtacgactccatccagaagctgcttaatgagagatcattgttccggcaggcgatttcaccatccgatagggtgaaactttttccacacagaaattcgtcgaagtgcaagtccaaaccgcagatcgcggccttgaaagaagagactgaagaagaagttcaagacacgcgtctttaa

SEQ ID NO: 11 - protein, 10 aa, synthetic construct
  REGION 1..10: note = Synthetic 10xHis tag
HHHHHHHHHH

SEQ ID NO: 12 - DNA, 500 bp, synthetic construct
  misc_feature 1..500: note = Synthetic polynucleotide
  variation 1..500: note = This sequence may encompass 10-500 nucleotides
  source 1..500: mol_type = other DNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 13 - DNA, 300 bp, synthetic construct
  misc_feature 1..300: note = Synthetic polynucleotide
  variation 1..300: note = This sequence may encompass 10-300 nucleotides
  source 1..300: mol_type = other DNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 14 - DNA, 200 bp, synthetic construct
  misc_feature 1..200: note = Synthetic polynucleotide
  variation 1..200: note = This sequence may encompass 10-200 nucleotides
  source 1..200: mol_type = other DNA
cccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccc

SEQ ID NO: 15 - DNA, 250 bp, synthetic construct
  misc_feature 1..250: note = Synthetic polynucleotide
  misc_feature 1..250: note = See specification as filed for detailed description of substitutions and preferred embodiments
  source 1..250: mol_type = other DNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2024/0293319 A1
(43) Pub. Date: Sep. 5, 2024

(54) Lipid Formulations for Delivery of Messenger RNA

(71) Applicants: Translate Bio, Inc., Waltham, MA (US); Massachusetts Institute of Technology, Cambridge, MA (US)

(72) Inventors: Michael Heartlein, Cambridge, MA (US); Daniel Anderson, Cambridge, MA (US); Yizhou Dong, Cambridge, MA (US); Frank DeRosa, Cambridge, MA (US)

(73) Assignees: Translate Bio, Inc., Waltham, MA (US); Massachusetts Institute of Technology, Cambridge, MA (US)

(21) Appl. No.: 18/545,858

(22) Filed: Dec. 19, 2023

Related U.S. Application Data
(63) Continuation of application No. 17/162,656, filed on Jan. 29, 2021, now Pat. No. 11,890,377.
Application No. 17/162,656 is a continuation of application No. 16/685,287, filed on Nov. 15, 2019, now Pat. No. 10,959,953.
Application No. 16/685,287 is a continuation of application No. 16/026,577, filed on Jul. 3, 2018, now Pat. No. 10,493,031.
Application No. 16/026,577 is a continuation of application No. 15/451,312, filed on Mar. 6, 2017, now Pat. No. 10,052,284.
Application No. 15/451,312 is a division of application No. 14/521,161, filed on Oct. 22, 2014, now Pat. No. 9,629,804.
(60) Provisional application No. 61/894,299, filed on Oct. 22, 2013.
(60) Provisional application No. 61/953,516, filed on Mar. 14, 2014.

(51) Int. Cl. A61K 9/127; A61K 38/17; A61K 38/48; A61K 38/53; A61K 48/00

(52) U.S. Cl. CPC A61K 9/1272; A61K 38/1709; A61K 38/177; A61K 38/4846; A61K 38/53; A61K 48/0033; A61K 48/005; A61K 48/00

(57) ABSTRACT

The present invention provides, among other things, methods of delivering mRNA in vivo, including administering to a subject in need of delivery a composition comprising an mRNA encoding a protein, encapsulated within a liposome such that the administering of the composition results in the expression of the protein encoded by the mRNA in vivo, wherein the liposome comprises a cationic lipid of formula I-c:

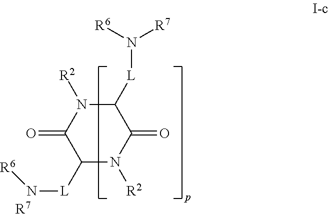

or a pharmaceutically acceptable salt thereof.

### RELATED APPLICATIONS

This application claims priority to U.S. Provisional Application Ser. No. 61/894,299, filed Oct. 22, 2013 and U.S. Provisional Application Ser. No. 61/953,516, filed Mar. 14, 2014, the disclosures of which are hereby incorporated by reference.

### SEQUENCE LISTING

The present specification makes reference to a Sequence Listing (submitted electronically as a .txt file named “2006685-0686_SL.txt” on Oct. 22, 2014). The .txt file was generated on Oct. 22, 2014 and is 33,306 bytes in size. The entire contents of the Sequence Listing are herein incorporated by reference.

### BACKGROUND

Delivery of nucleic acids has been explored extensively as a potential therapeutic option for certain disease states. In particular, RNA interference (RNAi) has been the subject of significant research and clinical development. While RNAi, such as short interfering RNA (siRNA), may have therapeutic potential, it is of little use in treating diseases involving deficiency of one or more proteins. messenger RNA (mRNA) therapy has become an increasingly important option for treatment of various diseases, in particular, for those associated with deficiency of one or more proteins.

### SUMMARY OF THE INVENTION

The present invention provides improved methods and compositions for highly efficient delivery and expression of mRNA and encoded protein in vivo. The invention is based, in part, on the surprising discovery that liposomes based on a particular class of cationic lipids, such as, those having a structure of formula I-c described herein, are unexpectedly effective in delivering mRNA and producing encoded protein in vivo, more effective even as compared to those cationic lipids that were considered to be among the best in delivering mRNA in the prior art. Indeed, prior to the present invention, cationic lipids have been extensively explored as an important component of liposomes typically used to encapsulate nucleic acids including mRNA for in vivo delivery. Due to the uniquely fragile and long structure of mRNA and the complicated in vivo translation process, cationic lipids used in the liposomes typically play two roles. First, cationic lipids promote interaction with negatively charged mRNA during encapsulation, circulation and endocytosis, thereby capturing and protecting the mRNA. Then, once inside cytosol, cationic lipids need to be able to release the mRNA so that the mRNA can be translated to produce encoded protein. Some cationic lipids, in particular, those known as titratable cationic lipids are particularly effective in delivering mRNA. One example of such cationic lipids known to be capable of efficient delivery of mRNA is C12-200. Surprisingly, the present inventors found that cationic lipids described herein can be even more effective in delivering various mRNA in vivo, than those best known in the prior art including C12-200. For example, as shown in the Examples below, liposome particles incorporating a cationic lipid described herein (e.g., cKK-E12) resulted in close to 50% higher protein expression of human Factor IX protein detected in the plasma of administered mice, as compared to C12-200-based liposome particles. Furthermore, the plasma residence time of different proteins expressed from mRNA delivered by cKK-E12 based liposomes is sustained up to 7 days or longer post a single administration. Thus, the present inventors have demonstrated that this class of cationic lipids having a structure of formula I-c described herein (e.g., cKK-E12) can be uniquely useful in delivering mRNA for highly efficient and sustained production of protein (e.g., therapeutic protein) in vivo. The present invention therefore permits an improved mRNA therapy that can significantly reduce required amount of mRNA and associated lipids, administration frequency, and possible side effects, providing more potent, safer, and patient friendly mRNA therapy for various diseases.

In one aspect, the present invention provides methods of delivering messenger RNA (mRNA) in vivo, including administering to a subject in need of delivery a composition comprising an mRNA encoding a protein, encapsulated within a liposome such that the administering of the composition results in the expression of the protein encoded by the mRNA in vivo, wherein the liposome comprises a cationic lipid of formula I-c:

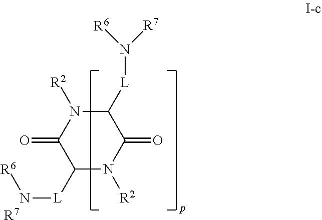

or a pharmaceutically acceptable salt thereof,
wherein:
p is an integer of between 1 and 9, inclusive;
each instance of R² is independently hydrogen or optionally substituted C₁₋₆ alkyl; each instance of R⁶ and R⁷ is independently a group of the formula (i), (ii), or (iii); Formulae (i), (ii), and (iii) are:

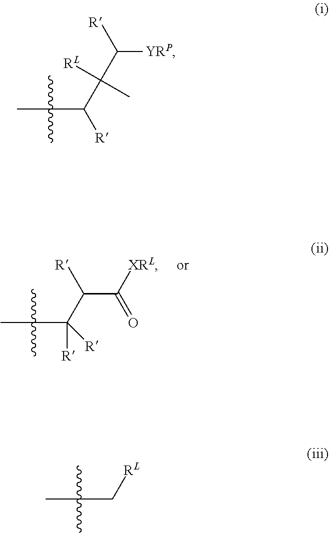

wherein:
each instance of R′ is independently hydrogen or optionally substituted alkyl;
X is O, S, or NR^{X}, wherein R^{X} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group;
Y is O, S, or NR^{Y}, wherein R^{Y} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group;
R^{P} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, a sulfur protecting group when attached to a sulfur atom, or a nitrogen protecting group when attached to a nitrogen atom; and
R^{L} is optionally substituted C₁₋₅₀ alkyl, optionally substituted C₂₋₅₀ alkenyl, optionally substituted C₂₋₅₀ alkynyl, optionally substituted heteroC₁₋₅₀ alkyl, optionally substituted heteroC₂₋₅₀ alkenyl, optionally substituted heteroC₂₋₅₀ alkynyl, or a polymer.

In another aspect, the present invention provides methods of treating a disease or disorder including administering to subject in need of treatment a composition comprising an mRNA encoding a therapeutic protein encapsulated within a liposome such that the administering of the composition results in the expression of the protein encoded by the mRNA in one or more tissues affected by the disease or disorder, wherein the liposome comprises a cationic lipid having a structure of formula I-c.

In another aspect, the present invention provides compositions for delivery of messenger RNA (mRNA) comprising an mRNA encoding a protein encapsulated within a liposome, wherein the liposome comprises a cationic lipid having a structure of formula I-c.

In some embodiments, a suitable cationic lipid is cKK-E12:

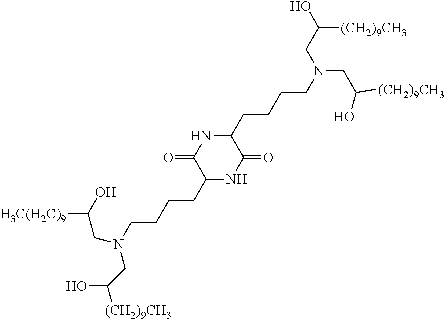

In some embodiments, a suitable liposome further comprises one or more non-cationic lipids, one or more cholesterol-based lipids and/or one or more PEG-modified lipids. In some embodiments, the one or more non-cationic lipids are selected from distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), or a mixture thereof.

In some embodiments, a suitable liposome further comprises one or more cholesterol-based lipids. In some embodiments, the one or more cholesterol-based lipids are selected from cholesterol, PEGylated cholesterol and DC-Chol (N,N-dimethyl-N-ethylcarboxamidocholesterol), 1,4-bis(3-N-oleylamino-propyl)piperazine.

In some embodiments, a suitable liposome further comprises one or more PEG-modified lipids. In some embodiments, the one or more PEG-modified lipids comprise a poly(ethylene) glycol chain of up to 5 kDa in length covalently attached to a lipid with alkyl chain(s) of C₆-C₂₀ length. In some embodiments, a PEG-modified lipid is a derivatized ceramide such as N-Octanoyl-Sphingosine-1-[Succinyl(Methoxy Polyethylene Glycol)-2000]. In some embodiments, a PEG-modified or PEGylated lipid is PEGylated cholesterol or Dimyristoylglycerol (DMG)-PEG-2K.

In some embodiments, a suitable liposome comprises cKK-E12, DOPE, cholesterol and DMG-PEG2K.

In some embodiments, the cationic lipid (e.g., cKK-E12) constitutes about 30-50% (e.g., about 30-45%, about 30-40%, about 35-50%, about 35-45%, or about 35-40%) of the liposome by molar ratio. In some embodiments, the cationic lipid (e.g., cKK-E12) constitutes about 30%, about 35%, about 40%, about 45%, or about 50% of the liposome by molar ratio.

In particular embodiments, the ratio of cKK-E12:DOPE:cholesterol:DMG-PEG2K is approximately 40:30:20:10 by molar ratio. In particular embodiments, the ratio of cKK-E12:DOPE:cholesterol:DMG-PEG2K is approximately 40:30:25:5 by molar ratio. In particular embodiments, the ratio of cKK-E12:DOPE:cholesterol:DMG-PEG2K is approximately 40:32:25:3 by molar ratio.

In some embodiments, a suitable liposome has a size of or less than about 500 nm, 450 nm, 400 nm, 350 nm, 300 nm, 250 nm, 200 nm, 150 nm, 125 nm, 110 nm, 100 nm, 95 nm, 90 nm, 85 nm, 80 nm, 75 nm, 70 nm, 65 nm, 60 nm, 55 nm, or 50 nm.

In some embodiments, a composition according to the invention is administered intravenously. In some embodiments, a composition according to the invention is administered via pulmonary delivery. In some embodiments, the pulmonary delivery is by aerosolization, inhalation, nebulization or instillation. In some embodiments, a composition according to the invention is administered intrathecally. In some embodiments, the composition is formulated as respirable particles, nebulizable lipid, or inhalable dry powder.

In some embodiments, the expression of the protein encoded by the mRNA is detectable in liver, kidney, heart, spleen, serum, brain, skeletal muscle, lymph nodes, skin, and/or cerebrospinal fluid.

In some embodiments, the expression of the protein encoded by the mRNA is detectable 6, 12, 18, 24, 30, 36, 42, 48, 54, 60, 66, and/or 72 hours after the administration. In some embodiments, the expression of the protein encoded by the mRNA is detectable 1 day, 2 days, 3 days, 4 days, 5 days, 6 days, and/or 7 days after the administration. In some embodiments, the expression of the protein encoded by the mRNA is detectable 1 week, 2 weeks, 3 weeks, and/or 4 weeks after the administration. In some embodiments, the expression of the protein encoded by the mRNA is detectable after a month after the administration.

In some embodiments, the protein encoded by the mRNA is a cytosolic protein. In some embodiments, the protein encoded by the mRNA is a secreted protein. In some embodiments, the protein encoded by the mRNA is an enzyme. In some embodiments, the mRNA has a length of or greater than about 0.5 kb, 1 kb, 1.5 kb, 2 kb, 2.5 kb, 3 kb, 3.5 kb, 4 kb, 4.5 kb, or 5 kb. In some embodiments, the protein encoded by the mRNA is Argininosuccinate Synthetase (ASS1), Factor IX, survival of motor neuron 1, or phenylalanine hydroxylase.

In some embodiments, the mRNA is administered at a dose ranging from about 0.1-5.0 mg/kg body weight, for example about 0.1-4.5, 0.1-4.0, 0.1-3.5, 0.1-3.0, 0.1-2.5, 0.1-2.0, 0.1-1.5, 0.1-1.0, 0.1-0.5, 0.1-0.3, 0.3-5.0, 0.3-4.5, 0.3-4.0, 0.3-3.5, 0.3-3.0, 0.3-2.5, 0.3-2.0, 0.3-1.5, 0.3-1.0, 0.3-0.5, 0.5-5.0, 0.5-4.5, 0.5-4.0, 0.5-3.5, 0.5-3.0, 0.5-2.5, 0.5-2.0, 0.5-1.5, or 0.5-1.0 mg/kg body weight. In some embodiments, the mRNA is administered at a dose of or less than about 5.0, 4.5, 4.0, 3.5, 3.0, 2.5, 2.0, 1.5, 1.0, 0.8, 0.6, 0.5, 0.4, 0.3, 0.2, or 0.1 mg/kg body weight.

In some embodiments, the mRNA comprises one or more modified nucleotides. In some embodiments, the one or more modified nucleotides comprise pseudouridine, N-1-methyl-pseudouridine, 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and/or 2-thiocytidine. In some embodiments, the mRNA is unmodified.

Other features, objects, and advantages of the present invention are apparent in the detailed description, drawings and claims that follow. It should be understood, however, that the detailed description, the drawings, and the claims, while indicating embodiments of the present invention, are given by way of illustration only, not limitation. Various changes and modifications within the scope of the invention will become apparent to those skilled in the art.

### BRIEF DESCRIPTION OF THE DRAWING

The drawings are for illustration purposes only not for limitation.

FIG. 1 shows an exemplary graph of the levels of human factor IX (FIX) detected in the serum of treated mice 24 hours after administration of C12-200 or cKK-E12 liposomes containing FIX mRNA.

FIG. 2 shows an exemplary graph of FIX detected in the plasma of mice treated with 0.1, 0.3, 0.6, 1.0, or 3.0 mg/kg of one of two ratios of FIX mRNA containing cKK-E12 liposomes either 6 or 24 hours after administration.

FIG. 3 shows an exemplary graph of the level of ASS1 protein detected in the livers of mice treated with 0.1, 0.3, 0.6, 1.0, or 3.0 mg/kg of ASS1 mRNA-containing cKK-E12 liposomes 24 hours after administration.

FIG. 4 shows exemplary western blot analyses of ASS1 protein levels in the liver 24 hours post administration of 0.1, 0.3, 0.6, 1.0, or 3.0 mg/kg of cKK-E12 liposomes containing ASS1 mRNA.

FIG. 5 shows an exemplary graph of ASS1 protein levels in the liver of mice 0.5, 3, 6, 12, 24, 48, 72 hours after a single IV injection of ASS1 mRNA containing cKK-E12 liposomes (1 mg/kg). Also shown is the level of ASS1 protein 7 days after administration.

FIG. 6 shows exemplary western blot analyses of ASS1 protein levels in the liver 0.5, 3, 6, 12, 24, 48, 72 hours after a single IV injection of 1 mg/kg ASS1 mRNA containing cKK-E12 liposomes. Also shown is the level of ASS1 protein 7 days after administration.

FIG. 7—shows detection of human ASS1 messenger RNA via in situ hybridization in the livers of treated mice. Exogenous mRNA is observable for at least 72 hr post-administration after a single dose (1.0 mg/kg) of ASS1 mRNA-loaded MD1-based lipid nanoparticles. Human ASS1 mRNA is detectable in sinusoidal cells as well as hepatocytes.

FIG. 8—shows exemplary immunohistochemical staining of ASS1 protein levels in mouse liver 24 hours after administration of 1 mg/kg ASS1 mRNA containing cKK-E12 lipid nanoparticles. Human ASS1 protein is detectable in sinusoidal cells as well as hepatocytes.

FIG. 9 shows low magnification (4×) immunohistochemical staining of ASS1 protein levels in mouse liver 24 hours after administration of 1 mg/kg ASS1 mRNA containing cKK-E12 liposomes. A comparison to untreated mouse liver (left) demonstrates the widespread distribution of human ASS1 protein throughout the liver.

FIG. 10 shows exemplary results illustrating that cKK-E12 lipid nanoparticles efficiently delivered FL mRNA via nebulization. Mice were exposed to milligram of encapsulated FL mRNA and analysis was performed 24 hours post-exposure.

FIG. 11 illustrates detection via western blot of human SMN-1 protein derived from exogenous hSMN-1 mRNA that was transfected into BHK-21 cells. Various antibodies specific to human SMN were employed: (A) anti-SMN 4F11 antibody at 1:1,000 dilution; (B) Pierce PA5-27309 a-SMN antibody at 1:10,000 dilution; and (C) LSBio C138149 a-SMN antibody at 1:10,000 dilution.

FIG. 12A-C illustrates multiplex nucleic acid in situ detection of human Survival of Motor Neuron (hSMN-1) mRNA in (A) Cervical, (B) Thoracic and (C) Lumbar spinal tissue, 24 hours post intrathecal delivery.

FIG. 13 illustrates positive detection of human SMN-1 protein produced in the spinal cord of a rat 24 hours post-intrathecal administration of human SMN-1 mRNA-loaded lipid nanoparticles. Anti-human SMN 4F11 antibody was employed at 1:2500 dilution. Panel A represents treated rat spinal cord tissue and panel B represents untreated rat spinal cord tissue.

FIG. 14 In vivo transfection of CFTR knockout mice with C-terminal His₁₀ tagged (SEQ ID NO: 11) codon-optimized human CFTR mRNA encapsulated within either a lipid (cKK-E12) or polymeric (PEI) nanoparticle formulation. Following nebulized delivery of each respective mRNA formulation, Right and Left lung tissue lysate was collected and analyzed for CFTR expression by Western blot using anti-His antibody 1187. Control CFTR knockout lung tissue and CFTR-His₁₀ HEK293 lysate (“His₁₀” disclosed as SEQ ID NO: 11) was used as a negative and positive controls respectively.

FIG. 15 illustrates positive detection of active firefly luciferase (FFL) protein in a treated pig lung via luminescence upon exposure to FFL/CO-CFTR—C-His₁₀ mRNA (“His₁₀” disclosed as SEQ ID NO: 11) encapsulated cKK-E12 lipid nanoparticles. Pigs were treated with 1 mg FFL+9 mg CO-CFTR—C-His₁₀ mRNA (“His₁₀” disclosed as SEQ ID NO: 11) encapsulated lipid nanoparticles via nebulization using a Pari jet nebulizer and sacrificed 24 hours post-treatment. FFL luminescence was visualized using an IVIS bioluminometer.

### DEFINITIONS

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

Amino acid: As used herein, term “amino acid,” in its broadest sense, refers to any compound and/or substance that can be incorporated into a polypeptide chain. In some embodiments, an amino acid has the general structure H_{E}N—C(H)(R)—COHO. In some embodiments, an amino acid is a naturally occurring amino acid. In some embodiments, an amino acid is a synthetic amino acid; in some embodiments, an amino acid is a d-amino acid; in some embodiments, an amino acid is an 1-amino acid. “Standard amino acid” refers to any of the twenty standard 1-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxyl- and/or amino-terminal amino acids in peptides, can be modified by methylation, amidation, acetylation, protecting groups, and/or substitution with other chemical groups that can change the peptide's circulating half-life without adversely affecting their activity. Amino acids may participate in a disulfide bond. Amino acids may comprise one or posttranslational modifications, such as association with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, etc.). The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 1%, 90%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Delivery: As used herein, the term “delivery” encompasses both local and systemic delivery. For example, delivery of mRNA encompasses situations in which an mRNA is delivered to a target tissue and the encoded protein is expressed and retained within the target tissue (also referred to as “local distribution” or “local delivery”), and situations in which an mRNA is delivered to a target tissue and the encoded protein is expressed and secreted into patient's circulation system (e.g., serum) and systematically distributed and taken up by other tissues (also referred to as “systemic distribution” or “systemic delivery).

Expression: As used herein, “expression” of a nucleic acid sequence refers to translation of an mRNA into a polypeptide, assemble multiple polypeptides (e.g., heavy chain or light chain of antibody) into an intact protein (e.g., antibody) and/or post-translational modification of a polypeptide or fully assembled protein (e.g., antibody). In this application, the terms “expression” and “production,” and grammatical equivalent, are used inter-changeably.

Functional: As used herein, a “functional” biological molecule is a biological molecule in a form in which it exhibits a property and/or activity by which it is characterized.

Half-life: As used herein, the term “half-life” is the time required for a quantity such as nucleic acid or protein concentration or activity to fall to half of its value as measured at the beginning of a time period.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. As used herein, calculation of percent purity of isolated substances and/or entities should not include excipients (e.g., buffer, solvent, water, etc.).

Local distribution or delivery: As used herein, the terms “local distribution,” “local delivery,” or grammatical equivalent, refer to tissue specific delivery or distribution. Typically, local distribution or delivery requires a protein (e.g., enzyme) encoded by mRNAs be translated and expressed intracellularly or with limited secretion that avoids entering the patient's circulation system.

messenger RNA (mRNA): As used herein, the term “messenger RNA (mRNA)” refers to a polynucleotide that encodes at least one polypeptide. mRNA as used herein encompasses both modified and unmodified RNA. mRNA may contain one or more coding and non-coding regions. mRNA can be purified from natural sources, produced using recombinant expression systems and optionally purified, chemically synthesized, etc. Where appropriate, e.g., in the case of chemically synthesized molecules, mRNA can comprise nucleoside analogs such as analogs having chemically modified bases or sugars, backbone modifications, etc. An mRNA sequence is presented in the 5′ to 3′ direction unless otherwise indicated. In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

In some embodiments, the mRNA comprises one or more nonstandard nucleotide residues. The nonstandard nucleotide residues may include, e.g., 5-methyl-cytidine (“5mC”), pseudouridine (“ψU”), and/or 2-thio-uridine (“2sU”). See, e.g., U.S. Pat. No. 8,278,036 or WO2011012316 for a discussion of such residues and their incorporation into mRNA. The mRNA may be RNA, which is defined as RNA in which 25% of U residues are 2-thio-uridine and 25% of C residues are 5-methylcytidine. Teachings for the use of RNA are disclosed US Patent Publication US20120195936 and internation publication WO2011012316, both of which are hereby incorporated by reference in their entirety. The presence of nonstandard nucleotide residues may render an mRNA more stable and/or less immunogenic than a control mRNA with the same sequence but containing only standard residues. In further embodiments, the mRNA may comprise one or more nonstandard nucleotide residues chosen from isocytosine, pseudoisocytosine, 5-bromouracil, 5-propynyluracil, 6-aminopurine, 2-aminopurine, inosine, diaminopurine and 2-chloro-6-aminopurine cytosine, as well as combinations of these modifications and other nucleobase modifications. Certain embodiments may further include additional modifications to the furanose ring or nucleobase. Additional modifications may include, for example, sugar modifications or substitutions (e.g., one or more of a 2′-O-alkyl modification, a locked nucleic acid (LNA)). In some embodiments, the RNAs may be complexed or hybridized with additional polynucleotides and/or peptide polynucleotides (PNA). In embodiments where the sugar modification is a 2′-O-alkyl modification, such modification may include, but are not limited to a 2′-deoxy-2′-fluoro modification, a 2′-O-methyl modification, a 2′-O-methoxyethyl modification and a 2′-deoxy modification. In certain embodiments, any of these modifications may be present in 0-100% of the nucleotides—for example, more than 0%, 1%, 10%, 25%, 50%, 75%, 85%, 90%, 95%, or 100% of the constituent nucleotides individually or in combination.

Nucleic acid. As used herein, the term “nucleic acid,” in its broadest sense, refers to any compound and/or substance that is or can be incorporated into a polynucleotide chain. In some embodiments, a nucleic acid is a compound and/or substance that is or can be incorporated into a polynucleotide chain via a phosphodiester linkage. In some embodiments, “nucleic acid” refers to individual nucleic acid residues (e.g., nucleotides and/or nucleosides). In some embodiments, “nucleic acid” refers to a polynucleotide chain comprising individual nucleic acid residues. In some embodiments, “nucleic acid” encompasses RNA as well as single and/or double-stranded DNA and/or cDNA.

Patient: As used herein, the term “patient” or “subject” refers to any organism to which a provided composition may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. A human includes pre and post natal forms.

Pharmaceutically acceptable: The term “pharmaceutically acceptable” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Systemic distribution or delivery: As used herein, the terms “systemic distribution,” “systemic delivery,” or grammatical equivalent, refer to a delivery or distribution mechanism or approach that affect the entire body or an entire organism. Typically, systemic distribution or delivery is accomplished via body's circulation system, e.g., blood stream. Compared to the definition of “local distribution or delivery.”

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Target tissues: As used herein, the term “target tissues” refers to any tissue that is affected by a disease to be treated. In some embodiments, target tissues include those tissues that display disease-associated pathology, symptom, or feature.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

### DETAILED DESCRIPTION

The present invention provides, among other things, methods and compositions for delivering mRNA in vivo using improved liposomes incorporating cationic lipids described herein.

Liposomes for mRNA Delivery

As used herein, the term “liposome” refers to any lamellar, multilamellar, or solid lipid nanoparticle vesicle. Typically, a liposome as used herein can be formed by mixing one or more lipids or by mixing one or more lipids and polymer(s). Thus, the term “liposome” as used herein encompasses both lipid and polymer based nanoparticles. In particular, a liposome according to the present invention incorporates a cationic lipid described herein. As a non-limiting example, a cationic lipid suitable for the present invention is cKK-E12, or (3,6-bis(4-(bis(2-hydroxydodecyl)amino)butyl)piperazine-2,5-dione), as described in more detail below. A suitable liposome may also contain second or additional cationic lipids, helper lipids (e.g., non-cationic lipids and/or cholesterol-based lipids), PEG-modified lipids, and/or polymers.

In some embodiments, cationic lipid(s) (e.g., cKK-E12) constitute(s) about 30-50% (e.g., about 30-45%, about 30-40%, about 35-50%, about 35-45%, or about 35-40%) of the liposome by molar ratio. In some embodiments, the cationic lipid (e.g., cKK-E12) constitutes about 30%, about 35%, about 40%, about 45%, or about 50% of the liposome by molar ratio.

### Cationic Lipids

In some embodiments, provided liposomes or compositions provided comprise a cationic lipid according to formula I:

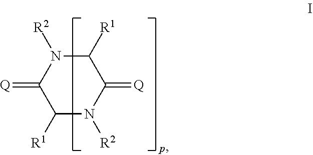

or a pharmaceutically acceptable salt thereof,
wherein:
p is an integer of between 1 and 9, inclusive;
each instance of Q is independently O, S, or NR^{Q};
R^{Q} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group, or a group of the formula (i), (ii) or (iii);
each instance of R¹ is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, halogen, —OR^{A1}, —N(R^{A1})₂, —SR^{A1}, or a group of formula (iv):

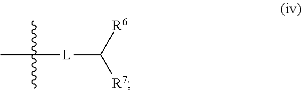

L is an optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, or optionally substituted heteroarylene, or combination thereof, and
each of R⁶ and R⁷ is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group, or a group of formula (i), (ii) or (iii);
each occurrence of R^{A1} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, a sulfur protecting group when attached to an sulfur atom, a nitrogen protecting group when attached to a nitrogen atom, or two R^{A1} groups, together with the nitrogen atom to which they are attached, are joined to form an optionally substituted heterocyclic or optionally substituted heteroaryl ring;
each instance of R² is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group, or a group of the formula (i), (ii), or (iii);
Formulae (i), (ii), and (iii) are:

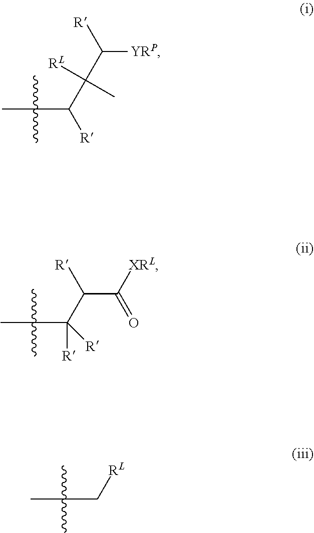

each instance of R′ is independently hydrogen or optionally substituted alkyl;
X is O, S, or NR^{X};
R^{X} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group;
Y is O, S, or NR^{Y};
R^{Y} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group;
R^{P} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, a sulfur protecting group when attached to a sulfur atom, or a nitrogen protecting group when attached to a nitrogen atom;
R^{L} is optionally substituted C₁₋₅₀ alkyl, optionally substituted C₂₋₅₀ alkenyl, optionally substituted C₂₋₅₀ alkynyl, optionally substituted heteroC₁₋₅₀ alkyl, optionally substituted heteroC₂₋₅₀ alkenyl, optionally substituted heteroC₂₋₅₀ alkynyl, or a polymer;
provided that at least one instance of R^{Q}, R², R⁶, or R⁷ is a group of the formula (i), (ii), or (iii).

In some embodiments, a cationic lipid in a provided composition or method is a compound of formula I. In some embodiments, a cationic lipid in a provided composition or method is a compound of formula I, wherein the compound comprises one or more basic groups. In some embodiments, a cationic lipid in a provided composition or method is a compound of formula I, wherein the compound comprises one or more amino groups.

In certain embodiments, a group of formula (i) represents a group of formula (i-a) or a group of formula (i-b):

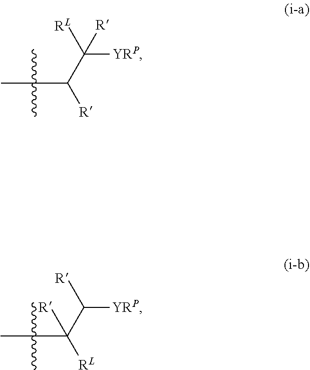

wherein each variable is independently as defined above and described herein. In some embodiments, a group of formula (i) is a group of formula (i-a). In some embodiments, a group of formula (i) is a group of formula (i-b).

In some embodiments, at least one instance of R¹ is a group of formula (iv). In some embodiments, at least one instance of R is a group of formula (iv), wherein at least one of R⁶ and R⁷ is a group of formula (i), (ii) or (iii). In some embodiments, at least one instance of R is a group of formula (iv), wherein each of R⁶ and R⁷ is independently a group of formula (i), (ii) or (iii).

In some embodiments, each Rt is independently a group of formula (iv). In some embodiments, each R¹ is independently a group of formula (iv), wherein at least one of R⁶ and R⁷ is a group of formula (i), (ii) or (iii). In some embodiments, each R¹ is independently a group of formula (iv), wherein each of R⁶ and R⁷ is independently a group of formula (i), (ii) or (iii). In some embodiments, each R¹ is independently a group of formula (iv), wherein each of R⁶ and R⁷ is independently a group of formula (i). In some embodiments, each R¹ is independently a group of formula (iv), wherein each of R⁶ and R⁷ is independently a group of formula (ii). In some embodiments, each R¹ is independently a group of formula (iv), wherein each of R⁶ and R⁷ is independently a group of formula (iii). In some embodiments, each R¹ is independently a group of formula (iv), wherein each of R⁶ and R⁷ is independently a group of formula (i-a). In some embodiments, each R¹ is independently a group of formula (iv), wherein each of R⁶ and R⁷ is independently a group of formula (i-b).

In some embodiments, each instance of R′ is hydrogen.

In some embodiments, L is an optionally substituted alkylene.

In some embodiments, a group of formula (iv) is of formula

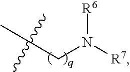

wherein q is an integer between 1 and 50, inclusive, and each of R⁶ and R⁷ is independently as defined above and described herein.

As generally defined above, p is an integer of between 1 and 9, inclusive. In certain embodiments, p is 1. In certain embodiments, p is 2. In certain embodiments, p is 3. In certain embodiments, p is 4. In certain embodiments, p is 5. In certain embodiments, p is 6. In certain embodiments, p is 7. In certain embodiments, p is 8. In certain embodiments, p is 9.

In some embodiments, p is 1. In some embodiments, a compound of formula I is a compound of formula (I-a):

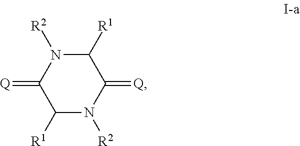

or a pharmaceutically acceptable salt thereof, wherein each variable is independently as defined above and described herein.

In some embodiments, p is 2. In some embodiments, a compound of formula I is a compound of formula (I-p2):

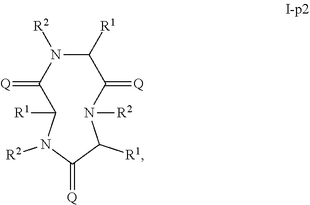

or a pharmaceutically acceptable salt thereof, wherein each variable is independently as defined above and described herein.

In some embodiments, p is 3. In some embodiments, a compound of formula I is a compound of formula (I-p3):

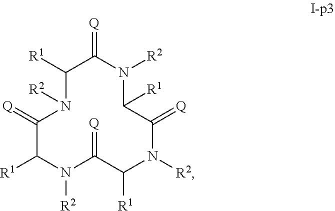

or a pharmaceutically acceptable salt thereof, wherein each variable is independently as defined above and described herein.

In some embodiments, p is 4. In some embodiments, a compound of formula I is a compound of formula (I-p4):

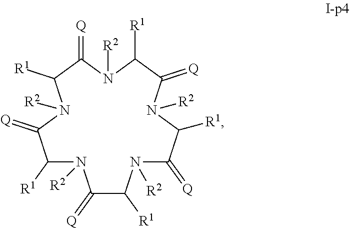

or a pharmaceutically acceptable salt thereof, wherein each variable is independently as defined above and described herein.

In some embodiments, p is 5. In some embodiments, a compound of formula I is a compound of formula (I-p5):

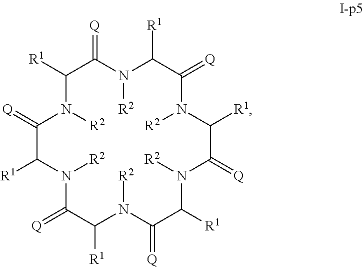

or a pharmaceutically acceptable salt thereof, wherein each variable is independently as defined above and described herein.

In some embodiments, p is 6. In some embodiments, a compound of formula I is a compound of formula (I-p6):

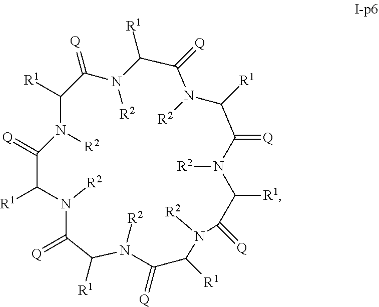

or a pharmaceutically acceptable salt thereof, wherein each variable is independently as defined above and described herein.

In some embodiments, p is 7. In some embodiments, a compound of formula I is a compound of formula (I-p7):

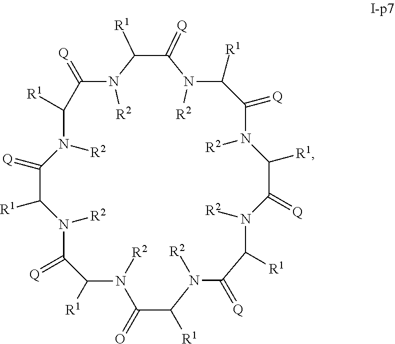

or a pharmaceutically acceptable salt thereof, wherein each variable is independently as defined above and described herein.

In some embodiments, p is 8. In some embodiments, a compound of formula I is a compound of formula (I-p8):

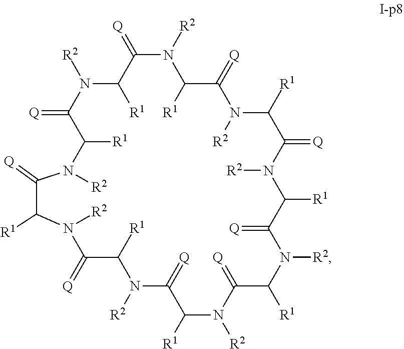

or a pharmaceutically acceptable salt thereof, wherein each variable is independently as defined above and described herein.

In some embodiments, p is 9. In some embodiments, a compound of formula I is a compound of formula (I-p9):

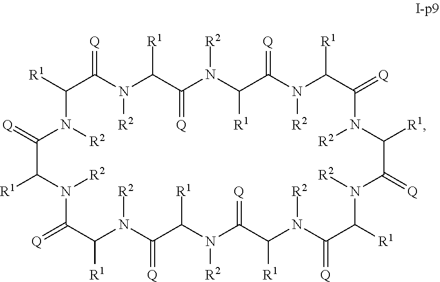

or a pharmaceutically acceptable salt thereof, wherein each variable is independently as defined above and described herein.

As generally defined above, each instance of Q is independently O, S, or NR^{Q}, wherein R^{Q} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group, or a group of the formula (i), (ii), or (iii).

In certain embodiments, at least one instance of Q is O. In certain embodiments, each instance of Q is O. In certain embodiments, at least one instance of Q is S. In certain embodiments, each instance of Q is S. In certain embodiments, at least one instance of Q is NR^{Q}, wherein R^{Q} is as defined above and described herein. In certain embodiments, each instance of Q is NR^{Q}, wherein each R^{Q} is independently as defined above and described herein. In certain embodiments, each instance of R^{Q} is independently hydrogen or a group of the formula (i), (ii), or (iii).

As generally defined above, R^{Q} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group, or a group of the formula (i), (ii) or (iii).

In some embodiments, R^{Q} is hydrogen. In some embodiments, R^{Q} is optionally substituted alkyl. In some embodiments, R^{Q} is optionally substituted alkenyl. In some embodiments, R^{Q} is optionally substituted alkynyl. In some embodiments, R^{Q} is carbocyclyl. In some embodiments, R^{Q} is optionally substituted heterocyclyl. In some embodiments, R^{Q} is optionally substituted aryl. In some embodiments, R^{Q} is optionally substituted heteroaryl. In some embodiments, R^{Q} is a nitrogen protecting group. In some embodiments, R^{Q} is a group of formula (i), (ii) or (iii). In some embodiments, R^{Q} is a group of formula (i). In some embodiments, R^{Q} is a group of formula (ii). In some embodiments, R^{Q} is a group of formula (iii).

As generally defined above, each instance of R¹ is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, halogen, —OR^{A1}, —N(R^{A1})₂, or —SR^{A1}, or a group of formula (iv), wherein each of R^{A1} and formula (iv) is independently as defined above and described herein.

In some embodiments, R¹ is hydrogen.

In certain embodiments, R¹ is optionally substituted alkyl, optionally substituted alkenyl, optionally substituted alkynyl, optionally substituted carbocyclyl, optionally substituted heterocyclyl, optionally substituted aryl, or optionally substituted heteroaryl. In certain embodiments, at least one instance of R¹ is optionally substituted alkyl, optionally substituted alkenyl, optionally substituted alkynyl, optionally substituted carbocyclyl, optionally substituted heterocyclyl, optionally substituted aryl, or optionally substituted heteroaryl.

In certain embodiments, R¹ is optionally substituted alkyl; e.g., optionally substituted C₁₋₆alkyl, optionally substituted C₂₋₆alkyl, optionally substituted C₃₋₆alkyl, optionally substituted C₄₋₆alkyl, optionally substituted C₄₋₅alkyl, or optionally substituted C₃₋₄alkyl. In certain embodiments, at least one instance of R¹ is optionally substituted alkyl; e.g., optionally substituted C₁₋₆alkyl, optionally substituted C₂₋₆alkyl, optionally substituted C₃₋₆alkyl, optionally substituted C₄₋₆alkyl, optionally substituted C₄₋₅alkyl, or optionally substituted C₃₋₄alkyl.

In certain embodiments, R¹ is optionally substituted alkenyl, e.g., optionally substituted C₂₋₆alkenyl, optionally substituted C₃₋₆alkenyl, optionally substituted C₄₋₆alkenyl, optionally substituted C₄₋₅alkenyl, or optionally substituted C₃₋₄alkenyl. In certain embodiments, at least one instance of R is optionally substituted alkenyl, e.g., optionally substituted C₂₋₆alkenyl, optionally substituted C₃₋₆alkenyl, optionally substituted C₄₋₆alkenyl, optionally substituted C₄₋₅alkenyl, or optionally substituted C₃₋₄alkenyl.

In certain embodiments, R¹ is optionally substituted alkynyl, e.g., optionally substituted C₂₋₆alkynyl, optionally substituted C₃₋₆alkynyl, optionally substituted C₄₋₆alkynyl, optionally substituted C₄₋₅alkynyl, or optionally substituted C₃₋₄alkynyl. In certain embodiments, at least one instance of R is optionally substituted alkynyl, e.g., optionally substituted C₂₋₆alkynyl, optionally substituted C₃₋₆alkynyl, optionally substituted C₄₋₆alkynyl, optionally substituted C₄₋₅alkynyl, or optionally substituted C₃₋₄alkynyl.

In certain embodiments, R¹ is optionally substituted carbocyclyl, e.g., optionally substituted C₃₋₁₀ carbocyclyl, optionally substituted C₅₋₈ carbocyclyl, optionally substituted C₅₋₆ carbocyclyl, optionally substituted C₅ carbocyclyl, or optionally substituted C₆ carbocyclyl. In certain embodiments, at least one instance of R¹ is optionally substituted carbocyclyl, e.g., optionally substituted C₃₋₁₀ carbocyclyl, optionally substituted C₅₋₈ carbocyclyl, optionally substituted C₅₋₆ carbocyclyl, optionally substituted C₅ carbocyclyl, or optionally substituted C₆ carbocyclyl.

In some embodiments, R¹ is optionally substituted heterocyclyl, e.g., optionally substituted 3-14 membered heterocyclyl, optionally substituted 3-10 membered heterocyclyl, optionally substituted 5-8 membered heterocyclyl, optionally substituted 5-6 membered heterocyclyl, optionally substituted 5-membered heterocyclyl, or optionally substituted 6-membered heterocyclyl. In certain embodiments, at least one instance of R is optionally substituted heterocyclyl, e.g., optionally substituted 3-14 membered heterocyclyl, optionally substituted 3-10 membered heterocyclyl, optionally substituted 5-8 membered heterocyclyl, optionally substituted 5-6 membered heterocyclyl, optionally substituted 5-membered heterocyclyl, or optionally substituted 6-membered heterocyclyl.

In some embodiments, R¹ is optionally substituted aryl. In some embodiments, R¹ is optionally substituted phenyl. In some embodiments, R¹ is phenyl. In some embodiments, R¹ is substituted phenyl. In certain embodiments, at least one instance of R is optionally substituted aryl, e.g., optionally substituted phenyl.

In some embodiments, R¹ is optionally substituted heteroaryl, e.g., optionally substituted 5-14 membered heteroaryl, optionally substituted 5-10 membered heteroaryl, optionally substituted 5-6 membered heteroaryl, optionally substituted 5 membered heteroaryl, or optionally substituted 6 membered heteroaryl. In certain embodiments, at least one instance of R¹ is optionally substituted heteroaryl, e.g., optionally substituted 5-14 membered heteroaryl, optionally substituted 5-10 membered heteroaryl, optionally substituted 5-6 membered heteroaryl, optionally substituted 5 membered heteroaryl, or optionally substituted 6 membered heteroaryl.

In some embodiments, R¹ is halogen. In some embodiments, R¹ is —F. In some embodiments, R¹ is —Cl. In some embodiments, R¹ is —Br. In some embodiments, R¹ is —I.

In some embodiments, R¹ is —OR^{A1}, wherein R^{A1} is as defined above and described herein. In some embodiments, R is —N(R^{A1})₂, wherein each R^{A1} is independently as defined above and described herein. In some embodiments, R¹ is —SR^{A1}, wherein R^{A1} is as defined above and described herein.

In some embodiments, an R¹ alkyl, alkenyl, alkynyl, carbocyclyl, heterocyclyl, aryl, or heteroaryl group may be substituted. In some embodiments, an R¹ alkyl, alkenyl, alkynyl, carbocyclyl, heterocyclyl, aryl, or heteroaryl group may be substituted with an optionally substituted amino group. In some embodiments, an R¹ alkyl, alkenyl, alkynyl, carbocyclyl, heterocyclyl, aryl, or heteroaryl group may be substituted with an optionally substituted hydroxyl group. In some embodiments, an R¹ alkyl, alkenyl, alkynyl, carbocyclyl, heterocyclyl, aryl, or heteroaryl group may be substituted with an optionally substituted thiol group. In any of the above embodiments, an R¹ alkyl, alkenyl, alkynyl, carbocyclyl, heterocyclyl, aryl, or heteroaryl group may be substituted, for example, with an optionally substituted amino group (e.g., —NR⁶R⁷), an optionally substituted hydroxyl group (e.g., —OR), an optionally substituted thiol group (e.g., —SR⁶), or with a group of formula (i), (ii), or (iii), wherein each instance of R⁶ and R⁷ is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, and a sulfur protecting group when attached to a sulfur atom, or a group of formula (i), (ii), or (iii).

In some embodiments, R¹ is an optionally substituted natural amino acid side chain. In some embodiments, R¹ is a natural amino acid side chain. In some embodiments, R¹ is an optionally substituted unnatural amino acid side chain. In some embodiments, R is an unnatural amino acid side chain.

In certain embodiments, each instance of R¹ is the same. In certain embodiments, at least one R¹ group is different. In certain embodiments, each R¹ group is different.

In certain embodiments, R¹ is an alkyl, alkenyl, alkynyl, carbocyclyl, heterocyclyl, aryl, or heteroaryl group substituted with an amino group of the formula —NR⁶R⁷.

In certain embodiments, R¹ is a group of formula (iv):

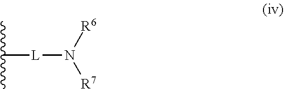

wherein:
L is an optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, or optionally substituted heteroarylene, or combination thereof, and
each of R⁶ and R⁷ is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group, or a group of formula (i), (ii) or (iii):

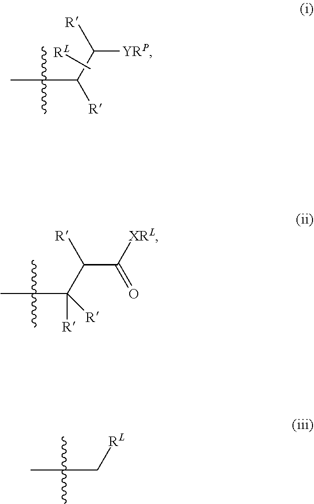

wherein each of R′, Y, R^{P}, R^{L} and X is independently as defined above and described herein.

In some embodiments, at least one instance of R¹ is an alkyl, alkenyl, alkynyl, carbocyclyl, heterocyclyl, aryl, or heteroaryl group substituted with an amino group of the formula —NR⁶R⁷. In some embodiments, at least one instance of R¹ is a group of formula (iv). In some embodiments, at least one instance of R¹ is a group of formula (iv), wherein at least one instance of R⁶ and R⁷ is a group of the formula (i), (ii) or (iii). In some embodiments, at least one instance of R¹ is a group of formula (iv), wherein each instance of R⁶ and R⁷ is a group of the formula (i), (ii) or (iii). In some embodiments, at least one instance of R¹ is a group of formula (iv), wherein each instance of R⁶ and R⁷ is a group of the formula (i). In some embodiments, at least one instance of R¹ is a group of formula (iv), wherein each instance of R⁶ and R⁷ is a group of the formula (ii). In some embodiments, at least one instance of R¹ is a group of formula (iv), wherein each instance of R⁶ and R⁷ is a group of the formula (iii).

In some embodiments, each instance of R¹ is a group of formula (iv). In some embodiments, each instance of R¹ is a group of formula (iv), wherein each instance of R⁶ and R⁷ is a group of the formula (i), (ii) or (iii). In some embodiments, each instance of R¹ is a group of formula (iv), wherein each instance of R⁶ and R⁷ is a group of the formula (i), (ii) or (iii). In some embodiments, each instance of R¹ is a group of formula (iv), wherein each instance of R⁶ and R⁷ is a group of the formula (i). In some embodiments, each instance of R¹ is a group of formula (iv), wherein each instance of R⁶ and R⁷ is a group of the formula (ii). In some embodiments, each instance of R¹ is a group of formula (iv), wherein each instance of R⁶ and R⁷ is a group of the formula (iii).

In certain embodiments, at least two instances of R¹ is a group of formula (iv). In certain embodiments, at least three instances of R¹ is a group of formula (iv). In certain embodiments, at least four instances of R¹ is a group of formula (iv). In certain embodiments, at least five instances of R¹ is a group of formula (iv). In certain embodiments, at least six instances of R¹ is a group of formula (iv). In certain embodiments, at least seven instances of R is a group of formula (iv). In certain embodiments, at least eight instances of R¹ is a group of formula (iv). In certain embodiments, at least nine instances of R is a group of formula (iv). In certain embodiments, each instance of R¹ is a group of formula (iv).

In certain embodiments, L is an optionally substituted alkylene; e.g., optionally substituted C₁₋₅₀ alkylene, optionally substituted C₁₋₄₀alkylene, optionally substituted C₁₋₃₀ alkylene, optionally substituted C₁₋₂₀ alkylene, optionally substituted C₄₋₂₀ alkylene, optionally substituted C₆₋₂₀ alkylene, optionally substituted C₈₋₂₀alkylene, optionally substituted C₁₀₋₂₀alkylene, optionally substituted C₁₋₆alkylene, optionally substituted C₂₋₆alkylene, optionally substituted C₃₋₆alkylene, optionally substituted C₄₋₆alkylene, optionally substituted C₄₋₅alkylene, or optionally substituted C₃₋₄alkylene. In some embodiments, L is optionally substituted C₁ alkylene. In some embodiments, L is optionally substituted C₂ alkylene. In some embodiments, L is optionally substituted C₃ alkylene. In some embodiments, L is optionally substituted C₄ alkylene. In some embodiments, L is optionally substituted C₅ alkylene. In some embodiments, L is optionally substituted C₆ alkylene. In some embodiments, L is optionally substituted C₇ alkylene. In some embodiments, L is optionally substituted C₈ alkylene. In some embodiments, L is optionally substituted C₉ alkylene. In some embodiments, L is optionally substituted C₁₀ alkylene. In some embodiments, L is —CH₂—. In some embodiments, L is —(CH₂)₂—. In some embodiments, L is —(CH₂)₃—. In some embodiments, L is —(CH₂)₄—. In some embodiments, L is —(CH₂)₅—. In some embodiments, L is —(CH₂)₆—. In some embodiments, L is —(CH₂)₇—. In some embodiments, L is —(CH₂)₈—. In some embodiments, L is —(CH₂)₉—. In some embodiments, L is —(CH₂)₁₀—.

In certain embodiments, L is an optionally substituted alkenylene, e.g., optionally substituted C₂₋₅₀alkenylene, optionally substituted C₂₋₄₀alkenylene, optionally substituted C₂₋₃₀alkenylene, optionally substituted C₂₋₂₀alkenylene, optionally substituted C₄₋₂₀alkenylene, optionally substituted C₆₋₂₀alkenylene, optionally substituted C₈₋₂₀alkenylene, optionally substituted C₁₀₋₂₀alkenylene, optionally substituted C₂₋₆alkenylene, optionally substituted C₃₋₆ alkenylene, optionally substituted C₄₋₆alkenylene, optionally substituted C₄₋₅alkenylene, or optionally substituted C₃₋₄alkenylene.

In certain embodiments, L is an optionally substituted alkynylene, e.g., optionally substituted C₂₋₅₀alkynylene, optionally substituted C₂₋₄₀alkynylene, optionally substituted C₂₋₃₀ alkynylene, optionally substituted C₂₋₂₀alkynylene, optionally substituted C₄₋₂₀alkynylene, optionally substituted C₆₋₂₀alkynylene, optionally substituted C₈₋₂₀alkynylene, optionally substituted C₁₀₋₂₀alkynylene, optionally substituted C₂₋₆alkynylene, optionally substituted C₃₋₆ alkynylene, optionally substituted C₄₋₆alkynylene, optionally substituted C₄₋₅alkynylene, or optionally substituted C₃₋₄alkynylene.

In certain embodiments, L is an optionally substituted heteroalkylene; e.g., optionally substituted heteroC₁₋₅₀alkylene, optionally substituted heteroC₁₋₄₀alkylene, optionally substituted heteroC₁₋₃₀alkylene, optionally substituted heteroC₁₋₂₀alkylene, optionally substituted heteroC₄₋₂₀alkylene, optionally substituted heteroC₆₋₂₀alkylene, optionally substituted heteroC₈₋₂₀ alkylene, optionally substituted heteroC₁₀₋₂₀alkylene, optionally substituted heteroC₁₋₆alkylene, optionally substituted heteroC₂₋₆alkylene, optionally substituted heteroC₃₋₆alkylene, optionally substituted heteroC₄₋₆alkylene, optionally substituted heteroC₄₋₅alkylene, or optionally substituted heteroC₃₋₄alkylene. In some embodiments, L is optionally substituted heteroC₂alkylene. In some embodiments, L is optionally substituted heteroC₃alkylene. In some embodiments, L is optionally substituted heteroC₄alkylene. In some embodiments, L is optionally substituted heteroC₅alkylene. In some embodiments, L is optionally substituted heteroC₆alkylene. In some embodiments, L is optionally substituted heteroC₇alkylene. In some embodiments, L is optionally substituted heteroC₈alkylene. In some embodiments, L is optionally substituted heteroC₉alkylene. In some embodiments, L is optionally substituted heteroC₁₀alkylene.

In certain embodiments, L is an optionally substituted heteroalkenylene, e.g., optionally substituted heteroC₂₋₅₀alkenylene, optionally substituted heteroC₂₋₄₀alkenylene, optionally substituted heteroC₂₋₃₀alkenylene, optionally substituted heteroC₂₋₂₀alkenylene, optionally substituted heteroC₄₋₂₀alkenylene, optionally substituted heteroC₆₋₂₀alkenylene, optionally substituted heteroC₈₋₂₀alkenylene, optionally substituted heteroC₁₀₋₂₀alkenylene, optionally substituted heteroC₂₋₆alkenylene, optionally substituted heteroC₃₋₆alkenylene, optionally substituted heteroC₄₋₆alkenylene, optionally substituted heteroC₄₋₅alkenylene, or optionally substituted heteroC₃₋₄alkenylene.

In certain embodiments, L is an optionally substituted heteroalkynylene, e.g., optionally substituted heteroC₂₋₅₀alkynylene, optionally substituted heteroC₂₋₄₀alkynylene, optionally substituted heteroC₂₋₃₀alkynylene, optionally substituted heteroC₂₋₂₀alkynylene, optionally substituted heteroC₄₋₂₀alkynylene, optionally substituted heteroC₆₋₂₀alkynylene, optionally substituted heteroC₈₋₂₀alkynylene, optionally substituted heteroC₁₀₋₂₀alkynylene, optionally substituted heteroC₂₋₆alkynylene, optionally substituted heteroC₃₋₆alkynylene, optionally substituted heteroC₄₋₆alkynylene, optionally substituted heteroC₄₋₅alkynylene, or optionally substituted heteroC₃₋₄alkynylene.

In certain embodiments, L is an optionally substituted carbocyclylene, e.g., optionally substituted C₃₋₁₀carbocyclylene, optionally substituted C₅₋₈carbocyclylene, optionally substituted C₅₋₆carbocyclylene, optionally substituted C₅carbocyclylene, or optionally substituted C₆carbocyclylene.

In certain embodiments, L is an optionally substituted heterocyclylene, e.g., optionally substituted 3-14 membered heterocyclylene, optionally substituted 3-10 membered heterocyclylene, optionally substituted 5-8 membered heterocyclylene, optionally substituted 5-6 membered heterocyclylene, optionally substituted 5-membered heterocyclylene, or optionally substituted 6-membered heterocyclylene.

In certain embodiments, L is an optionally substituted arylene, e.g., optionally substituted phenylene. In some embodiments, L is optionally substituted phenylene. In some embodiments, L is substituted phenylene. In some embodiments, L is unsubstituted phenylene.

In certain embodiments, L is an optionally substituted heteroarylene, e.g., optionally substituted 5-14 membered heteroarylene, optionally substituted 5-10 membered heteroarylene, optionally substituted 5-6 membered heteroarylene, optionally substituted 5-membered heteroarylene, or optionally substituted 6-membered heteroarylene.

In certain embodiments, wherein L is an optionally substituted alkylene group, the group of formula (iv) is a group of the formula

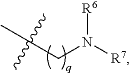

wherein q is an integer between 1 and 50, inclusive, and each of R⁶ and R⁷ is independently as defined above and described herein.

In certain embodiments, q is an integer between 1 and 40, inclusive. In certain embodiments, q is an integer between 1 and 30, inclusive. In certain embodiments, q is an integer between 1 and 20, inclusive. In certain embodiments, q is an integer between 1 and 10, inclusive. In certain embodiments, q is an integer between 4 and 20, inclusive. In certain embodiments, q is an integer between 6 and 20, inclusive. In certain embodiments, q is an integer between 2 and 10, inclusive. In certain embodiments, q is an integer between 2 and 9, inclusive. In certain embodiments, q is an integer between 2 and 8, inclusive. In certain embodiments, q is an integer between 2 and 7, inclusive. In certain embodiments, q is an integer between 2 and 6, inclusive. In certain embodiments, q is an integer between 2 and 5, inclusive. In certain embodiments, q is an integer between 2 and 4, inclusive. In certain embodiments, q is an integer between 3 and 10, inclusive. In certain embodiments, q is an integer between 3 and 8, inclusive. In certain embodiments, q is an integer between 3 and 7, inclusive. In certain embodiments, q is an integer between 3 and 6, inclusive. In certain embodiments, q is an integer between 3 and 5, inclusive. In certain embodiments, q is 3 or 4. In certain embodiments, q is an integer between 3 and 9, inclusive. In certain embodiments, q is an integer between 8 and 20, inclusive. In certain embodiments, q is 1. In certain embodiments, q is 2. In certain embodiments, q is 3. In certain embodiments, q is 4. In certain embodiments, q is 5. In certain embodiments, q is 6. In certain embodiments, q is 7. In certain embodiments, q is 8. In certain embodiments, q is 9. In certain embodiments, q is 10.

As generally defined above, each R⁶ is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group, or a group of formula (i), (ii) or (iii).

In some embodiments, R⁶ is hydrogen.

In some embodiments, R⁶ is optionally substituted alkyl. In some embodiments, R⁶ is optionally substituted C₂₋₅₀ alkyl. In some embodiments, R⁶ is optionally substituted C₂₋₄₀ alkyl. In some embodiments, R⁶ is optionally substituted C₂₋₃₀ alkyl. In some embodiments, R⁶ is optionally substituted C₂₋₂₀ alkyl. In some embodiments, R⁶ is optionally substituted C₂₋₁₉ alkyl. In some embodiments, R⁶ is optionally substituted C₂₋₁₈ alkyl. In some embodiments, R⁶ is optionally substituted C₂₋₇ alkyl. In some embodiments, R⁶ is optionally substituted C₂₋₁₆ alkyl. In some embodiments, R⁶ is optionally substituted C₂₋₁₅ alkyl. In some embodiments, R⁶ is optionally substituted C₂₋₁₄ alkyl. In some embodiments, R⁶ is optionally substituted C₂₋₃ alkyl. In some embodiments, R⁶ is optionally substituted C₂₋₁₂ alkyl. In some embodiments, R⁶ is optionally substituted C₂₋₁₁ alkyl. In some embodiments, R⁶ is optionally substituted C₂₋₁₀ alkyl. In some embodiments, R⁶ is optionally substituted C₂₋₉ alkyl. In some embodiments, R⁶ is optionally substituted C₂₋₈ alkyl. In some embodiments, R⁶ is optionally substituted C₂₋₇ alkyl. In some embodiments, R⁶ is optionally substituted C₂₋₆ alkyl.

In some embodiments, R⁶ is optionally substituted C₄₋₅₀ alkyl. In some embodiments, R⁶ is optionally substituted C₄₋₄₀ alkyl. In some embodiments, R⁶ is optionally substituted C₄₋₃₀ alkyl. In some embodiments, R⁶ is optionally substituted C₄₋₂₀ alkyl. In some embodiments, R⁶ is optionally substituted C₄₋₁₉ alkyl. In some embodiments, R⁶ is optionally substituted C₄₋₁₈ alkyl. In some embodiments, R⁶ is optionally substituted C₄₋₁₇ alkyl. In some embodiments, R⁶ is optionally substituted C₄₋₁₆ alkyl. In some embodiments, R⁶ is optionally substituted C₄₋₁₅ alkyl. In some embodiments, R⁶ is optionally substituted C₄₋₁₄ alkyl. In some embodiments, R⁶ is optionally substituted C₄₋₁₃ alkyl. In some embodiments, R⁶ is optionally substituted C₄₋₁₂ alkyl. In some embodiments, R⁶ is optionally substituted C₄₋₁₁ alkyl. In some embodiments, R⁶ is optionally substituted C₄₋₁₀ alkyl. In some embodiments, R⁶ is optionally substituted C₄₋₉ alkyl. In some embodiments, R⁶ is optionally substituted C₄₋₈ alkyl. In some embodiments, R⁶ is optionally substituted C₄₋₇ alkyl. In some embodiments, R⁶ is optionally substituted C₄₋₆ alkyl.

In some embodiments, R⁶ is optionally substituted C₆₋₅₀ alkyl. In some embodiments, R⁶ is optionally substituted C₆₋₄₀ alkyl. In some embodiments, R⁶ is optionally substituted C₆₋₃₀ alkyl. In some embodiments, R⁶ is optionally substituted C₆₋₂₀ alkyl. In some embodiments, R⁶ is optionally substituted C₆₋₁₉ alkyl. In some embodiments, R⁶ is optionally substituted C₆₋₁₈ alkyl. In some embodiments, R⁶ is optionally substituted C₆₋₇ alkyl. In some embodiments, R⁶ is optionally substituted C₆₋₁₆ alkyl. In some embodiments, R⁶ is optionally substituted C₆₋₁₅ alkyl. In some embodiments, R⁶ is optionally substituted C₆₋₁₄ alkyl. In some embodiments, R⁶ is optionally substituted C₆₋₁₃ alkyl. In some embodiments, R⁶ is optionally substituted C₆₋₁₂ alkyl. In some embodiments, R⁶ is optionally substituted C₆₋₁₁ alkyl. In some embodiments, R⁶ is optionally substituted C₆₋₁₀ alkyl. In some embodiments, R⁶ is optionally substituted C₆₋₉ alkyl. In some embodiments, R⁶ is optionally substituted C₆₋₈ alkyl. In some embodiments, R⁶ is optionally substituted C₆₋₇ alkyl.

In some embodiments, R⁶ is optionally substituted C₈₋₅₀ alkyl. In some embodiments, R⁶ is optionally substituted C₈₋₄₀ alkyl. In some embodiments, R⁶ is optionally substituted C₈₋₃₀ alkyl. In some embodiments, R⁶ is optionally substituted C₈₋₂₀ alkyl. In some embodiments, R⁶ is optionally substituted C₆₋₁₉ alkyl. In some embodiments, R⁶ is optionally substituted C₈₋₁₈ alkyl. In some embodiments, R⁶ is optionally substituted C₈₋₁₇ alkyl. In some embodiments, R⁶ is optionally substituted C₈₋₁₆ alkyl. In some embodiments, R⁶ is optionally substituted C₈₋₁₅ alkyl. In some embodiments, R⁶ is optionally substituted C₈₋₁₄ alkyl. In some embodiments, R⁶ is optionally substituted C₈₋₁₃ alkyl. In some embodiments, R⁶ is optionally substituted C₈₋₁₂ alkyl. In some embodiments, R⁶ is optionally substituted C₈₋₁₁ alkyl. In some embodiments, R⁶ is optionally substituted C₈₋₁₀ alkyl. In some embodiments, R⁶ is optionally substituted C₈₋₉ alkyl.

In some embodiments, R⁶ is optionally substituted C₉₋₅₀ alkyl. In some embodiments, R⁶ is optionally substituted C₉₋₄₀ alkyl. In some embodiments, R⁶ is optionally substituted C₉₋₃₀ alkyl. In some embodiments, R⁶ is optionally substituted C₉₋₂₀ alkyl. In some embodiments, R⁶ is optionally substituted C₉₋₁₉ alkyl. In some embodiments, R⁶ is optionally substituted C₉₋₁₅ alkyl. In some embodiments, R⁶ is optionally substituted C₉₋₇ alkyl. In some embodiments, R⁶ is optionally substituted C₉₋₁₆ alkyl. In some embodiments, R⁶ is optionally substituted C₉₋₁₅ alkyl. In some embodiments, R⁶ is optionally substituted C₉₋₁₄ alkyl. In some embodiments, R⁶ is optionally substituted C₉₋₁₃ alkyl. In some embodiments, R⁶ is optionally substituted C₉₋₁₂ alkyl. In some embodiments, R⁶ is optionally substituted C₉₋₁₁ alkyl. In some embodiments, R⁶ is optionally substituted C₉₋₁₀ alkyl.

In some embodiments, R⁶ is optionally substituted C₁₀₋₅₀ alkyl. In some embodiments, R⁶ is optionally substituted C₁₀₋₄₀ alkyl. In some embodiments, R⁶ is optionally substituted C₁₀₋₃₀ alkyl. In some embodiments, R⁶ is optionally substituted C₁₀₋₂₀ alkyl. In some embodiments, R⁶ is optionally substituted C₁₀₋₁₉ alkyl. In some embodiments, R⁶ is optionally substituted C₁₀₋₁₈ alkyl. In some embodiments, R⁶ is optionally substituted C₁₀₋₁₇ alkyl. In some embodiments, R⁶ is optionally substituted C₁₀₋₁₆ alkyl. In some embodiments, R⁶ is optionally substituted C₁₀₋₁₈ alkyl. In some embodiments, R⁶ is optionally substituted C₁₀₋₁₄ alkyl. In some embodiments, R⁶ is optionally substituted C₁₀₋₁₃ alkyl. In some embodiments, R⁶ is optionally substituted C₁₀₋₁₂ alkyl. In some embodiments, R⁶ is optionally substituted C₁₀₋₁₁ alkyl.

In some embodiments, R⁶ is optionally substituted C₁₁₋₅₀ alkyl. In some embodiments, R⁶ is optionally substituted C₁₁₋₄₀ alkyl. In some embodiments, R⁶ is optionally substituted C₁₁₋₃₀ alkyl. In some embodiments, R⁶ is optionally substituted C₁₁₋₂₀ alkyl. In some embodiments, R⁶ is optionally substituted C₁₁₋₁₉ alkyl. In some embodiments, R⁶ is optionally substituted C₁₁₋₁₈ alkyl. In some embodiments, R⁶ is optionally substituted C₁₁₋₁₇ alkyl. In some embodiments, R⁶ is optionally substituted C₁₁₋₁₆ alkyl. In some embodiments, R⁶ is optionally substituted C₁₁₋₁₅ alkyl. In some embodiments, R⁶ is optionally substituted C₁₁₋₁₄ alkyl. In some embodiments, R⁶ is optionally substituted C₁₁₋₁₃ alkyl. In some embodiments, R⁶ is optionally substituted C₁₁₋₁₂ alkyl.

In some embodiments, R⁶ is optionally substituted C₁₂₋₅₀ alkyl. In some embodiments, R⁶ is optionally substituted C₁₂₋₄₀ alkyl. In some embodiments, R⁶ is optionally substituted C₁₂₋₃₀ alkyl. In some embodiments, R⁶ is optionally substituted C₁₂₋₂₀ alkyl. In some embodiments, R⁶ is optionally substituted C₁₂₋₁₉ alkyl. In some embodiments, R⁶ is optionally substituted C₁₂₋₁₈ alkyl. In some embodiments, R⁶ is optionally substituted C₁₂₋₁₇ alkyl. In some embodiments, R⁶ is optionally substituted C₁₂₋₁₆ alkyl. In some embodiments, R⁶ is optionally substituted C₁₂₋₁₅ alkyl. In some embodiments, R⁶ is optionally substituted C₁₂₋₁₄ alkyl. In some embodiments, R⁶ is optionally substituted C₁₂₋₁₃ alkyl.

In some embodiments, R⁶ is optionally substituted C₆ alkyl. In some embodiments, R⁶ is optionally substituted C₇ alkyl. In some embodiments, R⁶ is optionally substituted C₈ alkyl. In some embodiments, R⁶ is optionally substituted C₉ alkyl. In some embodiments, R⁶ is optionally substituted C₁₀ alkyl. In some embodiments, R⁶ is optionally substituted C₁₁ alkyl. In some embodiments, R⁶ is optionally substituted C₁₂ alkyl. In some embodiments, R⁶ is optionally substituted C₁₃ alkyl. In some embodiments, R⁶ is optionally substituted C₁₄ alkyl. In some embodiments, R⁶ is optionally substituted C₁₅ alkyl. In some embodiments, R⁶ is optionally substituted C₁₆ alkyl. In some embodiments, R⁶ is optionally substituted C₁₇ alkyl. In some embodiments, R⁶ is optionally substituted C₁₈ alkyl. In some embodiments, R⁶ is optionally substituted C₁₉ alkyl. In some embodiments, R⁶ is optionally substituted C₂₀ alkyl.

In some embodiments, for example, in any of the above embodiments, R⁶ is a substituted alkyl group. In some embodiments, R⁶ is an unsubstituted alkyl group. In some embodiments, R⁶ is an optionally substituted straight-chain alkyl group. In some embodiments, R⁶ is a substituted straight-chain alkyl group. In some embodiments, R⁶ is an unsubstituted straight-chain alkyl group. In some embodiments, R⁶ is an optionally substituted branched alkyl group. In some embodiments, R⁶ is a substituted branched alkyl group. In some embodiments, R⁶ is an unsubstituted branched alkyl group.

In some embodiments, R⁶ is optionally substituted alkenyl. In some embodiments, R⁶ is optionally substituted C₂₋₅₀ alkenyl. In some embodiments, R⁶ is optionally substituted C₂₋₄₀ alkenyl. In some embodiments, R⁶ is optionally substituted C₂₋₃₀ alkenyl. In some embodiments, R⁶ is optionally substituted C₂₋₂₀ alkenyl. In some embodiments, R⁶ is optionally substituted C₂₋₁₉ alkenyl. In some embodiments, R⁶ is optionally substituted C₂₋₈ alkenyl. In some embodiments, R⁶ is optionally substituted C₂₋₇ alkenyl. In some embodiments, R⁶ is optionally substituted C₂₋₁₆ alkenyl. In some embodiments, R⁶ is optionally substituted C₂₋₁₅ alkenyl. In some embodiments, R⁶ is optionally substituted C₂₋₁₄ alkenyl. In some embodiments, R⁶ is optionally substituted C₂₋₁₃ alkenyl. In some embodiments, R⁶ is optionally substituted C₂₋₁₂ alkenyl. In some embodiments, R⁶ is optionally substituted C₂₋₁₁ alkenyl. In some embodiments, R⁶ is optionally substituted C₂₋₁₀ alkenyl. In some embodiments, R⁶ is optionally substituted C₂₋₉ alkenyl. In some embodiments, R⁶ is optionally substituted C_{z-g} alkenyl. In some embodiments, R⁶ is optionally substituted C₂₋₇ alkenyl. In some embodiments, R⁶ is optionally substituted C₂₋₆ alkenyl.

In some embodiments, R⁶ is optionally substituted C₄₋₅₀ alkenyl. In some embodiments, R⁶ is optionally substituted C₄₋₄₀ alkenyl. In some embodiments, R⁶ is optionally substituted C₄₋₃₀ alkenyl. In some embodiments, R⁶ is optionally substituted C₄₋₂₀ alkenyl. In some embodiments, R⁶ is optionally substituted C₄₋₁₉ alkenyl. In some embodiments, R⁶ is optionally substituted C₄₋₁₈ alkenyl. In some embodiments, R⁶ is optionally substituted C₄₋₇ alkenyl. In some embodiments, R⁶ is optionally substituted C₄₋₁₆ alkenyl. In some embodiments, R⁶ is optionally substituted C₄₋₁₅ alkenyl. In some embodiments, R⁶ is optionally substituted C₄₋₁₄ alkenyl. In some embodiments, R⁶ is optionally substituted C₄₋₁₃ alkenyl. In some embodiments, R⁶ is optionally substituted C₄₋₁₂ alkenyl. In some embodiments, R⁶ is optionally substituted C₄₋₁₁ alkenyl. In some embodiments, R⁶ is optionally substituted C₄₋₁₀ alkenyl. In some embodiments, R⁶ is optionally substituted C₄₋₉ alkenyl. In some embodiments, R⁶ is optionally substituted C₄₋₈ alkenyl. In some embodiments, R⁶ is optionally substituted C₄₋₇ alkenyl. In some embodiments, R⁶ is optionally substituted C₄₋₆ alkenyl.

In some embodiments, R⁶ is optionally substituted C₆₋₅₀ alkenyl. In some embodiments, R⁶ is optionally substituted C₆₋₄₀ alkenyl. In some embodiments, R⁶ is optionally substituted C₆₋₃₀ alkenyl. In some embodiments, R⁶ is optionally substituted C₆₋₂₀ alkenyl. In some embodiments, R⁶ is optionally substituted C₆₋₁₉ alkenyl. In some embodiments, R⁶ is optionally substituted C₆₋₁₅ alkenyl. In some embodiments, R⁶ is optionally substituted C₆₋₇ alkenyl. In some embodiments, R⁶ is optionally substituted C₆₋₁₆ alkenyl. In some embodiments, R⁶ is optionally substituted C₆₋₁₅ alkenyl. In some embodiments, R⁶ is optionally substituted C₆₋₁₄ alkenyl. In some embodiments, R⁶ is optionally substituted C₆₋₁₃ alkenyl. In some embodiments, R⁶ is optionally substituted C₆₋₁₂ alkenyl. In some embodiments, R⁶ is optionally substituted C₆₋₁₁ alkenyl. In some embodiments, R⁶ is optionally substituted C₆₋₁₀ alkenyl. In some embodiments, R⁶ is optionally substituted C₆₋₉ alkenyl. In some embodiments, R⁶ is optionally substituted C₆₋₈ alkenyl. In some embodiments, R⁶ is optionally substituted C₆₋₇ alkenyl.

In some embodiments, R⁶ is optionally substituted C₈₋₅₀ alkenyl. In some embodiments, R⁶ is optionally substituted C₈₋₄₀ alkenyl. In some embodiments, R⁶ is optionally substituted C₈₋₃₀ alkenyl. In some embodiments, R⁶ is optionally substituted C₈₋₂₀ alkenyl. In some embodiments, R⁶ is optionally substituted C₈₋₁₉ alkenyl. In some embodiments, R⁶ is optionally substituted C₈₋₁₈ alkenyl. In some embodiments, R⁶ is optionally substituted C₈₋₁₇ alkenyl. In some embodiments, R⁶ is optionally substituted C₈₋₁₆ alkenyl. In some embodiments, R⁶ is optionally substituted C₈₋₁₅ alkenyl. In some embodiments, R⁶ is optionally substituted C₈₋₁₄ alkenyl. In some embodiments, R⁶ is optionally substituted C₈₋₁₃ alkenyl. In some embodiments, R⁶ is optionally substituted C₉₋₁₂ alkenyl. In some embodiments, R⁶ is optionally substituted C₉₋₁₁ alkenyl. In some embodiments, R⁶ is optionally substituted C₉₋₁₀ alkenyl. In some embodiments, R⁶ is optionally substituted C₈₋₉ alkenyl.

In some embodiments, R⁶ is optionally substituted C₉₋₅₀ alkenyl. In some embodiments, R⁶ is optionally substituted C₉₋₄₀ alkenyl. In some embodiments, R⁶ is optionally substituted C₉₋₃₀ alkenyl. In some embodiments, R⁶ is optionally substituted C₉₋₂₀ alkenyl. In some embodiments, R⁶ is optionally substituted C₉₋₁₉ alkenyl. In some embodiments, R⁶ is optionally substituted C₉₋₁₈ alkenyl. In some embodiments, R⁶ is optionally substituted C₉₋₁₇ alkenyl. In some embodiments, R⁶ is optionally substituted C₉₋₁₆ alkenyl. In some embodiments, R⁶ is optionally substituted C₉₋₁₅ alkenyl. In some embodiments, R⁶ is optionally substituted C₉₋₁₄ alkenyl. In some embodiments, R⁶ is optionally substituted C₉₋₁₃ alkenyl. In some embodiments, R⁶ is optionally substituted C₉₋₁₂ alkenyl. In some embodiments, R⁶ is optionally substituted C₉₋₁₁ alkenyl. In some embodiments, R⁶ is optionally substituted C₉₋₁₀ alkenyl.

In some embodiments, R⁶ is optionally substituted C₁₀₋₅₀ alkenyl. In some embodiments, R⁶ is optionally substituted C₁₀₋₄₀ alkenyl. In some embodiments, R⁶ is optionally substituted C₁₀₋₃₀ alkenyl. In some embodiments, R⁶ is optionally substituted C₁₀₋₂₀ alkenyl. In some embodiments, R⁶ is optionally substituted C₁₀₋₁₉ alkenyl. In some embodiments, R⁶ is optionally substituted C₁₀₋₁₈ alkenyl. In some embodiments, R⁶ is optionally substituted C₁₀₋₁₇ alkenyl. In some embodiments, R⁶ is optionally substituted C₁₀₋₁₆ alkenyl. In some embodiments, R⁶ is optionally substituted C₁₀₋₁₂ alkenyl. In some embodiments, R⁶ is optionally substituted C₁₀₋₁₄ alkenyl. In some embodiments, R⁶ is optionally substituted C₁₀₋₁₃ alkenyl. In some embodiments, R⁶ is optionally substituted C₁₀₋₁₂ alkenyl. In some embodiments, R⁶ is optionally substituted C₁₀₋₁₁ alkenyl.

In some embodiments, R⁶ is optionally substituted C₁₁₋₅₀ alkenyl. In some embodiments, R⁶ is optionally substituted C₁₁₋₄₀ alkenyl. In some embodiments, R⁶ is optionally substituted C₁₁₋₃₀ alkenyl. In some embodiments, R⁶ is optionally substituted C₁₁₋₂₀ alkenyl. In some embodiments, R⁶ is optionally substituted C₁₁₋₁₉ alkenyl. In some embodiments, R⁶ is optionally substituted C₁₁₋₁₈ alkenyl. In some embodiments, R⁶ is optionally substituted C₁₁₋₁₇ alkenyl. In some embodiments, R⁶ is optionally substituted C₁₁₋₁₆ alkenyl. In some embodiments, R⁶ is optionally substituted C₁₁₋₁₅ alkenyl. In some embodiments, R⁶ is optionally substituted C₁₁₋₁₄ alkenyl. In some embodiments, R⁶ is optionally substituted C₁₁₋₁₃ alkenyl. In some embodiments, R⁶ is optionally substituted C₁₁₋₁₂ alkenyl.

In some embodiments, R⁶ is optionally substituted C₁₂₋₅₀ alkenyl. In some embodiments, R⁶ is optionally substituted C₁₂₋₄₀ alkenyl. In some embodiments, R⁶ is optionally substituted C₁₂₋₃₀ alkenyl. In some embodiments, R⁶ is optionally substituted C₁₂₋₂₀ alkenyl. In some embodiments, R⁶ is optionally substituted C₁₂₋₁₉ alkenyl. In some embodiments, R⁶ is optionally substituted C₁₂₋₁₈ alkenyl. In some embodiments, R⁶ is optionally substituted C₁₂₋₁₇ alkenyl. In some embodiments, R⁶ is optionally substituted C₁₂₋₁₆ alkenyl. In some embodiments, R⁶ is optionally substituted C₁₂₋₁₅ alkenyl. In some embodiments, R⁶ is optionally substituted C₁₂₋₁₄ alkenyl. In some embodiments, R⁶ is optionally substituted C₁₂₋₁₃ alkenyl.

In some embodiments, R⁶ is optionally substituted C₆ alkenyl. In some embodiments, R⁶ is optionally substituted C₇ alkenyl. In some embodiments, R⁶ is optionally substituted C₈ alkenyl. In some embodiments, R⁶ is optionally substituted C₉ alkenyl. In some embodiments, R⁶ is optionally substituted C₁₀ alkenyl. In some embodiments, R⁶ is optionally substituted C₁₁ alkenyl. In some embodiments, R⁶ is optionally substituted C₁₂ alkenyl. In some embodiments, R⁶ is optionally substituted C₁₃ alkenyl. In some embodiments, R⁶ is optionally substituted C₁₄ alkenyl. In some embodiments, R⁶ is optionally substituted C₁₂ alkenyl. In some embodiments, R⁶ is optionally substituted C₁₆ alkenyl. In some embodiments, R⁶ is optionally substituted C₁₇ alkenyl. In some embodiments, R⁶ is optionally substituted C₁₈ alkenyl. In some embodiments, R⁶ is optionally substituted C₁₉ alkenyl. In some embodiments, R⁶ is optionally substituted C₂₀ alkenyl.

In some embodiments, for example, in any of the above embodiments, R⁶ is a substituted alkenyl group. In some embodiments, R⁶ is an unsubstituted alkenyl group. In some embodiments, R⁶ is an optionally substituted straight-chain alkenyl group. In some embodiments, R⁶ is a substituted straight-chain alkenyl group. In some embodiments, R⁶ is an unsubstituted straight-chain alkenyl group. In some embodiments, R⁶ is an optionally substituted branched alkenyl group. In some embodiments, R⁶ is a substituted branched alkenyl group. In some embodiments, R⁶ is an unsubstituted branched alkenyl group.

In some embodiments, R⁶ is optionally substituted alkynyl. In some embodiments, R⁶ is optionally substituted C₂₋₅₀ alkynyl. In some embodiments, R⁶ is optionally substituted C₂₋₄₀ alkynyl. In some embodiments, R⁶ is optionally substituted C₂₋₃₀ alkynyl. In some embodiments, R⁶ is optionally substituted C₂₋₂₀ alkynyl. In some embodiments, R⁶ is optionally substituted C₂₋₁₉ alkynyl. In some embodiments, R⁶ is optionally substituted C₂₋₈ alkynyl. In some embodiments, R⁶ is optionally substituted C₂₋₇ alkynyl. In some embodiments, R⁶ is optionally substituted C₂₋₁₆ alkynyl. In some embodiments, R⁶ is optionally substituted C₂₋₁₅ alkynyl. In some embodiments, R⁶ is optionally substituted C₂₋₁₄ alkynyl. In some embodiments, R⁶ is optionally substituted C₂₋₁₃ alkynyl. In some embodiments, R⁶ is optionally substituted C₂₋₁₂ alkynyl. In some embodiments, R⁶ is optionally substituted C₂₋₁₁ alkynyl. In some embodiments, R⁶ is optionally substituted C₂₋₁₀ alkynyl. In some embodiments, R⁶ is optionally substituted C₂₋₉ alkynyl. In some embodiments, R⁶ is optionally substituted C_{z-g} alkynyl. In some embodiments, R⁶ is optionally substituted C₂₋₇ alkynyl. In some embodiments, R⁶ is optionally substituted C₂₋₆ alkynyl.

In some embodiments, R⁶ is optionally substituted C₄₋₅₀ alkynyl. In some embodiments, R⁶ is optionally substituted C₄₋₄₀ alkynyl. In some embodiments, R⁶ is optionally substituted C₄₋₃₀ alkynyl. In some embodiments, R⁶ is optionally substituted C₄₋₂₀ alkynyl. In some embodiments, R⁶ is optionally substituted C₄₋₁₉ alkynyl. In some embodiments, R⁶ is optionally substituted C₄₋₁₈ alkynyl. In some embodiments, R⁶ is optionally substituted C₄₋₇ alkynyl. In some embodiments, R⁶ is optionally substituted C₄₋₁₆ alkynyl. In some embodiments, R⁶ is optionally substituted C₄₋₁₅ alkynyl. In some embodiments, R⁶ is optionally substituted C₄₋₁₄ alkynyl. In some embodiments, R⁶ is optionally substituted C₄₋₁₃ alkynyl. In some embodiments, R⁶ is optionally substituted C₄₋₁₂ alkynyl. In some embodiments, R⁶ is optionally substituted C₄₋₁₁ alkynyl. In some embodiments, R⁶ is optionally substituted C₄₋₁₀ alkynyl. In some embodiments, R⁶ is optionally substituted C₄₋₉ alkynyl. In some embodiments, R⁶ is optionally substituted C_{4-g} alkynyl. In some embodiments, R⁶ is optionally substituted C₄₋₇ alkynyl. In some embodiments, R⁶ is optionally substituted C₄₋₆ alkynyl.

In some embodiments, R⁶ is optionally substituted C₆₋₅₀ alkynyl. In some embodiments, R⁶ is optionally substituted C₆₋₄₀ alkynyl. In some embodiments, R⁶ is optionally substituted C₆₋₃₀ alkynyl. In some embodiments, R⁶ is optionally substituted C₆₋₂₀ alkynyl. In some embodiments, R⁶ is optionally substituted C₆₋₁₉ alkynyl. In some embodiments, R⁶ is optionally substituted C₆₋₁₈ alkynyl. In some embodiments, R⁶ is optionally substituted C₆₋₁₇ alkynyl. In some embodiments, R⁶ is optionally substituted C₆₋₁₆ alkynyl. In some embodiments, R⁶ is optionally substituted C₆₋₁₅ alkynyl. In some embodiments, R⁶ is optionally substituted C₆₋₁₄ alkynyl. In some embodiments, R⁶ is optionally substituted C₆₋₁₃ alkynyl. In some embodiments, R⁶ is optionally substituted C₆₋₁₂ alkynyl. In some embodiments, R⁶ is optionally substituted C₆₋₁₁ alkynyl. In some embodiments, R⁶ is optionally substituted C₆₋₁₀ alkynyl. In some embodiments, R⁶ is optionally substituted C₆₋₉ alkynyl. In some embodiments, R⁶ is optionally substituted C_{6-g} alkynyl. In some embodiments, R⁶ is optionally substituted C₆₋₇ alkynyl.

In some embodiments, R⁶ is optionally substituted C₈₋₅₀ alkynyl. In some embodiments, R⁶ is optionally substituted C₈₋₄₀ alkynyl. In some embodiments, R⁶ is optionally substituted C₈₋₃₀ alkynyl. In some embodiments, R⁶ is optionally substituted C₈₋₂₀ alkynyl. In some embodiments, R⁶ is optionally substituted C₆₋₁₉ alkynyl. In some embodiments, R⁶ is optionally substituted C₈₋₁₈ alkynyl. In some embodiments, R⁶ is optionally substituted C₈₋₁₇ alkynyl. In some embodiments, R⁶ is optionally substituted C₈₋₁₆ alkynyl. In some embodiments, R⁶ is optionally substituted C₈₋₁₅ alkynyl. In some embodiments, R⁶ is optionally substituted C₈₋₁₄ alkynyl. In some embodiments, R⁶ is optionally substituted C₈₋₁₃ alkynyl. In some embodiments, R⁶ is optionally substituted C₈₋₁₂ alkynyl. In some embodiments, R⁶ is optionally substituted C₈₋₁₁ alkynyl. In some embodiments, R⁶ is optionally substituted C₈₋₁₀ alkynyl. In some embodiments, R⁶ is optionally substituted C₈₋₉ alkynyl.

In some embodiments, R⁶ is optionally substituted C₉₋₅₀ alkynyl. In some embodiments, R⁶ is optionally substituted C₉₋₄₀ alkynyl. In some embodiments, R⁶ is optionally substituted C₉₋₃₀ alkynyl. In some embodiments, R⁶ is optionally substituted C₉₋₂₀ alkynyl. In some embodiments, R⁶ is optionally substituted C₉₋₁₉ alkynyl. In some embodiments, R⁶ is optionally substituted C₉₋₁₈ alkynyl. In some embodiments, R⁶ is optionally substituted C₉₋₇ alkynyl. In some embodiments, R⁶ is optionally substituted C₉₋₁₆ alkynyl. In some embodiments, R⁶ is optionally substituted C₉₋₁₅ alkynyl. In some embodiments, R⁶ is optionally substituted C₉₋₁₄ alkynyl. In some embodiments, R⁶ is optionally substituted C₉₋₁₃ alkynyl. In some embodiments, R⁶ is optionally substituted C₉₋₁₂ alkynyl. In some embodiments, R⁶ is optionally substituted C₉₋₁₁ alkynyl. In some embodiments, R⁶ is optionally substituted C₉₋₁₀ alkynyl.

In some embodiments, R⁶ is optionally substituted C₁₀₋₅₀ alkynyl. In some embodiments, R⁶ is optionally substituted C₁₀₋₄₀ alkynyl. In some embodiments, R⁶ is optionally substituted C₁₀₋₃₀ alkynyl. In some embodiments, R⁶ is optionally substituted C₁₀₋₂₀ alkynyl. In some embodiments, R⁶ is optionally substituted C₁₀₋₁₉ alkynyl. In some embodiments, R⁶ is optionally substituted C₁₀₋₁₈ alkynyl. In some embodiments, R⁶ is optionally substituted C₁₀₋₁₇ alkynyl. In some embodiments, R⁶ is optionally substituted C₁₀₋₁₆ alkynyl. In some embodiments, R⁶ is optionally substituted C₁₀₋₁₅ alkynyl. In some embodiments, R⁶ is optionally substituted C₁₀₋₁₄ alkynyl. In some embodiments, R⁶ is optionally substituted C₁₀₋₁₃ alkynyl. In some embodiments, R⁶ is optionally substituted C₁₀₋₁₂ alkynyl. In some embodiments, R⁶ is optionally substituted C₁₀₋₁₁ alkynyl.

In some embodiments, R⁶ is optionally substituted C₁₁₋₅₀ alkynyl. In some embodiments, R⁶ is optionally substituted C₁₁₋₄₀ alkynyl. In some embodiments, R⁶ is optionally substituted C₁₁₋₃₀ alkynyl. In some embodiments, R⁶ is optionally substituted C₁₁₋₂₀ alkynyl. In some embodiments, R⁶ is optionally substituted C₁₁₋₁₉ alkynyl. In some embodiments, R⁶ is optionally substituted C₁₁₋₁₈ alkynyl. In some embodiments, R⁶ is optionally substituted C₁₁₋₁₇ alkynyl. In some embodiments, R⁶ is optionally substituted C₁₁₋₁₆ alkynyl. In some embodiments, R⁶ is optionally substituted C₁₁₋₁₅ alkynyl. In some embodiments, R⁶ is optionally substituted C₁₁₋₁₄ alkynyl. In some embodiments, R⁶ is optionally substituted C₁₁₋₁₃ alkynyl. In some embodiments, R⁶ is optionally substituted C₁₁₋₁₂ alkynyl.

In some embodiments, R⁶ is optionally substituted C₁₂₋₅₀ alkynyl. In some embodiments, R⁶ is optionally substituted C₁₂₋₄₀ alkynyl. In some embodiments, R⁶ is optionally substituted C₁₂₋₃₀ alkynyl. In some embodiments, R⁶ is optionally substituted C₁₂₋₂₀ alkynyl. In some embodiments, R⁶ is optionally substituted C₁₂₋₁₉ alkynyl. In some embodiments, R⁶ is optionally substituted C₁₂₋₁₈ alkynyl. In some embodiments, R⁶ is optionally substituted C₁₂₋₁₇ alkynyl. In some embodiments, R⁶ is optionally substituted C₁₂₋₁₆ alkynyl. In some embodiments, R⁶ is optionally substituted C₁₂₋₁₅ alkynyl. In some embodiments, R⁶ is optionally substituted C₁₂₋₁₄ alkynyl. In some embodiments, R⁶ is optionally substituted C₁₂₋₁₃ alkynyl.

In some embodiments, R⁶ is optionally substituted C₆ alkynyl. In some embodiments, R⁶ is optionally substituted C₇ alkynyl. In some embodiments, R⁶ is optionally substituted C₈ alkynyl. In some embodiments, R⁶ is optionally substituted C₉ alkynyl. In some embodiments, R⁶ is optionally substituted C₁₀ alkynyl. In some embodiments, R⁶ is optionally substituted C₁₁ alkynyl. In some embodiments, R⁶ is optionally substituted C₁₂ alkynyl. In some embodiments, R⁶ is optionally substituted C₁₃ alkynyl. In some embodiments, R⁶ is optionally substituted C₁₄ alkynyl. In some embodiments, R⁶ is optionally substituted C₁₅ alkynyl. In some embodiments, R⁶ is optionally substituted C₁₆ alkynyl. In some embodiments, R⁶ is optionally substituted C₁₇ alkynyl. In some embodiments, R⁶ is optionally substituted C₁₈ alkynyl. In some embodiments, R⁶ is optionally substituted C₁₉ alkynyl. In some embodiments, R⁶ is optionally substituted C₂₀ alkynyl.

In some embodiments, for example, in any of the above embodiments, R⁶ is a substituted alkynyl group. In some embodiments, R⁶ is an unsubstituted alknyl group. In some embodiments, R⁶ is an optionally substituted straight-chain alkynyl group. In some embodiments, R⁶ is a substituted straight-chain alkynyl group. In some embodiments, R⁶ is an unsubstituted straight-chain alkynyl group. In some embodiments, R⁶ is an optionally substituted branched alkynyl group. In some embodiments, R⁶ is a substituted branched alkynyl group. In some embodiments, R⁶ is an unsubstituted branched alkynyl group.

In some embodiments, R⁶ is optionally substituted carbocyclyl. In some embodiments, R⁶ is optionally substituted heterocyclyl. In some embodiments, R⁶ is optionally substituted aryl. In some embodiments, R⁶ is optionally substituted heteroaryl. In some embodiments, R⁶ is a nitrogen protecting group.

In some embodiments, R⁶ is a group of formula (i). In some embodiments, R⁶ is a group of formula (i-a). In some embodiments, R⁶ is a group of formula

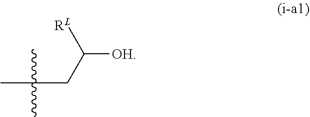

In some embodiments, R⁶ is a group of formula (i-b). In some embodiments, R^{b} is a group of formula (ii). In some embodiments, R⁶ is a group of formula (iii).

In some embodiments, R⁶ is substituted with one or more hydroxyl groups. In some embodiments, R⁶ is substituted with one hydroxyl group. In some embodiments, R⁶ is substituted with one 2-hydroxyl group (C1 is the carbon atom directly bonded to the nitrogen atom depicted in formula (iv)).

As generally defined above, each R⁷ is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group, or a group of formula (i), (ii) or (iii).

In some embodiments, R⁷ is hydrogen.

In some embodiments, R⁷ is optionally substituted alkyl. In some embodiments, R⁷ is optionally substituted C₂₋₅₀ alkyl. In some embodiments, R⁷ is optionally substituted C₂₋₄₀ alkyl. In some embodiments, R⁷ is optionally substituted C₂₋₃₀ alkyl. In some embodiments, R⁷ is optionally substituted C₂₋₂₀ alkyl. In some embodiments, R⁷ is optionally substituted C₂₋₁₉ alkyl. In some embodiments, R⁷ is optionally substituted C₂₋₁₈ alkyl. In some embodiments, R⁷ is optionally substituted C₂₋₇ alkyl. In some embodiments, R⁷ is optionally substituted C₂₋₁₆ alkyl. In some embodiments, R⁷ is optionally substituted C₂₋₁₅ alkyl. In some embodiments, R⁷ is optionally substituted C₂₋₁₄ alkyl. In some embodiments, R⁷ is optionally substituted C₂₋₃ alkyl. In some embodiments, R⁷ is optionally substituted C₂₋₁₂ alkyl. In some embodiments, R⁷ is optionally substituted C₂₋₁₁ alkyl. In some embodiments, R⁷ is optionally substituted C₂₋₁₀ alkyl. In some embodiments, R⁷ is optionally substituted C₂₋₉ alkyl. In some embodiments, R⁷ is optionally substituted C₂₋₈ alkyl. In some embodiments, R⁷ is optionally substituted C₂₋₇ alkyl. In some embodiments, R⁷ is optionally substituted C₂₋₆ alkyl.

In some embodiments, R⁷ is optionally substituted C₄₋₅₀ alkyl. In some embodiments, R⁷ is optionally substituted C₄₋₄₀ alkyl. In some embodiments, R⁷ is optionally substituted C₄₋₃₀ alkyl. In some embodiments, R⁷ is optionally substituted C₄₋₂₀ alkyl. In some embodiments, R⁷ is optionally substituted C₄₋₁₉ alkyl. In some embodiments, R⁷ is optionally substituted C₄₋₁₈ alkyl. In some embodiments, R⁷ is optionally substituted C₄₋₇ alkyl. In some embodiments, R⁷ is optionally substituted C₄₋₁₆ alkyl. In some embodiments, R⁷ is optionally substituted C₄₋₁₅ alkyl. In some embodiments, R⁷ is optionally substituted C₄₋₁₄ alkyl. In some embodiments, R⁷ is optionally substituted C₄₋₁₃ alkyl. In some embodiments, R⁷ is optionally substituted C₄₋₁₂ alkyl. In some embodiments, R⁷ is optionally substituted C₄₋₁₁ alkyl. In some embodiments, R⁷ is optionally substituted C₄₋₁₀ alkyl. In some embodiments, R⁷ is optionally substituted C₄₋₉ alkyl. In some embodiments, R⁷ is optionally substituted C₄₋₈ alkyl. In some embodiments, R⁷ is optionally substituted C₄₋₇ alkyl. In some embodiments, R⁷ is optionally substituted C₄₋₆ alkyl.

In some embodiments, R⁷ is optionally substituted C₆₋₅₀ alkyl. In some embodiments, R⁷ is optionally substituted C₆₋₄₀ alkyl. In some embodiments, R⁷ is optionally substituted C₆₋₃₀ alkyl. In some embodiments, R⁷ is optionally substituted C₆₋₂₀ alkyl. In some embodiments, R⁷ is optionally substituted C₆₋₁₉ alkyl. In some embodiments, R⁷ is optionally substituted C₆₋₁₈ alkyl. In some embodiments, R⁷ is optionally substituted C₆₋₇ alkyl. In some embodiments, R⁷ is optionally substituted C₆₋₁₆ alkyl. In some embodiments, R⁷ is optionally substituted C₆₋₁₅ alkyl. In some embodiments, R⁷ is optionally substituted C₆₋₁₄ alkyl. In some embodiments, R⁷ is optionally substituted C₆₋₁₃ alkyl. In some embodiments, R⁷ is optionally substituted C₆₋₁₂ alkyl. In some embodiments, R⁷ is optionally substituted C₆₋₁₁ alkyl. In some embodiments, R⁷ is optionally substituted C₆₋₁₀ alkyl. In some embodiments, R⁷ is optionally substituted C₆₋₉ alkyl. In some embodiments, R⁷ is optionally substituted C_{6-g} alkyl. In some embodiments, R⁷ is optionally substituted C₆₋₇ alkyl.

In some embodiments, R⁷ is optionally substituted C₈₋₅₀ alkyl. In some embodiments, R⁷ is optionally substituted C₈₋₄₀ alkyl. In some embodiments, R⁷ is optionally substituted C₈₋₃₀ alkyl. In some embodiments, R⁷ is optionally substituted C₈₋₂₀ alkyl. In some embodiments, R⁷ is optionally substituted C₈₋₁₉ alkyl. In some embodiments, R⁷ is optionally substituted C₈₋₁₈ alkyl. In some embodiments, R⁷ is optionally substituted C₈₋₇ alkyl. In some embodiments, R⁷ is optionally substituted C₈₋₁₆ alkyl. In some embodiments, R⁷ is optionally substituted C₈₋₁₅ alkyl. In some embodiments, R⁷ is optionally substituted C₈₋₁₄ alkyl. In some embodiments, R⁷ is optionally substituted C₈₋₁₃ alkyl. In some embodiments, R⁷ is optionally substituted C₈₋₁₂ alkyl. In some embodiments, R⁷ is optionally substituted C₈₋₁₁ alkyl. In some embodiments, R⁷ is optionally substituted C₈₋₁₀ alkyl. In some embodiments, R⁷ is optionally substituted C₈₋₉ alkyl.

In some embodiments, R⁷ is optionally substituted C₉₋₅₀ alkyl. In some embodiments, R⁷ is optionally substituted C₉₋₄₀ alkyl. In some embodiments, R⁷ is optionally substituted C₉₋₃₀ alkyl. In some embodiments, R⁷ is optionally substituted C₉₋₂₀ alkyl. In some embodiments, R⁷ is optionally substituted C₉₋₁₉ alkyl. In some embodiments, R⁷ is optionally substituted C₉₋₁₈ alkyl. In some embodiments, R⁷ is optionally substituted C₉₋₁₇ alkyl. In some embodiments, R⁷ is optionally substituted C₉₋₁₆ alkyl. In some embodiments, R⁷ is optionally substituted C₉₋₁₈ alkyl. In some embodiments, R⁷ is optionally substituted C₉₋₁₄ alkyl. In some embodiments, R⁷ is optionally substituted C₉₋₁₃ alkyl. In some embodiments, R⁷ is optionally substituted C₉₋₁₂ alkyl. In some embodiments, R⁷ is optionally substituted C₉₋₁₁ alkyl. In some embodiments, R⁷ is optionally substituted C₉₋₁₀ alkyl.

In some embodiments, R⁷ is optionally substituted C₁₀₋₅₀ alkyl. In some embodiments, R⁷ is optionally substituted C₁₀₋₄₀ alkyl. In some embodiments, R⁷ is optionally substituted C₁₀₋₃₀ alkyl. In some embodiments, R⁷ is optionally substituted C₁₀₋₂₀ alkyl. In some embodiments, R⁷ is optionally substituted C₁₀₋₁₉ alkyl. In some embodiments, R⁷ is optionally substituted C₁₀₋₁₈ alkyl. In some embodiments, R⁷ is optionally substituted C₁₀₋₁₇ alkyl. In some embodiments, R⁷ is optionally substituted C₁₀₋₁₆ alkyl. In some embodiments, R⁷ is optionally substituted C₁₀₋₁₅ alkyl. In some embodiments, R⁷ is optionally substituted C₁₀₋₁₄ alkyl. In some embodiments, R⁷ is optionally substituted C₁₀₋₁₃ alkyl. In some embodiments, R⁷ is optionally substituted C₁₀₋₁₂ alkyl. In some embodiments, R⁷ is optionally substituted C₁₀₋₁₁ alkyl.

In some embodiments, R⁷ is optionally substituted C₁₁₋₅₀ alkyl. In some embodiments, R⁷ is optionally substituted C₁₁₋₄₀ alkyl. In some embodiments, R⁷ is optionally substituted C₁₁₋₃₀ alkyl. In some embodiments, R⁷ is optionally substituted C₁₁₋₂₀ alkyl. In some embodiments, R⁷ is optionally substituted C₁₁₋₁₉ alkyl. In some embodiments, R⁷ is optionally substituted C₁₁₋₁₈ alkyl. In some embodiments, R⁷ is optionally substituted C₁₁₋₁₇ alkyl. In some embodiments, R⁷ is optionally substituted C₁₁₋₁₆ alkyl. In some embodiments, R⁷ is optionally substituted C₁₁₋₁₅ alkyl. In some embodiments, R⁷ is optionally substituted C₁₁₋₁₄ alkyl. In some embodiments, R⁷ is optionally substituted C₁₁₋₁₃ alkyl. In some embodiments, R⁷ is optionally substituted C₁₁₋₁₂ alkyl.

In some embodiments, R⁷ is optionally substituted C₁₂₋₅₀ alkyl. In some embodiments, R⁷ is optionally substituted C₁₂₋₄₀ alkyl. In some embodiments, R⁷ is optionally substituted C₁₂₋₃₀ alkyl. In some embodiments, R⁷ is optionally substituted C₁₂₋₂₀ alkyl. In some embodiments, R⁷ is optionally substituted C₁₂₋₁₉ alkyl. In some embodiments, R⁷ is optionally substituted C₁₂₋₁₈ alkyl. In some embodiments, R⁷ is optionally substituted C₁₂₋₁₇ alkyl. In some embodiments, R⁷ is optionally substituted C₁₂₋₁₆ alkyl. In some embodiments, R⁷ is optionally substituted C₁₂₋₁₅ alkyl. In some embodiments, R⁷ is optionally substituted C₁₂₋₁₄ alkyl. In some embodiments, R⁷ is optionally substituted C₁₂₋₁₃ alkyl.

In some embodiments, R⁷ is optionally substituted C₆ alkyl. In some embodiments, R⁷ is optionally substituted C₇ alkyl. In some embodiments, R⁷ is optionally substituted C₈ alkyl. In some embodiments, R⁷ is optionally substituted C₉ alkyl. In some embodiments, R⁷ is optionally substituted C₁₀ alkyl. In some embodiments, R⁷ is optionally substituted C₁₁ alkyl. In some embodiments, R⁷ is optionally substituted C₁₂ alkyl. In some embodiments, R⁷ is optionally substituted C₁₃ alkyl. In some embodiments, R⁷ is optionally substituted C₁₄ alkyl. In some embodiments, R⁷ is optionally substituted C₁₅ alkyl. In some embodiments, R⁷ is optionally substituted C₁₆ alkyl. In some embodiments, R⁷ is optionally substituted C₁₇ alkyl. In some embodiments, R⁷ is optionally substituted C₁₈ alkyl. In some embodiments, R⁷ is optionally substituted C₁₉ alkyl. In some embodiments, R⁷ is optionally substituted C₂₀ alkyl.

In some embodiments, for example, in any of the above embodiments, R⁷ is a substituted alkyl group. In some embodiments, R⁷ is an unsubstituted alkyl group. In some embodiments, R⁷ is an optionally substituted straight-chain alkyl group. In some embodiments, R⁷ is a substituted straight-chain alkyl group. In some embodiments, R⁷ is an unsubstituted straight-chain alkyl group. In some embodiments, R⁷ is an optionally substituted branched alkyl group. In some embodiments, R⁷ is a substituted branched alkyl group. In some embodiments, R⁷ is an unsubstituted branched alkyl group.

In some embodiments, R⁷ is optionally substituted alkenyl. In some embodiments, R⁷ is optionally substituted C₂₋₅₀ alkenyl. In some embodiments, R⁷ is optionally substituted C₂₋₄₀ alkenyl. In some embodiments, R⁷ is optionally substituted C₂₋₃₀ alkenyl. In some embodiments, R⁷ is optionally substituted C₂₋₂₀ alkenyl. In some embodiments, R⁷ is optionally substituted C₂₋₁₉ alkenyl. In some embodiments, R⁷ is optionally substituted C₂₋₈ alkenyl. In some embodiments, R⁷ is optionally substituted C₂₋₇ alkenyl. In some embodiments, R⁷ is optionally substituted C₂₋₁₆ alkenyl. In some embodiments, R⁷ is optionally substituted C₂₋₁₅ alkenyl. In some embodiments, R⁷ is optionally substituted C₂₋₁₄ alkenyl. In some embodiments, R⁷ is optionally substituted C₂₋₁₃ alkenyl. In some embodiments, R⁷ is optionally substituted C₂₋₁₂ alkenyl. In some embodiments, R⁷ is optionally substituted C₂₋₁₁ alkenyl. In some embodiments, R⁷ is optionally substituted C₂₋₁₀ alkenyl. In some embodiments, R⁷ is optionally substituted C₂₋₉ alkenyl. In some embodiments, R⁷ is optionally substituted C₂₋₈ alkenyl. In some embodiments, R⁷ is optionally substituted C₂₋₇ alkenyl. In some embodiments, R⁷ is optionally substituted C₂₋₆ alkenyl.

In some embodiments, R⁷ is optionally substituted C₄₋₅₀ alkenyl. In some embodiments, R⁷ is optionally substituted C₄₋₄₀ alkenyl. In some embodiments, R⁷ is optionally substituted C₄₋₃₀ alkenyl. In some embodiments, R⁷ is optionally substituted C₄₋₂₀ alkenyl. In some embodiments, R⁷ is optionally substituted C₄₋₁₉ alkenyl. In some embodiments, R⁷ is optionally substituted C₄₋₁₈ alkenyl. In some embodiments, R⁷ is optionally substituted C₄₋₇ alkenyl. In some embodiments, R⁷ is optionally substituted C₄₋₁₆ alkenyl. In some embodiments, R⁷ is optionally substituted C₄₋₁₅ alkenyl. In some embodiments, R⁷ is optionally substituted C₄₋₁₄ alkenyl. In some embodiments, R⁷ is optionally substituted C₄₋₁₃ alkenyl. In some embodiments, R⁷ is optionally substituted C₄₋₁₂ alkenyl. In some embodiments, R⁷ is optionally substituted C₄₋₁₁ alkenyl. In some embodiments, R⁷ is optionally substituted C₄₋₁₀ alkenyl. In some embodiments, R⁷ is optionally substituted C₄₋₉ alkenyl. In some embodiments, R⁷ is optionally substituted C₄₋₈ alkenyl. In some embodiments, R⁷ is optionally substituted C₄₋₇ alkenyl. In some embodiments, R⁷ is optionally substituted C₄₋₆ alkenyl.

In some embodiments, R⁷ is optionally substituted C₆₋₅₀ alkenyl. In some embodiments, R⁷ is optionally substituted C₆₋₄₀ alkenyl. In some embodiments, R⁷ is optionally substituted C₆₋₃₀ alkenyl. In some embodiments, R⁷ is optionally substituted C₆₋₂₀ alkenyl. In some embodiments, R⁷ is optionally substituted C₆₋₁₉ alkenyl. In some embodiments, R⁷ is optionally substituted C₆₋₁₈ alkenyl. In some embodiments, R⁷ is optionally substituted C₆₋₇ alkenyl. In some embodiments, R⁷ is optionally substituted C₆₋₁₆ alkenyl. In some embodiments, R⁷ is optionally substituted C₆₋₁₅ alkenyl. In some embodiments, R⁷ is optionally substituted C₆₋₁₄ alkenyl. In some embodiments, R⁷ is optionally substituted C₆₋₁₃ alkenyl. In some embodiments, R⁷ is optionally substituted C₆₋₁₂ alkenyl. In some embodiments, R⁷ is optionally substituted C₆₋₁₁ alkenyl. In some embodiments, R⁷ is optionally substituted C₆₋₁₀ alkenyl. In some embodiments, R⁷ is optionally substituted C₆₋₉ alkenyl. In some embodiments, R⁷ is optionally substituted C₆₋₈ alkenyl. In some embodiments, R⁷ is optionally substituted C₆₋₇ alkenyl.

In some embodiments, R⁷ is optionally substituted C₈₋₅₀ alkenyl. In some embodiments, R⁷ is optionally substituted C₈₋₄₀ alkenyl. In some embodiments, R⁷ is optionally substituted C₈₋₃₀ alkenyl. In some embodiments, R⁷ is optionally substituted C₈₋₂₀ alkenyl. In some embodiments, R⁷ is optionally substituted C₈₋₁₉ alkenyl. In some embodiments, R⁷ is optionally substituted C₈₋₁₈ alkenyl. In some embodiments, R⁷ is optionally substituted C₈₋₁₇ alkenyl. In some embodiments, R⁷ is optionally substituted C₈₋₁₆ alkenyl. In some embodiments, R⁷ is optionally substituted C₈₋₁₂ alkenyl. In some embodiments, R⁷ is optionally substituted C₈₋₁₄ alkenyl. In some embodiments, R⁷ is optionally substituted C₈₋₁₃ alkenyl. In some embodiments, R⁷ is optionally substituted C₈₋₁₂ alkenyl. In some embodiments, R⁷ is optionally substituted C₈₋₁₁ alkenyl. In some embodiments, R⁷ is optionally substituted C₈₋₁₀ alkenyl. In some embodiments, R⁷ is optionally substituted C₈₋₉ alkenyl.

In some embodiments, R⁷ is optionally substituted C₉₋₅₀ alkenyl. In some embodiments, R⁷ is optionally substituted C₉₋₄₀ alkenyl. In some embodiments, R⁷ is optionally substituted C₉₋₃₀ alkenyl. In some embodiments, R⁷ is optionally substituted C₉₋₂₀ alkenyl. In some embodiments, R⁷ is optionally substituted C₉₋₁₉ alkenyl. In some embodiments, R⁷ is optionally substituted C₉₋₁₈ alkenyl. In some embodiments, R⁷ is optionally substituted C₉₋₇ alkenyl. In some embodiments, R⁷ is optionally substituted C₉₋₁₆ alkenyl. In some embodiments, R⁷ is optionally substituted C₉₋₁₅ alkenyl. In some embodiments, R⁷ is optionally substituted C₉₋₁₄ alkenyl. In some embodiments, R⁷ is optionally substituted C₉₋₁₃ alkenyl. In some embodiments, R⁷ is optionally substituted C₉₋₁₂ alkenyl. In some embodiments, R⁷ is optionally substituted C₉₋₁₁ alkenyl. In some embodiments, R⁷ is optionally substituted C₉₋₁₀ alkenyl.

In some embodiments, R⁷ is optionally substituted C₁₀₋₅₀ alkenyl. In some embodiments, R⁷ is optionally substituted C₁₀₋₄₀ alkenyl. In some embodiments, R⁷ is optionally substituted C₁₀₋₃₀ alkenyl. In some embodiments, R⁷ is optionally substituted C₁₀₋₂₀ alkenyl. In some embodiments, R⁷ is optionally substituted C₁₀₋₁₉ alkenyl. In some embodiments, R⁷ is optionally substituted C₁₀₋₁₈ alkenyl. In some embodiments, R⁷ is optionally substituted C₁₀₋₁₇ alkenyl. In some embodiments, R⁷ is optionally substituted C₁₀₋₁₆ alkenyl. In some embodiments, R⁷ is optionally substituted C₁₀₋₁₅ alkenyl. In some embodiments, R⁷ is optionally substituted C₁₀₋₁₄ alkenyl. In some embodiments, R⁷ is optionally substituted C₁₀₋₁₃ alkenyl. In some embodiments, R⁷ is optionally substituted C₁₀₋₁₂ alkenyl. In some embodiments, R⁷ is optionally substituted C₁₀₋₁₁ alkenyl.

In some embodiments, R⁷ is optionally substituted C₁₁₋₅₀ alkenyl. In some embodiments, R⁷ is optionally substituted C₁₁₋₄₀ alkenyl. In some embodiments, R⁷ is optionally substituted C₁₁₋₃₀ alkenyl. In some embodiments, R⁷ is optionally substituted C₁₁₋₂₀ alkenyl. In some embodiments, R⁷ is optionally substituted C₁₁₋₁₉ alkenyl. In some embodiments, R⁷ is optionally substituted C₁₁₋₁₈ alkenyl. In some embodiments, R⁷ is optionally substituted C_{II-17} alkenyl. In some embodiments, R⁷ is optionally substituted C₁₁₋₁₆ alkenyl. In some embodiments, R⁷ is optionally substituted C₁₁₋₁₅ alkenyl. In some embodiments, R⁷ is optionally substituted C₁₁₋₁₄ alkenyl. In some embodiments, R⁷ is optionally substituted C₁₁₋₁₃ alkenyl. In some embodiments, R⁷ is optionally substituted C₁₁₋₁₂ alkenyl.

In some embodiments, R⁷ is optionally substituted C₁₂₋₅₀ alkenyl. In some embodiments, R⁷ is optionally substituted C₁₂₋₄₀ alkenyl. In some embodiments, R⁷ is optionally substituted C₁₂₋₃₀ alkenyl. In some embodiments, R⁷ is optionally substituted C₁₂₋₂₀ alkenyl. In some embodiments, R⁷ is optionally substituted C₁₂₋₁₉ alkenyl. In some embodiments, R⁷ is optionally substituted C₁₂₋₁₈ alkenyl. In some embodiments, R⁷ is optionally substituted C₁₂₋₁₇ alkenyl. In some embodiments, R⁷ is optionally substituted C₁₂₋₁₆ alkenyl. In some embodiments, R⁷ is optionally substituted C₁₂₋₁₅ alkenyl. In some embodiments, R⁷ is optionally substituted C₁₂₋₁₄ alkenyl. In some embodiments, R⁷ is optionally substituted C₁₂₋₁₃ alkenyl.

In some embodiments, R⁷ is optionally substituted C₆ alkenyl. In some embodiments, R⁷ is optionally substituted C₇ alkenyl. In some embodiments, R⁷ is optionally substituted C₈ alkenyl. In some embodiments, R⁷ is optionally substituted C₉ alkenyl. In some embodiments, R⁷ is optionally substituted C₁₀ alkenyl. In some embodiments, R⁷ is optionally substituted C₁₁ alkenyl. In some embodiments, R⁷ is optionally substituted C₁₂ alkenyl. In some embodiments, R⁷ is optionally substituted C₁₃ alkenyl. In some embodiments, R⁷ is optionally substituted C₁₄ alkenyl. In some embodiments, R⁷ is optionally substituted C₁₅ alkenyl. In some embodiments, R⁷ is optionally substituted C₁₆ alkenyl. In some embodiments, R⁷ is optionally substituted C₁₇ alkenyl. In some embodiments, R⁷ is optionally substituted C₁₈ alkenyl. In some embodiments, R⁷ is optionally substituted C₁₉ alkenyl. In some embodiments, R⁷ is optionally substituted C₂₀ alkenyl.

In some embodiments, for example, in any of the above embodiments, R⁷ is a substituted alkenyl group. In some embodiments, R⁷ is an unsubstituted alkenyl group. In some embodiments, R⁷ is an optionally substituted straight-chain alkenyl group. In some embodiments, R⁷ is a substituted straight-chain alkenyl group. In some embodiments, R⁷ is an unsubstituted straight-chain alkenyl group. In some embodiments, R⁷ is an optionally substituted branched alkenyl group. In some embodiments, R⁷ is a substituted branched alkenyl group. In some embodiments, R⁷ is an unsubstituted branched alkenyl group.

In some embodiments, R⁷ is optionally substituted alkynyl. In some embodiments, R⁷ is optionally substituted C₂₋₅₀ alkynyl. In some embodiments, R⁷ is optionally substituted C₂₋₄₀ alkynyl. In some embodiments, R⁷ is optionally substituted C₂₋₃₀ alkynyl. In some embodiments, R⁷ is optionally substituted C₂₋₂₀ alkynyl. In some embodiments, R⁷ is optionally substituted C₂₋₁₉ alkynyl. In some embodiments, R⁷ is optionally substituted C₂₋₁₈ alkynyl. In some embodiments, R⁷ is optionally substituted C₂₋₁₇ alkynyl. In some embodiments, R⁷ is optionally substituted C₂₋₁₆ alkynyl. In some embodiments, R⁷ is optionally substituted C₂₋₁₅ alkynyl. In some embodiments, R⁷ is optionally substituted C₂₋₁₄ alkynyl. In some embodiments, R⁷ is optionally substituted C₂₋₁₃ alkynyl. In some embodiments, R⁷ is optionally substituted C₂₋₁₂ alkynyl. In some embodiments, R⁷ is optionally substituted C₂₋₁₁ alkynyl. In some embodiments, R⁷ is optionally substituted C₂₋₁₀ alkynyl. In some embodiments, R⁷ is optionally substituted C₂₋₉ alkynyl. In some embodiments, R⁷ is optionally substituted C₂₋₈ alkynyl. In some embodiments, R⁷ is optionally substituted C₂₋₇ alkynyl. In some embodiments, R⁷ is optionally substituted C₂₋₆ alkynyl.

In some embodiments, R⁷ is optionally substituted C₄₋₅₀ alkynyl. In some embodiments, R⁷ is optionally substituted C₄₋₄₀ alkynyl. In some embodiments, R⁷ is optionally substituted C₄₋₃₀ alkynyl. In some embodiments, R⁷ is optionally substituted C₄₋₂₀ alkynyl. In some embodiments, R⁷ is optionally substituted C₄₋₁₉ alkynyl. In some embodiments, R⁷ is optionally substituted C₄₋₁₈ alkynyl. In some embodiments, R⁷ is optionally substituted C₄₋₇ alkynyl. In some embodiments, R⁷ is optionally substituted C₄₋₁₆ alkynyl. In some embodiments, R⁷ is optionally substituted C₄₋₁₅ alkynyl. In some embodiments, R⁷ is optionally substituted C₄₋₁₄ alkynyl. In some embodiments, R⁷ is optionally substituted C₄₋₁₃ alkynyl. In some embodiments, R⁷ is optionally substituted C₄₋₁₂ alkynyl. In some embodiments, R⁷ is optionally substituted C₄₋₁₁ alkynyl. In some embodiments, R⁷ is optionally substituted C₄₋₁₀ alkynyl. In some embodiments, R⁷ is optionally substituted C₄₋₉ alkynyl. In some embodiments, R⁷ is optionally substituted C₄₋₈ alkynyl. In some embodiments, R⁷ is optionally substituted C₄₋₇ alkynyl. In some embodiments, R⁷ is optionally substituted C₄₋₆ alkynyl.

In some embodiments, R⁷ is optionally substituted C₆₋₅₀ alkynyl. In some embodiments, R⁷ is optionally substituted C₆₋₄₀ alkynyl. In some embodiments, R⁷ is optionally substituted C₆₋₃₀ alkynyl. In some embodiments, R⁷ is optionally substituted C₆₋₂₀ alkynyl. In some embodiments, R⁷ is optionally substituted C₆₋₁₉ alkynyl. In some embodiments, R⁷ is optionally substituted C₆₋₁₈ alkynyl. In some embodiments, R⁷ is optionally substituted C₆₋₇ alkynyl. In some embodiments, R⁷ is optionally substituted C₆₋₁₆ alkynyl. In some embodiments, R⁷ is optionally substituted C₆₋₁₅ alkynyl. In some embodiments, R⁷ is optionally substituted C₆₋₁₄ alkynyl. In some embodiments, R⁷ is optionally substituted C₆₋₁₃ alkynyl. In some embodiments, R⁷ is optionally substituted C₆₋₁₂ alkynyl. In some embodiments, R⁷ is optionally substituted C₆₋₁₁ alkynyl. In some embodiments, R⁷ is optionally substituted C₆₋₁₀ alkynyl. In some embodiments, R⁷ is optionally substituted C₆₋₉ alkynyl. In some embodiments, R⁷ is optionally substituted C₆₋₈ alkynyl. In some embodiments, R⁷ is optionally substituted C₆₋₇ alkynyl.

In some embodiments, R⁷ is optionally substituted C₈₋₅₀ alkynyl. In some embodiments, R⁷ is optionally substituted C₈₋₄₀ alkynyl. In some embodiments, R⁷ is optionally substituted C₈₋₃₀ alkynyl. In some embodiments, R⁷ is optionally substituted C₈₋₂₀ alkynyl. In some embodiments, R⁷ is optionally substituted C₈₋₁₉ alkynyl. In some embodiments, R⁷ is optionally substituted C₈₋₁₈ alkynyl. In some embodiments, R⁷ is optionally substituted C₈₋₇ alkynyl. In some embodiments, R⁷ is optionally substituted C₈₋₁₆ alkynyl. In some embodiments, R⁷ is optionally substituted C₈₋₁₅ alkynyl. In some embodiments, R⁷ is optionally substituted C₈₋₁₄ alkynyl. In some embodiments, R⁷ is optionally substituted C₈₋₁₃ alkynyl. In some embodiments, R⁷ is optionally substituted C₈₋₁₂ alkynyl. In some embodiments, R⁷ is optionally substituted C₈₋₁₁ alkynyl. In some embodiments, R⁷ is optionally substituted C₈₋₁₀ alkynyl. In some embodiments, R⁷ is optionally substituted C₈₋₉ alkynyl.

In some embodiments, R⁷ is optionally substituted C₉₋₅₀ alkynyl. In some embodiments, R⁷ is optionally substituted C₉₋₄₀ alkynyl. In some embodiments, R⁷ is optionally substituted C₉₋₃₀ alkynyl. In some embodiments, R⁷ is optionally substituted C₉₋₂₀ alkynyl. In some embodiments, R⁷ is optionally substituted C₉₋₁₉ alkynyl. In some embodiments, R⁷ is optionally substituted C₉₋₁₈ alkynyl. In some embodiments, R⁷ is optionally substituted C₉₋₇ alkynyl. In some embodiments, R⁷ is optionally substituted C₉₋₁₆ alkynyl. In some embodiments, R⁷ is optionally substituted C₉₋₁₅ alkynyl. In some embodiments, R⁷ is optionally substituted C₉₋₁₄ alkynyl. In some embodiments, R⁷ is optionally substituted C₉₋₁₃ alkynyl. In some embodiments, R⁷ is optionally substituted C₉₋₁₂ alkynyl. In some embodiments, R⁷ is optionally substituted C₉₋₁₁ alkynyl. In some embodiments, R⁷ is optionally substituted C₉₋₁₀ alkynyl.

In some embodiments, R⁷ is optionally substituted C₁₀₋₅₀ alkynyl. In some embodiments, R⁷ is optionally substituted C₁₀₋₄₀ alkynyl. In some embodiments, R⁷ is optionally substituted C₁₀₋₃₀ alkynyl. In some embodiments, R⁷ is optionally substituted C₁₀₋₂₀ alkynyl. In some embodiments, R⁷ is optionally substituted C₁₀₋₁₉ alkynyl. In some embodiments, R⁷ is optionally substituted C₁₀₋₁₈ alkynyl. In some embodiments, R⁷ is optionally substituted C₁₀₋₁₇ alkynyl. In some embodiments, R⁷ is optionally substituted C₁₀₋₁₆ alkynyl. In some embodiments, R⁷ is optionally substituted C₁₀₋₁₅ alkynyl. In some embodiments, R⁷ is optionally substituted C₁₀₋₁₄ alkynyl. In some embodiments, R⁷ is optionally substituted C₁₀₋₁₃ alkynyl. In some embodiments, R⁷ is optionally substituted C₁₀₋₁₂ alkynyl. In some embodiments, R⁷ is optionally substituted C₁₀₋₁₁ alkynyl.

In some embodiments, R⁷ is optionally substituted C₁₁₋₅₀ alkynyl. In some embodiments, R⁷ is optionally substituted C₁₁₋₄₀ alkynyl. In some embodiments, R⁷ is optionally substituted C₁₁₋₃₀ alkynyl. In some embodiments, R⁷ is optionally substituted C₁₁₋₂₀ alkynyl. In some embodiments, R⁷ is optionally substituted C₁₁₋₁₉ alkynyl. In some embodiments, R⁷ is optionally substituted C₁₁₋₁₈ alkynyl. In some embodiments, R⁷ is optionally substituted C₁₁₋₁₇ alkynyl. In some embodiments, R⁷ is optionally substituted C₁₁₋₁₆ alkynyl. In some embodiments, R⁷ is optionally substituted C₁₁₋₁₅ alkynyl. In some embodiments, R⁷ is optionally substituted C₁₁₋₁₄ alkynyl. In some embodiments, R⁷ is optionally substituted C₁₁₋₁₃ alkynyl. In some embodiments, R⁷ is optionally substituted C₁₁₋₁₂ alkynyl.

In some embodiments, R⁷ is optionally substituted C₁₂₋₅₀ alkynyl. In some embodiments, R⁷ is optionally substituted C₁₂₋₄₀ alkynyl. In some embodiments, R⁷ is optionally substituted C₁₂₋₃₀ alkynyl. In some embodiments, R⁷ is optionally substituted C₁₂₋₂₀ alkynyl. In some embodiments, R⁷ is optionally substituted C₁₂₋₁₉ alkynyl. In some embodiments, R⁷ is optionally substituted C₁₂₋₁₈ alkynyl. In some embodiments, R⁷ is optionally substituted C₁₂₋₁₇ alkynyl. In some embodiments, R⁷ is optionally substituted C₁₂₋₁₆ alkynyl. In some embodiments, R⁷ is optionally substituted C₁₂₋₁₅ alkynyl. In some embodiments, R⁷ is optionally substituted C₁₂₋₁₄ alkynyl. In some embodiments, R⁷ is optionally substituted C₁₂₋₁₃ alkynyl.

In some embodiments, R⁷ is optionally substituted C₆ alkynyl. In some embodiments, R⁷ is optionally substituted C₇ alkynyl. In some embodiments, R⁷ is optionally substituted C₈ alkynyl. In some embodiments, R⁷ is optionally substituted C₉ alkynyl. In some embodiments, R⁷ is optionally substituted C₁₀ alkynyl. In some embodiments, R⁷ is optionally substituted C₁₁ alkynyl. In some embodiments, R⁷ is optionally substituted C₁₂ alkynyl. In some embodiments, R⁷ is optionally substituted C₁₃ alkynyl. In some embodiments, R⁷ is optionally substituted C₁₄ alkynyl. In some embodiments, R⁷ is optionally substituted C₁₅ alkynyl. In some embodiments, R⁷ is optionally substituted C₁₆ alkynyl. In some embodiments, R⁷ is optionally substituted C₁₇ alkynyl. In some embodiments, R⁷ is optionally substituted C₁₈ alkynyl. In some embodiments, R⁷ is optionally substituted C₁₉ alkynyl. In some embodiments, R⁷ is optionally substituted C₂₀ alkynyl.

In some embodiments, for example, in any of the above embodiments, R⁷ is a substituted alkynyl group. In some embodiments, R⁷ is an unsubstituted alkynyl group. In some embodiments, R⁷ is an optionally substituted straight-chain alkynyl group. In some embodiments, R⁷ is a substituted straight-chain alkynyl group. In some embodiments, R⁷ is an unsubstituted straight-chain alkynyl group. In some embodiments, R⁷ is an optionally substituted branched alkynyl group. In some embodiments, R⁷ is a substituted branched alkynyl group. In some embodiments, R⁷ is an unsubstituted branched alkynyl group.

In some embodiments, R⁷ is optionally substituted carbocyclyl. In some embodiments, R⁷ is optionally substituted heterocyclyl. In some embodiments, R⁷ is optionally substituted aryl. In some embodiments, R⁷ is optionally substituted heteroaryl. In some embodiments, R⁷ is a nitrogen protecting group.

In some embodiments, R⁷ is a group of formula (i). In some embodiments, R⁷ is a group of formula (i-a). In some embodiments, R⁷ is a group of formula

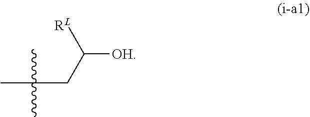

In some embodiments, R⁷ is a group of formula (i-b). In some embodiments, R⁷ is a group of formula (ii). In some embodiments, R⁷ is a group of formula (iii).

In some embodiments, at least one instance of R⁶ and R⁷ is a group of the formula (i), (ii) or (iii). In some embodiments, each instance of R⁶ and R⁷ is independently a group of the formula (i), (ii) or (iii). In some embodiments, each instance of R⁶ and R⁷ is independently a group of the formula (i). In some embodiments, each instance of R⁶ and R⁷ is independently a group of the formula (i-a). In some embodiments, each instance of R⁶ and R⁷ is independently a group of the formula (i-b). In some embodiments, each instance of R⁶ and R⁷ is independently a group of the formula (ii). In some embodiments, each instance of R⁶ and R⁷ is independently a group of the formula (iii).

In some embodiments, R⁶ and R⁷ are the same. In some embodiments, R⁶ and R⁷ are different.

In certain embodiments, both R⁶ and R⁷ are hydrogen. In certain embodiments, R⁶ is hydrogen and R⁷ is a group of the formula (i), (ii), or (iii). In certain embodiments, R⁶ is hydrogen and R⁷ is a group of the formula (i). In certain embodiments, R⁶ is hydrogen and R⁷ is a group of the formula (ii). In certain embodiments, R⁶ is hydrogen and R⁷ is a group of the formula (iii). In certain embodiments, each of R⁶ and R⁷ is independently a group of the formula (i), (ii), or (iii). In certain embodiments, each of R⁶ and R⁷ is independently a group of the formula (i). In certain embodiments, each of R⁶ and R⁷ is independently a group of the formula (ii). In certain embodiments, each of R⁶ and R⁷ is independently a group of the formula (iii). In certain embodiments, R⁶ and R⁷ are the same group, which is selected from formulas (i), (ii), and (iii). In some embodiments, R⁶ and R⁷ are the same group of formula (i). In some embodiments, R⁶ and R⁷ are the same group of formula (i-a). In some embodiments, R⁶ and R⁷ are the same group of formula (i-al). In some embodiments, R⁶ and R⁷ are the same group of formula (i-b).

In some embodiments, R⁶ and R⁷ are the same group of formula

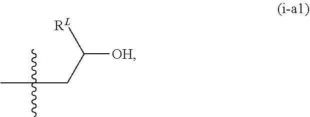

wherein R^{L} is as defined above and described herein. In some embodiments, R⁶ and R⁷ are the same group of formula

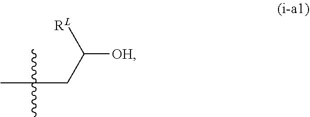

wherein R^{L} is optionally substituted C₁₋₅₀alkyl, optionally substituted C₂₋₅₀alkenyl, optionally substituted C₂₋₅₀alkynyl, optionally substituted heteroC₁₋₅₀alkyl, optionally substituted heteroC₂₋₅₀alkenyl, or optionally substituted heteroC₂₋₅₀alkynyl. In some embodiments, R⁶ and R⁷ are the same group of formula

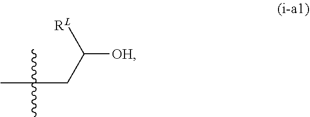

wherein R^{L} is optionally substituted C₅₋₅₀alkyl, optionally substituted C₅₋₅₀alkenyl, optionally substituted C₅₋₅₀alkynyl, optionally substituted heteroC₅₋₅₀alkyl, optionally substituted heteroC₅₋₅₀alkenyl, or optionally substituted heteroC₅₋₅₀alkynyl. In some embodiments, R⁶ and R⁷ are the same group of formula

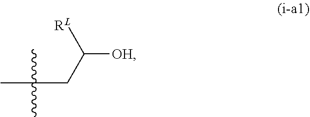

wherein R^{L} is optionally substituted C₅₋₄₀alkyl, optionally substituted C₅₋₄₀alkenyl, optionally substituted C₅₋₄₀alkynyl, optionally substituted heteroC₅₋₄₀alkyl, optionally substituted heteroC₅₋₄₀alkenyl, or optionally substituted heteroC₅₋₄₀alkynyl. In some embodiments, R⁶ and R⁷ are the same group of formula

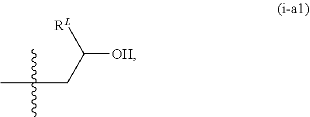

wherein R^{L} is optionally substituted C₅₋₃₀alkyl, optionally substituted C₅₋₃₀alkenyl, optionally substituted C₅₋₃₀alkynyl, optionally substituted heteroC₅₋₃₀alkyl, optionally substituted heteroC₅₋₃₀alkenyl, or optionally substituted heteroC₅₋₃₀alkynyl. In some embodiments, R⁶ and R⁷ are the same group of formula

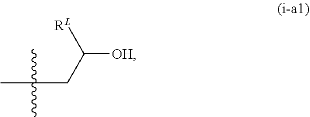

wherein R^{L} is optionally substituted C₅₋₂₅alkyl, optionally substituted C₅₋₂₅alkenyl, optionally substituted C₅₋₂₅alkynyl, optionally substituted heteroC₅₋₂₅alkyl, optionally substituted heteroC₅₋₂₅alkenyl, or optionally substituted heteroC₅₋₂₅alkynyl. In some embodiments, R⁶ and R⁷ are the same group of formula

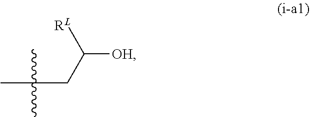

wherein R^{L} is optionally substituted C₅₋₂₀alkyl, optionally substituted C₅₋₂₀alkenyl, optionally substituted C₅₋₂₀alkynyl, optionally substituted heteroC₅₋₂₀alkyl, optionally substituted heteroC₅₋₂₀alkenyl, or optionally substituted heteroC₅₋₂₀alkynyl. In some embodiments, R⁶ and R⁷ are the same group of formula

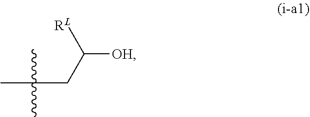

wherein R^{L} is optionally substituted C₅₋₁₅alkyl, optionally substituted C₅₋₁₅ alkenyl, optionally substituted C₅₋₁₅alkynyl, optionally substituted heteroC₅₋₁₅ alkyl, optionally substituted heteroC₅₋₁₅alkenyl, or optionally substituted heteroC₅₋₁₅alkynyl. In some embodiments, R⁶ and R⁷ are the same group of formula

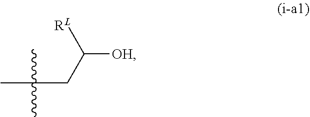

wherein R^{L} is optionally substituted C₅ alkyl, optionally substituted C₅ alkenyl, optionally substituted C₅ alkynyl, optionally substituted heteroC₅alkyl, optionally substituted heteroC₅alkenyl, or optionally substituted heteroC₅alkynyl. In some embodiments, R⁶ and R⁷ are the same group of formula

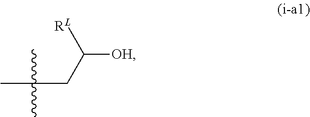

wherein R^{L} is optionally substituted C₆ alkyl, optionally substituted C₆ alkenyl, optionally substituted C₆ alkynyl, optionally substituted heteroC₆alkyl, optionally substituted heteroC₆alkenyl, or optionally substituted heteroC₆alkynyl. In some embodiments, R⁶ and R⁷ are the same group of formula

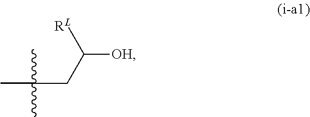

wherein R^{L} is optionally substituted C₇ alkyl, optionally substituted C₇ alkenyl, optionally substituted C₇ alkynyl, optionally substituted heteroC₇alkyl, optionally substituted heteroC₇alkenyl, or optionally substituted heteroC₇alkynyl. In some embodiments, R⁶ and R⁷ are the same group of formula

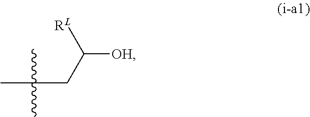

wherein R^{L} is optionally substituted C₈ alkyl, optionally substituted C₈ alkenyl, optionally substituted C₈ alkynyl, optionally substituted heteroC₈alkyl, optionally substituted heteroC₈alkenyl, or optionally substituted heteroC₈alkynyl. In some embodiments, R⁶ and R⁷ are the same group of formula

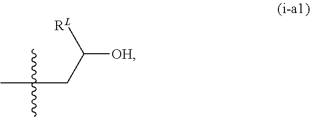

wherein R^{L} is optionally substituted C₉ alkyl, optionally substituted C₉ alkenyl, optionally substituted C₉ alkynyl, optionally substituted heteroC₉alkyl, optionally substituted heteroC₉alkenyl, or optionally substituted heteroC₉alkynyl. In some embodiments, R⁶ and R⁷ are the same group of formula

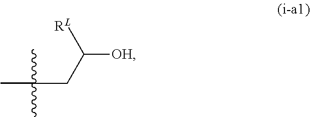

wherein R^{L} is optionally substituted C₁₀ alkyl, optionally substituted C₁₀ alkenyl, optionally substituted C₁₀ alkynyl, optionally substituted heteroC₁₀alkyl, optionally substituted heteroC₁₀alkenyl, or optionally substituted heteroC₁₀alkynyl. In some embodiments, R⁶ and R⁷ are the same group of formula

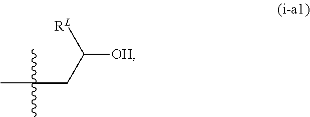

wherein R^{L} is optionally substituted C₁₁ alkyl, optionally substituted C₁₁ alkenyl, optionally substituted C₁₁ alkynyl, optionally substituted heteroC₁₁alkyl, optionally substituted heteroC₁₁alkenyl, or optionally substituted heteroC₁₁alkynyl. In some embodiments, R⁶ and R⁷ are the same group of formula

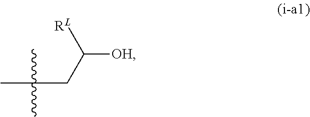

wherein R^{L} is optionally substituted C₁₂ alkyl, optionally substituted C₁₂ alkenyl, optionally substituted C₁₂ alkynyl, optionally substituted heteroC₁₂ alkyl, optionally substituted heteroC₁₂alkenyl, or optionally substituted heteroC₁₂alkynyl. In some embodiments, R⁶ and R⁷ are the same group of formula

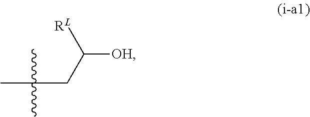

wherein R^{L} is optionally substituted C₁₃ alkyl, optionally substituted C₁₃ alkenyl, optionally substituted C₁₃ alkynyl, optionally substituted heteroC₁₃alkyl, optionally substituted heteroC₁₃alkenyl, or optionally substituted heteroC₁₃alkynyl. In some embodiments, R⁶ and R⁷ are the same group of formula

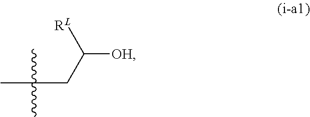

wherein R is optionally substituted C₁₄ alkyl, optionally substituted C₁₄ alkenyl, optionally substituted C₁₄ alkynyl, optionally substituted heteroC₁₄alkyl, optionally substituted heteroC₁₄alkenyl, or optionally substituted heteroC₁₄alkynyl. In some embodiments, R⁶ and R⁷ are the same group of formula

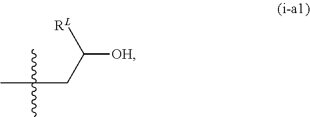

wherein R is optionally substituted C₁₅ alkyl, optionally substituted C₁₅ alkenyl, optionally substituted C₁₅ alkynyl, optionally substituted heteroC₁₅alkyl, optionally substituted heteroC₁₅alkenyl, or optionally substituted heteroC₁₅alkynyl.

In some embodiments, R⁶ and R⁷ are the same group of formula

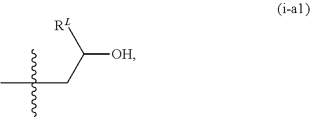

wherein R^{L} is as defined above and described herein. In some embodiments, R⁶ and R⁷ are the same group of formula

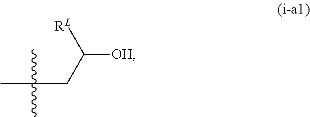

wherein R^{L} is optionally substituted C₁₋₅₀alkyl. In some embodiments, R⁶ and R⁷ are the same group of formula

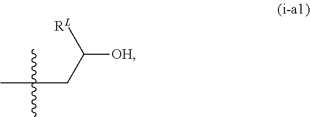

(wherein R^{L} is optionally substituted C₅₋₅₀alkyl. In some embodiments, R⁶ and R⁷ are the same group of formula

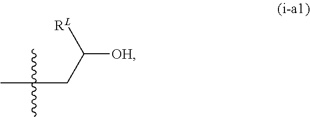

wherein R^{L} is optionally substituted C₅₋₄₀alkyl. In some embodiments, R⁶ and R⁷ are the same group of formula

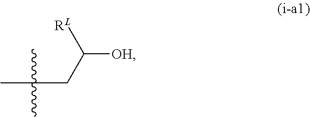

wherein R^{L} is optionally substituted C₅₋₃₀alkyl. In some embodiments, R⁶ and R⁷ are the same group of formulas

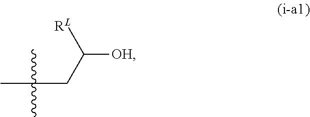

wherein R^{L} is optionally substituted C₅₋₂₅alkyl. In some embodiments, R⁶ and R⁷ are the same group of formula

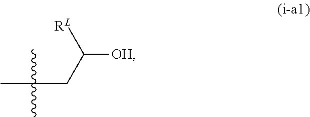

wherein R^{L} is optionally substituted C₅₋₂₀alkyl. In some embodiments, R⁶ and R⁷ are the same group of formula

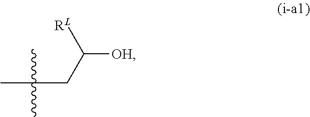

wherein R^{L} is optionally substituted C₅₋₁₅alkyl. In some embodiments, R⁶ and R⁷ are the same group of formula

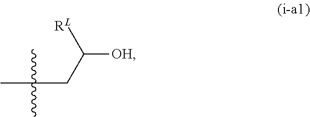

wherein R is optionally substituted C₅ alkyl. In some embodiments, R⁶ and R⁷ are the same group of formula

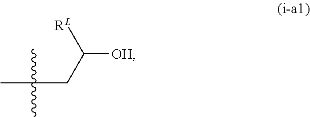

wherein R^{L} is optionally substituted C₆ alkyl. In some embodiments, R⁶ and R⁷ are the same group of formula

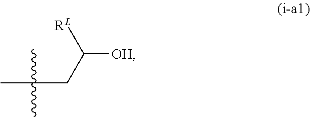

wherein R^{L} is optionally substituted C₇ alkyl. In some embodiments, R⁶ and R⁷ are the same group of formula

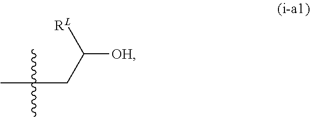

wherein R^{L} is optionally substituted C₈ alkyl. In some embodiments, R⁶ and R⁷ are the same group of formula

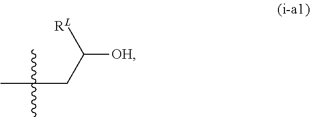

wherein R^{L} is optionally substituted C₉ alkyl. In some embodiments, R⁶ and R⁷ are the same group of formula

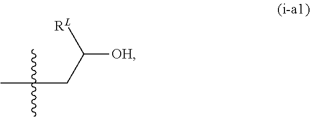

wherein R^{L} is optionally substituted C₁₀ alkyl. In some embodiments, R⁶ and R⁷ are the same group of formula

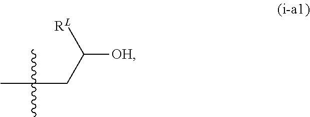

wherein R^{L} is optionally substituted C₁₁ alkyl. In some embodiments, R⁶ and R⁷ are the same group of formula

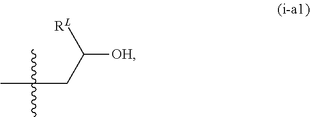

wherein R^{L} is optionally substituted C₁₂ alkyl. In some embodiments, R⁶ and R⁷ are the same group of formula

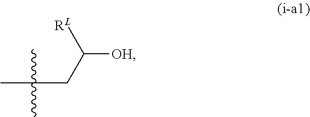

wherein R^{L} is optionally substituted C₁₃ alkyl. In some embodiments, R⁶ and R⁷ are the same group of formula

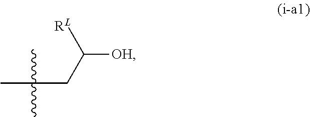

wherein R^{L} is optionally substituted C₁₄ alkyl. In some embodiments, R⁶ and R⁷ are the same group of formula

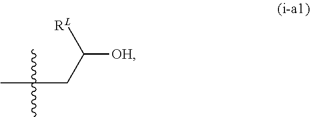

wherein R^{L} is optionally substituted C₁₅ alkyl.

As generally defined above, each occurrence of R^{A1} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, a sulfur protecting group when attached to an sulfur atom, a nitrogen protecting group when attached to a nitrogen atom, or two R^{A1} groups, together with the nitrogen atom to which they are attached, are joined to form an optionally substituted heterocyclic or optionally substituted heteroaryl ring.

In some embodiments, R^{A1} is hydrogen. In some embodiments, R^{A1} is optionally substituted alkyl. In some embodiments, R^{A1} is optionally substituted alkenyl. In some embodiments, R^{A1} is optionally substituted alkynyl. In some embodiments, R^{A1} is optionally substituted carbocyclyl. In some embodiments, R is optionally substituted heterocyclyl. In some embodiments, R^{A1} is optionally substituted aryl. In some embodiments, R^{A1} is optionally substituted heteroaryl. In some embodiments, R^{A1} is an oxygen protecting group when attached to an oxygen atom. In some embodiments, R^{A1} is a sulfur protecting group when attached to a sulfur atom. In some embodiments, R^{A1} is a nitrogen protecting group when attached to a nitrogen atom. In some embodiments, two R^{A1} groups, together with the nitrogen atom to which they are attached, are joined to form an optionally substituted heterocyclic or optionally substituted heteroaryl ring.

As generally defined above, each instance of R² is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group, or a group of the formula (i), (ii), or (iii):

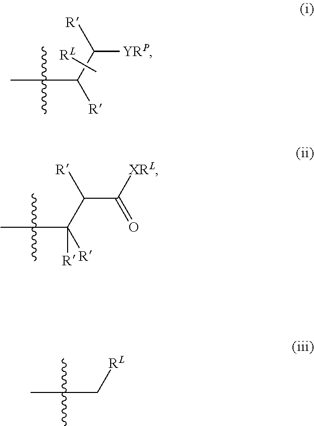

wherein each of R′, Y, R^{R}, R^{L} and X is independently as defined above and described herein.

In some embodiments, R² is hydrogen. In some embodiments, at least one instance of R² is hydrogen. In some embodiments, each instance of R² is hydrogen.

In certain embodiments, R² is optionally substituted alkyl; e.g., optionally substituted C₁₋₆alkyl, optionally substituted C₂₋₆alkyl, optionally substituted C₃₋₆alkyl, optionally substituted C₄₋₆alkyl, optionally substituted C₄₋₅alkyl, or optionally substituted C₃₋₄alkyl. In certain embodiments, at least one instance of R² is optionally substituted alkyl; e.g., optionally substituted C₁₋₆alkyl, optionally substituted C₂₋₆alkyl, optionally substituted C₃₋₆alkyl, optionally substituted C₄₋₆alkyl, optionally substituted C₄₋₅alkyl, or optionally substituted C₃₋₄alkyl.

In certain embodiments, R² is optionally substituted alkenyl, e.g., optionally substituted C₂₋₆alkenyl, optionally substituted C₃₋₆alkenyl, optionally substituted C₄₋₆alkenyl, optionally substituted C₄₋₅alkenyl, or optionally substituted C₃₋₄alkenyl. In certain embodiments, at least one instance of R² is optionally substituted alkenyl, e.g., optionally substituted C₂₋₆ alkenyl, optionally substituted C₃₋₆alkenyl, optionally substituted C₄₋₆alkenyl, optionally substituted C₄₋₅alkenyl, or optionally substituted C₃₋₄alkenyl.

In certain embodiments, R² is optionally substituted alkynyl, e.g., optionally substituted C₂₋₆alkynyl, optionally substituted C₃₋₆alkynyl, optionally substituted C₄₋₆alkynyl, optionally substituted C₄₋₅alkynyl, or optionally substituted C₃₋₄alkynyl. In certain embodiments, at least one instance of R² is optionally substituted alkynyl, e.g., optionally substituted C₂₋₆alkynyl, optionally substituted C₃₋₆alkynyl, optionally substituted C₄₋₆alkynyl, optionally substituted C₄₋₅alkynyl, or optionally substituted C₃₋₄alkynyl.

In certain embodiments, R² is optionally substituted carbocyclyl, e.g., optionally substituted C₃₋₁₀carbocyclyl, optionally substituted C₅₋₈carbocyclyl, optionally substituted C₅₋₆carbocyclyl, optionally substituted C₅ carbocyclyl, or optionally substituted C₆ carbocyclyl. In certain embodiments, at least one instance of R² is optionally substituted carbocyclyl, e.g., optionally substituted C₃₋₁₀carbocyclyl, optionally substituted C₅₋₈carbocyclyl, optionally substituted C₅₋₆carbocyclyl, optionally substituted C₅ carbocyclyl, or optionally substituted C₆ carbocyclyl.

In certain embodiments, R² is optionally substituted heterocyclyl, e.g., optionally substituted 3-14 membered heterocyclyl, optionally substituted 3-10 membered heterocyclyl, optionally substituted 5-8 membered heterocyclyl, optionally substituted 5-6 membered heterocyclyl, optionally substituted 5-membered heterocyclyl, or optionally substituted 6-membered heterocyclyl. In certain embodiments, at least one instance of R² is optionally substituted heterocyclyl, e.g., optionally substituted 3-14 membered heterocyclyl, optionally substituted 3-10 membered heterocyclyl, optionally substituted 5-8 membered heterocyclyl, optionally substituted 5-6 membered heterocyclyl, optionally substituted 5-membered heterocyclyl, or optionally substituted 6-membered heterocyclyl.

In certain embodiments, R² is optionally substituted aryl, e.g., optionally substituted phenyl. In some embodiments, R² is optionally substituted phenyl. In some embodiments, R² is substituted phenyl. In some embodiments, R² is unsubstituted phenyl. In certain embodiments, at least one instance of R² is optionally substituted aryl, e.g., optionally substituted phenyl. In some embodiments, at least one instance of R² is optionally substituted phenyl. In some embodiments, at least one instance of R² is substituted phenyl. In some embodiments, at least one instance of R² is unsubstituted phenyl.

In certain embodiments, R² is optionally substituted heteroaryl, e.g., optionally substituted 5-14 membered heteroaryl, optionally substituted 5-10 membered heteroaryl, optionally substituted 5-6 membered heteroaryl, optionally substituted 5-membered heteroaryl, or optionally substituted 6-membered heteroaryl. In certain embodiments, at least one instance of R² is optionally substituted heteroaryl, e.g., optionally substituted 5-14 membered heteroaryl, optionally substituted 5-10 membered heteroaryl, optionally substituted 5-6 membered heteroaryl, optionally substituted 5-membered heteroaryl, or optionally substituted 6-membered heteroaryl.

In some embodiments, R² is a nitrogen protecting group. In some embodiments, at least one R² is a nitrogen protecting group.

In certain embodiments, R² is a group of the formula (i). In certain embodiments, R² is a group of the formula (ii). In certain embodiments, R² is a group of the formula (iii). In certain embodiments, at least one instance of R² is a group of the formula (i). In certain embodiments, at least one instance of R² is a group of the formula (ii). In certain embodiments, at least one instance of R² is a group of the formula (iii).

In certain embodiments, each instance of R² is a group other than formula (i), (ii), or (iii); in that instance, it follows that at least one R^{Q} is a group of the formula (i), (ii), or (iii), or at least one R¹ is a group of formula (iv), and at least one of R⁶ or R⁷ encompassed by R¹ is a group of the formula (i), (ii), or (iii). For example, in certain embodiments, both instances of R² are hydrogen, and thus at least one R^{Q} is a group of the formula (i), (ii), or (iii), or at least one R¹ is a group of formula (iv), and at least one of R⁶ or R⁷ encompassed by R¹ is a group of the formula (i), (ii), or (iii).

As generally defined above, each instance of R′ is independently hydrogen or optionally substituted alkyl. In some embodiments, R′ is hydrogen. In some embodiments, R′ is substituted alkyl. In certain embodiments, at least one instance of R′ is hydrogen. In certain embodiments, at least two instances of R′ is hydrogen. In certain embodiments, each instance of R′ is hydrogen. In certain embodiments, at least one instance of R′ is optionally substituted alkyl, e.g., methyl. In certain embodiments, at least two instances of R′ is optionally substituted alkyl, e.g., methyl. In some embodiments, at least one instance of R′ is hydrogen, and at least one instance of R′ is optionally substituted alkyl. In certain embodiments, one instance of R′ is optionally substituted alkyl, and the rest are hydrogen.

As generally defined above, X is O, S, or NR^{X}. In some embodiments, X is O. In some embodiments, X is S. In some embodiments, X is NR^{X}, wherein R^{X} is as defined above and described herein.

As generally defined above, R^{X} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group. In some embodiments, R^{X} is hydrogen. In some embodiments, R^{X} is optionally substituted alkyl. In some embodiments, R^{X} is optionally substituted alkenyl. In some embodiments, R^{X} is optionally substituted alkynyl. In some embodiments, R^{X} is optionally substituted carbocyclyl. In some embodiments, R^{X} is optionally substituted heterocyclyl. In some embodiments, R^{X} is optionally substituted aryl. In some embodiments, R^{X} is optionally substituted heteroaryl. In some embodiments, R^{X} is a nitrogen protecting group.

As generally defined above, Y is O, S, or NR^{Y}. In some embodiments, Y is O. In some embodiments, Y is S. In some embodiments, Y is NR^{Y}, wherein R^{R} is as defined above and described herein.

As generally defined above, R^{Y} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group. In some embodiments, R^{Y} is hydrogen. In some embodiments, R^{Y} is optionally substituted alkyl. In some embodiments, R^{Y} is optionally substituted alkenyl. In some embodiments, R^{Y} is optionally substituted alkynyl. In some embodiments, R^{Y} is optionally substituted carbocyclyl. In some embodiments, R^{Y} is optionally substituted heterocyclyl. In some embodiments, R^{Y} is optionally substituted aryl. In some embodiments, R^{Y} is optionally substituted heteroaryl. In some embodiments, R^{Y} is a nitrogen protecting group.

As generally defined above, R^{P} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, a sulfur protecting group when attached to a sulfur atom, or a nitrogen protecting group when attached to a nitrogen atom. In some embodiments, R^{P} is hydrogen. In some embodiments, R^{P} is optionally substituted alkyl. In some embodiments, R^{P} is optionally substituted alkenyl. In some embodiments, R^{P} is optionally substituted alkynyl. In some embodiments, R^{P} is optionally substituted carbocyclyl. In some embodiments, R^{P} is optionally substituted heterocyclyl. In some embodiments, R^{P} is optionally substituted aryl. In some embodiments, R^{P} is optionally substituted heteroaryl. In some embodiments, R^{P} is an oxygen protecting group when attached to an oxygen atom. In some embodiments, R^{P} is a sulfur protecting group when attached to a sulfur atom. In some embodiments, R^{P} is a nitrogen protecting group when attached to a nitrogen atom.

As generally defined above, R^{L} is optionally substituted C₁₋₅₀ alkyl, optionally substituted C₂₋₅₀ alkenyl, optionally substituted C₂₋₅₀ alkynyl, optionally substituted heteroC₁₋₅₀ alkyl, optionally substituted heteroC₂₋₅₀ alkenyl, optionally substituted heteroC₂₋₅₀ alkynyl, or a polymer.

In some embodiments, R^{L} is optionally substituted C₁₋₅₀ alkyl. In some embodiments, R^{L} is optionally substituted C₂₋₅₀ alkyl. In some embodiments, R^{L} is optionally substituted C₂₋₄₀ alkyl. In some embodiments, R^{L} is optionally substituted C₂₋₃₀ alkyl. In some embodiments, R^{L} is optionally substituted C₂₋₂₀ alkyl. In some embodiments, R^{L} is optionally substituted C₂₋₁₉ alkyl. In some embodiments, R^{L} is optionally substituted C₂₋₁₈ alkyl. In some embodiments, R^{L} is optionally substituted C₂₋₇ alkyl. In some embodiments, R^{L} is optionally substituted C₂₋₁₆ alkyl. In some embodiments, R^{L} is optionally substituted C₂₋₁₅ alkyl. In some embodiments, R^{L} is optionally substituted C₂₋₁₄ alkyl. In some embodiments, R^{L} is optionally substituted C₂₋₁₃ alkyl. In some embodiments, R^{L} is optionally substituted C₂₋₁₂ alkyl. In some embodiments, R^{L} is optionally substituted C₂₋₁₁ alkyl. In some embodiments, R^{L} is optionally substituted C₂₋₁₀ alkyl. In some embodiments, R^{L} is optionally substituted C₂₋₉ alkyl. In some embodiments, R^{L} is optionally substituted C_{z-g} alkyl. In some embodiments, R^{L} is optionally substituted C₂₋₇ alkyl. In some embodiments, R^{L} is optionally substituted C₂₋₆ alkyl.

In some embodiments, R^{L} is optionally substituted C₄₋₅₀ alkyl. In some embodiments, R^{L} is optionally substituted C₄₋₄₀ alkyl. In some embodiments, R^{L} is optionally substituted C₄₋₃₀ alkyl. In some embodiments, R^{L} is optionally substituted C₄₋₂₀ alkyl. In some embodiments, R^{L} is optionally substituted C₄₋₁₉ alkyl. In some embodiments, R^{L} is optionally substituted C₄₋₁₈ alkyl. In some embodiments, R^{L} is optionally substituted C₄₋₁₇ alkyl. In some embodiments, R^{L} is optionally substituted C₄₋₁₆ alkyl. In some embodiments, R^{L} is optionally substituted C₄₋₁₅ alkyl. In some embodiments, R^{L} is optionally substituted C₄₋₁₄ alkyl. In some embodiments, R^{L} is optionally substituted C₄₋₁₃ alkyl. In some embodiments, R^{L} is optionally substituted C₄₋₁₂ alkyl. In some embodiments, R^{L} is optionally substituted C₄₋₁₁ alkyl. In some embodiments, R^{L} is optionally substituted C₄₋₁₀ alkyl. In some embodiments, R^{L} is optionally substituted C₄₋₉ alkyl. In some embodiments, R^{L} is optionally substituted C₄₋₈ alkyl. In some embodiments, R^{L} is optionally substituted C₄₋₇ alkyl. In some embodiments, R^{L} is optionally substituted C₄₋₆ alkyl.

In some embodiments, R^{L} is optionally substituted C₆₋₅₀ alkyl. In some embodiments, R^{L} is optionally substituted C₆₋₄₀ alkyl. In some embodiments, R^{L} is optionally substituted C₆₋₃₀ alkyl. In some embodiments, R^{L} is optionally substituted C₆₋₂₀ alkyl. In some embodiments, R^{L} is optionally substituted C₆₋₁₉ alkyl. In some embodiments, R^{L} is optionally substituted C₆₋₁₈ alkyl. In some embodiments, R^{L} is optionally substituted C₆₋₇ alkyl. In some embodiments, R^{L} is optionally substituted C₆₋₁₆ alkyl. In some embodiments, R^{L} is optionally substituted C₆₋₁₅ alkyl. In some embodiments, R^{L} is optionally substituted C₆₋₁₄ alkyl. In some embodiments, R^{L} is optionally substituted C₆₋₁₃ alkyl. In some embodiments, R^{L} is optionally substituted C₆₋₁₂ alkyl. In some embodiments, R^{L} is optionally substituted C₆₋₁₁ alkyl. In some embodiments, R^{L} is optionally substituted C₆₋₁₀ alkyl. In some embodiments, R^{L} is optionally substituted C₆₋₉ alkyl. In some embodiments, R^{L} is optionally substituted C₆₋₈ alkyl. In some embodiments, R^{L} is optionally substituted C₆₋₇ alkyl.

In some embodiments, R^{L} is optionally substituted C₈₋₅₀ alkyl. In some embodiments, R^{L} is optionally substituted C₈₋₄₀ alkyl. In some embodiments, R^{L} is optionally substituted C₈₋₃₀ alkyl. In some embodiments, R^{L} is optionally substituted C₈₋₂₀ alkyl. In some embodiments, R^{L} is optionally substituted C₈₋₁₉ alkyl. In some embodiments, R^{L} is optionally substituted C₈₋₁₈ alkyl. In some embodiments, R^{L} is optionally substituted C₈₋₁₇ alkyl. In some embodiments, R^{L} is optionally substituted C₈₋₁₆ alkyl. In some embodiments, R^{L} is optionally substituted C₈₋₁₅ alkyl. In some embodiments, R^{L} is optionally substituted C₈₋₁₄ alkyl. In some embodiments, R^{L} is optionally substituted C₈₋₁₃ alkyl. In some embodiments, R^{L} is optionally substituted C₈₋₁₂ alkyl. In some embodiments, R^{L} is optionally substituted C₈₋₁₁ alkyl. In some embodiments, R^{L} is optionally substituted C₈₋₁₀ alkyl. In some embodiments, R^{L} is optionally substituted C₈₋₉ alkyl.

In some embodiments, R^{L} is optionally substituted C₉₋₅₀ alkyl. In some embodiments, R^{L} is optionally substituted C₉₋₄₀ alkyl. In some embodiments, R^{L} is optionally substituted C₉₋₃₀ alkyl. In some embodiments, R^{L} is optionally substituted C₉₋₂₀ alkyl. In some embodiments, R^{L} is optionally substituted C₉₋₁₉ alkyl. In some embodiments, R^{L} is optionally substituted C₉₋₁₈ alkyl. In some embodiments, R^{L} is optionally substituted C₉₋₇ alkyl. In some embodiments, R^{L} is optionally substituted C₉₋₁₆ alkyl. In some embodiments, R^{L} is optionally substituted C₉₋₁₅ alkyl. In some embodiments, R^{L} is optionally substituted C₉₋₁₄ alkyl. In some embodiments, R^{L} is optionally substituted C₉₋₁₃ alkyl. In some embodiments, R^{L} is optionally substituted C₉₋₁₂ alkyl. In some embodiments, R^{L} is optionally substituted C₉₋₁₁ alkyl. In some embodiments, R^{L} is optionally substituted C₉₋₁₀ alkyl.

In some embodiments, R^{L} is optionally substituted C₁₀₋₅₀ alkyl. In some embodiments, R^{L} is optionally substituted C₁₀₋₄₀ alkyl. In some embodiments, R^{L} is optionally substituted C₁₀₋₃₀ alkyl. In some embodiments, R^{L} is optionally substituted C₁₀₋₂₀ alkyl. In some embodiments, R^{L} is optionally substituted C₁₀₋₁₉ alkyl. In some embodiments, R^{L} is optionally substituted C₁₀₋₁₈ alkyl. In some embodiments, R^{L} is optionally substituted C₁₀₋₁₇ alkyl. In some embodiments, R^{L} is optionally substituted C₁₀₋₁₆ alkyl. In some embodiments, R^{L} is optionally substituted C₁₀₋₁₅ alkyl. In some embodiments, R^{L} is optionally substituted C₁₀₋₁₄ alkyl. In some embodiments, R^{L} is optionally substituted C₁₀₋₁₃ alkyl. In some embodiments, R^{L} is optionally substituted C₁₀₋₁₂ alkyl. In some embodiments, R^{L} is optionally substituted C₁₀₋₁₁ alkyl.

In some embodiments, R^{L} is optionally substituted C₁₁₋₅₀ alkyl. In some embodiments, R^{L} is optionally substituted C₁₁₋₄₀ alkyl. In some embodiments, R^{L} is optionally substituted C₁₁₋₃₀ alkyl. In some embodiments, R^{L} is optionally substituted C₁₁₋₂₀ alkyl. In some embodiments, R^{L} is optionally substituted C₁₁₋₁₉ alkyl. In some embodiments, R^{L} is optionally substituted C₁₁₋₁₈ alkyl. In some embodiments, R^{L} is optionally substituted C₁₁₋₁₇ alkyl. In some embodiments, R^{L} is optionally substituted C₁₁₋₁₆ alkyl. In some embodiments, R^{L} is optionally substituted C₁₁₋₁₅ alkyl. In some embodiments, R^{L} is optionally substituted C₁₁₋₁₄ alkyl. In some embodiments, R^{L} is optionally substituted C₁₁₋₁₃ alkyl. In some embodiments, R^{L} is optionally substituted C₁₁₋₁₂ alkyl.

In some embodiments, R^{L} is optionally substituted C₁₂₋₅₀ alkyl. In some embodiments, R^{L} is optionally substituted C₁₂₋₄₀ alkyl. In some embodiments, R^{L} is optionally substituted C₁₂₋₃₀ alkyl. In some embodiments, R^{L} is optionally substituted C₁₂₋₂₀ alkyl. In some embodiments, R^{L} is optionally substituted C₁₂₋₁₉ alkyl. In some embodiments, R^{L} is optionally substituted C₁₂₋₁₈ alkyl. In some embodiments, R^{L} is optionally substituted C₁₂₋₁₇ alkyl. In some embodiments, R^{L} is optionally substituted C₁₂₋₁₆ alkyl. In some embodiments, R^{L} is optionally substituted C₁₂₋₁₅ alkyl. In some embodiments, R^{L} is optionally substituted C₁₂₋₁₄ alkyl. In some embodiments, R^{L} is optionally substituted C₁₂₋₁₃ alkyl.

In some embodiments, R^{L} is optionally substituted C₆ alkyl. In some embodiments, R^{L} is optionally substituted C₇ alkyl. In some embodiments, R^{L} is optionally substituted C₈ alkyl. In some embodiments, R^{L} is optionally substituted C₉ alkyl. In some embodiments, R^{L} is optionally substituted C₁₀ alkyl. In some embodiments, R^{L} is optionally substituted C₁₁ alkyl. In some embodiments, R^{L} is optionally substituted C₁₂ alkyl. In some embodiments, R^{L} is optionally substituted C₁₃ alkyl. In some embodiments, R^{L} is optionally substituted C₁₄ alkyl. In some embodiments, R^{L} is optionally substituted C₁₅ alkyl. In some embodiments, R^{L} is optionally substituted C₁₆ alkyl. In some embodiments, R^{L} is optionally substituted C₁₇ alkyl. In some embodiments, R^{L} is optionally substituted C₁₈ alkyl. In some embodiments, R^{L} is optionally substituted C₁₉ alkyl. In some embodiments, R^{L} is optionally substituted C₂₀ alkyl.

In some embodiments, for example, in any of the above embodiments, R^{L} is a substituted alkyl group. In some embodiments, R^{L} is an unsubstituted alkyl group. In some embodiments, R^{L} is an optionally substituted straight-chain alkyl group. In some embodiments, R^{L} is a substituted straight-chain alkyl group. In some embodiments, R^{L} is an unsubstituted straight-chain alkyl group. In some embodiments, R^{L} is an optionally substituted branched alkyl group. In some embodiments, R^{L} is a substituted branched alkyl group. In some embodiments, R^{L} is an unsubstituted branched alkyl group.

In certain embodiments, at least one instance of R^{L} is an unsubstituted alkyl. Exemplary unsubstituted alkyl groups include, but are not limited to, —CH₃, —C₂H₅, —C₃H₇, —C₄H₉, —C₅H₁₁, —C₆H₁₃, —C₇H₁₅, —C₈H₁₇, —C₉H₁₉, —C₁₀H₂₁, —C₁₁H₂₃, —C₁₂H₂₅, —C₁₃H₂₇, —C₁₄H₂₉, —C₁₅H₃₁, —C₁₆H₃₃, —C₁₇H₃₅, —C₁₈H₃₇, —C₁₉H₃₉, —C₂₀H₄₁, —C₂₁H₄₃, —C₂₂H₄₅, —C₂₃H₄₇, —C₂₄H₄₉, and —C₂₅H₅₁.

In certain embodiments, at least one instance of R^{L} is a substituted alkyl. For example, in certain embodiments, at least one instance of R^{L} is an alkyl substituted with one or more fluorine substituents. Exemplary fluorinated alkyl groups include, but are not limited to:

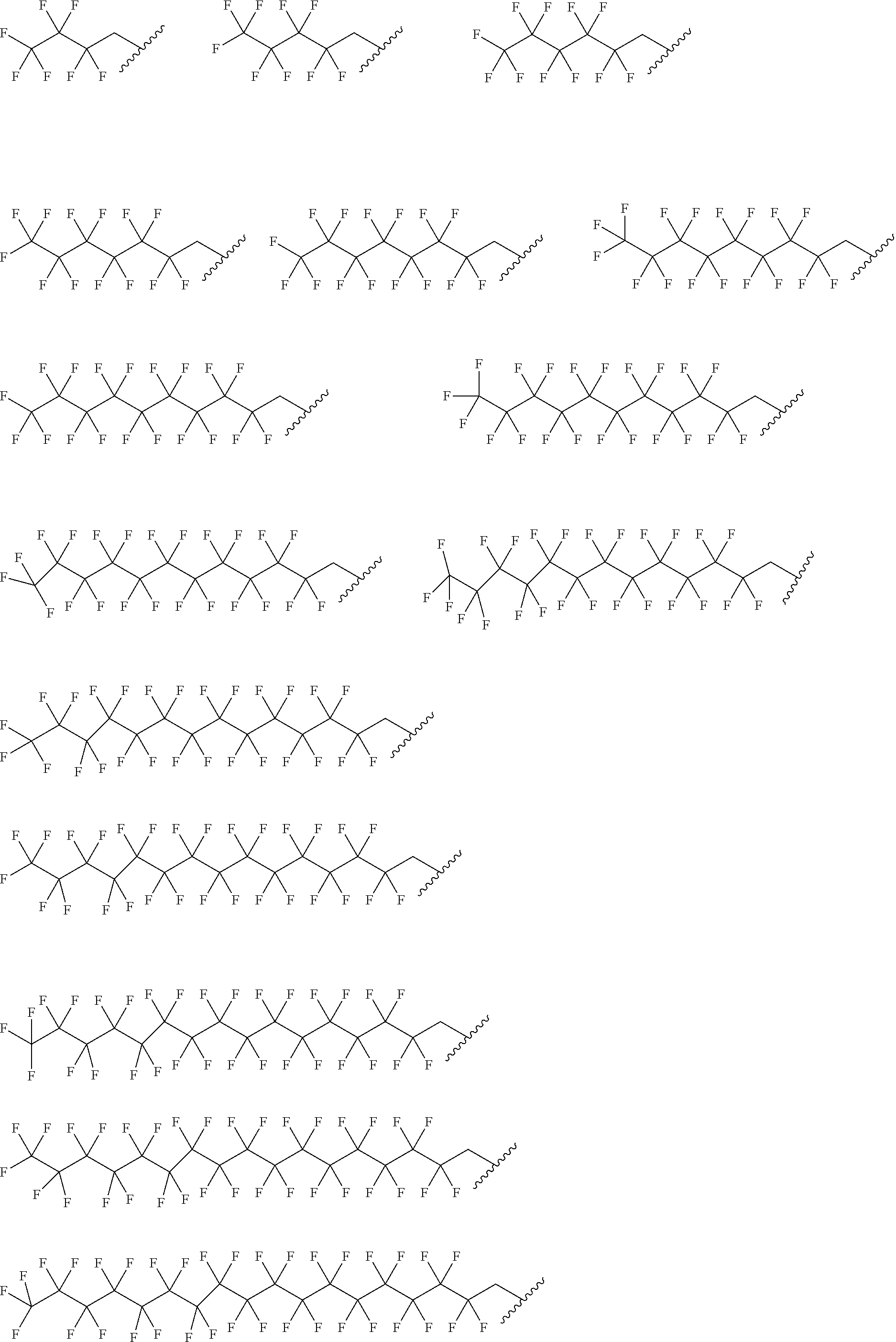

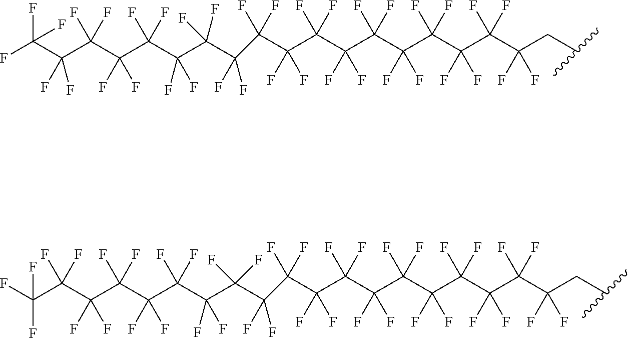 [SMILES: *CC(F)(F)C(F)(F)C(F)(F)C(F)(F)C(F)(F)C(F)(F)C(F)(F)C(F)(F)C(F)(F)C(F)(F)C(F)(F)C(F)(F)C(F)(F)C(F)(F)C(F)(F)C(F)(F)C(F)(F)C(F)(F)F ; *CC(F)(F)C(F)(F)C(F)(F)C(F)(F)C(F)(F)C(F)(F)C(F)(F)C(F)(F)C(F)(F)C(F)(F)C(F)(F)C(F)(F)C(F)(F)C(F)(F)C(F)(F)C(F)(F)C(F)(F)C(F)(F)C(F)(F)F]

In some embodiments, R^{L} is optionally substituted C₂₋₅₀ alkenyl. In some embodiments, R^{L} is optionally substituted C₂₋₄₀ alkenyl. In some embodiments, R^{L} is optionally substituted C₂₋₃₀ alkenyl. In some embodiments, R^{L} is optionally substituted C₂₋₂₀ alkenyl. In some embodiments, R^{L} is optionally substituted C₂₋₁₉ alkenyl. In some embodiments, R^{L} is optionally substituted C₂₋₁₈ alkenyl. In some embodiments, R^{L} is optionally substituted C₂₋₁₇ alkenyl. In some embodiments, R^{L} is optionally substituted C₂₋₁₆ alkenyl. In some embodiments, R^{L} is optionally substituted C₂₋₁₅ alkenyl. In some embodiments, R^{L} is optionally substituted C₂₋₁₄ alkenyl. In some embodiments, R^{L} is optionally substituted C₂₋₁₃ alkenyl. In some embodiments, R^{L} is optionally substituted C₂₋₁₂ alkenyl. In some embodiments, R^{L} is optionally substituted C₂₋₁₁ alkenyl. In some embodiments, R^{L} is optionally substituted C₂₋₁₀ alkenyl. In some embodiments, R^{L} is optionally substituted C₂₋₉ alkenyl. In some embodiments, R^{L} is optionally substituted C₂₋₈ alkenyl. In some embodiments, R^{L} is optionally substituted C₂₋₇ alkenyl. In some embodiments, R^{L} is optionally substituted C₂₋₆ alkenyl.

In some embodiments, R^{L} is optionally substituted C₄₋₅₀ alkenyl. In some embodiments, R^{L} is optionally substituted C₄₋₄₀ alkenyl. In some embodiments, R^{L} is optionally substituted C₄₋₃₀ alkenyl. In some embodiments, R^{L} is optionally substituted C₄₋₂₀ alkenyl. In some embodiments, R^{L} is optionally substituted C₄₋₁₉ alkenyl. In some embodiments, R^{L} is optionally substituted C₄₋₁₈ alkenyl. In some embodiments, R^{L} is optionally substituted C₄₋₁₇ alkenyl. In some embodiments, R^{L} is optionally substituted C₄₋₁₆ alkenyl. In some embodiments, R^{L} is optionally substituted C₄₋₁₅ alkenyl. In some embodiments, R^{L} is optionally substituted C₄₋₁₄ alkenyl. In some embodiments, R^{L} is optionally substituted C₄₋₁₃ alkenyl. In some embodiments, R^{L} is optionally substituted C₄₋₁₂ alkenyl. In some embodiments, R^{L} is optionally substituted C₄₋₁₁ alkenyl. In some embodiments, R^{L} is optionally substituted C₄₋₁₀ alkenyl. In some embodiments, R^{L} is optionally substituted C₄₋₉ alkenyl. In some embodiments, R^{L} is optionally substituted C_{4-g} alkenyl. In some embodiments, R^{L} is optionally substituted C₄₋₇ alkenyl. In some embodiments, R^{L} is optionally substituted C₄₋₆ alkenyl.

In some embodiments, R^{L} is optionally substituted C₆₋₅₀ alkenyl. In some embodiments, R^{L} is optionally substituted C₆₋₄₀ alkenyl. In some embodiments, R^{L} is optionally substituted C₆₋₃₀ alkenyl. In some embodiments, R^{L} is optionally substituted C₆₋₂₀ alkenyl. In some embodiments, R^{L} is optionally substituted C₆₋₁₉ alkenyl. In some embodiments, R^{L} is optionally substituted C₆₋₁₈ alkenyl. In some embodiments, R^{L} is optionally substituted C₆₋₁₇ alkenyl. In some embodiments, R^{L} is optionally substituted C₆₋₁₆ alkenyl. In some embodiments, R^{L} is optionally substituted C₆₋₁₅ alkenyl. In some embodiments, R^{L} is optionally substituted C₆₋₁₄ alkenyl. In some embodiments, R^{L} is optionally substituted C₆₋₁₃ alkenyl. In some embodiments, R^{L} is optionally substituted C₆₋₁₂ alkenyl. In some embodiments, R^{L} is optionally substituted C₆₋₁₁ alkenyl. In some embodiments, R^{L} is optionally substituted C₆₋₁₀ alkenyl. In some embodiments, R^{L} is optionally substituted C₆₋₉ alkenyl. In some embodiments, R^{L} is optionally substituted C₆₋₈ alkenyl. In some embodiments, R^{L} is optionally substituted C₆₋₇ alkenyl.

In some embodiments, R^{L} is optionally substituted C₈₋₅₀ alkenyl. In some embodiments, R^{L} is optionally substituted C₈₋₄₀ alkenyl. In some embodiments, R^{L} is optionally substituted C₈₋₃₀ alkenyl. In some embodiments, R^{L} is optionally substituted C₈₋₂₀ alkenyl. In some embodiments, R^{L} is optionally substituted C₈₋₁₉ alkenyl. In some embodiments, R^{L} is optionally substituted C₈₋₁₈ alkenyl. In some embodiments, R^{L} is optionally substituted C₈₋₁₇ alkenyl. In some embodiments, R^{L} is optionally substituted C₈₋₁₆ alkenyl. In some embodiments, R^{L} is optionally substituted C₈₋₁₅ alkenyl. In some embodiments, R^{L} is optionally substituted C₈₋₁₄ alkenyl. In some embodiments, R^{L} is optionally substituted C₈₋₁₃ alkenyl. In some embodiments, R^{L} is optionally substituted C₈₋₁₂ alkenyl. In some embodiments, R^{L} is optionally substituted C₈₋₁₁ alkenyl. In some embodiments, R^{L} is optionally substituted C₈₋₁₀ alkenyl. In some embodiments, R^{L} is optionally substituted C₈₋₉ alkenyl.

In some embodiments, R^{L} is optionally substituted C₉₋₅₀ alkenyl. In some embodiments, R^{L} is optionally substituted C₉₋₄₀ alkenyl. In some embodiments, R^{L} is optionally substituted C₉₋₃₀ alkenyl. In some embodiments, R^{L} is optionally substituted C₉₋₂₀ alkenyl. In some embodiments, R^{L} is optionally substituted C₉₋₁₉ alkenyl. In some embodiments, R^{L} is optionally substituted C₉₋₁₈ alkenyl. In some embodiments, R^{L} is optionally substituted C₉₋₁₇ alkenyl. In some embodiments, R^{L} is optionally substituted C₉₋₁₆ alkenyl. In some embodiments, R^{L} is optionally substituted C₉₋₁₅ alkenyl. In some embodiments, R^{L} is optionally substituted C₉₋₁₄ alkenyl. In some embodiments, R^{L} is optionally substituted C₉₋₁₃ alkenyl. In some embodiments, R^{L} is optionally substituted C₉₋₁₂ alkenyl. In some embodiments, R^{L} is optionally substituted C₉₋₁₁ alkenyl. In some embodiments, R^{L} is optionally substituted C₉₋₁₀ alkenyl.

In some embodiments, R^{L} is optionally substituted C₁₀₋₅₀ alkenyl. In some embodiments, R^{L} is optionally substituted C₁₀₋₄₀ alkenyl. In some embodiments, R^{L} is optionally substituted C₁₀₋₃₀ alkenyl. In some embodiments, R^{L} is optionally substituted C₁₀₋₂₀ alkenyl. In some embodiments, R^{L} is optionally substituted C₁₀₋₁₉ alkenyl. In some embodiments, R^{L} is optionally substituted C₁₀₋₁₈ alkenyl. In some embodiments, R^{L} is optionally substituted C₁₀₋₁₇ alkenyl. In some embodiments, R^{L} is optionally substituted C₁₀₋₁₆ alkenyl. In some embodiments, R^{L} is optionally substituted C₁₀₋₁₅ alkenyl. In some embodiments, R^{L} is optionally substituted C₁₀₋₁₄ alkenyl. In some embodiments, R^{L} is optionally substituted C₁₀₋₁₃ alkenyl. In some embodiments, R^{L} is optionally substituted C₁₀₋₁₂ alkenyl. In some embodiments, R^{L} is optionally substituted C₁₀₋₁₁ alkenyl.

In some embodiments, R^{L} is optionally substituted C₁₁₋₅₀ alkenyl. In some embodiments, R^{L} is optionally substituted C₁₁₋₄₀ alkenyl. In some embodiments, R^{L} is optionally substituted C₁₁₋₃₀ alkenyl. In some embodiments, R^{L} is optionally substituted C₁₁₋₂₀ alkenyl. In some embodiments, R^{L} is optionally substituted C₁₁₋₁₉ alkenyl. In some embodiments, R^{L} is optionally substituted C₁₁₋₁₈ alkenyl. In some embodiments, R^{L} is optionally substituted C₁₁₋₁₇ alkenyl. In some embodiments, R^{L} is optionally substituted C₁₁₋₁₆ alkenyl. In some embodiments, R^{L} is optionally substituted C₁₁₋₁₅ alkenyl. In some embodiments, R^{L} is optionally substituted C₁₁₋₁₄ alkenyl. In some embodiments, R^{L} is optionally substituted C₁₁₋₁₃ alkenyl. In some embodiments, R^{L} is optionally substituted C₁₁₋₁₂ alkenyl.

In some embodiments, R^{L} is optionally substituted C₁₂₋₅₀ alkenyl. In some embodiments, R^{L} is optionally substituted C₁₂₋₄₀ alkenyl. In some embodiments, R^{L} is optionally substituted C₁₂₋₃₀ alkenyl. In some embodiments, R^{L} is optionally substituted C₁₂₋₂₀ alkenyl. In some embodiments, R^{L} is optionally substituted C₁₂₋₁₉ alkenyl. In some embodiments, R^{L} is optionally substituted C₁₂₋₁₈ alkenyl. In some embodiments, R^{L} is optionally substituted C₁₂₋₁₇ alkenyl. In some embodiments, R^{L} is optionally substituted C₁₂₋₁₆ alkenyl. In some embodiments, R^{L} is optionally substituted C₁₂₋₁₅ alkenyl. In some embodiments, R^{L} is optionally substituted C₁₂₋₁₄ alkenyl. In some embodiments, R^{L} is optionally substituted C₁₂₋₁₃ alkenyl.

In some embodiments, R^{L} is optionally substituted C₆ alkenyl. In some embodiments, R^{L} is optionally substituted C₇ alkenyl. In some embodiments, R^{L} is optionally substituted C₈ alkenyl. In some embodiments, R^{L} is optionally substituted C₉ alkenyl. In some embodiments, R^{L} is optionally substituted C₁₀ alkenyl. In some embodiments, R^{L} is optionally substituted C₁₁ alkenyl. In some embodiments, R^{L} is optionally substituted C₁₂ alkenyl. In some embodiments, R^{L} is optionally substituted C₁₃ alkenyl. In some embodiments, R^{L} is optionally substituted C₁₄ alkenyl. In some embodiments, R^{L} is optionally substituted C₁₅ alkenyl. In some embodiments, R^{L} is optionally substituted C₁₆ alkenyl. In some embodiments, R^{L} is optionally substituted C₁₇ alkenyl. In some embodiments, R^{L} is optionally substituted Cig alkenyl. In some embodiments, R^{L} is optionally substituted C₁₉ alkenyl. In some embodiments, R^{L} is optionally substituted C₂₀ alkenyl.

In some embodiments, for example, in any of the above embodiments, R^{L} is a substituted alkyl group. In some embodiments, R^{L} is an unsubstituted alkyl group. In some embodiments, R^{L} is an optionally substituted straight-chain alkenyl group. In some embodiments, R^{L} is a substituted straight-chain alkenyl group. In some embodiments, R^{L} is an unsubstituted straight-chain alkenyl group. In some embodiments, R^{L} is an optionally substituted branched alkenyl group. In some embodiments, R^{L} is a substituted branched alkenyl group. In some embodiments, R^{L} is an unsubstituted branched alkenyl group.

Exemplary unsubstituted alkenyl group include, but are not limited to:

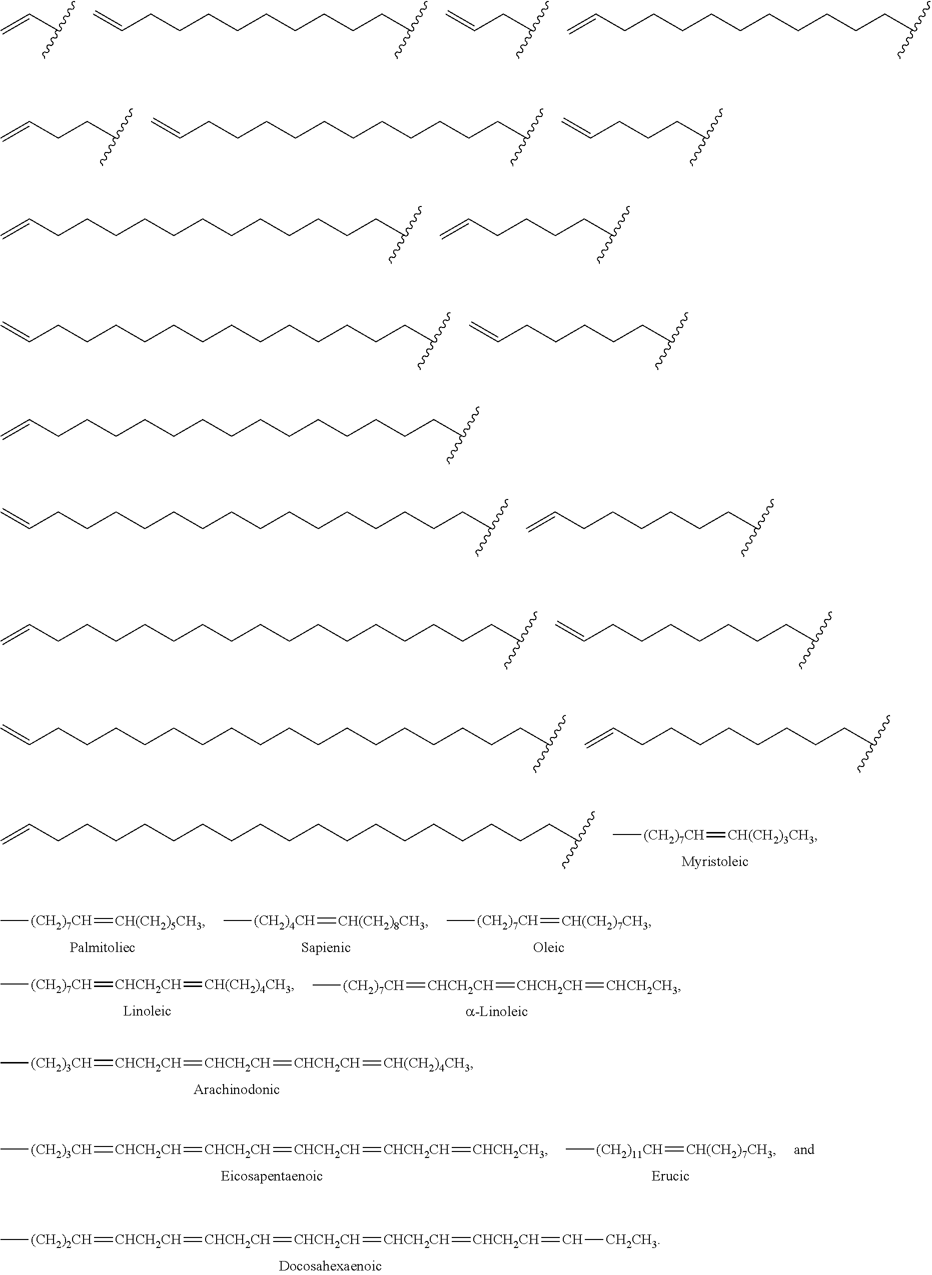

In some embodiments, wherein R^{L} is defined as a C₆₋₅₀alkyl or C₆₋₅₀alkenyl groups, such groups are meant to encompass lipophilic groups (also referred to as a “lipid tail”). Lipophilic groups comprise a group of molecules that include fats, waxes, oils, fatty acids, and the like. Lipid tails present in these lipid groups can be saturated and unsaturated, depending on whether or not the lipid tail comprises double bonds. The lipid tail can also comprise different lengths, often categorized as medium (i.e., with tails between 7-12 carbons, e.g., C₇₋₁₂ alkyl or C₇₋₁₂ alkenyl), long (i.e., with tails greater than 12 carbons and up to 22 carbons, e.g., C₁₃₋₂₂alkyl or C₁₃₋₂₂ alkenyl), or very long (i.e., with tails greater than 22 carbons, e.g., C₂₃₋₃₀ alkyl or C₂₃₋₃₀ alkenyl).

In some embodiments, R^{L} is optionally substituted C₂₋₅₀ alkynyl. In some embodiments, R^{L} is optionally substituted C₂₋₄₀ alkynyl. In some embodiments, R^{L} is optionally substituted C₂₋₃₀ alkynyl. In some embodiments, R^{L} is optionally substituted C₂₋₂₀ alkynyl. In some embodiments, R^{L} is optionally substituted C₂₋₁₉ alkynyl. In some embodiments, R^{L} is optionally substituted C₂₋₁₈ alkynyl. In some embodiments, R^{L} is optionally substituted C₂₋₁₇ alkynyl. In some embodiments, R^{L} is optionally substituted C₂₋₁₆ alkynyl. In some embodiments, R^{L} is optionally substituted C₂₋₁₅ alkynyl. In some embodiments, R^{L} is optionally substituted C₂₋₁₄ alkynyl. In some embodiments, R^{L} is optionally substituted C₂₋₁₃ alkynyl. In some embodiments, R^{L} is optionally substituted C₂₋₁₂ alkynyl. In some embodiments, R^{L} is optionally substituted C₂₋₁₁ alkynyl. In some embodiments, R^{L} is optionally substituted C₂₋₁₀ alkynyl. In some embodiments, R^{L} is optionally substituted C₂₋₉ alkynyl. In some embodiments, R^{L} is optionally substituted C₂₋₈ alkynyl. In some embodiments, R^{L} is optionally substituted C₂₋₇ alkynyl. In some embodiments, R^{L} is optionally substituted C₂₋₆ alkynyl.

In some embodiments, R^{L} is optionally substituted C₄₋₅₀ alkynyl. In some embodiments, R^{L} is optionally substituted C₄₋₄₀ alkynyl. In some embodiments, R^{L} is optionally substituted C₄₋₃₀ alkynyl. In some embodiments, R^{L} is optionally substituted C₄₋₂₀ alkynyl. In some embodiments, R^{L} is optionally substituted C₄₋₁₉ alkynyl. In some embodiments, R^{L} is optionally substituted C₄₋₁₈ alkynyl. In some embodiments, R^{L} is optionally substituted C₄₋₁₇ alkynyl. In some embodiments, R^{L} is optionally substituted C₄₋₁₆ alkynyl. In some embodiments, R^{L} is optionally substituted C₄₋₁₅ alkynyl. In some embodiments, R^{L} is optionally substituted C₄₋₁₄ alkynyl. In some embodiments, R^{L} is optionally substituted C₄₋₁₃ alkynyl. In some embodiments, R^{L} is optionally substituted C₄₋₁₂ alkynyl. In some embodiments, R^{L} is optionally substituted C₄₋₁₁ alkynyl. In some embodiments, R^{L} is optionally substituted C₄₋₁₀ alkynyl. In some embodiments, R^{L} is optionally substituted C₄₋₉ alkynyl. In some embodiments, R^{L} is optionally substituted C₄₋₈ alkynyl. In some embodiments, R^{L} is optionally substituted C₄₋₇ alkynyl. In some embodiments, R^{L} is optionally substituted C₄₋₆ alkynyl.

In some embodiments, R^{L} is optionally substituted C₆₋₅₀ alkynyl. In some embodiments, R^{L} is optionally substituted C₆₋₄₀ alkynyl. In some embodiments, R^{L} is optionally substituted C₆₋₃₀ alkynyl. In some embodiments, R^{L} is optionally substituted C₆₋₂₀ alkynyl. In some embodiments, R^{L} is optionally substituted C₆₋₁₉ alkynyl. In some embodiments, R^{L} is optionally substituted C₆₋₁₈ alkynyl. In some embodiments, R^{L} is optionally substituted C₆₋₁₇ alkynyl. In some embodiments, R^{L} is optionally substituted C₆₋₁₆ alkynyl. In some embodiments, R^{L} is optionally substituted C₆₋₁₅ alkynyl. In some embodiments, R^{L} is optionally substituted C₆₋₁₄ alkynyl. In some embodiments, R^{L} is optionally substituted C₆₋₁₃ alkynyl. In some embodiments, R^{L} is optionally substituted C₆₋₁₂ alkynyl. In some embodiments, R^{L} is optionally substituted C₆₋₁₁ alkynyl. In some embodiments, R^{L} is optionally substituted C₆₋₁₀ alkynyl. In some embodiments, R^{L} is optionally substituted C₆₋₉ alkynyl. In some embodiments, R^{L} is optionally substituted C₆₋₈ alkynyl. In some embodiments, R^{L} is optionally substituted C₆₋₇ alkynyl.

In some embodiments, R^{L} is optionally substituted C₈₋₅₀ alkynyl. In some embodiments, R^{L} is optionally substituted C₈₋₄₀ alkynyl. In some embodiments, R^{L} is optionally substituted C₈₋₃₀ alkynyl. In some embodiments, R^{L} is optionally substituted C₈₋₂₀ alkynyl. In some embodiments, R^{L} is optionally substituted C₈₋₁₉ alkynyl. In some embodiments, R^{L} is optionally substituted C₈₋₁₈ alkynyl. In some embodiments, R^{L} is optionally substituted C₈₋₁₇ alkynyl. In some embodiments, R^{L} is optionally substituted C₈₋₁₆ alkynyl. In some embodiments, R^{L} is optionally substituted C₈₋₁₅ alkynyl. In some embodiments, R^{L} is optionally substituted C₈₋₁₄ alkynyl. In some embodiments, R^{L} is optionally substituted C₈₋₁₃ alkynyl. In some embodiments, R^{L} is optionally substituted C₈₋₁₂ alkynyl. In some embodiments, R^{L} is optionally substituted C₈₋₁₁ alkynyl. In some embodiments, R^{L} is optionally substituted C₈₋₁₀ alkynyl. In some embodiments, R^{L} is optionally substituted C₈₋₉ alkynyl.

In some embodiments, R^{L} is optionally substituted C₉₋₅₀ alkynyl. In some embodiments, R^{L} is optionally substituted C₉₋₄₀ alkynyl. In some embodiments, R^{L} is optionally substituted C₉₋₃₀ alkynyl. In some embodiments, R^{L} is optionally substituted C₉₋₂₀ alkynyl. In some embodiments, R^{L} is optionally substituted C₉₋₁₉ alkynyl. In some embodiments, R^{L} is optionally substituted C₉₋₁₈ alkynyl. In some embodiments, R^{L} is optionally substituted C₉₋₁₇ alkynyl. In some embodiments, R^{L} is optionally substituted C₉₋₁₆ alkynyl. In some embodiments, R^{L} is optionally substituted C₉₋₁₅ alkynyl. In some embodiments, R^{L} is optionally substituted C₉₋₁₄ alkynyl. In some embodiments, R^{L} is optionally substituted C₉₋₁₃ alkynyl. In some embodiments, R^{L} is optionally substituted C₉₋₁₂ alkynyl. In some embodiments, R^{L} is optionally substituted C₉₋₁₁ alkynyl. In some embodiments, R^{L} is optionally substituted C₉₋₁₀ alkynyl.

In some embodiments, R^{L} is optionally substituted C₁₀₋₅₀ alkynyl. In some embodiments, R^{L} is optionally substituted C₁₀₋₄₀ alkynyl. In some embodiments, R^{L} is optionally substituted C₁₀₋₃₀ alkynyl. In some embodiments, R^{L} is optionally substituted C₁₀₋₂₀ alkynyl. In some embodiments, R^{L} is optionally substituted C₁₀₋₁₉ alkynyl. In some embodiments, R^{L} is optionally substituted C₁₀₋₁₈ alkynyl. In some embodiments, R^{L} is optionally substituted C₁₀₋₁₇ alkynyl. In some embodiments, R^{L} is optionally substituted C₁₀₋₁₆ alkynyl. In some embodiments, R^{L} is optionally substituted C₁₀₋₁₅ alkynyl. In some embodiments, R^{L} is optionally substituted C₁₀₋₁₄ alkynyl. In some embodiments, R^{L} is optionally substituted C₁₀₋₁₃ alkynyl. In some embodiments, R^{L} is optionally substituted C₁₀₋₁₂ alkynyl. In some embodiments, R^{L} is optionally substituted C₁₀₋₁₁ alkynyl.

In some embodiments, R^{L} is optionally substituted C₁₁₋₅₀ alkynyl. In some embodiments, R^{L} is optionally substituted C₁₁₋₄₀ alkynyl. In some embodiments, R^{L} is optionally substituted C₁₁₋₃₀ alkynyl. In some embodiments, R^{L} is optionally substituted C₁₁₋₂₀ alkynyl. In some embodiments, R^{L} is optionally substituted C₁₁₋₁₉ alkynyl. In some embodiments, R^{L} is optionally substituted C₁₁₋₁₈ alkynyl. In some embodiments, R^{L} is optionally substituted C₁₁₋₁₇ alkynyl. In some embodiments, R^{L} is optionally substituted C₁₁₋₁₆ alkynyl. In some embodiments, R^{L} is optionally substituted C₁₁₋₁₅ alkynyl. In some embodiments, R^{L} is optionally substituted C₁₁₋₁₄ alkynyl. In some embodiments, R^{L} is optionally substituted C₁₁₋₁₃ alkynyl. In some embodiments, R^{L} is optionally substituted C₁₁₋₁₂ alkynyl.

In some embodiments, R^{L} is optionally substituted C₁₂₋₅₀ alkynyl. In some embodiments, R^{L} is optionally substituted C₁₂₋₄₀ alkynyl. In some embodiments, R^{L} is optionally substituted C₁₂₋₃₀ alkynyl. In some embodiments, R^{L} is optionally substituted C₁₂₋₂₀ alkynyl. In some embodiments, R^{L} is optionally substituted C₁₂₋₁₉ alkynyl. In some embodiments, R^{L} is optionally substituted C₁₂₋₁₈ alkynyl. In some embodiments, R^{L} is optionally substituted C₁₂₋₁₇ alkynyl. In some embodiments, R^{L} is optionally substituted C₁₂₋₁₆ alkynyl. In some embodiments, R^{L} is optionally substituted C₁₂₋₁₅ alkynyl. In some embodiments, R^{L} is optionally substituted C₁₂₋₁₄ alkynyl. In some embodiments, R^{L} is optionally substituted C₁₂₋₁₃ alkynyl.

In some embodiments, R^{L} is optionally substituted C₆ alkynyl. In some embodiments, R^{L} is optionally substituted C₇ alkynyl. In some embodiments, R^{L} is optionally substituted C₈ alkynyl. In some embodiments, R^{L} is optionally substituted C₉ alkynyl. In some embodiments, R^{L} is optionally substituted C₁₀ alkynyl. In some embodiments, R^{L} is optionally substituted C₁₁ alkynyl. In some embodiments, R^{L} is optionally substituted C₁₂ alkynyl. In some embodiments, R^{L} is optionally substituted C₁₃ alkynyl. In some embodiments, R^{L} is optionally substituted C₁₄ alkynyl. In some embodiments, R^{L} is optionally substituted C₁₅ alkynyl. In some embodiments, R^{L} is optionally substituted C₁₆ alkynyl. In some embodiments, R^{L} is optionally substituted C₁₇ alkynyl. In some embodiments, R^{L} is optionally substituted C₁₈ alkynyl. In some embodiments, R^{L} is optionally substituted C₁₉ alkynyl. In some embodiments, R^{L} is optionally substituted C₂₀ alkynyl.

In some embodiments, for example, in any of the above embodiments, R^{L} is a substituted alkynyl group. In some embodiments, R^{L} is an unsubstituted alkynyl group. In some embodiments, R^{L} is an optionally substituted straight-chain alkyl group. In some embodiments, R^{L} is an optionally substituted straight-chain alkynyl group. In some embodiments, R^{L} is a substituted straight-chain alkynyl group. In some embodiments, R^{L} is an unsubstituted straight-chain alkynyl group. In some embodiments, R^{L} is an optionally substituted branched alkynyl group. In some embodiments, R^{L} is a substituted branched alkynyl group. In some embodiments, R^{L} is an unsubstituted branched alkynyl group.

In some embodiments, R^{L} is optionally substituted heteroC₁₋₅₀alkyl. In some embodiments, R^{L} is optionally substituted heteroC₂₋₅₀alkyl. In some embodiments, R^{L} is optionally substituted heteroC₂₋₄₀alkyl. In some embodiments, R^{L} is optionally substituted heteroC₂₋₃₀alkyl. In some embodiments, R^{L} is optionally substituted heteroC₂₋₂₀alkyl. In some embodiments, R^{L} is optionally substituted heteroC₂₋₁₉alkyl. In some embodiments, R^{L} is optionally substituted heteroC₂₋₁₈alkyl. In some embodiments, R^{L} is optionally substituted heteroC₂₋₁₇alkyl. In some embodiments, R^{L} is optionally substituted heteroC₂₋₁₆alkyl. In some embodiments, R^{L} is optionally substituted heteroC₂₋₁₅alkyl. In some embodiments, R^{L} is optionally substituted heteroC₂₋₁₄alkyl. In some embodiments, R^{L} is optionally substituted heteroC₂₋₁₃alkyl. In some embodiments, R^{L} is optionally substituted heteroC₂₋₁₂alkyl. In some embodiments, R^{L} is optionally substituted heteroC₂₋₁₁alkyl. In some embodiments, R^{L} is optionally substituted heteroC₂₋₁₀ alkyl. In some embodiments, R^{L} is optionally substituted heteroC₂₋₉alkyl. In some embodiments, R^{L} is optionally substituted heteroC₂₋₈ alkyl. In some embodiments, R^{L} is optionally substituted heteroC₂₋₇alkyl. In some embodiments, R^{L} is optionally substituted heteroC₂₋₆alkyl.

In some embodiments, R^{L} is optionally substituted heteroC₄₋₅₀alkyl. In some embodiments, R^{L} is optionally substituted heteroC₄₋₄₀alkyl. In some embodiments, R^{L} is optionally substituted heteroC₄₋₃₀alkyl. In some embodiments, R^{L} is optionally substituted heteroC₄₋₂₀alkyl. In some embodiments, R^{L} is optionally substituted heteroC₄₋₁₉alkyl. In some embodiments, R^{L} is optionally substituted heteroC₄₋₁₈ alkyl. In some embodiments, R^{L} is optionally substituted heteroC₄₋₁₇alkyl. In some embodiments, R^{L} is optionally substituted heteroC₄₋₁₆alkyl. In some embodiments, R^{L} is optionally substituted heteroC₄₋₁₅alkyl. In some embodiments, R^{L} is optionally substituted heteroC₄₋₁₄alkyl. In some embodiments, R^{L} is optionally substituted heteroC₄₋₁₃alkyl. In some embodiments, R^{L} is optionally substituted heteroC₄₋₁₂alkyl. In some embodiments, R^{L} is optionally substituted heteroC₄₋₁₁alkyl. In some embodiments, R^{L} is optionally substituted heteroC₄₋₁₀alkyl. In some embodiments, R^{L} is optionally substituted heteroC₄₋₉alkyl. In some embodiments, R^{L} is optionally substituted heteroC₄₋₈alkyl. In some embodiments, R^{L} is optionally substituted heteroC₄₋₇alkyl. In some embodiments, R^{L} is optionally substituted heteroC₄₋₆alkyl.

In some embodiments, R^{L} is optionally substituted heteroC₆₋₅₀alkyl. In some embodiments, R^{L} is optionally substituted heteroC₆₋₄₀alkyl. In some embodiments, R^{L} is optionally substituted heteroC₆₋₃₀alkyl. In some embodiments, R^{L} is optionally substituted heteroC₆₋₂₀alkyl. In some embodiments, R^{L} is optionally substituted heteroC₆₋₁₉alkyl. In some embodiments, R^{L} is optionally substituted heteroC₆₋₁₈alkyl. In some embodiments, R^{L} is optionally substituted heteroC₆₋₁₇alkyl. In some embodiments, R^{L} is optionally substituted heteroC₆₋₁₆alkyl. In some embodiments, R^{L} is optionally substituted heteroC₆₋₁₅alkyl. In some embodiments, R^{L} is optionally substituted heteroC₆₋₁₄alkyl. In some embodiments, R^{L} is optionally substituted heteroC₆₋₁₃alkyl. In some embodiments, R^{L} is optionally substituted heteroC₆₋₁₂alkyl. In some embodiments, R^{L} is optionally substituted heteroC₆₋₁₁alkyl. In some embodiments, R^{L} is optionally substituted heteroC₆₋₁₀alkyl. In some embodiments, R^{L} is optionally substituted heteroC₆₋₉alkyl. In some embodiments, R^{L} is optionally substituted heteroC₆₋₈alkyl. In some embodiments, R^{L} is optionally substituted heteroC₆₋₇alkyl.

In some embodiments, R^{L} is optionally substituted heteroC₈₋₅₀alkyl. In some embodiments, R^{L} is optionally substituted heteroC₈₋₄₀alkyl. In some embodiments, R^{L} is optionally substituted heteroC₈₋₃₀alkyl. In some embodiments, R^{L} is optionally substituted heteroC₈₋₂₀alkyl. In some embodiments, R^{L} is optionally substituted heteroC₈₋₁₉alkyl. In some embodiments, R^{L} is optionally substituted heteroC₈₋₁₈alkyl. In some embodiments, R^{L} is optionally substituted heteroC₈₋₁₇alkyl. In some embodiments, R^{L} is optionally substituted heteroC₈₋₁₆alkyl. In some embodiments, R^{L} is optionally substituted heteroC₈₋₁₅alkyl. In some embodiments, R^{L} is optionally substituted heteroC₈₋₁₄alkyl. In some embodiments, R^{L} is optionally substituted heteroC₈₋₁₃alkyl. In some embodiments, R^{L} is optionally substituted heteroC₈₋₁₂alkyl. In some embodiments, R^{L} is optionally substituted heteroC₈₋₁₁alkyl. In some embodiments, R^{L} is optionally substituted heteroC₈₋₁₀alkyl. In some embodiments, R^{L} is optionally substituted heteroC₈₋₉alkyl.

In some embodiments, R^{L} is optionally substituted heteroC₉₋₅₀alkyl. In some embodiments, R^{L} is optionally substituted heteroC₉₋₄₀alkyl. In some embodiments, R^{L} is optionally substituted heteroC₉₋₃₀alkyl. In some embodiments, R^{L} is optionally substituted heteroC₉₋₂₀alkyl. In some embodiments, R^{L} is optionally substituted heteroC₉₋₁₉alkyl. In some embodiments, R^{L} is optionally substituted heteroC₉₋₁₈alkyl. In some embodiments, R^{L} is optionally substituted heteroC₉₋₁₇alkyl. In some embodiments, R^{L} is optionally substituted heteroC₉₋₁₆alkyl. In some embodiments, R^{L} is optionally substituted heteroC₉₋₁₅alkyl. In some embodiments, R^{L} is optionally substituted heteroC₉₋₁₄alkyl. In some embodiments, R^{L} is optionally substituted heteroC₉₋₁₃alkyl. In some embodiments, R^{L} is optionally substituted heteroC₉₋₁₂alkyl. In some embodiments, R^{L} is optionally substituted heteroC₉₋₁₁alkyl. In some embodiments, R^{L} is optionally substituted heteroC₉₋₁₀alkyl.

In some embodiments, R^{L} is optionally substituted heteroC₁₀₋₅₀alkyl. In some embodiments, R^{L} is optionally substituted heteroC₁₀₋₄₀alkyl. In some embodiments, R^{L} is optionally substituted heteroC₁₀₋₃₀alkyl. In some embodiments, R^{L} is optionally substituted heteroC₁₀₋₂₀alkyl. In some embodiments, R^{L} is optionally substituted heteroC₁₀₋₁₉alkyl. In some embodiments, R^{L} is optionally substituted heteroC₁₀₋₁₈alkyl. In some embodiments, R^{L} is optionally substituted heteroC₁₀₋₁₇alkyl. In some embodiments, R^{L} is optionally substituted heteroC₁₀₋₁₆alkyl. In some embodiments, R^{L} is optionally substituted heteroC₁₀₋₁₅alkyl. In some embodiments, R^{L} is optionally substituted heteroC₁₀₋₁₄alkyl. In some embodiments, R^{L} is optionally substituted heteroC₁₀₋₁₃alkyl. In some embodiments, R^{L} is optionally substituted heteroC₁₀₋₁₂alkyl. In some embodiments, R^{L} is optionally substituted heteroC₁₀₋₁₁alkyl.

In some embodiments, R^{L} is optionally substituted heteroC₁₁₋₅₀alkyl. In some embodiments, R^{L} is optionally substituted heteroC₁₁₋₄₀alkyl. In some embodiments, R^{L} is optionally substituted heteroC₁₁₋₃₀alkyl. In some embodiments, R^{L} is optionally substituted heteroC₁₁₋₂₀alkyl. In some embodiments, R^{L} is optionally substituted heteroC₁₁₋₁₉alkyl. In some embodiments, R^{L} is optionally substituted heteroC₁₁₋₁₈alkyl. In some embodiments, R^{L} is optionally substituted heteroC₁₁₋₁₇alkyl. In some embodiments, R^{L} is optionally substituted heteroC₁₁₋₁₆alkyl. In some embodiments, R^{L} is optionally substituted heteroC₁₁₋₁₅alkyl. In some embodiments, R^{L} is optionally substituted heteroC₁₁₋₁₄alkyl. In some embodiments, R^{L} is optionally substituted heteroC₁₁₋₁₃alkyl. In some embodiments, R^{L} is optionally substituted heteroC₁₁₋₁₂alkyl.

In some embodiments, R^{L} is optionally substituted heteroC₁₂₋₅₀alkyl. In some embodiments, R^{L} is optionally substituted heteroC₁₂₋₄₀alkyl. In some embodiments, R^{L} is optionally substituted heteroC₁₂₋₃₀alkyl. In some embodiments, R^{L} is optionally substituted heteroC₁₂₋₂₀alkyl. In some embodiments, R^{L} is optionally substituted heteroC₁₂₋₁₉alkyl. In some embodiments, R^{L} is optionally substituted heteroC₁₂₋₁₈alkyl. In some embodiments, R^{L} is optionally substituted heteroC₁₂₋₁₇alkyl. In some embodiments, R^{L} is optionally substituted heteroC₁₂₋₁₆alkyl. In some embodiments, R^{L} is optionally substituted heteroC₁₂₋₁₅alkyl. In some embodiments, R^{L} is optionally substituted heteroC₁₂₋₁₄alkyl. In some embodiments, R^{L} is optionally substituted heteroC₁₂₋₁₃alkyl.

In some embodiments, R^{L} is optionally substituted heteroC₆alkyl. In some embodiments, R^{L} is optionally substituted heteroC₇alkyl. In some embodiments, R^{L} is optionally substituted heteroC₈alkyl. In some embodiments, R^{L} is optionally substituted heteroC₉alkyl. In some embodiments, R^{L} is optionally substituted heteroC₁₀alkyl. In some embodiments, R^{L} is optionally substituted heteroC₁₁alkyl. In some embodiments, R^{L} is optionally substituted heteroC₁₂alkyl. In some embodiments, R^{L} is optionally substituted heteroC₁₃alkyl. In some embodiments, R^{L} is optionally substituted heteroC₁₄alkyl. In some embodiments, R^{L} is optionally substituted heteroCᵢₛalkyl. In some embodiments, R^{L} is optionally substituted heteroC₁₆alkyl. In some embodiments, R^{L} is optionally substituted heteroC₁₇alkyl. In some embodiments, R^{L} is optionally substituted heteroC₁₈alkyl. In some embodiments, R^{L} is optionally substituted heteroC₁₉alkyl. In some embodiments, R^{L} is optionally substituted heteroC₂₀alkyl.

In some embodiments, for example, in any of the above embodiments, R^{L} is a substituted heteroalkyl group. In some embodiments, R^{L} is an unsubstituted heteroalkyl group. In some embodiments, R^{L} is an optionally substituted straight-chain heteroalkyl group. In some embodiments, R^{L} is a substituted straight-chain heteroalkyl group. In some embodiments, R^{L} is an unsubstituted straight-chain heteroalkyl group. In some embodiments, R^{L} is an optionally substituted branched heteroalkyl group. In some embodiments, R^{L} is a substituted branched heteroalkyl group. In some embodiments, R^{L} is an unsubstituted branched heteroalkyl group.

Exemplary unsubstituted heteroalkyl groups include, but are not limited to:

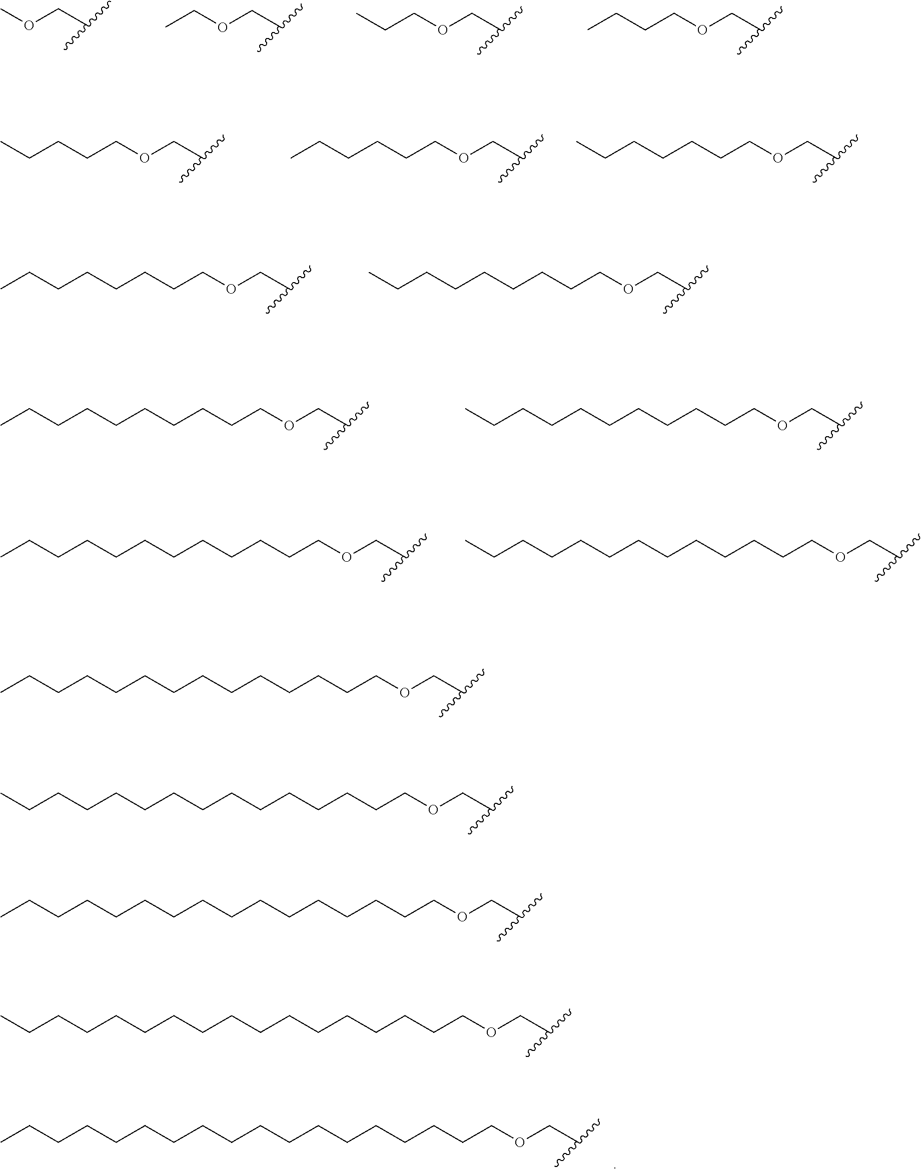

In some embodiments, R^{L} is optionally substituted heteroC₂₋₅₀alkenyl. In some embodiments, R^{L} is optionally substituted heteroC₂₋₄₀alkenyl. In some embodiments, R^{L} is optionally substituted heteroC₂₋₃₀alkenyl. In some embodiments, R^{L} is optionally substituted heteroC₂₋₂₀alkenyl. In some embodiments, R^{L} is optionally substituted heteroC₂₋₁₉alkenyl. In some embodiments, R^{L} is optionally substituted heteroC₂₋₁₈ alkenyl. In some embodiments, R^{L} is optionally substituted heteroC₂₋₁₇alkenyl. In some embodiments, R^{L} is optionally substituted heteroC₂₋₁₆alkenyl. In some embodiments, R^{L} is optionally substituted heteroC₂₋₁₅alkenyl. In some embodiments, R^{L} is optionally substituted heteroC₂₋₁₄alkenyl. In some embodiments, R^{L} is optionally substituted heteroC₂₋₁₃alkenyl. In some embodiments, R^{L} is optionally substituted heteroC₂₋₁₂alkenyl. In some embodiments, R^{L} is optionally substituted heteroC₂₋₁₁alkenyl. In some embodiments, R^{L} is optionally substituted heteroC₂₋₁₀ alkenyl. In some embodiments, R^{L} is optionally substituted heteroC₂₋₉alkenyl. In some embodiments, R^{L} is optionally substituted heteroC₂₋₈ alkenyl. In some embodiments, R^{L} is optionally substituted heteroC₂₋₇alkenyl. In some embodiments, R^{L} is optionally substituted heteroC₂₋₆alkenyl.

In some embodiments, R^{L} is optionally substituted heteroC₄₋₅₀alkenyl. In some embodiments, R^{L} is optionally substituted heteroC₄₋₄₀alkenyl. In some embodiments, R^{L} is optionally substituted heteroC₄₋₃₀alkenyl. In some embodiments, R^{L} is optionally substituted heteroC₄₋₂₀alkenyl. In some embodiments, R^{L} is optionally substituted heteroC₄₋₁₉alkenyl. In some embodiments, R^{L} is optionally substituted heteroC₄₋₁₈ alkenyl. In some embodiments, R^{L} is optionally substituted heteroC₄₋₁₇alkenyl. In some embodiments, R^{L} is optionally substituted heteroC₄₋₁₆alkenyl. In some embodiments, R^{L} is optionally substituted heteroC₄₋₁₅alkenyl. In some embodiments, R^{L} is optionally substituted heteroC₄₋₁₄alkenyl. In some embodiments, R^{L} is optionally substituted heteroC₄₋₁₃alkenyl. In some embodiments, R^{L} is optionally substituted heteroC₄₋₁₂alkenyl. In some embodiments, R^{L} is optionally substituted heteroC₄₋₁₁ alkenyl. In some embodiments, R^{L} is optionally substituted heteroC₄₋₁₀ alkenyl. In some embodiments, R^{L} is optionally substituted heteroC₄₋₉alkenyl. In some embodiments, R^{L} is optionally substituted heteroC₄₋₈alkenyl. In some embodiments, R^{L} is optionally substituted heteroC₄₋₇alkenyl. In some embodiments, R^{L} is optionally substituted heteroC₄₋₆alkenyl.

In some embodiments, R^{L} is optionally substituted heteroC₆₋₅₀alkenyl. In some embodiments, R^{L} is optionally substituted heteroC₆₋₄₀alkenyl. In some embodiments, R^{L} is optionally substituted heteroC₆₋₃₀alkenyl. In some embodiments, R^{L} is optionally substituted heteroC₆₋₂₀alkenyl. In some embodiments, R^{L} is optionally substituted heteroC₆₋₁₉alkenyl. In some embodiments, R^{L} is optionally substituted heteroC₆₋₁₈alkenyl. In some embodiments, R^{L} is optionally substituted heteroC₆₋₁₇alkenyl. In some embodiments, R^{L} is optionally substituted heteroC₆₋₁₆alkenyl. In some embodiments, R^{L} is optionally substituted heteroC₆₋₁₅alkenyl. In some embodiments, R^{L} is optionally substituted heteroC₆₋₁₄alkenyl. In some embodiments, R^{L} is optionally substituted heteroC₆₋₁₃alkenyl. In some embodiments, R^{L} is optionally substituted heteroC₆₋₁₂alkenyl. In some embodiments, R^{L} is optionally substituted heteroC₆₋₁₁ alkenyl. In some embodiments, R^{L} is optionally substituted heteroC₆₋₁₀alkenyl. In some embodiments, R^{L} is optionally substituted heteroC₆₋₉alkenyl. In some embodiments, R^{L} is optionally substituted heteroC₆₋₈ alkenyl. In some embodiments, R^{L} is optionally substituted heteroC₆₋₇alkenyl.

In some embodiments, R^{L} is optionally substituted heteroC₈₋₅₀alkenyl. In some embodiments, R^{L} is optionally substituted heteroC₈₋₄₀alkenyl. In some embodiments, R^{L} is optionally substituted heteroC₈₋₃₀alkenyl. In some embodiments, R^{L} is optionally substituted heteroC₈₋₂₀alkenyl. In some embodiments, R^{L} is optionally substituted heteroC₈₋₁₉alkenyl. In some embodiments, R^{L} is optionally substituted heteroC₈₋₁₈ alkenyl. In some embodiments, R^{L} is optionally substituted heteroC₈₋₁₇alkenyl. In some embodiments, R^{L} is optionally substituted heteroC₈₋₁₆alkenyl. In some embodiments, R^{L} is optionally substituted heteroC₈₋₁₅alkenyl. In some embodiments, R^{L} is optionally substituted heteroC₈₋₁₄alkenyl. In some embodiments, R^{L} is optionally substituted heteroC₈₋₁₃alkenyl. In some embodiments, R^{L} is optionally substituted heteroC₈₋₁₂alkenyl. In some embodiments, R^{L} is optionally substituted heteroC₈₋₁₁alkenyl. In some embodiments, R^{L} is optionally substituted heteroC₈₋₁₀alkenyl. In some embodiments, R^{L} is optionally substituted heteroC₈₋₉alkenyl.

In some embodiments, R^{L} is optionally substituted heteroC₉₋₅₀alkenyl. In some embodiments, R^{L} is optionally substituted heteroC₉₋₄₀alkenyl. In some embodiments, R^{L} is optionally substituted heteroC₉₋₃₀alkenyl. In some embodiments, R^{L} is optionally substituted heteroC₉₋₂₀alkenyl. In some embodiments, R^{L} is optionally substituted heteroC₉₋₁₉alkenyl. In some embodiments, R^{L} is optionally substituted heteroC₉₋₁₈alkenyl. In some embodiments, R^{L} is optionally substituted heteroC₉₋₁₇alkenyl. In some embodiments, R^{L} is optionally substituted heteroC₉₋₁₆alkenyl. In some embodiments, R^{L} is optionally substituted heteroC₉₋₁₅alkenyl. In some embodiments, R^{L} is optionally substituted heteroC₉₋₁₄alkenyl. In some embodiments, R^{L} is optionally substituted heteroC₉₋₁₃alkenyl. In some embodiments, R^{L} is optionally substituted heteroC₉₋₁₂alkenyl. In some embodiments, R^{L} is optionally substituted heteroC₉₋₁₁alkenyl. In some embodiments, R^{L} is optionally substituted heteroC₉₋₁₀alkenyl.

In some embodiments, R^{L} is optionally substituted heteroC₁₀₋₅₀alkenyl. In some embodiments, R^{L} is optionally substituted heteroC₁₀₋₄₀alkenyl. In some embodiments, R^{L} is optionally substituted heteroC₁₀₋₃₀alkenyl. In some embodiments, R^{L} is optionally substituted heteroC₁₀₋₂₀alkenyl. In some embodiments, R^{L} is optionally substituted heteroC₁₀₋₁₉alkenyl. In some embodiments, R^{L} is optionally substituted heteroC₁₀₋₁₈alkenyl. In some embodiments, R^{L} is optionally substituted heteroC₁₀₋₁₇alkenyl. In some embodiments, R^{L} is optionally substituted heteroC₁₀₋₁₆alkenyl. In some embodiments, R^{L} is optionally substituted heteroC₁₀₋₁₅alkenyl. In some embodiments, R^{L} is optionally substituted heteroC₁₀₋₁₄alkenyl. In some embodiments, R^{L} is optionally substituted heteroC₁₀₋₁₃alkenyl. In some embodiments, R^{L} is optionally substituted heteroC₁₀₋₁₂alkenyl. In some embodiments, R^{L} is optionally substituted heteroC₁₀₋₁₁alkenyl.

In some embodiments, R^{L} is optionally substituted heteroC₁₁₋₅₀alkenyl. In some embodiments, R^{L} is optionally substituted heteroC₁₁₋₄₀alkenyl. In some embodiments, R^{L} is optionally substituted heteroC₁₁₋₃₀alkenyl. In some embodiments, R^{L} is optionally substituted heteroC₁₁₋₂₀alkenyl. In some embodiments, R^{L} is optionally substituted heteroC₁₁₋₁₉alkenyl. In some embodiments, R^{L} is optionally substituted heteroC₁₁₋₁₈alkenyl. In some embodiments, R^{L} is optionally substituted heteroC₁₁₋₁₇alkenyl. In some embodiments, R^{L} is optionally substituted heteroC₁₁₋₁₆alkenyl. In some embodiments, R^{L} is optionally substituted heteroC₁₁₋₁₅alkenyl. In some embodiments, R^{L} is optionally substituted heteroC₁₁₋₁₄alkenyl. In some embodiments, R^{L} is optionally substituted heteroC₁₁₋₁₃alkenyl. In some embodiments, R^{L} is optionally substituted heteroC₁₁₋₁₂alkenyl.

In some embodiments, R^{L} is optionally substituted heteroC₁₂₋₅₀alkenyl. In some embodiments, R^{L} is optionally substituted heteroC₁₂₋₄₀alkenyl. In some embodiments, R^{L} is optionally substituted heteroC₁₂₋₃₀alkenyl. In some embodiments, R^{L} is optionally substituted heteroC₁₂₋₂₀alkenyl. In some embodiments, R^{L} is optionally substituted heteroC₁₂₋₁₉alkenyl. In some embodiments, R^{L} is optionally substituted heteroC₁₂₋₁₈alkenyl. In some embodiments, R^{L} is optionally substituted heteroC₁₂₋₁₇alkenyl. In some embodiments, R^{L} is optionally substituted heteroC₁₂₋₁₆alkenyl. In some embodiments, R^{L} is optionally substituted heteroC₁₂₋₁₅alkenyl. In some embodiments, R^{L} is optionally substituted heteroC₁₂₋₁₄alkenyl. In some embodiments, R^{L} is optionally substituted heteroC₁₂₋₁₃alkenyl.

In some embodiments, R^{L} is optionally substituted heteroC₆alkenyl. In some embodiments, R^{L} is optionally substituted heteroC₇alkenyl. In some embodiments, R^{L} is optionally substituted heteroC₈alkenyl. In some embodiments, R^{L} is optionally substituted heteroC₉alkenyl. In some embodiments, R^{L} is optionally substituted heteroC₁₀alkenyl. In some embodiments, R^{L} is optionally substituted heteroC₁₁alkenyl. In some embodiments, R^{L} is optionally substituted heteroC₁₂alkenyl. In some embodiments, R^{L} is optionally substituted heteroC₁₃alkenyl. In some embodiments, R^{L} is optionally substituted heteroC₁₄alkenyl. In some embodiments, R^{L} is optionally substituted heteroCᵢₛalkenyl. In some embodiments, R^{L} is optionally substituted heteroC₁₆alkenyl. In some embodiments, R^{L} is optionally substituted heteroC₁₇alkenyl. In some embodiments, R^{L} is optionally substituted heteroC₁₈alkenyl. In some embodiments, R^{L} is optionally substituted heteroC₁₉alkenyl. In some embodiments, R^{L} is optionally substituted heteroC₂₀alkenyl.

In some embodiments, for example, in any of the above embodiments, R^{L} is a substituted heteroalkenyl group. In some embodiments, R^{L} is an unsubstituted heteroalkenyl group. In some embodiments, R^{L} is an optionally substituted straight-chain heteroalkenyl group. In some embodiments, R^{L} is a substituted straight-chain heteroalkenyl group. In some embodiments, R^{L} is an unsubstituted straight-chain heteroalkenyl group. In some embodiments, R^{L} is an optionally substituted branched heteroalkenyl group. In some embodiments, R^{L} is a substituted branched heteroalkenyl group. In some embodiments, R^{L} is an unsubstituted branched heteroalkenyl group.

In some embodiments, R^{L} is optionally substituted heteroC₂₋₅₀alkynyl. In some embodiments, R^{L} is optionally substituted heteroC₂₋₄₀alkynyl. In some embodiments, R^{L} is optionally substituted heteroC₂₋₃₀alkynyl. In some embodiments, R^{L} is optionally substituted heteroC₂₋₂₀alkynyl. In some embodiments, R^{L} is optionally substituted heteroC₂₋₁₉alkynyl. In some embodiments, R^{L} is optionally substituted heteroC₂₋₁₈ alkynyl. In some embodiments, R^{L} is optionally substituted heteroC₂₋₁₇alkynyl. In some embodiments, R^{L} is optionally substituted heteroC₂₋₁₆alkynyl. In some embodiments, R^{L} is optionally substituted heteroC₂₋₁₅alkynyl. In some embodiments, R^{L} is optionally substituted heteroC₂₋₁₄alkynyl. In some embodiments, R^{L} is optionally substituted heteroC₂₋₁₃alkynyl. In some embodiments, R^{L} is optionally substituted heteroC₂₋₁₂alkynyl. In some embodiments, R^{L} is optionally substituted heteroC₂₋₁₁ alkynyl. In some embodiments, R^{L} is optionally substituted heteroC₂₋₁₀alkynyl. In some embodiments, R^{L} is optionally substituted heteroC₂₋₉alkynyl. In some embodiments, R^{L} is optionally substituted heteroC₂₋₈ alkynyl. In some embodiments, R^{L} is optionally substituted heteroC₂₋₇alkynyl. In some embodiments, R^{L} is optionally substituted heteroC₂₋₆alkynyl.

In some embodiments, R^{L} is optionally substituted heteroC₄₋₅₀alkynyl. In some embodiments, R^{L} is optionally substituted heteroC₄₋₄₀alkynyl. In some embodiments, R^{L} is optionally substituted heteroC₄₋₃₀alkynyl. In some embodiments, R^{L} is optionally substituted heteroC₄₋₂₀alkynyl. In some embodiments, R^{L} is optionally substituted heteroC₄₋₁₉alkynyl. In some embodiments, R^{L} is optionally substituted heteroC₄₋₁₈alkynyl. In some embodiments, R^{L} is optionally substituted heteroC₄₋₁₇alkynyl. In some embodiments, R^{L} is optionally substituted heteroC₄₋₁₆alkynyl. In some embodiments, R^{L} is optionally substituted heteroC₄₋₁₅alkynyl. In some embodiments, R^{L} is optionally substituted heteroC₄₋₁₄alkynyl. In some embodiments, R^{L} is optionally substituted heteroC₄₋₁₃alkynyl. In some embodiments, R^{L} is optionally substituted heteroC₄₋₁₂alkynyl. In some embodiments, R^{L} is optionally substituted heteroC₄₋₁₁alkynyl. In some embodiments, R^{L} is optionally substituted heteroC₄₋₁₀alkynyl. In some embodiments, R^{L} is optionally substituted heteroC₄₋₉alkynyl. In some embodiments, R^{L} is optionally substituted heteroC₄₋₈alkynyl. In some embodiments, R^{L} is optionally substituted heteroC₄₋₇alkynyl. In some embodiments, R^{L} is optionally substituted heteroC₄₋₆alkynyl.

In some embodiments, R^{L} is optionally substituted heteroC₆₋₅₀alkynyl. In some embodiments, R^{L} is optionally substituted heteroC₆₋₄₀alkynyl. In some embodiments, R^{L} is optionally substituted heteroC₆₋₃₀alkynyl. In some embodiments, R^{L} is optionally substituted heteroC₆₋₂₀alkynyl. In some embodiments, R^{L} is optionally substituted heteroC₆₋₁₉alkynyl. In some embodiments, R^{L} is optionally substituted heteroC₆₋₁₈alkynyl. In some embodiments, R^{L} is optionally substituted heteroC₆₋₁₇alkynyl. In some embodiments, R^{L} is optionally substituted heteroC₆₋₁₆alkynyl. In some embodiments, R^{L} is optionally substituted heteroC₆₋₁₅alkynyl. In some embodiments, R^{L} is optionally substituted heteroC₆₋₁₄alkynyl. In some embodiments, R^{L} is optionally substituted heteroC₆₋₁₃alkynyl. In some embodiments, R^{L} is optionally substituted heteroC₆₋₁₂alkynyl. In some embodiments, R^{L} is optionally substituted heteroC₆₋₁₁alkynyl. In some embodiments, R^{L} is optionally substituted heteroC₆₋₁₀alkynyl. In some embodiments, R^{L} is optionally substituted heteroC₆₋₉alkynyl. In some embodiments, R^{L} is optionally substituted heteroC₆₋₈alkynyl. In some embodiments, R^{L} is optionally substituted heteroC₆₋₇alkynyl.

In some embodiments, R^{L} is optionally substituted heteroC₈₋₅₀alkynyl. In some embodiments, R^{L} is optionally substituted heteroC₈₋₄₀alkynyl. In some embodiments, R^{L} is optionally substituted heteroC₈₋₃₀alkynyl. In some embodiments, R^{L} is optionally substituted heteroC₈₋₂₀alkynyl. In some embodiments, R^{L} is optionally substituted heteroC₈₋₁₉alkynyl. In some embodiments, R^{L} is optionally substituted heteroC₈₋₁₈alkynyl. In some embodiments, R^{L} is optionally substituted heteroC₈₋₁₇alkynyl. In some embodiments, R^{L} is optionally substituted heteroC₈₋₁₆alkynyl. In some embodiments, R^{L} is optionally substituted heteroC₈₋₁₅alkynyl. In some embodiments, R^{L} is optionally substituted heteroC₈₋₁₄alkynyl. In some embodiments, R^{L} is optionally substituted heteroC₈₋₁₃alkynyl. In some embodiments, R^{L} is optionally substituted heteroC₈₋₁₂alkynyl. In some embodiments, R^{L} is optionally substituted heteroC₈₋₁₁alkynyl. In some embodiments, R^{L} is optionally substituted heteroC₈₋₁₀alkynyl. In some embodiments, R^{L} is optionally substituted heteroC₈₋₉alkynyl.

In some embodiments, R^{L} is optionally substituted heteroC₉₋₅₀alkynyl. In some embodiments, R^{L} is optionally substituted heteroC₉₋₄₀alkynyl. In some embodiments, R^{L} is optionally substituted heteroC₉₋₃₀alkynyl. In some embodiments, R^{L} is optionally substituted heteroC₉₋₂₀alkynyl. In some embodiments, R^{L} is optionally substituted heteroC₉₋₁₉alkynyl. In some embodiments, R^{L} is optionally substituted heteroC₉₋₁₈alkynyl. In some embodiments, R^{L} is optionally substituted heteroC₉₋₁₇alkynyl. In some embodiments, R^{L} is optionally substituted heteroC₉₋₁₆alkynyl. In some embodiments, R^{L} is optionally substituted heteroC₉₋₁₅alkynyl. In some embodiments, R^{L} is optionally substituted heteroC₉₋₁₄alkynyl. In some embodiments, R^{L} is optionally substituted heteroC₉₋₁₃alkynyl. In some embodiments, R^{L} is optionally substituted heteroC₉₋₁₂alkynyl. In some embodiments, R^{L} is optionally substituted heteroC₉₋₁₁alkynyl. In some embodiments, R^{L} is optionally substituted heteroC₉₋₁₀alkynyl.

In some embodiments, R^{L} is optionally substituted heteroC₁₀₋₅₀alkynyl. In some embodiments, R^{L} is optionally substituted heteroC₁₀₋₄₀alkynyl. In some embodiments, R^{L} is optionally substituted heteroC₁₀₋₃₀alkynyl. In some embodiments, R^{L} is optionally substituted heteroC₁₀₋₂₀alkynyl. In some embodiments, R^{L} is optionally substituted heteroC₁₀₋₁₉alkynyl. In some embodiments, R^{L} is optionally substituted heteroC₁₀₋₁₈alkynyl. In some embodiments, R^{L} is optionally substituted heteroC₁₀₋₁₇alkynyl. In some embodiments, R^{L} is optionally substituted heteroC₁₀₋₁₆alkynyl. In some embodiments, R^{L} is optionally substituted heteroC₁₀₋₁₅alkynyl. In some embodiments, R^{L} is optionally substituted heteroC₁₀₋₁₄alkynyl. In some embodiments, R^{L} is optionally substituted heteroC₁₀₋₁₃alkynyl. In some embodiments, R^{L} is optionally substituted heteroC₁₀₋₁₂alkynyl. In some embodiments, R^{L} is optionally substituted heteroC₁₀₋₁₁alkynyl.

In some embodiments, R^{L} is optionally substituted heteroC₁₁₋₅₀alkynyl. In some embodiments, R^{L} is optionally substituted heteroC₁₁₋₄₀alkynyl. In some embodiments, R^{L} is optionally substituted heteroC₁₁₋₃₀alkynyl. In some embodiments, R^{L} is optionally substituted heteroC₁₁₋₂₀alkynyl. In some embodiments, R^{L} is optionally substituted heteroC₁₁₋₁₉alkynyl. In some embodiments, R^{L} is optionally substituted heteroC₁₁₋₁₈alkynyl. In some embodiments, R^{L} is optionally substituted heteroC₁₁₋₁₇alkynyl. In some embodiments, R^{L} is optionally substituted heteroC₁₁₋₁₆alkynyl. In some embodiments, R^{L} is optionally substituted heteroC₁₁₋₁₅alkynyl. In some embodiments, R^{L} is optionally substituted heteroC₁₁₋₁₄alkynyl. In some embodiments, R^{L} is optionally substituted heteroC₁₁₋₁₃alkynyl. In some embodiments, R^{L} is optionally substituted heteroC₁₁₋₁₂alkynyl.

In some embodiments, R^{L} is optionally substituted heteroC₁₂₋₅₀alkynyl. In some embodiments, R^{L} is optionally substituted heteroC₁₂₋₄₀alkynyl. In some embodiments, R^{L} is optionally substituted heteroC₁₂₋₃₀alkynyl. In some embodiments, R^{L} is optionally substituted heteroC₁₂₋₂₀alkynyl. In some embodiments, R^{L} is optionally substituted heteroC₁₂₋₁₉alkynyl. In some embodiments, R^{L} is optionally substituted heteroC₁₂₋₁₈alkynyl. In some embodiments, R^{L} is optionally substituted heteroC₁₂₋₁₇alkynyl. In some embodiments, R^{L} is optionally substituted heteroC₁₂₋₁₆alkynyl. In some embodiments, R^{L} is optionally substituted heteroC₁₂₋₁₅alkynyl. In some embodiments, R^{L} is optionally substituted heteroC₁₂₋₁₄alkynyl. In some embodiments, R^{L} is optionally substituted heteroC₁₂₋₁₃alkynyl.

In some embodiments, R^{L} is optionally substituted heteroC₆alkynyl. In some embodiments, R^{L} is optionally substituted heteroC₇alkynyl. In some embodiments, R^{L} is optionally substituted heteroC₈alkynyl. In some embodiments, R^{L} is optionally substituted heteroC₉alkynyl. In some embodiments, R^{L} is optionally substituted heteroC₁₀alkynyl. In some embodiments, R^{L} is optionally substituted heteroC₁₁alkynyl. In some embodiments, R^{L} is optionally substituted heteroC₁₂alkynyl. In some embodiments, R^{L} is optionally substituted heteroC₁₃alkynyl. In some embodiments, R^{L} is optionally substituted heteroC₁₄alkynyl. In some embodiments, R^{L} is optionally substituted heteroC₁₅alkynyl. In some embodiments, R^{L} is optionally substituted heteroC₁₆alkynyl. In some embodiments, R^{L} is optionally substituted heteroC₁₇alkynyl. In some embodiments, R^{L} is optionally substituted heteroC₁₈alkynyl. In some embodiments, R^{L} is optionally substituted heteroC₁₉alkynyl. In some embodiments, R^{L} is optionally substituted heteroC₂₀alkynyl.

In some embodiments, for example, in any of the above embodiments, R^{L} is a substituted heteroalkynyl group. In some embodiments, R^{L} is an unsubstituted heteroalkynyl group. In some embodiments, R^{L} is an optionally substituted straight-chain heteroalkynyl group. In some embodiments, R^{L} is a substituted straight-chain heteroalkynyl group. In some embodiments, R^{L} is an unsubstituted straight-chain heteroalkynyl group. In some embodiments, R^{L} is an optionally substituted branched heteroalkynyl group. In some embodiments, R^{L} is a substituted branched heteroalkynyl group. In some embodiments, R^{L} is an unsubstituted branched heteroalkynyl group.

In some embodiments, R^{L} is a polymer. As used herein, a “polymer”, in some embodiments, refers to a compound comprised of at least 3 (e.g., at least 10, 20, 30, 40, 50, 60, 70, 80, 90, 100, etc.) repeating covalently bound structural units. The polymer is in certain embodiments biocompatible (i.e., non-toxic). Exemplary polymers include, but are not limited to, cellulose polymers (e.g., hydroxyethylcellulose, ethylcellulose, carboxymethylcellulose, methylc cellulose, hydroxypropylmethylcellulose (HPMC)), dextran polymers, polymaleic acid polymers, poly(acrylic acid) polymers, poly(vinylalcohol) polymers, polyvinylpyrrolidone (PVP) polymers, and polyethyleneglycol (PEG) polymers, and combinations thereof.

In some embodiments, R^{L} is a lipophilic, hydrophobic and/or non-polar group. In some embodiments, R^{L} is a lipophilic group. In some embodiments, R^{L} is a hydrophobic group. In some embodiments, R^{L} is a non-polar group.

In some embodiments, when an R^{L} group is depicted as bisecting a carbon-carbon bond, e.g., of the formula (i), it is understood that R^{L} may be bonded to either carbon.

In some embodiments, at least one instance of R^{Q}, R², R⁶, or R⁷ is a group of the formula (i), (ii), or (iii). In some embodiments, at least one instance of R⁶ or R⁷ of R¹ is a group of formula (i), (ii) or (iii). In some embodiments, at least one instance of R⁶ or R⁷ of R¹ is a group of formula (i). In some embodiments, at least one instance of R⁶ or R⁷ of R¹ is a group of formula (i-a). In some embodiments, at least one instance of R⁶ or R⁷ of R¹ is a group of formula (i-al). In some embodiments, at least one instance of R⁶ or R⁷ of R¹ is a group of formula (i-b). In some embodiments, at least one instance of R⁶ or R⁷ of R¹ is a group of formula (ii). In some embodiments, at least one instance of R⁶ or R⁷ of R¹ is a group of formula (iii).

Various combinations of the above embodiments of Formula I are contemplated herein.

In some embodiments, wherein each instance of Q is 0, the compound of formula I is a compound of formula I-a:

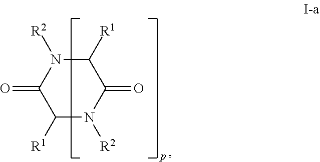

or a pharmaceutically acceptable salt thereof, wherein each variable is independently as defined above and described herein.

In certain embodiments, at least one R¹ is a group of formula (iv). In certain embodiments, each instance of R¹ is a group of formula (iv). In certain embodiments, each instance of R² is independently hydrogen or optionally substituted C₁₋₆alkyl. In certain embodiments, each instance of R² is hydrogen. In certain embodiments, at least one instance of R² is a group of formula (i). In certain embodiments, at least one instance of R² is a group of formula (ii). In certain embodiments, at least one instance of R² is a group of formula (iii). In certain embodiments, p is 1. In certain embodiments, p is 2. In certain embodiments, p is 3.

In some embodiments, wherein at least one R¹ is a group of formula (iv), a compound of formula I is a compound of formula I-b:

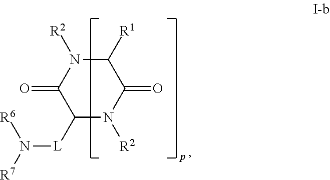

or a pharmaceutically acceptable salt thereof, wherein each variable is independently as defined above and described herein.

In certain embodiments, each instance of R¹ is a group of formula (iv). In certain embodiments, each instance of R² is independently hydrogen or optionally substituted C₁₋₆alkyl. In certain embodiments, each instance of R² is hydrogen. In certain embodiments, at least one instance of R² is a group of formula (i). In certain embodiments, at least one instance of R² is a group of formula (ii). In certain embodiments, at least one instance of R² is a group of formula (iii). In certain embodiments, p is 1. In certain embodiments, p is 2. In certain embodiments, p is 3. In certain embodiments, L is an optionally substituted alkylene. In certain embodiments, R⁶ is a group of formula (i). In certain embodiments, R⁶ is a group of formula (ii). In certain embodiments, R⁶ is a group of formula (iii). In certain embodiments, R⁷ is a group of formula (i). In certain embodiments, R⁷ is a group of formula (ii). In certain embodiments, R⁷ is a group of formula (iii). In certain embodiments, both R⁶ and R⁷ are independently groups of formula (i), (ii), or (iii).

In some embodiments, wherein each instance of R¹ is a group the formula (iv), a compound of Formula I is a compound of formula I-c:

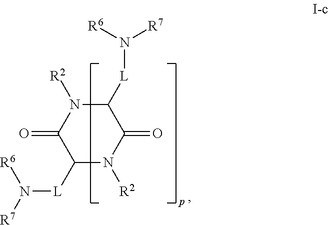

or a pharmaceutically acceptable salt thereof, wherein each variable is independently as defined above and described herein.

In certain embodiments, each instance of R² is independently hydrogen or optionally substituted C₁₋₆alkyl. In certain embodiments, each instance of R² is hydrogen. In certain embodiments, at least one instance of R² is a group of formula (i). In certain embodiments, at least one instance of R² is a group of formula (ii). In certain embodiments, at least one instance of R² is a group of formula (iii). In certain embodiments, p is 1. In certain embodiments, p is 2. In certain embodiments, p is 3. In certain embodiments, L is an optionally substituted alkylene. In certain embodiments, R⁶ is a group of formula (i). In certain embodiments, R⁶ is a group of formula (ii). In certain embodiments, R⁶ is a group of formula (iii). In certain embodiments, R⁷ is a group of formula (i). In certain embodiments, R⁷ is a group of formula (ii). In certain embodiments, R⁷ is a group of formula (iii). In certain embodiments, both R⁶ and R⁷ are independently groups of formula (i), (ii), or (iii).

In some embodiments, p=1. In some embodiments, a compound of formula I-c is a compound of formula I-c1:

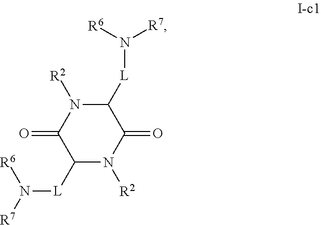

or a pharmaceutically acceptable salt thereof, wherein each variable is independently as defined above and described herein.

In certain embodiments, each instance of R² is independently hydrogen or optionally substituted C₁₋₆alkyl. In certain embodiments, each instance of R² is hydrogen. In certain embodiments, at least one instance of R² is a group of formula (i). In certain embodiments, at least one instance of R² is a group of formula (ii). In certain embodiments, at least one instance of R² is a group of formula (iii). In certain embodiments, L is an optionally substituted alkylene. In certain embodiments, R⁶ is a group of formula (i). In certain embodiments, R⁶ is a group of formula (ii). In certain embodiments, R⁶ is a group of formula (iii). In certain embodiments, R⁷ is a group of formula (i). In certain embodiments, R⁷ is a group of formula (ii). In certain embodiments, R⁷ is a group of formula (iii). In certain embodiments, both R⁶ and R⁷ are independently groups of formula (i), (ii), or (iii). In some embodiments, R⁶ and R⁷ are the same group of formula (i). In some embodiments, R⁶ and R⁷ are the same group of formula (i-a). In some embodiments, R⁶ and R⁷ are the same group of formula (i-al). In some embodiments, R⁶ and R⁷ are the same group of formula (i-b). In some embodiments, R⁶ and R⁷ are the same group of formula (ii). In some embodiments, R⁶ and R⁷ are the same group of formula (iii).

In some embodiments, each instance of R² is hydrogen. In some embodiments, a compound of formula I-c is a compound of formula I-c2:

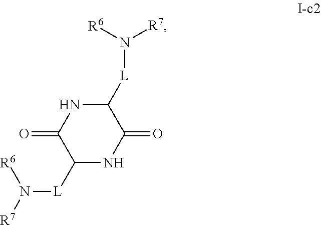

or a pharmaceutically acceptable salt thereof, wherein each variable is independently as defined above and described herein.

In certain embodiments, L is an optionally substituted alkylene. In certain embodiments, R⁶ is a group of formula (i). In certain embodiments, R⁶ is a group of formula (ii). In certain embodiments, R⁶ is a group of formula (iii). In certain embodiments, R⁷ is a group of formula (i). In certain embodiments, R⁷ is a group of formula (ii). In certain embodiments, R⁷ is a group of formula (iii). In certain embodiments, both R⁶ and R⁷ are independently groups of formula (i), (ii), or (iii). In some embodiments, R⁶ and R⁷ are the same group of formula (i). In some embodiments, R⁶ and R⁷ are the same group of formula (i-a). In some embodiments, R⁶ and R⁷ are the same group of formula (i-al). In some embodiments, R⁶ and R⁷ are the same group of formula (i-b). In some embodiments, R⁶ and R⁷ are the same group of formula (ii). In some embodiments, R⁶ and R⁷ are the same group of formula (iii).

In some embodiments, L is an optionally substituted alkylene. In some embodiments, a compound of formula I-c is a compound of formula I-c3:

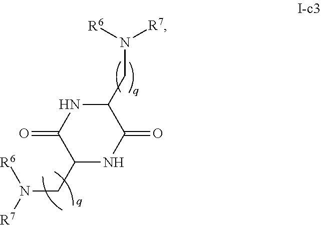

or a pharmaceutically acceptable salt thereof, wherein each variable is independently as defined above and described herein.

In some embodiments, q is an integer between 1 and 10, inclusive. In certain embodiments, R⁶ is a group of formula (i). In certain embodiments, R⁶ is a group of formula (ii). In certain embodiments, R⁶ is a group of formula (iii). In certain embodiments, R⁷ is a group of formula (i). In certain embodiments, R⁷ is a group of formula (ii). In certain embodiments, R⁷ is a group of formula (iii). In certain embodiments, both R⁶ and R⁷ are independently groups of formula (i), (ii), or (iii). In some embodiments, R⁶ and R⁷ are the same group of formula (i). In some embodiments, R⁶ and R⁷ are the same group of formula (i-a). In some embodiments, R⁶ and R⁷ are the same group of formula (i-al). In some embodiments, R⁶ and R⁷ are the same group of formula (i-b). In some embodiments, R⁶ and R⁷ are the same group of formula (ii). In some embodiments, R⁶ and R⁷ are the same group of formula (iii).

In some embodiments, a compound of formula I is a compound of formula I-d:

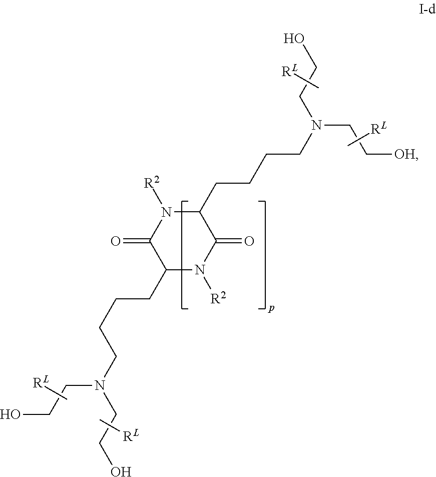

wherein each of p, R² and R^{L} is independently as defined above and described herein.

In some embodiments, a compound of formula I is a compound of formula I-e:

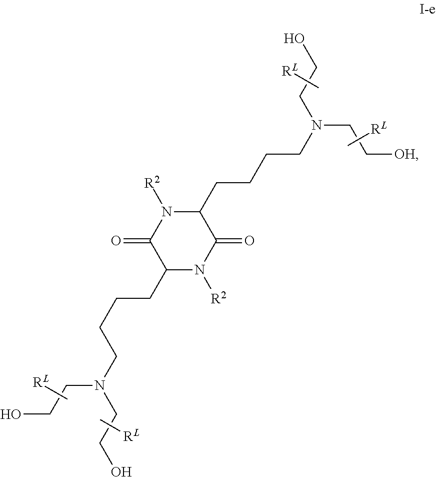

wherein each of R² and R^{L} is independently as defined above and described herein.

In some embodiments, a compound of formula I is a compound of formula I-f:

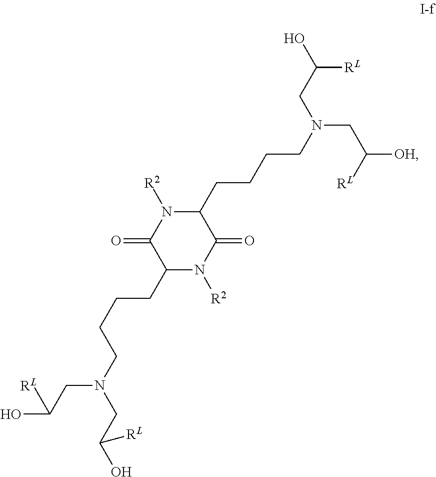

wherein each of R² and R^{L} is independently as defined above and described herein.

In some embodiments, provided liposomes include a cationic lipid described in WO 2013063468 and in U.S. provisional application entitled “Lipid Formulations for Delivery of Messenger RNA” filed concurrently with the present application on even date, both of which are incorporated by reference herein. In some embodiments, a compound of formula I is a compound of formula I-c1-a:

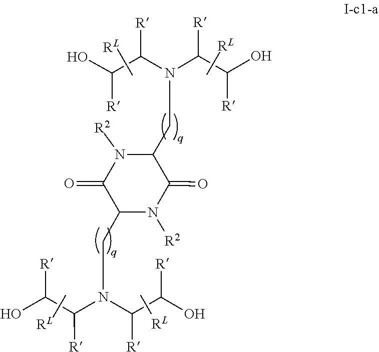

or a pharmaceutically acceptable salt thereof, wherein:
   each R² independently is hydrogen or C₁₋₃ alkyl;
   each q independently is 2 to 6;
   each R′ independently is hydrogen or C₁₋₃ alkyl;
and each R^{L} independently is C₈₋₁₂ alkyl.

In some embodiments, each R² independently is hydrogen, methyl or ethyl. In some embodiments, each R² independently is hydrogen or methyl. In some embodiments, each R² is hydrogen.

In some embodiments, each q independently is 3 to 6. In some embodiments, each q independently is 3 to 5. In some embodiments, each q is 4.

In some embodiments, each R′ independently is hydrogen, methyl or ethyl. In some embodiments, each R′ independently is hydrogen or methyl. In some embodiments, each R′ independently is hydrogen.

In some embodiments, each R^{L} independently is C₈₋₁₂ alkyl. In some embodiments, each R^{L} independently is n-C₈₋₁₂ alkyl. In some embodiments, each R^{L} independently is C₉₋₁₁ alkyl. In some embodiments, each R^{L} independently is n-C₉₋₁₁ alkyl. In some embodiments, each R^{L} independently is C₁₀ alkyl. In some embodiments, each R^{L} independently is n-C₁₀ alkyl.

In some embodiments, each R² independently is hydrogen or methyl; each q independently is 3 to 5; each R′ independently is hydrogen or methyl; and each R^{L} independently is C₈₋₁₂ alkyl.

In some embodiments, each R² is hydrogen; each q independently is 3 to 5; each R′ is hydrogen; and each R^{L} independently is C₈₋₁₂ alkyl.

In some embodiments, each R² is hydrogen; each q is 4; each R′ is hydrogen; and each R^{L} independently is C₈₋₁₂ alkyl.

In some embodiments, a compound of formula I is a compound of formula I-g:

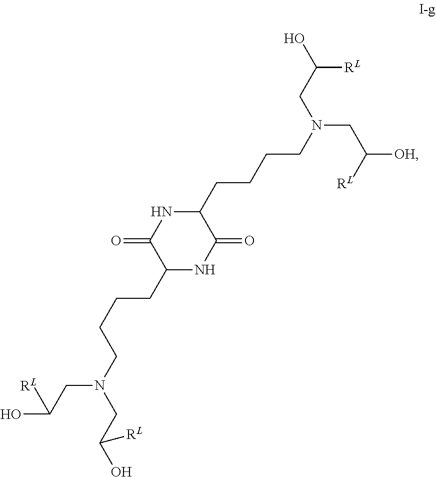

wherein each of R^{L} is independently as defined above and described herein.

In some embodiments, a compound of formula I is a compound of formula X:

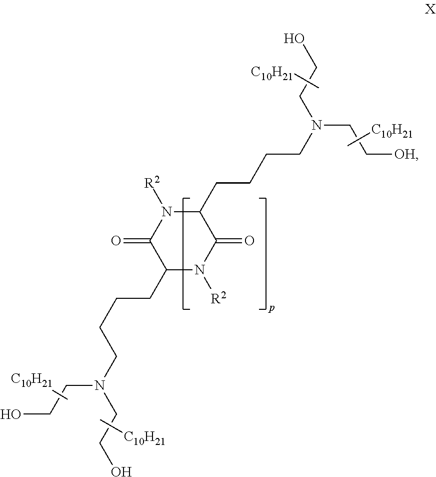

or a pharmaceutically acceptable salt thereof, wherein each variable is independently as defined above and described herein.

In some embodiments, a compound of formula I is a compound of formula X-1:

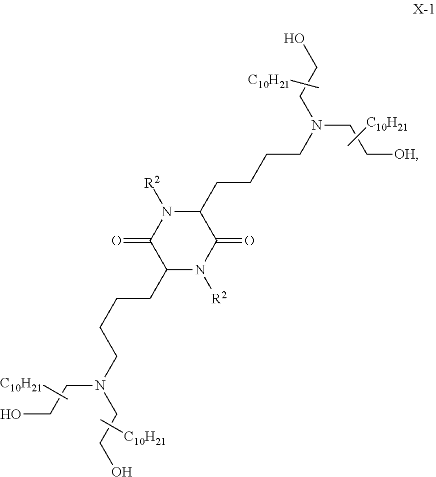

or a pharmaceutically acceptable salt thereof, wherein each R² is independently as defined above and described herein.

In some embodiments, a compound of formula I is

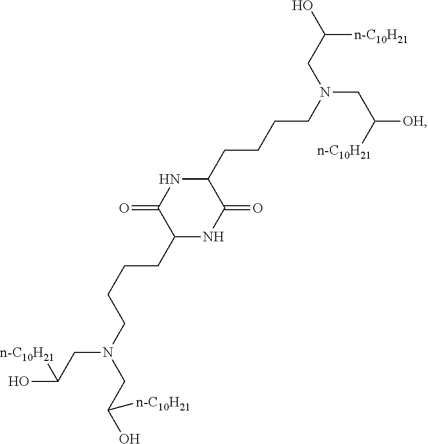

or a pharmaceutically acceptable salt thereof.

Additional examples of cationic lipids suitable for the present invention are described in WO 2013063468, which is incorporated by reference herein in its entirety.

### Chemical Definitions

Definitions of specific functional groups and chemical terms are described in more detail below. The chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5th Edition, John Wiley & Sons, Inc., New York, 2001; Larock, Comprehensive Organic Transformations, VCH Publishers, Inc., New York, 1989; and Carruthers, Some Modern Methods of Organic Synthesis, 3rd Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high performance liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, N Y, 1962); and Wilen, S. H. Tables of Resolving Agents and Optical Resolutions p. 268 (E. L. Eliel, Ed., Univ. of Notre Dame Press, Notre Dame, IN 1972). The invention additionally contemplates compounds as individual isomers substantially free of other isomers, and alternatively, as mixtures of various isomers.

When a range of values is listed, it is intended to encompass each value and sub-range within the range. For example “C1-6 alkyl” is intended to encompass, C1, C2, C3, C4, C5, C6, C1-6, C1-5, C1-4, C1-3, C1-2, C2-6, C2-5, C2-4, C2-3, C3-6, C3-5, C3-4, C4-6, C4-5, and C5-6 alkyl.

As used herein, “alkyl” refers to a radical of a straight-chain or branched saturated hydrocarbon group having from 1 to 50 carbon atoms (“C1-50 alkyl”). In some embodiments, an alkyl group has 1 to 40 carbon atoms (“C1-40 alkyl”). In some embodiments, an alkyl group has 1 to 30 carbon atoms (“C1-30 alkyl”). In some embodiments, an alkyl group has 1 to 20 carbon atoms (“C1-20 alkyl”). In some embodiments, an alkyl group has 1 to 10 carbon atoms (“C1-10 alkyl”). In some embodiments, an alkyl group has 1 to 9 carbon atoms (“C1-9 alkyl”). In some embodiments, an alkyl group has 1 to 8 carbon atoms (“C1-8 alkyl”). In some embodiments, an alkyl group has 1 to 7 carbon atoms (“C1-7 alkyl”). In some embodiments, an alkyl group has 1 to 6 carbon atoms (“C1-6 alkyl”). In some embodiments, an alkyl group has 1 to 5 carbon atoms (“C1-5 alkyl”). In some embodiments, an alkyl group has 1 to 4 carbon atoms (“C1-4 alkyl”). In some embodiments, an alkyl group has 1 to 3 carbon atoms (“C1-3 alkyl”). In some embodiments, an alkyl group has 1 to 2 carbon atoms (“C1-2 alkyl”). In some embodiments, an alkyl group has 1 carbon atom (“C1 alkyl”). In some embodiments, an alkyl group has 2 to 6 carbon atoms (“C2-6 alkyl”). Examples of C1-6 alkyl groups include, without limitation, methyl (C1), ethyl (C2), n-propyl (C3), isopropyl (C3), n-butyl (C4), tert-butyl (C4), sec-butyl (C4), iso-butyl (C4), n-pentyl (C5), 3-pentanyl (C5), amyl (C5), neopentyl (C5), 3-methyl-2-butanyl (C5), tertiary amyl (C5), and n-hexyl (C6). Additional examples of alkyl groups include n-heptyl (C7), n-octyl (C8) and the like. Unless otherwise specified, each instance of an alkyl group is independently unsubstituted (an “unsubstituted alkyl”) or substituted (a “substituted alkyl”) with one or more substituents. In certain embodiments, the alkyl group is an unsubstituted C1-50 alkyl. In certain embodiments, the alkyl group is a substituted C1-50 alkyl.

As used herein, “heteroalkyl” refers to an alkyl group as defined herein which further includes at least one heteroatom (e.g., 1 to 25, e.g., 1, 2, 3, or 4 heteroatoms) selected from oxygen, sulfur, nitrogen, boron, silicon, and phosphorus within (i.e., inserted between adjacent carbon atoms of) and/or placed at one or more terminal position(s) of the parent chain. In certain embodiments, a heteroalkyl group refers to a saturated group having from 1 to 50 carbon atoms and 1 or more heteroatoms within the parent chain (“heteroC1-50 alkyl”). In certain embodiments, a heteroalkyl group refers to a saturated group having from 1 to 40 carbon atoms and 1 or more heteroatoms within the parent chain (“heteroC1-40 alkyl”). In certain embodiments, a heteroalkyl group refers to a saturated group having from 1 to 30 carbon atoms and 1 or more heteroatoms within the parent chain (“heteroC1-30 alkyl”). In certain embodiments, a heteroalkyl group refers to a saturated group having from 1 to 20 carbon atoms and 1 or more heteroatoms within the parent chain (“heteroC1-20 alkyl”). In certain embodiments, a heteroalkyl group refers to a saturated group having from 1 to 10 carbon atoms and 1 or more heteroatoms within the parent chain (“heteroC1-10 alkyl”). In some embodiments, a heteroalkyl group is a saturated group having 1 to 9 carbon atoms and 1 or more heteroatoms within the parent chain (“heteroC1-9 alkyl”). In some embodiments, a heteroalkyl group is a saturated group having 1 to 8 carbon atoms and 1 or more heteroatoms within the parent chain (“heteroC1-8 alkyl”). In some embodiments, a heteroalkyl group is a saturated group having 1 to 7 carbon atoms and 1 or more heteroatoms within the parent chain (“heteroC1-7 alkyl”). In some embodiments, a heteroalkyl group is a saturated group having 1 to 6 carbon atoms and 1 or more heteroatoms within the parent chain (“heteroC1-6 alkyl”). In some embodiments, a heteroalkyl group is a saturated group having 1 to 5 carbon atoms and 1 or 2 heteroatoms within the parent chain (“heteroC1-5 alkyl”). In some embodiments, a heteroalkyl group is a saturated group having 1 to 4 carbon atoms and 1 or 2 heteroatoms within the parent chain (“heteroC1-4 alkyl”). In some embodiments, a heteroalkyl group is a saturated group having 1 to 3 carbon atoms and 1 heteroatom within the parent chain (“heteroC1-3 alkyl”). In some embodiments, a heteroalkyl group is a saturated group having 1 to 2 carbon atoms and 1 heteroatom within the parent chain (“heteroC1-2 alkyl”). In some embodiments, a heteroalkyl group is a saturated group having 1 carbon atom and 1 heteroatom (“heteroC1 alkyl”). In some embodiments, a heteroalkyl group is a saturated group having 2 to 6 carbon atoms and 1 or 2 heteroatoms within the parent chain (“heteroC2-6 alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC1-50 alkyl. In certain embodiments, the heteroalkyl group is a substituted heteroC1-50 alkyl.

As used herein, “alkenyl” refers to a radical of a straight-chain or branched hydrocarbon group having from 2 to 50 carbon atoms and one or more carbon-carbon double bonds (e.g., 1, 2, 3, or 4 double bonds) (“C2-50 alkenyl”). In some embodiments, an alkenyl group has 2 to 40 carbon atoms (“C2-40 alkenyl”). In some embodiments, an alkenyl group has 2 to 30 carbon atoms (“C2-30 alkenyl”). In some embodiments, an alkenyl group has 2 to 20 carbon atoms (“C2-20 alkenyl”). In some embodiments, an alkenyl group has 2 to 10 carbon atoms (“C2-10 alkenyl”). In some embodiments, an alkenyl group has 2 to 9 carbon atoms (“C2-9 alkenyl”). In some embodiments, an alkenyl group has 2 to 8 carbon atoms (“C2-8 alkenyl”). In some embodiments, an alkenyl group has 2 to 7 carbon atoms (“C2-7 alkenyl”). In some embodiments, an alkenyl group has 2 to 6 carbon atoms (“C2-6 alkenyl”). In some embodiments, an alkenyl group has 2 to 5 carbon atoms (“C2-5 alkenyl”). In some embodiments, an alkenyl group has 2 to 4 carbon atoms (“C2-4 alkenyl”). In some embodiments, an alkenyl group has 2 to 3 carbon atoms (“C2-3 alkenyl”). In some embodiments, an alkenyl group has 2 carbon atoms (“C2 alkenyl”). The one or more carbon-carbon double bonds can be internal (such as in 2-butenyl) or terminal (such as in 1-butenyl). Examples of C2-4 alkenyl groups include, without limitation, ethenyl (C2), 1-propenyl (C3), 2-propenyl (C3), 1-butenyl (C4), 2-butenyl (C4), butadienyl (C4), and the like. Examples of C2-6 alkenyl groups include the aforementioned C2-4 alkenyl groups as well as pentenyl (C5), pentadienyl (C5), hexenyl (C6), and the like. Additional examples of alkenyl include heptenyl (C7), octenyl (C8), octatrienyl (C8), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C2-50 alkenyl. In certain embodiments, the alkenyl group is a substituted C2-50 alkenyl.

As used herein, “heteroalkenyl” refers to an alkenyl group as defined herein which further includes at least one heteroatom (e.g., 1 to 25, e.g., 1, 2, 3, or 4 heteroatoms) selected from oxygen, sulfur, nitrogen, boron, silicon, and phosphorus within (i.e., inserted between adjacent carbon atoms of) and/or placed at one or more terminal position(s) of the parent chain. In certain embodiments, a heteroalkenyl group refers to a group having from 2 to 50 carbon atoms, at least one double bond, and 1 or more heteroatoms within the parent chain (“heteroC2-50 alkenyl”). In certain embodiments, a heteroalkenyl group refers to a group having from 2 to 40 carbon atoms, at least one double bond, and 1 or more heteroatoms within the parent chain (“heteroC2-40 alkenyl”). In certain embodiments, a heteroalkenyl group refers to a group having from 2 to 30 carbon atoms, at least one double bond, and 1 or more heteroatoms within the parent chain (“heteroC2-30 alkenyl”). In certain embodiments, a heteroalkenyl group refers to a group having from 2 to 20 carbon atoms, at least one double bond, and 1 or more heteroatoms within the parent chain (“heteroC2-20 alkenyl”). In certain embodiments, a heteroalkenyl group refers to a group having from 2 to 10 carbon atoms, at least one double bond, and 1 or more heteroatoms within the parent chain (“heteroC2-10 alkenyl”). In some embodiments, a heteroalkenyl group has 2 to 9 carbon atoms at least one double bond, and 1 or more heteroatoms within the parent chain (“heteroC2-9 alkenyl”). In some embodiments, a heteroalkenyl group has 2 to 8 carbon atoms, at least one double bond, and 1 or more heteroatoms within the parent chain (“heteroC2-8 alkenyl”). In some embodiments, a heteroalkenyl group has 2 to 7 carbon atoms, at least one double bond, and 1 or more heteroatoms within the parent chain (“heteroC2-7 alkenyl”). In some embodiments, a heteroalkenyl group has 2 to 6 carbon atoms, at least one double bond, and 1 or more heteroatoms within the parent chain (“heteroC2-6 alkenyl”). In some embodiments, a heteroalkenyl group has 2 to 5 carbon atoms, at least one double bond, and 1 or 2 heteroatoms within the parent chain (“heteroC2-5 alkenyl”). In some embodiments, a heteroalkenyl group has 2 to 4 carbon atoms. at least one double bond, and for 2 heteroatoms within the parent chain (“heteroC2-4 alkenyl”). In some embodiments, a heteroalkenyl group has 2 to 3 carbon atoms, at least one double bond, and 1 heteroatom within the parent chain (“heteroC2-3 alkenyl”). In some embodiments, a heteroalkenyl group has 2 to 6 carbon atoms, at least one double, bond, and 1 or 2 heteroatoms within the parent chain (“heteroC2-6 alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC2-50 alkenyl. In certain embodiments, the heteroalkenyl group is a substituted heteroC2-50 alkenyl.

As used herein, “alkynyl” refers to a radical of a straight-chain or branched hydrocarbon group having from 2 to 50 carbon atoms and one or more carbon-carbon triple bonds (e.g., 1, 2, 3, or 4 triple bonds) and optionally one or more double bonds (e.g., 1, 2, 3, or 4 double bonds) (“C2-50 alkynyl”). An alkynyl group that has one or more triple bonds and one or more double bonds is also referred to as an “ene-yne”. In some embodiments, an alkynyl group has 2 to 40 carbon atoms (“C2-40 alkynyl”). In some embodiments, an alkynyl group has 2 to 30 carbon atoms (“C2-30 alkynyl”). In some embodiments, an alkynyl group has 2 to 20 carbon atoms (“C2-20 alkynyl”). In some embodiments, an alkynyl group has 2 to 10 carbon atoms (“C2-10 alkynyl”). In some embodiments, an alkynyl group has 2 to 9 carbon atoms (“C2-9 alkynyl”). In some embodiments, an alkynyl group has 2 to 8 carbon atoms (“C2-8 alkynyl”). In some embodiments, an alkynyl group has 2 to 7 carbon atoms (“C2-7 alkynyl”). In some embodiments, an alkynyl group has 2 to 6 carbon atoms (“C2-6 alkynyl”). In some embodiments, an alkynyl group has 2 to 5 carbon atoms (“C2-5 alkynyl”). In some embodiments, an alkynyl group has 2 to 4 carbon atoms (“C2-4 alkynyl”). In some embodiments, an alkynyl group has 2 to 3 carbon atoms (“C2-3 alkynyl”). In some embodiments, an alkynyl group has 2 carbon atoms (“C2 alkynyl”). The one or more carbon-carbon triple bonds can be internal (such as in 2-butynyl) or terminal (such as in 1-butynyl). Examples of C2-4 alkynyl groups include, without limitation, ethynyl (C2), 1-propynyl (C3), 2-propynyl (C3), 1-butynyl (C4), 2-butynyl (C4), and the like. Examples of C2-6 alkenyl groups include the aforementioned C2-4 alkynyl groups as well as pentynyl (C5), hexynyl (C6), and the like. Additional examples of alkynyl include heptynyl (C7), octynyl (C8), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C2-50 alkynyl. In certain embodiments, the alkynyl group is a substituted C2-50 alkynyl.

As used herein, “heteroalkynyl” refers to an alkynyl group as defined herein which further includes at least one heteroatom (e.g., 1 to 25, e.g., 1, 2, 3, or 4 heteroatoms) selected from oxygen, sulfur, nitrogen, boron, silicon, and phosphorus within (i.e., inserted between adjacent carbon atoms of) and/or placed at one or more terminal position(s) of the parent chain. In certain embodiments, a heteroalkynyl group refers to a group having from 2 to 50 carbon atoms, at least one triple bond, and 1 or more heteroatoms within the parent chain (“heteroC2-50 alkynyl”). In certain embodiments, a heteroalkynyl group refers to a group having from 2 to 40 carbon atoms, at least one triple bond, and 1 or more heteroatoms within the parent chain (“heteroC2-40 alkynyl”). In certain embodiments, a heteroalkynyl group refers to a group having from 2 to 30 carbon atoms, at least one triple bond, and 1 or more heteroatoms within the parent chain (“heteroC2-30 alkynyl”). In certain embodiments, a heteroalkynyl group refers to a group having from 2 to 20 carbon atoms, at least one triple bond, and 1 or more heteroatoms within the parent chain (“heteroC2-20 alkynyl”). In certain embodiments, a heteroalkynyl group refers to a group having from 2 to 10 carbon atoms, at least one triple bond, and 1 or more heteroatoms within the parent chain (“heteroC2-10 alkynyl”). In some embodiments, a heteroalkynyl group has 2 to 9 carbon atoms, at least one triple bond, and 1 or more heteroatoms within the parent chain (“heteroC2-9 alkynyl”). In some embodiments, a heteroalkynyl group has 2 to 8 carbon atoms, at least one triple bond, and 1 or more heteroatoms within the parent chain (“heteroC2-8 alkynyl”). In some embodiments, a heteroalkynyl group has 2 to 7 carbon atoms, at least one triple bond, and 1 or more heteroatoms within the parent chain (“heteroC2-7 alkynyl”). In some embodiments, a heteroalkynyl group has 2 to 6 carbon atoms, at least one triple bond, and 1 or more heteroatoms within the parent chain (“heteroC2-6 alkynyl”). In some embodiments, a heteroalkynyl group has 2 to 5 carbon atoms, at least one triple bond, and 1 or 2 heteroatoms within the parent chain (“heteroC2-5 alkynyl”). In some embodiments, a heteroalkynyl group has 2 to 4 carbon atoms, at least one triple bond, and for 2 heteroatoms within the parent chain (“heteroC2-4 alkynyl”). In some embodiments, a heteroalkynyl group has 2 to 3 carbon atoms, at least one triple bond, and 1 heteroatom within the parent chain (“heteroC2-3 alkynyl”). In some embodiments, a heteroalkynyl group has 2 to 6 carbon atoms, at least one triple bond. and 1 or 2 heteroatoms within the parent chain (“heteroC2-6 alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC2-50 alkynyl. In certain embodiments, the heteroalkynyl group is a substituted heteroC2-50 alkynyl.

As used herein, “carbocyclyl” or “carbocyclic” refers to a radical of a non-aromatic cyclic hydrocarbon group having from 3 to 10 ring carbon atoms (“C3-10 carbocyclyl”) and zero heteroatoms in the non-aromatic ring system. In some embodiments, a carbocyclyl group has 3 to 8 ring carbon atoms (“C3-8 carbocyclyl”). In some embodiments, a carbocyclyl group has 3 to 7 ring carbon atoms (“C3-7 carbocyclyl”). In some embodiments, a carbocyclyl group has 3 to 6 ring carbon atoms (“C3-6 carbocyclyl”). In some embodiments, a carbocyclyl group has 4 to 6 ring carbon atoms (“C4-6 carbocyclyl”). In some embodiments, a carbocyclyl group has 5 to 6 ring carbon atoms (“C5-6 carbocyclyl”). In some embodiments, a carbocyclyl group has 5 to 10 ring carbon atoms (“C5-10 carbocyclyl”). Exemplary C3-6 carbocyclyl groups include, without limitation, cyclopropyl (C3), cyclopropenyl (C3), cyclobutyl (C4), cyclobutenyl (C4), cyclopentyl (C5), cyclopentenyl (C5), cyclohexyl (C6), cyclohexenyl (C6), cyclohexadienyl (C6), and the like. Exemplary C3-8 carbocyclyl groups include, without limitation, the aforementioned C3-6 carbocyclyl groups as well as cycloheptyl (C7), cycloheptenyl (C7), cycloheptadienyl (C7), cycloheptatrienyl (C7), cyclooctyl (C8), cyclooctenyl (C8), bicyclo[2.2.1]heptanyl (C7), bicyclo[2.2.2]octanyl (C8), and the like. Exemplary C3-10 carbocyclyl groups include, without limitation, the aforementioned C3-8 carbocyclyl groups as well as cyclononyl (C9), cyclononenyl (C9), cyclodecyl (C10), cyclodecenyl (C10), octahydro-1H-indenyl (C9), decahydronaphthalenyl (C10), spiro[4.5]decanyl (C10), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C3-10 carbocyclyl. In certain embodiments, the carbocyclyl group is a substituted C3-10 carbocyclyl.

In some embodiments, “carbocyclyl” or “carbocyclic” is referred to as a “cycloalkyl”, i.e., a monocyclic, saturated carbocyclyl group having from 3 to 10 ring carbon atoms (“C3-10 cycloalkyl”). In some embodiments, a cycloalkyl group has 3 to 8 ring carbon atoms (“C3-8 cycloalkyl”). In some embodiments, a cycloalkyl group has 3 to 6 ring carbon atoms (“C3-6, cycloalkyl”). In some embodiments, a cycloalkyl group has 4 to 6 ring carbon atoms (“C4-6 cycloalkyl”). In some embodiments, a cycloalkyl group has 5 to 6 ring carbon atoms (“C5-6 cycloalkyl”). In some embodiments, a cycloalkyl group has 5 to 10 ring carbon atoms (“C5-10 cycloalkyl”). Examples of C5-6 cycloalkyl groups include cyclopentyl (C5) and cyclohexyl (C5). Examples of C3-6 cycloalkyl groups include the aforementioned C5-6 cycloalkyl groups as well as cyclopropyl (C3) and cyclobutyl (C4). Examples of C3-8 cycloalkyl groups include the aforementioned C3-6 cycloalkyl groups as well as cycloheptyl (C7) and cyclooctyl (C8). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C3-10 cycloalkyl. In certain embodiments, the cycloalkyl group is a substituted C3-10 cycloalkyl.

As used herein, “heterocyclyl” or “heterocyclic” refers to a radical of a 3- to 14-membered non-aromatic ring system having ring carbon atoms and 1 or more (e.g., 1, 2, 3, or 4) ring heteroatoms, wherein each heteroatom is independently selected from oxygen, sulfur, nitrogen, boron, silicon, and phosphorus (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)). and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1 or more (e.g., 1, 2, 3, or 4) ring heteroatoms, wherein each heteroatom is independently selected from oxygen, sulfur, nitrogen, boron, silicon, and phosphorus (“5-10 membered heterocyclyl”). In some embodiments, a heterocyclyl group is a 5-8 membered non-aromatic ring system having ring carbon atoms and 1 or more (e.g., 1, 2, 3, or 4) ring heteroatoms, wherein each heteroatom is independently selected from oxygen, sulfur, nitrogen, boron, silicon, and phosphorus (“5-8 membered heterocyclyl”). In some embodiments, a heterocyclyl group is a 5-6 membered non-aromatic ring system having ring carbon atoms and 1 or more (e.g., 1, 2, 3, or 4) ring heteroatoms, wherein each heteroatom is independently selected from oxygen, sulfur, nitrogen, boron, silicon, and phosphorus (“5-6 membered heterocyclyl”). In some embodiments, the 5-6 membered heterocyclyl has 1 or more (e.g., 1, 2, or 3) ring heteroatoms selected from oxygen, sulfur, nitrogen, boron, silicon, and phosphorus. In some embodiments, the 5-6 membered heterocyclyl has 1 or 2 ring heteroatoms selected from oxygen, sulfur, nitrogen, boron, silicon, and phosphorus. In some embodiments, the 5-6 membered heterocyclyl has 1 ring heteroatom selected from oxygen, sulfur, nitrogen, boron, silicon, and phosphorus.

Exemplary 3-membered heterocyclyl groups containing 1 heteroatom include, without limitation, azirdinyl, oxiranyl, thiorenyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation. tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, dioxanyl. Exemplary 6-membered heterocyclyl groups containing 2 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo-[2,3-b]pyridinyl, 4,5,6,7-tetrahydrofuro[3,2-c]pyridinyl, 4,5,6,7-tetrahydrothieno [3,2-b]pyridinyl, 1,2,3,4-tetrahydro-1,6-naphthyridinyl, and the like.

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C6-14 aryl”). In some embodiments, an aryl group has 6 ring carbon atoms (“C6 aryl”; e.g., phenyl). In some embodiments, an aryl group has 10 ring carbon atoms (“C10 aryl”; e.g., naphthyl such as 1-naphthyl and 2-naphthyl). In some embodiments, an aryl group has 14 ring carbon atoms (“C14 aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C6-14 aryl. In certain embodiments, the aryl group is a substituted C6-14 aryl.

As used herein, “heteroaryl” refers to a radical of a 5-14 membered monocyclic or polycyclic (e.g., bicyclic or tricyclic) 4n+2 aromatic ring system (e.g., having 6, 10, or 14 π electrons shared in a cyclic array) having ring carbon atoms and 1 or more (e.g., 1, 2, 3, or 4 ring heteroatoms) ring heteroatoms provided in the aromatic ring system, wherein each heteroatom is independently selected from oxygen, sulfur, nitrogen, boron, silicon, and phosphorus (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1 or more (e.g., 1, 2, 3, or 4) ring heteroatoms provided in the aromatic ring system, wherein each heteroatom is independently selected from oxygen, sulfur, nitrogen, boron, silicon, and phosphorus (“5-10 membered heteroaryl”). In some embodiments, a heteroaryl group is a 5-8 membered aromatic ring system having ring carbon atoms and 1 or more (e.g., 1, 2, 3, or 4) ring heteroatoms provided in the aromatic ring system, wherein each heteroatom is independently selected from oxygen, sulfur, nitrogen, boron, silicon, and phosphorus (“5-8 membered heteroaryl”). In some embodiments, a heteroaryl group is a 5-6 membered aromatic ring system having ring carbon atoms and 1 or more (e.g., 1, 2, 3, or 4) ring heteroatoms provided in the aromatic ring system, wherein each heteroatom is independently selected from oxygen, sulfur, nitrogen, boron, silicon, and phosphorus (“5-6 membered heteroaryl”). In some embodiments, the 5-6 membered heteroaryl has 1 or more (e.g., 1, 2, or 3) ring heteroatoms selected from oxygen, sulfur, nitrogen, boron, silicon, and phosphorus. In some embodiments, the 5-6 membered heteroaryl has 1 or 2 ring heteroatoms selected from oxygen, sulfur, nitrogen, boron, silicon, and phosphorus. In some embodiments, the 5-6 membered heteroaryl has 1 ring heteroatom selected from oxygen, sulfur, nitrogen, boron, silicon, and phosphorus. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation. pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

As used herein, the term “partially unsaturated” refers to a ring moiety that includes at least one double or triple bond. The term “partially unsaturated” is intended to encompass rings having multiple sites of unsaturation, but is not intended to include aromatic groups (e.g., aryl or heteroaryl moieties) as herein defined.

As used herein, the term “saturated” refers to a ring moiety that does not contain a double or triple bond, i.e., the ring contains all single bonds.

Affixing the suffix “-ene” to a group indicates the group is a divalent moiety, e.g., alkylene is the divalent moiety of alkyl, alkenylene is the divalent moiety of alkenyl,
alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

As understood from the above, alkyl, alkenyl, alkynyl, heteroalkyl, heteroalkenyl, heteroalkynyl, carbocyclyl, heterocyclyl, aryl, and heteroaryl groups, as defined herein, are, in certain embodiments, optionally substituted. Optionally substituted refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl. “substituted” or “unsubstituted” carbocyclyl. “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO2, —N3, —SO2H, —SO3H, —OH, -ORaa, —ON(Rbb)2, —N(Rbb)2, —N(Rbb)3+X—, —N(ORcc)Rbb, —SeH, -SeRaa, —SH, -SRaa, -SSRcc, —C(═O)Raa, —CO2H, —CHO, —C(ORcc)2, —CO2Raa, —OC(═O)Raa, -OCO2Raa, —C(═O)N(Rbb)2, —OC(═O)N(Rbb)2, -NRbbC(═O)Raa, —NRbbCO2Raa, -NRbbC(═O)N(Rbb)2, —C(═NRbb)Raa, —C(═NRbb)ORaa, —OC(═NRbb)Raa, —OC(=NRbb)ORaa, —C(=NRbb)N(Rbb)2, —OC(═NRbb)N(Rbb)2, -NRbbC(=NRbb)N(Rbb)2, —C(═O)NRbbSO2Raa, -NRbbSO2Raa, —SO2N(Rbb)2, -SO2Raa, -SO2ORaa, —OSO2Raa, —S(═O)Raa, —OS(═O)Raa, —Si(Raa)3-OSi(Raa)3 —C(═S)N(Rbb)2, —C(═O)SRaa, —C(═S)SRaa, —SC(═S)SRaa, —SC(═O)SRaa, —OC(═O)SRaa, —SC(═O)ORaa, —SC(═O)Raa, —P(═O)2Raa, —OP(═O)2Raa, —P(═O)(Raa)2, —OP(═O)(Raa)2, —OP(═O)(ORcc)2, —P(═O)2N(Rbb)2, —OP(═O)2N(Rbb)2, —P(═O)(NRbb)2, —OP(═O)(NRbb)2, -NRbbP(═O)(ORcc)2, -NRbbP(═O)(NRbb)2, —P(Rcc)2, —P(Rcc)3, —OP(Rcc)2, —OP(Rcc)3, —B(Raa)2, —B(ORcc)2, -BRaa(ORcc), C1-50 alkyl, C2-50 alkenyl, C2-50 alkynyl, C3-14 carbocyclyl, 3-14 membered heterocyclyl, C6-14 aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 Rdd groups;
or two geminal hydrogens on a carbon atom are replaced with the group ═O, ═S, NN(Rbb)2, =NNRbbC(═O)Raa, =NNRbbC(═O)ORaa, =NNRbbS(═O)2Raa, =NRbb, or =NORcc;
each instance of Raa is, independently, selected from C1-50 alkyl, C2-50 alkenyl, C2-50 alkynyl, C3-10 carbocyclyl, 3-14 membered heterocyclyl, C6-14 aryl, and 5-14 membered heteroaryl, or two Raa groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 Rdd groups;
each instance of Rbb is, independently, selected from hydrogen, —OH, -ORaa, —N(Rcc)2, —CN, —C(═O)Raa, —C(═O)N(Rcc)2, —CO2Raa, —SO2Raa, —C(═NRcc)ORaa, —C(=NRcc)N(Rcc)2, —SO2N(Rcc)2, —SO2Rcc, —SO2ORcc, -SORaa, —C(═S)N(Rcc)2, —C(═O)SRcc, —C(═S)SRcc, —P(═O)2Raa, —P(═O)(Raa)2, —P(═O)2N(Rcc)2, —P(═O)(NRcc)2, C1-50 alkyl, C2-50 alkenyl, C2-50 alkynyl, C3-10 carbocyclyl, 3-14 membered heterocyclyl, C6-14 aryl, and 5-14 membered heteroaryl, or two Rbb groups, together with the heteroatom to which they are attached, form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 Rdd groups;
each instance of Rcc is, independently, selected from hydrogen, C1-50 alkyl, C2-50 alkenyl, C2-50 alkynyl, C3-10 carbocyclyl, 3-14 membered heterocyclyl, C6-14 aryl, and 5-14 membered heteroaryl, or two Rcc groups, together with the heteroatom to which they are attached, form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 Rdd groups;
each instance of Rdd is, independently, selected from halogen, —CN, —NO2, —N3, —SO2H, —SO3H, —OH, -ORee, —ON(Rff)2, —N(Rff)2, —N(Rff)3+X—, —N(ORee)Rff, —SH, -SRee, —SSRee, —C(═O)Ree, —CO2H, —CO2Ree, —OC(═O)Ree, —OCO2Ree, —C(═O)N(Rff)2, —OC(═O)N(Rff)2, -NRffC(═O)Ree, -NRffCO2Ree, -NRffC(═O)N(Rff)2, —C(═NRff)ORee, —OC(=NRff)Ree, —OC(═NRff)ORee, —C(=NRff)N(Rff)2, —OC(═NRff)N(Rff)2, -NRffC(=NRff)N(Rff)2, -NRffS02Ree, —S02N(Rff)2, —SO2Ree, —SO2ORee, —OSO2Ree, —S(═O)Ree, —Si(Ree)3, —OSi(Ree)3, —C(═S)N(Rff)2, —C(═O)SRee, —C(═S)SRee, —SC(═S)SRee, —P(═O)2Ree, —P(═O)(Ree)2, —OP(═O)(Ree)2, —OP(═O)(ORee)2, C1-50 alkyl, C2-50 alkenyl, C2-50 alkynyl, C3-10 carbocyclyl, 3-10 membered heterocyclyl, C6-10 aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 Rgg groups, or two geminal Rdd substituents can be joined to form═O or ═S;
each instance of Ree is, independently, selected from C1-50 alkyl, C2-50 alkenyl, C2-50 alkynyl, C3-10 carbocyclyl, C6-10 aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 Rgg groups;
each instance of Rff is, independently, selected from hydrogen, C1-50 alkyl, C2-50 alkenyl, C2-50 alkynyl, C3-10 carbocyclyl, 3-10 membered heterocyclyl, C6-10 aryl and 5-10 membered heteroaryl, or two Rff groups, together with the heteroatom to which they are attached, form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 Rgg groups; and
each instance of Rgg is, independently, halogen, —CN, —NO2, —N3, —SO2H, —SO3H, —OH, —OC1-50 alkyl, —ON(C1-50 alkyl)2, —N(C1-50 alkyl)2, —N(C1-50 alkyl)3+X—, —NH(C1-50 alkyl)2+X—, —NH2(C1-50 alkyl)+X—, -NH3+X—, —N(OC1-50 alkyl)(C1-50 alkyl), —N(OH)(C1-50 alkyl), —NH(OH), —SH, -SC1-50 alkyl, —SS(C1-50 alkyl), —C(═O)(C1-50 alkyl), —CO2H, —CO2(C1-50 alkyl), —OC(═O)(C1-50 alkyl), —OCO2(C1-50 alkyl), —C(═O)NH2, —C(═O)N(C1-50 alkyl)2, —OC(═O)NH(C1-50 alkyl), —NHC(═O)(C1-50 alkyl), —N(C1-50 alkyl)C(═O)(C1-50 alkyl), —NHCO2(C1-50 alkyl), —NHC(═O)N(C1-50 alkyl)2, —NHC(═O)NH(C1-50 alkyl), —NHC(═O)NH2, —C(═NH)O(C1-50 alkyl), —OC(═NH)(C1-50 alkyl), —OC(═NH)OC1-50 alkyl, —C(═NH)N(C1-50 alkyl)2, —C(═NH)NH(C1-50 alkyl), —C(═NH)NH2, —OC(═NH)N(C1-50alkyl)2, —OC(NH)NH(C1-50 alkyl), —OC(NH)NH2, —NHC(NH)N(C1-50 alkyl)2, —NHC(═NH)NH2, —NHSO2 (C1-50 alkyl), —SO2N (C1-50 alkyl)2, —SO2NH (C1-50 alkyl), —SO2NH2, —SO2C1-50 alkyl, -SO2OC1-50 alkyl, -OSO2C1-6 alkyl, -SOC1-6 alkyl, —Si(C1-50 alkyl)3, —OSi(C1-6 alkyl)3 —C(═S)N(C1-50 alkyl)2, C(═S)NH(C1-50 alkyl), C(═S)NH2, —C(═O)S(C1-6 alkyl), —C(═S)SC1-6 alkyl, —SC(═S)SC1-6 alkyl, —P(═O)2(C1-50 alkyl), —P(═O)(C1-50 alkyl)2, —OP(═O)(C1-50 alkyl)2, —OP(═O)(OC1-50 alkyl)2, C1-50 alkyl, C2-50 alkenyl, C2-50 alkynyl, C3-10 carbocyclyl, C6-10 aryl, 3-10 membered heterocyclyl, 5-10 membered heteroaryl; or two geminal Rgg substituents can be joined to form═O or ═S;
wherein X— is a counterion.

As used herein, the term “halo” or “halogen” refers to fluorine (fluoro, —F), chlorine (chloro, —Cl), bromine (bromo, —Br), or iodine (iodo, —I).

As used herein, a “counterion” is a negatively charged group associated with a positively charged quarternary amine in order to maintain electronic neutrality. Exemplary counterions include halide ions (e.g., F—, Cl—, Br—, I—), NO3-, C1O4-, OH—, H2PO4-, HSO4-, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

Nitrogen atoms can be substituted or unsubstituted as valency permits, and include primary, secondary, tertiary, and quarternary nitrogen atoms. Exemplary nitrogen atom substitutents include, but are not limited to, hydrogen, —OH, -ORaa, —N(Rcc)2, —CN, —C(═O)Raa, —C(═O)N(Rcc)2, -CO2Raa, -SO2Raa, —C(═NRbb)Raa, —C(═NRcc)ORaa, —C(=NRcc)N(Rcc)2, —SO2N(Rcc)2, -SO2Rcc, -SO2ORcc, -SORaa, —C(═S)N(Rcc)2, —C(═O)SRcc, —C(═S)SRcc, —P(═O)2Raa, —P(═O)(Raa)2, —P(═O)2N(Rcc)2, —P(═O)(NRcc)2, C1-50 alkyl, C2-50 alkenyl, C2 50 alkynyl, C3-10 carbocyclyl, 3-14 membered heterocyclyl, C6-14 aryl, and 5-14 membered heteroaryl, or two Rcc groups, together with the N atom to which they are attached, form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 Rdd groups, and wherein Raa, Rbb, Rcc and Rdd are as defined above.

Nitrogen atoms can be substituted or unsubstituted as valency permits, and include primary, secondary, tertiary, and quarternary nitrogen atoms. Exemplary nitrogen atom substitutents include, but are not limited to, hydrogen, —OH, -ORaa, —N(Rcc)2, —CN, —C(═O)Raa, —C(═O)N(Rcc)2, -CO2Raa, -SO2Raa, —C(═NRbb)Raa, —C(═NRcc)ORaa, —C(=NRcc)N(Rcc)2, -SO2N(Rcc)2, -SO2Rcc, -SO2ORcc, -SORaa, —C(═S)N(Rcc)2, —C(═O)SRcc, —C(═S)SRcc, —P(═O)2Raa, —P(═O)(Raa)2, —P(═O)2N(Rcc)2, —P(═O)(NRcc)2, C1-10 alkyl, C1-10 perhaloalkyl, C2-10 alkenyl, C2-10 alkynyl, C3-10 carbocyclyl, 3-14 membered heterocyclyl, C6-14 aryl, and 5-14 membered heteroaryl, or two Rcc groups, together with the nitrogen atom to which they are attached, form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 Rdd groups, and wherein Raa, Rbb, Rcc and Rdd are as defined above.

In certain embodiments, the substituent present on a nitrogen atom is a nitrogen protecting group (also referred to as an amino protecting group). Nitrogen protecting groups include, but are not limited to, —OH, -ORaa, —N(Rcc)2, —C(═O)Raa, —C(═O)N(Rcc)2, -CO2Raa, -SO2Raa, —C(═NRcc)Raa, —C(═NRcc)ORaa, —C(═NRcc)N(Rcc)2, —SO2N(Rcc)2, -SO2Rcc, -SO2ORcc, -SORaa, —C(═S)N(Rcc)2, —C(═O)SRcc, —C(═S)SRcc, C1-10 alkyl (e.g., aralkyl, heteroaralkyl), C2-10 alkenyl, C2-10 alkynyl, C3-10 carbocyclyl, 3-14 membered heterocyclyl, C6-14 aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4 or 5 Rdd groups, and wherein Raa, Rbb, Rcc and Rdd are as defined herein. Nitrogen protecting groups are well known in the art and include those described in detail in Protecting Groups in Organic Synthesis, T. W. Greene and P. G. M. Wuts, 3rd edition, John Wiley & Sons, 1999, incorporated herein by reference.

For example, nitrogen protecting groups such as amide groups (e.g., —C(═O)Raa) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)ORaa) include, but are not limited to, methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methyl sulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)2Raa) include, but are not limited to, p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethyl silyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N—S-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to as a hydroxyl protecting group). Oxygen protecting groups include, but are not limited to, -Raa, —N(Rbb)2, —C(═O)SRaa, —C(═O)Raa, -CO2Raa, —C(═O)N(Rbb)2, —C(═NRbb)Raa, —C(═NRbb)ORaa, —C(═NRbb)N(Rbb)2, —S(═O)Raa, -SO2Raa, Si(Raa)3, —P(Rcc)2, —P(Rcc)3, —P(═O)2Raa, —P(═O)(Raa)2, —P(═O)(ORcc)2, —P(═O)2N(Rbb)2, and —P(═O)(NRbb)2, wherein Raa, Rbb, and Rcc are as defined herein. Oxygen protecting groups are well known in the art and include those described in detail in Protecting Groups in Organic Synthesis, T. W. Greene and P. G. M. Wuts, 3rd edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethyl silylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picoly1 N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenyl sily 1 (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on an sulfur atom is an sulfur protecting group (also referred to as a thiol protecting group). Sulfur protecting groups include, but are not limited to, -Raa, —N(Rbb)2, —C(═O)SRaa, —C(═O)Raa, -CO2Raa, C(═O)N(Rbb)2, —C(=NRbb)Raa, —C(═NRbb)ORaa, —C(═NRbb)N(Rbb)2, —S(═O)Raa, -SO2Raa, —Si(Raa)3, —P(Rcc)2, —P(Rcc)3, —P(═O)2Raa, —P(═O)(Raa)2, —P(═O)(ORcc)2, —P(═O)2N(Rbb)2, and —P(═O)(NRbb)2, wherein Raa, Rbb, and Rcc are as defined herein. Sulfur protecting groups are well known in the art and include those described in detail in Protecting Groups in Organic Synthesis, T. W. Greene and P. G. M. Wuts, 3rd edition, John Wiley & Sons, 1999, incorporated herein by reference.

As used herein, a “leaving group” is an art-understood term referring to a molecular fragment that departs with a pair of electrons in heterolytic bond cleavage, wherein the molecular fragment is an anion or neutral molecule. See, for example, Smith, March's Advanced Organic Chemistry 6th ed. (501-502). Exemplary leaving groups include, but are not limited to, halo (e.g., chloro, bromo, iodo) and sulfonyl substituted hydroxyl groups (e.g., tosyl, mesyl, besyl).

### Other Definitions

As used herein, use of the phrase “at least one instance” refers to one instance, but also encompasses more than one instance, e.g., for example, 1, 2, 3, 4, 5, 6, 7, 8, 9, or 10 instances, and up to 100 instances.

As used herein, a “polymer” refers to a compound comprised of at least 3 (e.g., at least 10, 20, 30, 40, 50, 60, 70, 80, 90, 100, etc.) repeating covalently bound structural units.

“Attached” refers to the covalent attachment of a group.

As used herein, “lipophilic” refers to the ability of a group to dissolve in fats, oils, lipids, and lipophilic non-polar solvents such as hexane or toluene. In general, a lipophilic group refers to an unsubstituted n-alkyl or unsubstituted n-alkenyl group having 6 to 50 carbon atoms, e.g., 6 to 40, 6 to 30, 6 to 20, 8 to 20, 8 to 19, 8 to 18, 8 to 17, 8 to 16, or 8 to 15 carbon atoms.

As used herein, the term “salt” or “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or rnalonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate. digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N+(C1-4alkyl)4 salts. Representative alkali or alkaline earth metal salts include sodium, lithium, potassium, calcium, magnesium, and the like. Further pharmaceutically acceptable salts include, when appropriate, nontoxic ammonium. quaternary ammonium, and amine cations formed using counterions such as halide, hydroxide, carboxylate, sulfate, phosphate, nitrate, sulfonate and aryl sulfonate. Further pharmaceutically acceptable salts include salts formed from the quarternization of an amine using an appropriate electrophile, e.g., an alkyl halide, to form a quarternized alkylated amino salt.

### Second or Additional Cationic Lipids

In some embodiments, liposomes may comprise a second or additional cationic lipid. As used herein, the phrase “cationic lipid” refers to any of a number of lipid species that have a net positive charge at a selected pH, such as physiological pH. Several cationic lipids have been described in the literature, many of which are commercially available. Particularly suitable cationic lipids for use in the compositions and methods of the invention include those described in international patent publications WO 2010/053572 (and particularly, C12-200 described at paragraph [00225]) and WO 2012/170930, both of which are incorporated herein by reference. In certain embodiments, the compositions and methods of the invention employ a lipid nanoparticles comprising an ionizable cationic lipid described in U.S. provisional patent application 61/617,468, filed Mar. 29, 2012 (incorporated herein by reference), such as, e.g, (15Z, 18Z)-N,N-dimethyl-6-(9Z, 12Z)-octadeca-9, 12-dien-1-yl)tetracosa-15,18-dien-1-amine (HGT5000), (15Z, 18Z)-N,N-dimethyl-6-((9Z, 12Z)-octadeca-9, 12-dien-1-yl)tetracosa-4,15,18-trien-1-amine (HGT5001), and (15Z,18Z)-N,N-dimethyl-6-((9Z, 12Z)-octadeca-9, 12-dien-1-yl)tetracosa-5, 15, 18-trien-1-amine (HGT5002).

In some embodiments, the second or additional cationic lipid N-[1-(2,3-dioleyloxy)propyl]-N,N,N-trimethylammonium chloride or “DOTMA” is used. (Feigner et al. (Proc. Nat'l Acad. Sci. 84, 7413 (1987); U.S. Pat. No. 4,897,355). DOTMA can be formulated alone or can be combined with the neutral lipid, dioleoylphosphatidyl-ethanolamine or “DOPE” or other cationic or non-cationic lipids into a liposomal transfer vehicle or a lipid nanoparticle, and such liposomes can be used to enhance the delivery of nucleic acids into target cells. Other suitable cationic lipids include, for example, 5-carboxyspermylglycinedioctadecylamide or “DOGS,” 2,3-dioleyloxy-N-[2(spermine-carboxamido)ethyl]-N,N-dimethyl-1-propanaminium or “DOSPA” (Behr et al. Proc. Nat.'l Acad. Sci. 86, 6982 (1989); U.S. Pat. Nos. 5,171,678; 5,334,761), 1,2-Dioleoyl-3-Dimethylammonium-Propane or “DODAP”, 1,2-Dioleoyl-3-Trimethylammonium-Propane or “DOTAP”. Additional exemplary cationic lipids also include 1,2-distearyloxy-N,N-dimethyl-3-aminopropane or “DSDMA”, 1,2-dioleyloxy-N,N-dimethyl-3-aminopropane or “DODMA”, 1,2-dilinoleyloxy-N,N-dimethyl-3-aminopropane or “DLinDMA”, 1,2-dilinolenyloxy-N,N-dimethyl-3-aminopropane or “DLenDMA”, N-dioleyl-N,N-dimethylammonium chloride or “DODAC”, N,N-distearyl-N,N-dimethylarnrnonium bromide or “DDAB”, N-(1,2-dimyristyloxyprop-3-yl)-N,N-dimethyl-N-hydroxyethyl ammonium bromide or “DMRIE”, 3-dimethylamino-2-(cholest-5-en-3-beta-oxybutan-4-oxy)-1-(cis,cis-9,12-octadecadienoxy)propane or “CLinDMA”, 2-[5′-(cholest-5-en-3-beta-oxy)-3′-oxapentoxy)-3-dimethy 1-1-(cis,cis-9′,1-2′-octadecadienoxy)propane or “CpLinDMA”, N,N-dimethyl-3,4-dioleyloxybenzylamine or “DMOBA”, 1,2-N,N′-dioleylcarbamyl-3-dimethylaminopropane or “DOcarbDAP”, 2,3-Dilinoleoyloxy-N,N-dimethylpropylamine or “DLinDAP”,1,2-N,N′-Dilinoleylcarbamyl-3-dimethylaminopropane or “DLincarbDAP”,1,2-Dilinoleoylcarbamyl-3-dimethylaminopropane or “DLinCDAP”, 2,2-dilinoleyl-4-dimethylaminomethyl-[1,3]-dioxolane or “DLin--DMA”, 2,2-dilinoleyl-4-dimethylaminoethyl-[1,3]-dioxolane or “DLin-K-XTC2-DMA”, and 2-(2,2-di((9Z, 12Z)-octadeca-9,12-dien-1-yl)-1,3-dioxolan-4-yl)-N,N-dimethylethanamine (DLin-KC2-DMA)) (See, WO 2010/042877; Semple et al., Nature Biotech. 28: 172-176 (2010)), or mixtures thereof. (Heyes, J., et al., J Controlled Release 107: 276-287 (2005); Morrissey, D V., et al., Nat. Biotechnol. 23(8): 1003-1007 (2005); PCT Publication WO2005/121348A1). In some embodiments, one or more of the cationic lipids comprise at least one of an imidazole, dialkylamino, or guanidinium moiety.

In some embodiments, the second or additional cationic lipid may be chosen from XTC (2,2-Dilinoley1-4-dimethylaminoethy1-[1,3]-dioxolane), MC3 (((6Z,9Z,28Z,31Z)-heptatriaconta-6,9,28,31-tetraen-19-yl 4-(dimethylamino)butanoate), ALNY-100 ((3aR,5s,6aS)-N,N-dimethyl-2,2-di((9Z,12Z)-octadeca-9,12-dienyl)tetrahydro-3aH-cyclopenta[d] [1, 3]dioxol-5-amine)), NC98-5 (4,7,13-tris(3-oxo-3-(undecylamino)propyl)-N1,N16-diundecyl-4,7,10,13-tetraazahexadecane-1,16-diamide), DODAP (1,2-dioleyl-3-dimethylammonium propane), HGT4003 (WO 2012/170889, the teachings of which are incorporated herein by reference in their entirety), ICE (WO 2011/068810, the teachings of which are incorporated herein by reference in their entirety), HGT5000 (U.S. Provisional Patent Application No. 61/617,468, the teachings of which are incorporated herein by reference in their entirety) or HGT5001 (cis or trans) (Provisional Patent Application No. 61/617,468), aminoalcohol lipidoids such as those disclosed in WO2010/053572, DOTAP (1,2-dioleyl-3-trimethylammonium propane), DOTMA (1,2-di-O-octadecenyl-3-trimethylammonium propane), DLinDMA (Heyes, J.; Palmer, L.; Bremner, K.; MacLachlan, I. “Cationic lipid saturation influences intracellular delivery of encapsulated nucleic acids” J. Contr. Rel. 2005, 107, 276-287), DLin-KC2-DMA (Semple, S. C. et al. “Rational Design of Cationic Lipids for siRNA Delivery” Nature Biotech. 2010, 28, 172-176), C12-200 (Love, K. T. et al. “Lipid-like materials for low-dose in vivo gene silencing” PNAS 2010, 107, 1864-1869).

### Non-cationic/Helper Lipids

In some embodiments, provided liposomes contain one or more non-cationic (“helper”) lipids. As used herein, the phrase “non-cationic lipid” refers to any neutral, zwitterionic or anionic lipid. As used herein, the phrase “anionic lipid” refers to any of a number of lipid species that carry a net negative charge at a selected H, such as physiological pH. Non-cationic lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-ma), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), or a mixture thereof.

In some embodiments, such non-cationic lipids may be used alone, but are preferably used in combination with other excipients, for example, cationic lipids. In some embodiments, the non-cationic lipid may comprise a molar ratio of about 5% to about 90%, or about 10% to about 70% of the total lipid present in a liposome. In some embodiments, a non-cationic lipid is a neutral lipid, i.e., a lipid that does not carry a net charge in the conditions under which the composition is formulated and/or administered. In some embodiments, the percentage of non-cationic lipid in a liposome may be greater than 5%, greater than 10%, greater than 20%, greater than 30%, or greater than 40%.

### Cholesterol-based Lipids

In some embodiments, provided liposomes comprise one or more cholesterol-based lipids. For example, suitable cholesterol-based cationic lipids include, for example, DC-Choi (N,N-dimethyl-N-ethylcarboxamidocholesterol),1,4-bis(3-N-oleylamino-propyl)piperazine (Gao, et al. Biochem. Biophys. Res. Comm. 179, 280 (1991); Wolf et al. BioTechniques 23, 139 (1997); U.S. Pat. No. 5,744,335), or ICE. In some embodiments, the cholesterol-based lipid may comprise a molar ration of about 2% to about 30%, or about 5% to about 20% of the total lipid present in a liposome. In some embodiments, The percentage of cholesterol-based lipid in the lipid nanoparticle may be greater than 5, %, 10%, greater than 20%, greater than 30%, or greater than 40%.

### PEGylated Lipids

In some embodiments, provided liposomes comprise one or more PEGylated lipids. For example, the use of polyethylene glycol (PEG)-modified phospholipids and derivatized lipids such as derivatized ceramides (PEG-CER), including N-Octanoyl-Sphingosine-1-[Succinyl(Methoxy Polyethylene Glycol)-2000] (C8 PEG-2000 ceramide) is also contemplated by the present invention in combination with one or more of the cationic and, in some embodiments, other lipids together which comprise the liposome. Contemplated PEG-modified lipids include, but are not limited to, a polyethylene glycol chain of up to 5 kDa in length covalently attached to a lipid with alkyl chain(s) of C₆-C₂₀ length. In some embodiments, a PEG-modified or PEGylated lipid is PEGylated cholesterol or PEG-2K. The addition of such components may prevent complex aggregation and may also provide a means for increasing circulation lifetime and increasing the delivery of the lipid-nucleic acid composition to the target cell, (Klibanov et al. (1990) FEBS Letters, 268 (1): 235-237), or they may be selected to rapidly exchange out of the formulation in vivo (see U.S. Pat. No. 5,885,613).

In some embodiments, particularly useful exchangeable lipids are PEG-ceramides having shorter acyl chains (e.g., C₁₄ or C₁₈). The PEG-modified phospholipid and derivitized lipids of the present invention may comprise a molar ratio from about 0% to about 15%, about 0.5% to about 15%, about 1% to about 15%, about 4% to about 10%, or about 2% of the total lipid present in the liposome.

According to various embodiments, the selection of second or additional cationic lipids, non-cationic lipids and/or PEG-modified lipids which comprise the lipid nanoparticle, as well as the relative molar ratio of such lipids to each other, is based upon the characteristics of the selected lipid(s), the nature of the intended target cells, the characteristics of the mRNA to be delivered. Additional considerations include, for example, the saturation of the alkyl chain, as well as the size, charge, pH, pKa, fusogenicity and toxicity of the selected lipid(s). Thus the molar ratios may be adjusted accordingly. In some embodiments, the percentage of PEG-modified lipid in a liposome may be greater than 1%, greater than 2%, greater than 5%, greater than 10%, or greater than 15%.

### Polymer

In some embodiments, a suitable liposome according to the present invention further includes a polymer, in combination with one or more cationic lipids as described and, in some embodiments, other carriers including various lipids described herein. Thus, in some embodiments, liposomal delivery vehicles, as used herein, also encompass polymer containing nanoparticles. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, protamine, PEGylated protamine, PLL, PEGylated PLL and polyethylenimine (PEI). When PEI is present, it may be branched PEI of a molecular weight ranging from 10 to 40 kDA, e.g., 25 kDa branched PEI (Sigma #408727).

In some embodiments, a suitable liposome formulation contains a combination of one or more cationic lipids, one or more non-cationic lipids, one or more cholesterol-based lipids one or more PEG-modified lipids, and/or one or more polymers. As a non-limiting example, a suitable liposome comprises cKK-E12, DOPE, cholesterol and DMG-PEG2K. In some embodiments, the ratio of cationic lipid to non-cationic lipid to cholesterol-based lipid to PEGylated lipid may be between about 30-50:25-35:20-30:1-15, respectively. In some embodiments, the ratio of cationic lipid to non-cationic lipid to cholesterol-based lipid to PEGylated lipid is approximately 40:30:20:10, respectively. In some embodiments, the ratio of cationic lipid to non-cationic lipid to cholesterol-based lipid to PEGylated lipid is approximately 40:30:25:5, respectively. In some embodiments, the ratio of cationic lipid to non-cationic lipid to cholesterol-based lipid to PEGylated lipid is approximately 40:32:25:3, respectively.

mRNA

The present invention can be used to deliver any mRNA. mRNA is typically thought of as the type of RNA that carries information from DNA to the ribosome. The existence of mRNA is usually very brief and includes processing and translation, followed by degradation. Typically, in eukaryotic organisms, mRNA processing comprises the addition of a “cap” on the N-terminal (5′) end, and a “tail” on the C-terminal (3′) end. A typical cap is a 7-methylguanosine cap, which is a guanosine that is linked through a 5′-5′-triphosphate bond to the first transcribed nucleotide. The presence of the cap is important in providing resistance to nucleases found in most eukaryotic cells. The tail is typically a polyadenylation event whereby a polyadenylyl moiety is added to the 3′ end of the mRNA molecule. The presence of this “tail” serves to protect the mRNA from exonuclease degradation. Messenger RNA typically is translated by the ribosomes into a series of amino acids that make up a protein.

Any mRNA capable of being translated into one or more peptides (e.g., proteins) or peptide fragments is contemplated as within the scope of the present invention. In some embodiments, an mRNA encodes one or more naturally occurring peptides. In some embodiments, an mRNA encodes one or more modified or non-natural peptides.

In some embodiments an mRNA encodes an intracellular protein. In some embodiments, an mRNA encodes a cytosolic protein. In some embodiments, an mRNA encodes a protein associated with the actin cytoskeleton. In some embodiments, an mRNA encodes a protein associated with the plasma membrane. In some specific embodiments, an mRNA encodes a transmembrane protein. In some specific embodiments an mRNA encodes an ion channel protein. In some embodiments, an mRNA encodes a perinuclear protein. In some embodiments, an mRNA encodes a nuclear protein. In some specific embodiments, an mRNA encodes a transcription factor. In some embodiments, an mRNA encodes a chaperone protein. In some embodiments, an mRNA encodes an intracellular enzyme (e.g., mRNA encoding an enzyme associated with urea cycle or lysosomal storage metabolic disorders). In some embodiments, an mRNA encodes a protein involved in cellular metabolism, DNA repair, transcription and/or translation. In some embodiments, an mRNA encodes an extracellular protein. In some embodiments, an mRNA encodes a protein associated with the extracellular matrix. In some embodiments an mRNA encodes a secreted protein. In specific embodiments, an mRNA used in the composition and methods of the invention may be used to express functional proteins or enzymes that are excreted or secreted by one or more target cells into the surrounding extracellular fluid (e.g., mRNA encoding hormones and/or neurotransmitters).

In some embodiments, the compositions and methods of the invention provide for delivery of mRNA encoding a secreted protein. In some embodiments, the compositions and methods of the invention provide for delivery of mRNA encoding one or more secreted proteins listed in Table 1; thus, compositions of the invention may comprise an mRNA encoding a protein listed in Table 1 (or a homolog thereof) along with other components set out herein, and methods of the invention may comprise preparing and/or administering a composition comprising an mRNA encoding a protein listed in Table 1 (or a homolog thereof) along with other components set out herein.

TABLE 1
Secreted Proteins

| Uniprot ID | Protein Name | Gene Name |
|---|---|---|
| A1E959 | Odontogenic ameloblast-associated protein | ODAM |
| A1KZ92 | Peroxidasin-like protein | PXDNL |
| A1L453 | Serine protease 38 | PRSS38 |
| A1L4H1 | Soluble scavenger receptor cysteine-rich domain- | SSC5D |
| | containing protein SSC5D | |
| A2RUU4 | Colipase-like protein 1 | CLPSL1 |
| A2VDF0 | Fucose mutarotase | FUOM |
| A2VEC9 | SCO-spondin | SSPO |
| A3KMH1 | von Willebrand factor A domain-containing | VWA8 |
| | protein 8 | |
| A4D0S4 | Laminin subunit beta-4 | LAMB4 |
| A4D1T9 | Probable inactive serine protease 37 | PRSS37 |
| A5D8T8 | C-type lectin domain family 18 member A | CLEC18A |
| A6NC86 | phospholipase A2 inhibitor and Ly6/PLAUR | PINLYP |
| | domain-containing protein | |
| A6NCI4 | von Willebrand factor A domain-containing | VWA3A |
| | protein 3A | |
| A6ND01 | Probable folate receptor delta | FOLR4 |
| A6NDD2 | Beta-defensin 108B-like | |
| A6NE02 | BTB/POZ domain-containing protein 17 | BTBD17 |
| A6NEF6 | Growth hormone 1 | GH1 |
| A6NF02 | NPIP-like protein LOC730153 | |
| A6NFB4 | HCG1749481, isoform CRA_k | CSH1 |
| A6NFZ4 | Protein FAM24A | FAM24A |
| A6NG13 | Glycosyltransferase 54 domain-containing protein | |
| A6NGN9 | IgLON family member 5 | IGLON5 |
| A6NHN0 | Otolin-1 | OTOL1 |
| A6NHN6 | Nuclear pore complex-interacting protein-like 2 | NPIPL2 |
| A6NI73 | Leukocyte immunoglobulin-like receptor | LILRA5 |
| | subfamily A member 5 | |
| A6NIT4 | Chorionic somatomammotropin hormone 2 | CSH2 |
| | isoform 2 | |
| A6NJ69 | IgA-inducing protein homolog | IGIP |
| A6NKQ9 | Choriogonadotropin subunit beta variant 1 | CGB1 |
| A6NMZ7 | Collagen alpha-6(VI) chain | COL6A6 |
| A6NNS2 | Dehydrogenase/reductase SDR family member 7C | DHRS7C |
| A6XGL2 | Insulin A chain | INS |
| A8K0G1 | Protein Wnt | WNT7B |
| A8K2U0 | Alpha-2-macroglobulin-like protein 1 | A2ML1 |
| A8K7I4 | Calcium-activated chloride channel regulator 1 | CLCA1 |
| A8MTL9 | Serpin-like protein HMSD | HMSD |
| A8MV23 | Serpin E3 | SERPINE3 |
| A8MZH6 | Oocyte-secreted protein 1 homolog | OOSP1 |
| A8TX70 | Collagen alpha-5(VI) chain | COL6A5 |
| B0ZBE8 | Natriuretic peptide | NPPA |
| B1A4G9 | Somatotropin | GH1 |
| B1A4H2 | HCG1749481, isoform CRA_d | CSH1 |
| B1A4H9 | Chorionic somatomammotropin hormone | CSH2 |
| B1AJZ6 | Protein Wnt | WNT4 |
| B1AKI9 | Isthmin-1 | ISM1 |
| B2RNN3 | Complement C1q and tumor necrosis factor- | C1QTNF9B |
| | related protein 9B | |
| B2RUY7 | von Willebrand factor C domain-containing | VWC2L |
| | protein 2-like | |
| B3GLJ2 | Prostate and testis expressed protein 3 | PATE3 |
| B4DI03 | SEC11-like 3 (S. cerevisiae), isoform CRA_a | SEC11L3 |
| B4DJF9 | Protein Wnt | WNT4 |
| B4DUL4 | SEC11-like 1 (S. cerevisiae), isoform CRA_d | SEC11L1 |
| B5MCC8 | Protein Wnt | WNT10B |
| B8A595 | Protein Wnt | WNT7B |
| B8A597 | Protein Wnt | WNT7B |
| B8A598 | Protein Wnt | WNT7B |
| B9A064 | Immunoglobulin lambda-like polypeptide 5 | IGLL5 |
| C9J3H3 | Protein Wnt | WNT10B |
| C9J8I8 | Protein Wnt | WNT5A |
| C9JAF2 | Insulin-like growth factor II Ala-25 Del | IGF2 |
| C9JCI2 | Protein Wnt | WNT10B |
| C9JL84 | HERV-H LTR-associating protein 1 | HHLA1 |
| C9JNR5 | Insulin A chain | INS |
| C9JUI2 | Protein Wnt | WNT2 |
| D6RF47 | Protein Wnt | WNT8A |
| D6RF94 | Protein Wnt | WNT8A |
| E2RYF7 | Protein PBMUCL2 | HCG22 |
| E5RFR1 | PENK(114-133) | PENK |
| E7EML9 | Serine protease 44 | PRSS44 |
| E7EPC3 | Protein Wnt | WNT9B |
| E7EVP0 | Nociceptin | PNOC |
| E9PD02 | Insulin-like growth factor I | IGF1 |
| E9PH60 | Protein Wnt | WNT16 |
| E9PJL6 | Protein Wnt | WNT11 |
| F5GYM2 | Protein Wnt | WNT5B |
| F5H034 | Protein Wnt | WNT5B |
| F5H364 | Protein Wnt | WNT5B |
| F5H7Q6 | Protein Wnt | WNT5B |
| F8WCM5 | Protein INS-IGF2 | INS-IGF2 |
| F8WDR1 | Protein Wnt | WNT2 |
| H0Y663 | Protein Wnt | WNT4 |
| H0YK72 | Signal peptidase complex catalytic subunit | SEC11A |
| | SEC11A | |
| H0YK83 | Signal peptidase complex catalytic subunit | SEC11A |
| | SEC11A | |
| H0YM39 | Chorionic somatomammotropin hormone | CSH2 |
| H0YMT7 | Chorionic somatomammotropin hormone | CSH1 |
| H0YN17 | Chorionic somatomammotropin hormone | CSH2 |
| H0YNA5 | Signal peptidase complex catalytic subunit | SEC11A |
| | SEC11A | |
| H0YNG3 | Signal peptidase complex catalytic subunit | SEC11A |
| | SEC11A | |
| H0YNX5 | Signal peptidase complex catalytic subunit | SEC11A |
| | SEC11A | |
| H7BZB8 | Protein Wnt | WNT10A |
| H9KV56 | Choriogonadotropin subunit beta variant 2 | CGB2 |
| I3L0L8 | Protein Wnt | WNT9B |
| J3KNZ1 | Choriogonadotropin subunit beta variant 1 | CGB1 |
| J3KP00 | Choriogonadotropin subunit beta | CGB7 |
| J3QT02 | Choriogonadotropin subunit beta variant 1 | CGB1 |
| O00175 | C-C motif chemokine 24 | CCL24 |
| O00182 | Galectin-9 | LGALS9 |
| O00187 | Mannan-binding lectin serine protease 2 | MASP2 |
| O00230 | Cortistatin | CORT |
| O00253 | Agouti-related protein | AGRP |
| O00270 | 12-(S)-hydroxy-5,8,10,14-eicosatetraenoic acid | GPR31 |
| | receptor | |
| O00292 | Left-right determination factor 2 | LEFTY2 |
| O00294 | Tubby-related protein 1 | TULP1 |
| O00295 | Tubby-related protein 2 | TULP2 |
| O00300 | Tumor necrosis factor receptor superfamily | TNFRSF11B |
| | member 11B | |
| O00339 | Matrilin-2 | MATN2 |
| O00391 | Sulfhydryl oxidase 1 | QSOX1 |
| O00468 | Agrin | AGRN |
| O00515 | Ladinin-1 | LAD1 |
| O00533 | Processed neural cell adhesion molecule L1-like | CHL1 |
| | protein | |
| O00584 | Ribonuclease T2 | RNASET2 |
| O00585 | C-C motif chemokine 21 | CCL21 |
| O00602 | Ficolin-1 | FCN1 |
| O00622 | Protein CYR61 | CYR61 |
| O00626 | MDC(5-69) | CCL22 |
| O00634 | Netrin-3 | NTN3 |
| O00744 | Protein Wnt-10b | WNT10B |
| O00755 | Protein Wnt-7a | WNT7A |
| O14498 | Immunoglobulin superfamily containing leucine- | ISLR |
| | rich repeat protein | |
| O14511 | Pro-neuregulin-2, membrane-bound isoform | NRG2 |
| O14594 | Neurocan core protein | NCAN |
| O14625 | C-X-C motif chemokine 11 | CXCL11 |
| O14638 | Ectonucleotide pyrophosphatase/phosphodiesterase | ENPP3 |
| | family member 3 | |
| O14656 | Torsin-1A | TOR1A |
| O14657 | Torsin-1B | TOR1B |
| O14786 | Neuropilin-1 | NRP1 |
| O14788 | Tumor necrosis factor ligand superfamily member | TNFSF11 |
| | 11, membrane form | |
| O14791 | Apolipoprotein L1 | APOL1 |
| O14793 | Growth/differentiation factor 8 | MSTN |
| O14904 | Protein Wnt-9a | WNT9A |
| O14905 | Protein Wnt-9b | WNT9B |
| O14944 | Proepiregulin | EREG |
| O14960 | Leukocyte cell-derived chemotaxin-2 | LECT2 |
| O15018 | Processed PDZ domain-containing protein 2 | PDZD2 |
| O15041 | Semaphorin-3E | SEMA3E |
| O15072 | A disintegrin and metalloproteinase with | ADAMTS3 |
| | thrombospondin motifs 3 | |
| O15123 | Angiopoietin-2 | ANGPT2 |
| O15130 | Neuropeptide FF | NPFF |
| O15197 | Ephrin type-B receptor 6 | EPHB6 |
| O15204 | ADAM DEC1 | ADAMDEC1 |
| O15230 | Laminin subunit alpha-5 | LAMA5 |
| O15232 | Matrilin-3 | MATN3 |
| O15240 | Neuroendocrine regulatory peptide-1 | VGF |
| O15263 | Beta-defensin 4A | DEFB4A |
| O15335 | Chondroadherin | CHAD |
| O15393 | Transmembrane protease serine 2 catalytic chain | TMPRSS2 |
| O15444 | C-C motif chemokine 25 | CCL25 |
| O15467 | C-C motif chemokine 16 | CCL16 |
| O15496 | Group 10 secretory phospholipase A2 | PLA2G10 |
| O15520 | Fibroblast growth factor 10 | FGF10 |
| O15537 | Retinoschisin | RS1 |
| O43157 | Plexin-B1 | PLXNB1 |
| O43184 | Disintegrin and metalloproteinase domain- | ADAM12 |
| | containing protein 12 | |
| O43240 | Kallikrein-10 | KLK10 |
| O43278 | Kunitz-type protease inhibitor 1 | SPINT1 |
| O43320 | Fibroblast growth factor 16 | FGF16 |
| O43323 | Desert hedgehog protein C-product | DHH |
| O43405 | Cochlin | COCH |
| O43508 | Tumor necrosis factor ligand superfamily member | TNFSF12 |
| | 12, membrane form | |
| O43555 | Progonadoliberin-2 | GNRH2 |
| O43557 | Tumor necrosis factor ligand superfamily member | TNFSF14 |
| | 14, soluble form | |
| O43692 | Peptidase inhibitor 15 | PI15 |
| O43699 | Sialic acid-binding Ig-like lectin 6 | SIGLEC6 |
| O43820 | Hyaluronidase-3 | HYAL3 |
| O43827 | Angiopoietin-related protein 7 | ANGPTL7 |
| O43852 | Calumenin | CALU |
| O43854 | EGF-like repeat and discoidin I-like domain- | EDIL3 |
| | containing protein 3 | |
| O43866 | CD5 antigen-like | CD5L |
| O43897 | Tolloid-like protein 1 | TLL1 |
| O43915 | Vascular endothelial growth factor D | FIGF |
| O43927 | C-X-C motif chemokine 13 | CXCL13 |
| O60218 | Aldo-keto reductase family 1 member B10 | AKR1B10 |
| O60235 | Transmembrane protease serine 11D | TMPRSS11D |
| O60258 | Fibroblast growth factor 17 | FGF17 |
| O60259 | Kallikrein-8 | KLK8 |
| O60383 | Growth/differentiation factor 9 | GDF9 |
| O60469 | Down syndrome cell adhesion molecule | DSCAM |
| O60542 | Persephin | PSPN |
| O60565 | Gremlin-1 | GREM1 |
| O60575 | Serine protease inhibitor Kazal-type 4 | SPINK4 |
| O60676 | Cystatin-8 | CST8 |
| O60687 | Sushi repeat-containing protein SRPX2 | SRPX2 |
| O60844 | Zymogen granule membrane protein 16 | ZG16 |
| O60882 | Matrix metalloproteinase-20 | MMP20 |
| O60938 | Keratocan | KERA |
| O75015 | Low affinity immunoglobulin gamma Fc region | FCGR3B |
| | receptor III-B | |
| O75077 | Disintegrin and metalloproteinase domain- | ADAM23 |
| | containing protein 23 | |
| O75093 | Slit homolog 1 protein | SLIT1 |
| O75094 | Slit homolog 3 protein | SLIT3 |
| O75095 | Multiple epidermal growth factor-like domains | MEGF6 |
| | protein 6 | |
| O75173 | A disintegrin and metalloproteinase with | ADAMTS4 |
| | thrombospondin motifs 4 | |
| O75200 | Nuclear pore complex-interacting protein-like 1 | NPIPL1 |
| O75339 | Cartilage intermediate layer protein 1 C1 | CILP |
| O75354 | Ectonucleoside triphosphate diphosphohydrolase 6 | ENTPD6 |
| O75386 | Tubby-related protein 3 | TULP3 |
| O75398 | Deformed epidermal autoregulatory factor 1 | DEAF1 |
| | homolog | |
| O75443 | Alpha-tectorin | TECTA |
| O75445 | Usherin | USH2A |
| O75462 | Cytokine receptor-like factor 1 | CRLF1 |
| O75487 | Glypican-4 | GPC4 |
| O75493 | Carbonic anhydrase-related protein 11 | CA11 |
| O75594 | Peptidoglycan recognition protein 1 | PGLYRP1 |
| O75596 | C-type lectin domain family 3 member A | CLEC3A |
| O75610 | Left-right determination factor 1 | LEFTY1 |
| O75629 | Protein CREG1 | CREG1 |
| O75636 | Ficolin-3 | FCN3 |
| O75711 | Scrapie-responsive protein 1 | SCRG1 |
| O75715 | Epididymal secretory glutathione peroxidase | GPX5 |
| O75718 | Cartilage-associated protein | CRTAP |
| O75829 | Chondrosurfactant protein | LECT1 |
| O75830 | Serpin I2 | SERPINI2 |
| O75882 | Attractin | ATRN |
| O75888 | Tumor necrosis factor ligand superfamily | TNFSF13 |
| | member 13 | |
| O75900 | Matrix metalloproteinase-23 | MMP23A |
| O75951 | Lysozyme-like protein 6 | LYZL6 |
| O75973 | C1q-related factor | C1QL1 |
| O76038 | Secretagogin | SCGN |
| O76061 | Stanniocalcin-2 | STC2 |
| O76076 | WNT1-inducible-signaling pathway protein 2 | WISP2 |
| O76093 | Fibroblast growth factor 18 | FGF18 |
| O76096 | Cystatin-F | CST7 |
| O94769 | Extracellular matrix protein 2 | ECM2 |
| O94813 | Slit homolog 2 protein C-product | SLIT2 |
| O94907 | Dickkopf-related protein 1 | DKK1 |
| O94919 | Endonuclease domain-containing 1 protein | ENDOD1 |
| O94964 | N-terminal form | SOGA1 |
| O95025 | Semaphorin-3D | SEMA3D |
| O95084 | Serine protease 23 | PRSS23 |
| O95150 | Tumor necrosis factor ligand superfamily | TNFSF15 |
| | member 15 | |
| O95156 | Neurexophilin-2 | NXPH2 |
| O95157 | Neurexophilin-3 | NXPH3 |
| O95158 | Neurexophilin-4 | NXPH4 |
| O95388 | WNT1-inducible-signaling pathway protein 1 | WISP1 |
| O95389 | WNT1-inducible-signaling pathway protein 3 | WISP3 |
| O95390 | Growth/differentiation factor 11 | GDF11 |
| O95393 | Bone morphogenetic protein 10 | BMP10 |
| O95399 | Urotensin-2 | UTS2 |
| O95407 | Tumor necrosis factor receptor superfamily | TNFRSF6B |
| | member 6B | |
| O95428 | Papilin | PAPLN |
| O95445 | Apolipoprotein M | APOM |
| O95450 | A disintegrin and metalloproteinase with | ADAMTS2 |
| | thrombospondin motifs 2 | |
| O95460 | Matrilin-4 | MATN4 |
| O95467 | LHAL tetrapeptide | GNAS |
| O95631 | Netrin-1 | NTN1 |
| O95633 | Follistatin-related protein 3 | FSTL3 |
| O95711 | Lymphocyte antigen 86 | LY86 |
| O95715 | C-X-C motif chemokine 14 | CXCL14 |
| O95750 | Fibroblast growth factor 19 | FGF19 |
| O95760 | Interleukin-33 | IL33 |
| O95813 | Cerberus | CER1 |
| O95841 | Angiopoietin-related protein 1 | ANGPTL1 |
| O95897 | Noelin-2 | OLFM2 |
| O95925 | Eppin | EPPIN |
| O95965 | Integrin beta-like protein 1 | ITGBL1 |
| O95967 | EGF-containing fibulin-like extracellular matrix | EFEMP2 |
| | protein 2 | |
| O95968 | Secretoglobin family 1D member 1 | SCGB1D1 |
| O95969 | Secretoglobin family 1D member 2 | SCGB1D2 |
| O95970 | Leucine-rich glioma-inactivated protein 1 | LGI1 |
| O95972 | Bone morphogenetic protein 15 | BMP15 |
| O95994 | Anterior gradient protein 2 homolog | AGR2 |
| O95998 | Interleukin-18-binding protein | IL18BP |
| O96009 | Napsin-A | NAPSA |
| O96014 | Protein Wnt-11 | WNT11 |
| P00450 | Ceruloplasmin | CP |
| P00451 | Factor VIIIa light chain | F8 |
| P00488 | Coagulation factor XIII A chain | F13A1 |
| P00533 | Epidermal growth factor receptor | EGFR |
| P00709 | Alpha-lactalbumin | LALBA |
| P00734 | Prothrombin | F2 |
| P00738 | Haptoglobin beta chain | HP |
| P00739 | Haptoglobin-related protein | HPR |
| P00740 | Coagulation factor IXa heavy chain | F9 |
| P00742 | Factor X heavy chain | F10 |
| P00746 | Complement factor D | CFD |
| P00747 | Plasmin light chain B | PLG |
| P00748 | Coagulation factor XIIa light chain | F12 |
| P00749 | Urokinase-type plasminogen activator long | PLAU |
| | chain A | |
| P00750 | Tissue-type plasminogen activator | PLAT |
| P00751 | Complement factor B Ba fragment | CFB |
| P00797 | Renin | REN |
| P00973 | 2′-5′-oligoadenylate synthase 1 | OAS1 |
| P00995 | Pancreatic secretory trypsin inhibitor | SPINK1 |
| P01008 | Antithrombin-III | SERPINC1 |
| P01009 | Alpha-1-antitrypsin | SERPINA1 |
| P01011 | Alpha-1-antichymotrypsin His-Pro-less | SERPINA3 |
| P01019 | Angiotensin-1 | AGT |
| P01023 | Alpha-2-macroglobulin | A2M |
| P01024 | Acylation stimulating protein | C3 |
| P01031 | Complement C5 beta chain | C5 |
| P01033 | Metalloproteinase inhibitor 1 | TIMP1 |
| P01034 | Cystatin-C | CST3 |
| P01036 | Cystatin-S | CST4 |
| P01037 | Cystatin-SN | CST1 |
| P01042 | Kininogen-1 light chain | KNG1 |
| P01127 | Platelet-derived growth factor subunit B | PDGFB |
| P01135 | Transforming growth factor alpha | TGFA |
| P01137 | Transforming growth factor beta-1 | TGFB1 |
| P01138 | Beta-nerve growth factor | NGF |
| P01148 | Gonadoliberin-1 | GNRH1 |
| P01160 | Atrial natriuretic factor | NPPA |
| P01178 | Oxytocin | OXT |
| P01185 | Vasopressin-neurophysin 2-copeptin | AVP |
| P01189 | Corticotropin | POMC |
| P01210 | PENK(237-258) | PENK |
| P01213 | Alpha-neoendorphin | PDYN |
| P01215 | Glycoprotein hormones alpha chain | CGA |
| P01222 | Thyrotropin subunit beta | TSHB |
| P01225 | Follitropin subunit beta | FSHB |
| P01229 | Lutropin subunit beta | LHB |
| P01233 | Choriogonadotropin subunit beta | CGB8 |
| P01236 | Prolactin | PRL |
| P01241 | Somatotropin | GH1 |
| P01242 | Growth hormone variant | GH2 |
| P01243 | Chorionic somatomammotropin hormone | CSH2 |
| P01258 | Katacalcin | CALCA |
| P01266 | Thyroglobulin | TG |
| P01270 | Parathyroid hormone | PTH |
| P01275 | Glucagon | GCG |
| P01282 | Intestinal peptide PHM-27 | VIP |
| P01286 | Somatoliberin | GHRH |
| P01298 | Pancreatic prohormone | PPY |
| P01303 | C-flanking peptide of NPY | NPY |
| P01308 | Insulin | INS |
| P01344 | Insulin-like growth factor II | IGF2 |
| P01350 | Big gastrin | GAST |
| P01374 | Lymphotoxin-alpha | LTA |
| P01375 | C-domain 1 | TNF |
| P01562 | Interferon alpha-1/13 | IFNA1 |
| P01563 | Interferon alpha-2 | IFNA2 |
| P01566 | Interferon alpha-10 | IFNA10 |
| P01567 | Interferon alpha-7 | IFNA7 |
| P01568 | Interferon alpha-21 | IFNA21 |
| P01569 | Interferon alpha-5 | IFNA5 |
| P01570 | Interferon alpha-14 | IFNA14 |
| P01571 | Interferon alpha-17 | IFNA17 |
| P01574 | Interferon beta | IFNB1 |
| P01579 | Interferon gamma | IFNG |
| P01583 | Interleukin-1 alpha | IL1A |
| P01584 | Interleukin-1 beta | IL1B |
| P01588 | Erythropoietin | EPO |
| P01591 | Immunoglobulin J chain | IGJ |
| P01732 | T-cell surface glycoprotein CD8 alpha chain | CD8A |
| P01833 | Polymeric immunoglobulin receptor | PIGR |
| P01857 | Ig gamma-1 chain C region | IGHG1 |
| P01859 | Ig gamma-2 chain C region | IGHG2 |
| P01860 | Ig gamma-3 chain C region | IGHG3 |
| P01861 | Ig gamma-4 chain C region | IGHG4 |
| P01871 | Ig mu chain C region | IGHM |
| P01880 | Ig delta chain C region | IGHD |
| P02452 | Collagen alpha-1(I) chain | COL1A1 |
| P02458 | Chondrocalcin | COL2A1 |
| P02461 | Collagen alpha-1(III) chain | COL3A1 |
| P02462 | Collagen alpha-1(IV) chain | COL4A1 |
| P02647 | Apolipoprotein A-I | APOA1 |
| P02649 | Apolipoprotein E | APOE |
| P02652 | Apolipoprotein A-II | APOA2 |
| P02654 | Apolipoprotein C-I | APOC1 |
| P02655 | Apolipoprotein C-II | APOC2 |
| P02656 | Apolipoprotein C-III | APOC3 |
| P02671 | Fibrinogen alpha chain | FGA |
| P02675 | Fibrinopeptide B | FGB |
| P02679 | Fibrinogen gamma chain | FGG |
| P02741 | C-reactive protein | CRP |
| P02743 | Serum amyloid P-component(1-203) | APCS |
| P02745 | Complement C1q subcomponent subunit A | C1QA |
| P02746 | Complement C1q subcomponent subunit B | C1QB |
| P02747 | Complement C1q subcomponent subunit C | C1QC |
| P02748 | Complement component C9b | C9 |
| P02749 | Beta-2-glycoprotein 1 | APOH |
| P02750 | Leucine-rich alpha-2-glycoprotein | LRG1 |
| P02751 | Ugl-Y2 | FN1 |
| P02753 | Retinol-binding protein 4 | RBP4 |
| P02760 | Trypstatin | AMBP |
| P02763 | Alpha-1-acid glycoprotein 1 | ORM1 |
| P02765 | Alpha-2-HS-glycoprotein chain A | AHSG |
| P02766 | Transthyretin | TTR |
| P02768 | Serum albumin | ALB |
| P02771 | Alpha-fetoprotein | AFP |
| P02774 | Vitamin D-binding protein | GC |
| P02775 | Connective tissue-activating peptide III | PPBP |
| P02776 | Platelet factor 4 | PF4 |
| P02778 | CXCL10(1-73) | CXCL10 |
| P02786 | Transferrin receptor protein 1 | TFRC |
| P02787 | Serotransferrin | TF |
| P02788 | Lactoferroxin-C | LTF |
| P02790 | Hemopexin | HPX |
| P02808 | Statherin | STATH |
| P02810 | Salivary acidic proline-rich phosphoprotein 1/2 | PRH2 |
| P02812 | Basic salivary proline-rich protein 2 | PRB2 |
| P02814 | Peptide D1A | SMR3B |
| P02818 | Osteocalcin | BGLAP |
| P03950 | Angiogenin | ANG |
| P03951 | Coagulation factor XIa heavy chain | F11 |
| P03952 | Plasma kallikrein | KLKB1 |
| P03956 | 27 kDa interstitial collagenase | MMP1 |
| P03971 | Muellerian-inhibiting factor | AMH |
| P03973 | Antileukoproteinase | SLPI |
| P04003 | C4b-binding protein alpha chain | C4BPA |
| P04004 | Somatomedin-B | VTN |
| P04054 | Phospholipase A2 | PLA2G1B |
| P04085 | Platelet-derived growth factor subunit A | PDGFA |
| P04090 | Relaxin A chain | RLN2 |
| P04114 | Apolipoprotein B-100 | APOB |
| P04118 | Colipase | CLPS |
| P04141 | Granulocyte-macrophage colony-stimulating | CSF2 |
| | factor | |
| P04155 | Trefoil factor 1 | TFF1 |
| P04180 | Phosphatidylcholine-sterol acyltransferase | LCAT |
| P04196 | Histidine-rich glycoprotein | HRG |
| P04217 | Alpha-1B-glycoprotein | A1BG |
| P04275 | von Willebrand antigen 2 | VWF |
| P04278 | Sex hormone-binding globulin | SHBG |
| P04279 | Alpha-inhibin-31 | SEMG1 |
| P04280 | Basic salivary proline-rich protein 1 | PRB1 |
| P04628 | Proto-oncogene Wnt-1 | WNT1 |
| P04745 | Alpha-amylase 1 | AMY1A |
| P04746 | Pancreatic alpha-amylase | AMY2A |
| P04808 | Prorelaxin H1 | RLN1 |
| P05000 | Interferon omega-1 | IFNW1 |
| P05013 | Interferon alpha-6 | IFNA6 |
| P05014 | Interferon alpha-4 | IFNA4 |
| P05015 | Interferon alpha-16 | IFNA16 |
| P05019 | Insulin-like growth factor I | IGF1 |
| P05060 | GAWK peptide | CHGB |
| P05090 | Apolipoprotein D | APOD |
| P05109 | Protein S100-A8 | S100A8 |
| P05111 | Inhibin alpha chain | INHA |
| P05112 | Interleukin-4 | IL4 |
| P05113 | Interleukin-5 | IL5 |
| P05120 | Plasminogen activator inhibitor 2 | SERPINB2 |
| P05121 | Plasminogen activator inhibitor 1 | SERPINE1 |
| P05154 | Plasma serine protease inhibitor | SERPINA5 |
| P05155 | Plasma protease C1 inhibitor | SERPING1 |
| P05156 | Complement factor I heavy chain | CFI |
| P05160 | Coagulation factor XIII B chain | F13B |
| P05161 | Ubiquitin-like protein ISG15 | ISG15 |
| P05230 | Fibroblast growth factor 1 | FGF1 |
| P05231 | Interleukin-6 | IL6 |
| P05305 | Big endothelin-1 | EDN1 |
| P05408 | C-terminal peptide | SCG5 |
| P05451 | Lithostathine-1-alpha | REG1A |
| P05452 | Tetranectin | CLEC3B |
| P05543 | Thyroxine-binding globulin | SERPINA7 |
| P05814 | Beta-casein | CSN2 |
| P05997 | Collagen alpha-2(V) chain | COL5A2 |
| P06276 | Cholinesterase | BCHE |
| P06307 | Cholecystokinin-12 | CCK |
| P06396 | Gelsolin | GSN |
| P06681 | Complement C2 | C2 |
| P06702 | Protein S100-A9 | S100A9 |
| P06727 | Apolipoprotein A-IV | APOA4 |
| P06734 | Low affinity immunoglobulin epsilon Fc receptor | FCER2 |
| | soluble form | |
| P06744 | Glucose-6-phosphate isomerase | GPI |
| P06850 | Corticoliberin | CRH |
| P06858 | Lipoprotein lipase | LPL |
| P06881 | Calcitonin gene-related peptide 1 | CALCA |
| P07093 | Glia-derived nexin | SERPINE2 |
| P07098 | Gastric triacylglycerol lipase | LIPF |
| P07225 | Vitamin K-dependent protein S | PROS1 |
| P07237 | Protein disulfide-isomerase | P4HB |
| P07288 | Prostate-specific antigen | KLK3 |
| P07306 | Asialoglycoprotein receptor 1 | ASGR1 |
| P07355 | Annexin A2 | ANXA2 |
| P07357 | Complement component C8 alpha chain | C8A |
| P07358 | Complement component C8 beta chain | C8B |
| P07360 | Complement component C8 gamma chain | C8G |
| P07477 | Alpha-trypsin chain 2 | PRSS1 |
| P07478 | Trypsin-2 | PRSS2 |
| P07492 | Neuromedin-C | GRP |
| P07498 | Kappa-casein | CSN3 |
| P07585 | Decorin | DCN |
| P07911 | Uromodulin | UMOD |
| P07942 | Laminin subunit beta-1 | LAMB1 |
| P07988 | Pulmonary surfactant-associated protein B | SFTPB |
| P07998 | Ribonuclease pancreatic | RNASE1 |
| P08118 | Beta-microseminoprotein | MSMB |
| P08123 | Collagen alpha-2(I) chain | COL1A2 |
| P08185 | Corticosteroid-binding globulin | SERPINA6 |
| P08217 | Chymotrypsin-like elastase family member 2A | CELA2A |
| P08218 | Chymotrypsin-like elastase family member 2B | CELA2B |
| P08253 | 72 kDa type IV collagenase | MMP2 |
| P08254 | Stromelysin-1 | MMP3 |
| P08294 | Extracellular superoxide dismutase [Cu—Zn] | SOD3 |
| P08476 | Inhibin beta A chain | INHBA |
| P08493 | Matrix Gla protein | MGP |
| P08572 | Collagen alpha-2(IV) chain | COL4A2 |
| P08581 | Hepatocyte growth factor receptor | MET |
| P08603 | Complement factor H | CFH |
| P08620 | Fibroblast growth factor 4 | FGF4 |
| P08637 | Low affinity immunoglobulin gamma Fc region | FCGR3A |
| | receptor III-A | |
| P08697 | Alpha-2-antiplasmin | SERPINF2 |
| P08700 | Interleukin-3 | IL3 |
| P08709 | Coagulation factor VII | F7 |
| P08833 | Insulin-like growth factor-binding protein 1 | IGFBP1 |
| P08887 | Interleukin-6 receptor subunit alpha | IL6R |
| P08949 | Neuromedin-B-32 | NMB |
| P08F94 | Fibrocystin | PKHD1 |
| P09038 | Fibroblast growth factor 2 | FGF2 |
| P09228 | Cystatin-SA | CST2 |
| P09237 | Matrilysin | MMP7 |
| P09238 | Stromelysin-2 | MMP10 |
| P09341 | Growth-regulated alpha protein | CXCL1 |
| P09382 | Galectin-1 | LGALS1 |
| P09466 | Glycodelin | PAEP |
| P09486 | SPARC | SPARC |
| P09529 | Inhibin beta B chain | INHBB |
| P09544 | Protein Wnt-2 | WNT2 |
| P09603 | Processed macrophage colony-stimulating factor 1 | CSF1 |
| P09681 | Gastric inhibitory polypeptide | GIP |
| P09683 | Secretin | SCT |
| P09919 | Granulocyte colony-stimulating factor | CSF3 |
| P0C091 | FRAS1-related extracellular matrix protein 3 | FREM3 |
| P0C0L4 | C4d-A | C4A |
| P0C0L5 | Complement C4-B alpha chain | C4B |
| P0C0P6 | Neuropeptide S | NPS |
| P0C7L1 | Serine protease inhibitor Kazal-type 8 | SPINK8 |
| P0C862 | Complement C1q and tumor necrosis factor- | C1QTNF9 |
| | related protein 9A | |
| P0C8F1 | Prostate and testis expressed protein 4 | PATE4 |
| P0CG01 | Gastrokine-3 | GKN3P |
| P0CG36 | Cryptic family protein 1B | CFC1B |
| P0CG37 | Cryptic protein | CFC1 |
| P0CJ68 | Humanin-like protein 1 | MTRNR2L1 |
| P0CJ69 | Humanin-like protein 2 | MTRNR2L2 |
| P0CJ70 | Humanin-like protein 3 | MTRNR2L3 |
| P0CJ71 | Humanin-like protein 4 | MTRNR2L4 |
| P0CJ72 | Humanin-like protein 5 | MTRNR2L5 |
| P0CJ73 | Humanin-like protein 6 | MTRNR2L6 |
| P0CJ74 | Humanin-like protein 7 | MTRNR2L7 |
| P0CJ75 | Humanin-like protein 8 | MTRNR2L8 |
| P0CJ76 | Humanin-like protein 9 | MTRNR2L9 |
| P0CJ77 | Humanin-like protein 10 | MTRNR2L10 |
| P0DJD7 | Pepsin A-4 | PGA4 |
| P0DJD8 | Pepsin A-3 | PGA3 |
| P0DJD9 | Pepsin A-5 | PGA5 |
| P0DJI8 | Amyloid protein A | SAA1 |
| P0DJI9 | Serum amyloid A-2 protein | SAA2 |
| P10082 | Peptide YY(3-36) | PYY |
| P10092 | Calcitonin gene-related peptide 2 | CALCB |
| P10124 | Serglycin | SRGN |
| P10145 | MDNCF-a | IL8 |
| P10147 | MIP-1-alpha(4-69) | CCL3 |
| P10163 | Peptide P-D | PRB4 |
| P10451 | Osteopontin | SPP1 |
| P10599 | Thioredoxin | TXN |
| P10600 | Transforming growth factor beta-3 | TGFB3 |
| P10643 | Complement component C7 | C7 |
| P10645 | Vasostatin-2 | CHGA |
| P10646 | Tissue factor pathway inhibitor | TFPI |
| P10720 | Platelet factor 4 variant(4-74) | PF4V1 |
| P10745 | Retinol-binding protein 3 | RBP3 |
| P10767 | Fibroblast growth factor 6 | FGF6 |
| P10909 | Clusterin alpha chain | CLU |
| P10912 | Growth hormone receptor | GHR |
| P10915 | Hyaluronan and proteoglycan link protein 1 | HAPLN1 |
| P10966 | T-cell surface glycoprotein CD8 beta chain | CD8B |
| P10997 | Islet amyloid polypeptide | IAPP |
| P11047 | Laminin subunit gamma-1 | LAMC1 |
| P11150 | Hepatic triacylglycerol lipase | LIPC |
| P11226 | Mannose-binding protein C | MBL2 |
| P11464 | Pregnancy-specific beta-1-glycoprotein 1 | PSG1 |
| P11465 | Pregnancy-specific beta-1-glycoprotein 2 | PSG2 |
| P11487 | Fibroblast growth factor 3 | FGF3 |
| P11597 | Cholesteryl ester transfer protein | CETP |
| P11684 | Uteroglobin | SCGB1A1 |
| P11686 | Pulmonary surfactant-associated protein C | SFTPC |
| P12034 | Fibroblast growth factor 5 | FGF5 |
| P12107 | Collagen alpha-1(XI) chain | COL11A1 |
| P12109 | Collagen alpha-1(VI) chain | COL6A1 |
| P12110 | Collagen alpha-2(VI) chain | COL6A2 |
| P12111 | Collagen alpha-3(VI) chain | COL6A3 |
| P12259 | Coagulation factor V | F5 |
| P12272 | PTHrP[1-36] | PTHLH |
| P12273 | Prolactin-inducible protein | PIP |
| P12544 | Granzyme A | GZMA |
| P12643 | Bone morphogenetic protein 2 | BMP2 |
| P12644 | Bone morphogenetic protein 4 | BMP4 |
| P12645 | Bone morphogenetic protein 3 | BMP3 |
| P12724 | Eosinophil cationic protein | RNASE3 |
| P12821 | Angiotensin-converting enzyme, soluble form | ACE |
| P12838 | Neutrophil defensin 4 | DEFA4 |
| P12872 | Motilin | MLN |
| P13232 | Interleukin-7 | IL7 |
| P13236 | C-C motif chemokine 4 | CCL4 |
| P13284 | Gamma-interferon-inducible lysosomal thiol | IFI30 |
| | reductase | |
| P13500 | C-C motif chemokine 2 | CCL2 |
| P13501 | C-C motif chemokine 5 | CCL5 |
| P13521 | Secretogranin-2 | SCG2 |
| P13591 | Neural cell adhesion molecule 1 | NCAM1 |
| P13611 | Versican core protein | VCAN |
| P13671 | Complement component C6 | C6 |
| P13688 | Carcinoembryonic antigen-related cell adhesion | CEACAM1 |
| | molecule 1 | |
| P13725 | Oncostatin-M | OSM |
| P13726 | Tissue factor | F3 |
| P13727 | Eosinophil granule major basic protein | PRG2 |
| P13942 | Collagen alpha-2(XI) chain | COL11A2 |
| P13987 | CD59 glycoprotein | CD59 |
| P14138 | Endothelin-3 | EDN3 |
| P14174 | Macrophage migration inhibitory factor | MIF |
| P14207 | Folate receptor beta | FOLR2 |
| P14222 | Perforin-1 | PRF1 |
| P14543 | Nidogen-1 | NID1 |
| P14555 | Phospholipase A2, membrane associated | PLA2G2A |
| P14625 | Endoplasmin | HSP90B1 |
| P14735 | Insulin-degrading enzyme | IDE |
| P14778 | Interleukin-1 receptor type 1, soluble form | IL1R1 |
| P14780 | 82 kDa matrix metalloproteinase-9 | MMP9 |
| P15018 | Leukemia inhibitory factor | LIF |
| P15085 | Carboxypeptidase A1 | CPA1 |
| P15086 | Carboxypeptidase B | CPB1 |
| P15151 | Poliovirus receptor | PVR |
| P15169 | Carboxypeptidase N catalytic chain | CPN1 |
| P15248 | Interleukin-9 | IL9 |
| P15291 | N-acetyllactosamine synthase | B4GALT1 |
| P15309 | PAPf39 | ACPP |
| P15328 | Folate receptor alpha | FOLR1 |
| P15374 | Ubiquitin carboxyl-terminal hydrolase isozyme L3 | UCHL3 |
| P15502 | Elastin | ELN |
| P15509 | Granulocyte-macrophage colony-stimulating | CSF2RA |
| | factor receptor subunit alpha | |
| P15515 | Histatin-1 | HTN1 |
| P15516 | His3-(31-51)-peptide | HTN3 |
| P15692 | Vascular endothelial growth factor A | VEGFA |
| P15814 | Immunoglobulin lambda-like polypeptide 1 | IGLL1 |
| P15907 | Beta-galactoside alpha-2,6-sialyltransferase 1 | ST6GAL1 |
| P15941 | Mucin-1 subunit beta | MUC1 |
| P16035 | Metalloproteinase inhibitor 2 | TIMP2 |
| P16112 | Aggrecan core protein 2 | ACAN |
| P16233 | Pancreatic triacylglycerol lipase | PNLIP |
| P16442 | Histo-blood group ABO system transferase | ABO |
| P16471 | Prolactin receptor | PRLR |
| P16562 | Cysteine-rich secretory protein 2 | CRISP2 |
| P16619 | C-C motif chemokine 3-like 1 | CCL3L1 |
| P16860 | BNP(3-29) | NPPB |
| P16870 | Carboxypeptidase E | CPE |
| P16871 | Interleukin-7 receptor subunit alpha | IL7R |
| P17213 | Bactericidal permeability-increasing protein | BPI |
| P17538 | Chymotrypsinogen B | CTRB1 |
| P17931 | Galectin-3 | LGALS3 |
| P17936 | Insulin-like growth factor-binding protein 3 | IGFBP3 |
| P17948 | Vascular endothelial growth factor receptor 1 | FLT1 |
| P18065 | Insulin-like growth factor-binding protein 2 | IGFBP2 |
| P18075 | Bone morphogenetic protein 7 | BMP7 |
| P18428 | Lipopolysaccharide-binding protein | LBP |
| P18509 | PACAP-related peptide | ADCYAP1 |
| P18510 | Interleukin-1 receptor antagonist protein | IL1RN |
| P18827 | Syndecan-1 | SDC1 |
| P19021 | Peptidylglycine alpha-hydroxylating | PAM |
| | monooxygenase | |
| P19235 | Erythropoietin receptor | EPOR |
| P19438 | Tumor necrosis factor-binding protein 1 | TNFRSF1A |
| P19652 | Alpha-1-acid glycoprotein 2 | ORM2 |
| P19801 | Amiloride-sensitive amine oxidase [copper- | ABP1 |
| | containing] | |
| P19823 | Inter-alpha-trypsin inhibitor heavy chain H2 | ITIH2 |
| P19827 | Inter-alpha-trypsin inhibitor heavy chain H1 | ITIH1 |
| P19835 | Bile salt-activated lipase | CEL |
| P19875 | C-X-C motif chemokine 2 | CXCL2 |
| P19876 | C-X-C motif chemokine 3 | CXCL3 |
| P19883 | Follistatin | FST |
| P19957 | Elafin | PI3 |
| P19961 | Alpha-amylase 2B | AMY2B |
| P20061 | Transcobalamin-1 | TCN1 |
| P20062 | Transcobalamin-2 | TCN2 |
| P20142 | Gastricsin | PGC |
| P20155 | Serine protease inhibitor Kazal-type 2 | SPINK2 |
| P20231 | Tryptase beta-2 | TPSB2 |
| P20333 | Tumor necrosis factor receptor superfamily | TNFRSF1B |
| | member 1B | |
| P20366 | Substance P | TAC1 |
| P20382 | Melanin-concentrating hormone | PMCH |
| P20396 | Thyroliberin | TRH |
| P20742 | Pregnancy zone protein | PZP |
| P20774 | Mimecan | OGN |
| P20783 | Neurotrophin-3 | NTF3 |
| P20800 | Endothelin-2 | EDN2 |
| P20809 | Interleukin-11 | IL11 |
| P20827 | Ephrin-A1 | EFNA1 |
| P20849 | Collagen alpha-1(IX) chain | COL9A1 |
| P20851 | C4b-binding protein beta chain | C4BPB |
| P20908 | Collagen alpha-1(V) chain | COL5A1 |
| P21128 | Poly(U)-specific endoribonuclease | ENDOU |
| P21246 | Pleiotrophin | PTN |
| P21583 | Kit ligand | KITLG |
| P21741 | Midkine | MDK |
| P21754 | Zona pellucida sperm-binding protein 3 | ZP3 |
| P21781 | Fibroblast growth factor 7 | FGF7 |
| P21802 | Fibroblast growth factor receptor 2 | FGFR2 |
| P21810 | Biglycan | BGN |
| P21815 | Bone sialoprotein 2 | IBSP |
| P21860 | Receptor tyrosine-protein kinase erbB-3 | ERBB3 |
| P21941 | Cartilage matrix protein | MATN1 |
| P22003 | Bone morphogenetic protein 5 | BMP5 |
| P22004 | Bone morphogenetic protein 6 | BMP6 |
| P22079 | Lactoperoxidase | LPO |
| P22105 | Tenascin-X | TNXB |
| P22301 | Interleukin-10 | IL10 |
| P22303 | Acetylcholinesterase | ACHE |
| P22352 | Glutathione peroxidase 3 | GPX3 |
| P22362 | C-C motif chemokine 1 | CCL1 |
| P22455 | Fibroblast growth factor receptor 4 | FGFR4 |
| P22466 | Galanin message-associated peptide | GAL |
| P22692 | Insulin-like growth factor-binding protein 4 | IGFBP4 |
| P22749 | Granulysin | GNLY |
| P22792 | Carboxypeptidase N subunit 2 | CPN2 |
| P22891 | Vitamin K-dependent protein Z | PROZ |
| P22894 | Neutrophil collagenase | MMP8 |
| P23142 | Fibulin-1 | FBLN1 |
| P23280 | Carbonic anhydrase 6 | CA6 |
| P23352 | Anosmin-1 | KAL1 |
| P23435 | Cerebellin-1 | CBLN1 |
| P23560 | Brain-derived neurotrophic factor | BDNF |
| P23582 | C-type natriuretic peptide | NPPC |
| P23946 | Chymase | CMA1 |
| P24043 | Laminin subunit alpha-2 | LAMA2 |
| P24071 | Immunoglobulin alpha Fc receptor | FCAR |
| P24347 | Stromelysin-3 | MMP11 |
| P24387 | Corticotropin-releasing factor-binding protein | CRHBP |
| P24592 | Insulin-like growth factor-binding protein 6 | IGFBP6 |
| P24593 | Insulin-like growth factor-binding protein 5 | IGFBP5 |
| P24821 | Tenascin | TNC |
| P24855 | Deoxyribonuclease-1 | DNASE1 |
| P25067 | Collagen alpha-2(VIII) chain | COL8A2 |
| P25311 | Zinc-alpha-2-glycoprotein | AZGP1 |
| P25391 | Laminin subunit alpha-1 | LAMA1 |
| P25445 | Tumor necrosis factor receptor superfamily | FAS |
| | member 6 | |
| P25940 | Collagen alpha-3(V) chain | COL5A3 |
| P25942 | Tumor necrosis factor receptor superfamily | CD40 |
| | member 5 | |
| P26022 | Pentraxin-related protein PTX3 | PTX3 |
| P26927 | Hepatocyte growth factor-like protein beta chain | MST1 |
| P27169 | Serum paraoxonase/arylesterase 1 | PON1 |
| P27352 | Gastric intrinsic factor | GIF |
| P27487 | Dipeptidyl peptidase 4 membrane form | DPP4 |
| P27539 | Embryonic growth/differentiation factor 1 | GDF1 |
| P27658 | Vastatin | COL8A1 |
| P27797 | Calreticulin | CALR |
| P27918 | Properdin | CFP |
| P28039 | Acyloxyacyl hydrolase | AOAH |
| P28300 | Protein-lysine 6-oxidase | LOX |
| P28325 | Cystatin-D | CST5 |
| P28799 | Granulin-1 | GRN |
| P29122 | Proprotein convertase subtilisin/kexin type 6 | PCSK6 |
| P29279 | Connective tissue growth factor | CTGF |
| P29320 | Ephrin type-A receptor 3 | EPHA3 |
| P29400 | Collagen alpha-5(IV) chain | COL4A5 |
| P29459 | Interleukin-12 subunit alpha | IL12A |
| P29460 | Interleukin-12 subunit beta | IL12B |
| P29508 | Serpin B3 | SERPINB3 |
| P29622 | Kallistatin | SERPINA4 |
| P29965 | CD40 ligand, soluble form | CD40LG |
| P30990 | Neurotensin/neuromedin N | NTS |
| P31025 | Lipocalin-1 | LCN1 |
| P31151 | Protein S100-A7 | S100A7 |
| P31371 | Fibroblast growth factor 9 | FGF9 |
| P31431 | Syndecan-4 | SDC4 |
| P31947 | 14-3-3 protein sigma | SFN |
| P32455 | Interferon-induced guanylate-binding protein 1 | GBP1 |
| P32881 | Interferon alpha-8 | IFNA8 |
| P34096 | Ribonuclease 4 | RNASE4 |
| P34130 | Neurotrophin-4 | NTF4 |
| P34820 | Bone morphogenetic protein 8B | BMP8B |
| P35030 | Trypsin-3 | PRSS3 |
| P35052 | Secreted glypican-1 | GPC1 |
| P35070 | Betacellulin | BTC |
| P35225 | Interleukin-13 | IL13 |
| P35247 | Pulmonary surfactant-associated protein D | SFTPD |
| P35318 | ADM | ADM |
| P35542 | Serum amyloid A-4 protein | SAA4 |
| P35555 | Fibrillin-1 | FBN1 |
| P35556 | Fibrillin-2 | FBN2 |
| P35625 | Metalloproteinase inhibitor 3 | TIMP3 |
| P35858 | Insulin-like growth factor-binding protein complex | IGFALS |
| | acid labile subunit | |
| P35916 | Vascular endothelial growth factor receptor 3 | FLT4 |
| P35968 | Vascular endothelial growth factor receptor 2 | KDR |
| P36222 | Chitinase-3-like protein 1 | CHI3L1 |
| P36952 | Serpin B5 | SERPINB5 |
| P36955 | Pigment epithelium-derived factor | SERPINF1 |
| P36980 | Complement factor H-related protein 2 | CFHR2 |
| P39059 | Collagen alpha-1(XV) chain | COL15A1 |
| P39060 | Collagen alpha-1(XVIII) chain | COL18A1 |
| P39877 | Calcium-dependent phospholipase A2 | PLA2G5 |
| P39900 | Macrophage metalloelastase | MMP12 |
| P39905 | Glial cell line-derived neurotrophic factor | GDNF |
| P40225 | Thrombopoietin | THPO |
| P40967 | M-alpha | PMEL |
| P41159 | Leptin | LEP |
| P41221 | Protein Wnt-5a | WNT5A |
| P41222 | Prostaglandin-H2 D-isomerase | PTGDS |
| P41271 | Neuroblastoma suppressor of tumorigenicity 1 | NBL1 |
| P41439 | Folate receptor gamma | FOLR3 |
| P42127 | Agouti-signaling protein | ASIP |
| P42702 | Leukemia inhibitory factor receptor | LIFR |
| P42830 | ENA-78(9-78) | CXCL5 |
| P43026 | Growth/differentiation factor 5 | GDF5 |
| P43251 | Biotinidase | BTD |
| P43652 | Afamin | AFM |
| P45452 | Collagenase 3 | MMP13 |
| P47710 | Casoxin-D | CSN1S1 |
| P47929 | Galectin-7 | LGALS7B |
| P47972 | Neuronal pentraxin-2 | NPTX2 |
| P47989 | Xanthine oxidase | XDH |
| P47992 | Lymphotactin | XCL1 |
| P48023 | Tumor necrosis factor ligand superfamily | FASLG |
| | member 6, membrane form | |
| P48052 | Carboxypeptidase A2 | CPA2 |
| P48061 | Stromal cell-derived factor 1 | CXCL12 |
| P48304 | Lithostathine-1-beta | REG1B |
| P48307 | Tissue factor pathway inhibitor 2 | TFPI2 |
| P48357 | Leptin receptor | LEPR |
| P48594 | Serpin B4 | SERPINB4 |
| P48645 | Neuromedin-U-25 | NMU |
| P48740 | Mannan-binding lectin serine protease 1 | MASP1 |
| P48745 | Protein NOV homolog | NOV |
| P48960 | CD97 antigen subunit beta | CD97 |
| P49223 | Kunitz-type protease inhibitor 3 | SPINT3 |
| P49747 | Cartilage oligomeric matrix protein | COMP |
| P49763 | Placenta growth factor | PGF |
| P49765 | Vascular endothelial growth factor B | VEGFB |
| P49767 | Vascular endothelial growth factor C | VEGFC |
| P49771 | Fms-related tyrosine kinase 3 ligand | FLT3LG |
| P49862 | Kallikrein-7 | KLK7 |
| P49863 | Granzyme K | GZMK |
| P49908 | Selenoprotein P | SEPP1 |
| P49913 | Antibacterial protein FALL-39 | CAMP |
| P50607 | Tubby protein homolog | TUB |
| P51124 | Granzyme M | GZMM |
| P51512 | Matrix metalloproteinase-16 | MMP16 |
| P51654 | Glypican-3 | GPC3 |
| P51671 | Eotaxin | CCL11 |
| P51884 | Lumican | LUM |
| P51888 | Prolargin | PRELP |
| P52798 | Ephrin-A4 | EFNA4 |
| P52823 | Stanniocalcin-1 | STC1 |
| P53420 | Collagen alpha-4(IV) chain | COL4A4 |
| P53621 | Coatomer subunit alpha | COPA |
| P54108 | Cysteine-rich secretory protein 3 | CRISP3 |
| P54315 | Pancreatic lipase-related protein 1 | PNLIPRP1 |
| P54317 | Pancreatic lipase-related protein 2 | PNLIPRP2 |
| P54793 | Arylsulfatase F | ARSF |
| P55000 | Secreted Ly-6/uPAR-related protein 1 | SLURP1 |
| P55001 | Microfibrillar-associated protein 2 | MFAP2 |
| P55056 | Apolipoprotein C-IV | APOC4 |
| P55058 | Phospholipid transfer protein | PLTP |
| P55075 | Fibroblast growth factor 8 | FGF8 |
| P55081 | Microfibrillar-associated protein 1 | MFAP1 |
| P55083 | Microfibril-associated glycoprotein 4 | MFAP4 |
| P55107 | Bone morphogenetic protein 3B | GDF10 |
| P55145 | Mesencephalic astrocyte-derived neurotrophic | MANF |
| | factor | |
| P55259 | Pancreatic secretory granule membrane major | GP2 |
| | glycoprotein GP2 | |
| P55268 | Laminin subunit beta-2 | LAMB2 |
| P55773 | CCL23(30-99) | CCL23 |
| P55774 | C-C motif chemokine 18 | CCL18 |
| P55789 | FAD-linked sulfhydryl oxidase ALR | GFER |
| P56703 | Proto-oncogene Wnt-3 | WNT3 |
| P56704 | Protein Wnt-3a | WNT3A |
| P56705 | Protein Wnt-4 | WNT4 |
| P56706 | Protein Wnt-7b | WNT7B |
| P56730 | Neurotrypsin | PRSS12 |
| P56851 | Epididymal secretory protein E3-beta | EDDM3B |
| P56975 | Neuregulin-3 | NRG3 |
| P58062 | Serine protease inhibitor Kazal-type 7 | SPINK7 |
| P58215 | Lysyl oxidase homolog 3 | LOXL3 |
| P58294 | Prokineticin-1 | PROK1 |
| P58335 | Anthrax toxin receptor 2 | ANTXR2 |
| P58397 | A disintegrin and metalloproteinase with | ADAMTS12 |
| | thrombospondin motifs 12 | |
| P58417 | Neurexophilin-1 | NXPH1 |
| P58499 | Protein FAM3B | FAM3B |
| P59510 | A disintegrin and metalloproteinase with | ADAMTS20 |
| | thrombospondin motifs 20 | |
| P59665 | Neutrophil defensin 1 | DEFA1B |
| P59666 | Neutrophil defensin 3 | DEFA3 |
| P59796 | Glutathione peroxidase 6 | GPX6 |
| P59826 | BPI fold-containing family B member 3 | BPIFB3 |
| P59827 | BPI fold-containing family B member 4 | BPIFB4 |
| P59861 | Beta-defensin 131 | DEFB131 |
| P60022 | Beta-defensin 1 | DEFB1 |
| P60153 | Inactive ribonuclease-like protein 9 | RNASE9 |
| P60827 | Complement C1q tumor necrosis factor-related | C1QTNF8 |
| | protein 8 | |
| P60852 | Zona pellucida sperm-binding protein 1 | ZP1 |
| P60985 | Keratinocyte differentiation-associated protein | KRTDAP |
| P61109 | Kidney androgen-regulated protein | KAP |
| P61278 | Somatostatin-14 | SST |
| P61366 | Osteocrin | OSTN |
| P61626 | Lysozyme C | LYZ |
| P61769 | Beta-2-microglobulin | B2M |
| P61812 | Transforming growth factor beta-2 | TGFB2 |
| P61916 | Epididymal secretory protein E1 | NPC2 |
| P62502 | Epididymal-specific lipocalin-6 | LCN6 |
| P62937 | Peptidyl-prolyl cis-trans isomerase A | PPIA |
| P67809 | Nuclease-sensitive element-binding protein 1 | YBX1 |
| P67812 | Signal peptidase complex catalytic subunit | SEC11A |
| | SEC11A | |
| P78310 | Coxsackievirus and adenovirus receptor | CXADR |
| P78333 | Secreted glypican-5 | GPC5 |
| P78380 | Oxidized low-density lipoprotein receptor 1 | OLR1 |
| P78423 | Processed fractalkine | CX3CL1 |
| P78509 | Reelin | RELN |
| P78556 | CCL20(2-70) | CCL20 |
| P80075 | MCP-2(6-76) | CCL8 |
| P80098 | C-C motif chemokine 7 | CCL7 |
| P80108 | Phosphatidylinositol-glycan-specific | GPLD1 |
| | phospholipase D | |
| P80162 | C-X-C motif chemokine 6 | CXCL6 |
| P80188 | Neutrophil gelatinase-associated lipocalin | LCN2 |
| P80303 | Nucleobindin-2 | NUCB2 |
| P80511 | Calcitermin | S100A12 |
| P81172 | Hepcidin-25 | HAMP |
| P81277 | Prolactin-releasing peptide | PRLH |
| P81534 | Beta-defensin 103 | DEFB103A |
| P81605 | Dermcidin | DCD |
| P82279 | Protein crumbs homolog 1 | CRB1 |
| P82987 | ADAMTS-like protein 3 | ADAMTSL3 |
| P83105 | Serine protease HTRA4 | HTRA4 |
| P83110 | Serine protease HTRA3 | HTRA3 |
| P83859 | Orexigenic neuropeptide QRFP | QRFP |
| P98088 | Mucin-5AC | MUC5AC |
| P98095 | Fibulin-2 | FBLN2 |
| P98160 | Basement membrane-specific heparan sulfate | HSPG2 |
| | proteoglycan core protein | |
| P98173 | Protein FAM3A | FAM3A |
| Q00604 | Norrin | NDP |
| Q00796 | Sorbitol dehydrogenase | SORD |
| Q00887 | Pregnancy-specific beta-1-glycoprotein 9 | PSG9 |
| Q00888 | Pregnancy-specific beta-1-glycoprotein 4 | PSG4 |
| Q00889 | Pregnancy-specific beta-1-glycoprotein 6 | PSG6 |
| Q01523 | HD5(56-94) | DEFA5 |
| Q01524 | Defensin-6 | DEFA6 |
| Q01955 | Collagen alpha-3(IV) chain | COL4A3 |
| Q02297 | Pro-neuregulin-1, membrane-bound isoform | NRG1 |
| Q02325 | Plasminogen-like protein B | PLGLB1 |
| Q02383 | Semenogelin-2 | SEMG2 |
| Q02388 | Collagen alpha-1(VII) chain | COL7A1 |
| Q02505 | Mucin-3A | MUC3A |
| Q02509 | Otoconin-90 | OC90 |
| Q02747 | Guanylin | GUCA2A |
| Q02763 | Angiopoietin-1 receptor | TEK |
| Q02817 | Mucin-2 | MUC2 |
| Q02985 | Complement factor H-related protein 3 | CFHR3 |
| Q03167 | Transforming growth factor beta receptor type 3 | TGFBR3 |
| Q03403 | Trefoil factor 2 | TFF2 |
| Q03405 | Urokinase plasminogen activator surface receptor | PLAUR |
| Q03591 | Complement factor H-related protein 1 | CFHR1 |
| Q03692 | Collagen alpha-1(X) chain | COL10A1 |
| Q04118 | Basic salivary proline-rich protein 3 | PRB3 |
| Q04756 | Hepatocyte growth factor activator short chain | HGFAC |
| Q04900 | Sialomucin core protein 24 | CD164 |
| Q05315 | Eosinophil lysophospholipase | CLC |
| Q05707 | Collagen alpha-1(XIV) chain | COL14A1 |
| Q05996 | Processed zona pellucida sperm-binding protein 2 | ZP2 |
| Q06033 | Inter-alpha-trypsin inhibitor heavy chain H3 | ITIH3 |
| Q06141 | Regenerating islet-derived protein 3-alpha | REG3A |
| Q06828 | Fibromodulin | FMOD |
| Q07092 | Collagen alpha-1(XVI) chain | COL16A1 |
| Q07325 | C-X-C motif chemokine 9 | CXCL9 |
| Q07507 | Dermatopontin | DPT |
| Q075Z2 | Binder of sperm protein homolog 1 | BSPH1 |
| Q07654 | Trefoil factor 3 | TFF3 |
| Q07699 | Sodium channel subunit beta-1 | SCN1B |
| Q08345 | Epithelial discoidin domain-containing receptor 1 | DDR1 |
| Q08380 | Galectin-3-binding protein | LGALS3BP |
| Q08397 | Lysyl oxidase homolog 1 | LOXL1 |
| Q08431 | Lactadherin | MFGE8 |
| Q08629 | Testican-1 | SPOCK1 |
| Q08648 | Sperm-associated antigen 11B | SPAG11B |
| Q08830 | Fibrinogen-like protein 1 | FGL1 |
| Q10471 | Polypeptide N-acetylgalactosaminyltransferase 2 | GALNT2 |
| Q10472 | Polypeptide N-acetylgalactosaminyltransferase 1 | GALNT1 |
| Q11201 | CMP-N-acetylneuraminate-beta-galactosamide- | ST3GAL1 |
| | alpha-2,3-sialyltransferase 1 | |
| Q11203 | CMP-N-acetylneuraminate-beta-1,4-galactoside | ST3GAL3 |
| | alpha-2,3-sialyltransferase | |
| Q11206 | CMP-N-acetylneuraminate-beta-galactosamide- | ST3GAL4 |
| | alpha-2,3-sialyltransferase 4 | |
| Q12794 | Hyaluronidase-1 | HYAL1 |
| Q12805 | EGF-containing fibulin-like extracellular matrix | EFEMP1 |
| | protein 1 | |
| Q12836 | Zona pellucida sperm-binding protein 4 | ZP4 |
| Q12841 | Follistatin-related protein 1 | FSTL1 |
| Q12904 | Aminoacyl tRNA synthase complex-interacting | AIMP1 |
| | multifunctional protein 1 | |
| Q13018 | Soluble secretory phospholipase A2 receptor | PLA2R1 |
| Q13072 | B melanoma antigen 1 | BAGE |
| Q13093 | Platelet-activating factor acetylhydrolase | PLA2G7 |
| Q13103 | Secreted phosphoprotein 24 | SPP2 |
| Q13162 | Peroxiredoxin-4 | PRDX4 |
| Q13201 | Platelet glycoprotein Ia* | MMRN1 |
| Q13214 | Semaphorin-3B | SEMA3B |
| Q13219 | Pappalysin-1 | PAPPA |
| Q13231 | Chitotriosidase-1 | CHIT1 |
| Q13253 | Noggin | NOG |
| Q13261 | Interleukin-15 receptor subunit alpha | IL15RA |
| Q13275 | Semaphorin-3F | SEMA3F |
| Q13291 | Signaling lymphocytic activation molecule | SLAMF1 |
| Q13316 | Dentin matrix acidic phosphoprotein 1 | DMP1 |
| Q13361 | Microfibrillar-associated protein 5 | MFAP5 |
| Q13410 | Butyrophilin subfamily 1 member A1 | BTN1A1 |
| Q13421 | Mesothelin, cleaved form | MSLN |
| Q13429 | Insulin-like growth factor I | IGF-I |
| Q13443 | Disintegrin and metalloproteinase domain- | ADAM9 |
| | containing protein 9 | |
| Q13519 | Neuropeptide 1 | PNOC |
| Q13751 | Laminin subunit beta-3 | LAMB3 |
| Q13753 | Laminin subunit gamma-2 | LAMC2 |
| Q13790 | Apolipoprotein F | APOF |
| Q13822 | Ectonucleotide pyrophosphatase/phosphodiesterase | ENPP2 |
| | family member 2 | |
| Q14031 | Collagen alpha-6(IV) chain | COL4A6 |
| Q14050 | Collagen alpha-3(IX) chain | COL9A3 |
| Q14055 | Collagen alpha-2(IX) chain | COL9A2 |
| Q14112 | Nidogen-2 | NID2 |
| Q14114 | Low-density lipoprotein receptor-related protein 8 | LRP8 |
| Q14118 | Dystroglycan | DAG1 |
| Q14314 | Fibroleukin | FGL2 |
| Q14393 | Growth arrest-specific protein 6 | GAS6 |
| Q14406 | Chorionic somatomammotropin hormone-like 1 | CSHL1 |
| Q14507 | Epididymal secretory protein E3-alpha | EDDM3A |
| Q14508 | WAP four-disulfide core domain protein 2 | WFDC2 |
| Q14512 | Fibroblast growth factor-binding protein 1 | FGFBP1 |
| Q14515 | SPARC-like protein 1 | SPARCL1 |
| Q14520 | Hyaluronan-binding protein 2 27 kDa light chain | HABP2 |
| Q14563 | Semaphorin-3A | SEMA3A |
| Q14623 | Indian hedgehog protein | IHH |
| Q14624 | Inter-alpha-trypsin inhibitor heavy chain H4 | ITIH4 |
| Q14667 | UPF0378 protein KIAA0100 | KIAA0100 |
| Q14703 | Membrane-bound transcription factor site-1 | MBTPS1 |
| | protease | |
| Q14766 | Latent-transforming growth factor beta-binding | LTBP1 |
| | protein 1 | |
| Q14767 | Latent-transforming growth factor beta-binding | LTBP2 |
| | protein 2 | |
| Q14773 | Intercellular adhesion molecule 4 | ICAM4 |
| Q14993 | Collagen alpha-1(XIX) chain | COL19A1 |
| Q14CN2 | Calcium-activated chloride channel regulator 4, | CLCA4 |
| | 110 kDa form | |
| Q15046 | Lysine--tRNA ligase | KARS |
| Q15063 | Periostin | POSTN |
| Q15109 | Advanced glycosylation end product-specific | AGER |
| | receptor | |
| Q15113 | Procollagen C-endopeptidase enhancer 1 | PCOLCE |
| Q15166 | Serum paraoxonase/lactonase 3 | PON3 |
| Q15195 | Plasminogen-like protein A | PLGLA |
| Q15198 | Platelet-derived growth factor receptor-like protein | PDGFRL |
| Q15223 | Poliovirus receptor-related protein 1 | PVRL1 |
| Q15238 | Pregnancy-specific beta-1-glycoprotein 5 | PSG5 |
| Q15363 | Transmembrane emp24 domain-containing protein 2 | TMED2 |
| Q15375 | Ephrin type-A receptor 7 | EPHA7 |
| Q15389 | Angiopoietin-1 | ANGPT1 |
| Q15465 | Sonic hedgehog protein | SHH |
| Q15485 | Ficolin-2 | FCN2 |
| Q15517 | Corneodesmosin | CDSN |
| Q15582 | Transforming growth factor-beta-induced protein ig-h3 | TGFBI |
| Q15661 | Tryptase alpha/beta-1 | TPSAB1 |
| Q15726 | Metastin | KISS1 |
| Q15782 | Chitinase-3-like protein 2 | CHI3L2 |
| Q15828 | Cystatin-M | CST6 |
| Q15846 | Clusterin-like protein 1 | CLUL1 |
| Q15848 | Adiponectin | ADIPOQ |
| Q16206 | Protein disulfide-thiol oxidoreductase | ENOX2 |
| Q16270 | Insulin-like growth factor-binding protein 7 | IGFBP7 |
| Q16363 | Laminin subunit alpha-4 | LAMA4 |
| Q16378 | Proline-rich protein 4 | PRR4 |
| Q16557 | Pregnancy-specific beta-1-glycoprotein 3 | PSG3 |
| Q16568 | CART(42-89) | CARTPT |
| Q16610 | Extracellular matrix protein 1 | ECM1 |
| Q16619 | Cardiotrophin-1 | CTF1 |
| Q16623 | Syntaxin-1A | STX1A |
| Q16627 | HCC-1(9-74) | CCL14 |
| Q16651 | Prostasin light chain | PRSS8 |
| Q16661 | Guanylate cyclase C-activating peptide 2 | GUCA2B |
| Q16663 | CCL15(29-92) | CCL15 |
| Q16674 | Melanoma-derived growth regulatory protein | MIA |
| Q16769 | Glutaminyl-peptide cyclotransferase | QPCT |
| Q16787 | Laminin subunit alpha-3 | LAMA3 |
| Q16842 | CMP-N-acetylneuraminate-beta-galactosamide- | ST3GAL2 |
| | alpha-2,3-sialyltransferase 2 | |
| Q17RR3 | Pancreatic lipase-related protein 3 | PNLIPRP3 |
| Q17RW2 | Collagen alpha-1(XXIV) chain | COL24A1 |
| Q17RY6 | Lymphocyte antigen 6K | LY6K |
| Q1L6U9 | Prostate-associated microseminoprotein | MSMP |
| Q1W4C9 | Serine protease inhibitor Kazal-type 13 | SPINK13 |
| Q1ZYL8 | Izumo sperm-egg fusion protein 4 | IZUMO4 |
| Q29960 | HLA class I histocompatibility antigen, Cw-16 | HLA-C |
| | alpha chain | |
| Q2I0M5 | R-spondin-4 | RSPO4 |
| Q2L4Q9 | Serine protease 53 | PRSS53 |
| Q2MKA7 | R-spondin-1 | RSPO1 |
| Q2MV58 | Tectonic-1 | TCTN1 |
| Q2TAL6 | Brorin | VWC2 |
| Q2UY09 | Collagen alpha-1(XXVIII) chain | COL28A1 |
| Q2VPA4 | Complement component receptor 1-like protein | CR1L |
| Q2WEN9 | Carcinoembryonic antigen-related cell adhesion | CEACAM16 |
| | molecule 16 | |
| Q30KP8 | Beta-defensin 136 | DEFB136 |
| Q30KP9 | Beta-defensin 135 | DEFB135 |
| Q30KQ1 | Beta-defensin 133 | DEFB133 |
| Q30KQ2 | Beta-defensin 130 | DEFB130 |
| Q30KQ4 | Beta-defensin 116 | DEFB116 |
| Q30KQ5 | Beta-defensin 115 | DEFB115 |
| Q30KQ6 | Beta-defensin 114 | DEFB114 |
| Q30KQ7 | Beta-defensin 113 | DEFB113 |
| Q30KQ8 | Beta-defensin 112 | DEFB112 |
| Q30KQ9 | Beta-defensin 110 | DEFB110 |
| Q30KR1 | Beta-defensin 109 | DEFB109P1 |
| Q32P28 | Prolyl 3-hydroxylase 1 | LEPRE1 |
| Q3B7J2 | Glucose-fructose oxidoreductase domain- | GFOD2 |
| | containing protein 2 | |
| Q3SY79 | Protein Wnt | WNT3A |
| Q3T906 | N-acetylglucosamine-1-phosphotransferase | GNPTAB |
| | subunits alpha/beta | |
| Q495T6 | Membrane metallo-endopeptidase-like 1 | MMEL1 |
| Q49AH0 | Cerebral dopamine neurotrophic factor | CDNF |
| Q4G0G5 | Secretoglobin family 2B member 2 | SCGB2B2 |
| Q4G0M1 | Protein FAM132B | FAM132B |
| Q4LDE5 | Sushi, von Willebrand factor type A, EGF and | SVEP1 |
| | pentraxin domain-containing protein 1 | |
| Q4QY38 | Beta-defensin 134 | DEFB134 |
| Q4VAJ4 | Protein Wnt | WNT10B |
| Q4W5P6 | Protein TMEM155 | TMEM155 |
| Q4ZHG4 | Fibronectin type III domain-containing protein 1 | FNDC1 |
| Q53H76 | Phospholipase A1 member A | PLA1A |
| Q53RD9 | Fibulin-7 | FBLN7 |
| Q53S33 | BolA-like protein 3 | BOLA3 |
| Q5BLP8 | Neuropeptide-like protein C4orf48 | C4orf48 |
| Q5DT21 | Serine protease inhibitor Kazal-type 9 | SPINK9 |
| Q5EBL8 | PDZ domain-containing protein 11 | PDZD11 |
| Q5FYB0 | Arylsulfatase J | ARSJ |
| Q5FYB1 | Arylsulfatase I | ARSI |
| Q5GAN3 | Ribonuclease-like protein 13 | RNASE13 |
| Q5GAN4 | Ribonuclease-like protein 12 | RNASE12 |
| Q5GAN6 | Ribonuclease-like protein 10 | RNASE10 |
| Q5GFL6 | von Willebrand factor A domain-containing | VWA2 |
| | protein 2 | |
| Q5H8A3 | Neuromedin-S | NMS |
| Q5H8C1 | FRAS1-related extracellular matrix protein 1 | FREM1 |
| Q5IJ48 | Protein crumbs homolog 2 | CRB2 |
| Q5J5C9 | Beta-defensin 121 | DEFB121 |
| Q5JS37 | NHL repeat-containing protein 3 | NHLRC3 |
| Q5JTB6 | Placenta-specific protein 9 | PLAC9 |
| Q5JU69 | Torsin-2A | TOR2A |
| Q5JXM2 | Methyltransferase-like protein 24 | METTL24 |
| Q5JZY3 | Ephrin type-A receptor 10 | EPHA10 |
| Q5K4E3 | Polyserase-2 | PRSS36 |
| Q5SRR4 | Lymphocyte antigen 6 complex locus protein G5c | LY6G5C |
| Q5T1H1 | Protein eyes shut homolog | EYS |
| Q5T4F7 | Secreted frizzled-related protein 5 | SFRP5 |
| Q5T4W7 | Artemin | ARTN |
| Q5T7M4 | Protein FAM132A | FAM132A |
| Q5TEH8 | Protein Wnt | WNT2B |
| Q5TIE3 | von Willebrand factor A domain-containing | VWA5B1 |
| | protein 5B1 | |
| Q5UCC4 | ER membrane protein complex subunit 10 | EMC10 |
| Q5VST6 | Abhydrolase domain-containing protein | FAM108B1 |
| | FAM108B1 | |
| Q5VTL7 | Fibronectin type III domain-containing protein 7 | FNDC7 |
| Q5VUM1 | UPF0369 protein C6orf57 | C6orf57 |
| Q5VV43 | Dyslexia-associated protein KIAA0319 | KIAA0319 |
| Q5VWW1 | Complement C1q-like protein 3 | C1QL3 |
| Q5VXI9 | Lipase member N | LIPN |
| Q5VXJ0 | Lipase member K | LIPK |
| Q5VXM1 | CUB domain-containing protein 2 | CDCP2 |
| Q5VYX0 | Renalase | RNLS |
| Q5VYY2 | Lipase member M | LIPM |
| Q5W186 | Cystatin-9 | CST9 |
| Q5W5W9 | Regulated endocrine-specific protein 18 | RESP18 |
| Q5XG92 | Carboxylesterase 4A | CES4A |
| Q63HQ2 | Pikachurin | EGFLAM |
| Q641Q3 | Meteorin-like protein | METRNL |
| Q66K79 | Carboxypeptidase Z | CPZ |
| Q685J3 | Mucin-17 | MUC17 |
| Q68BL7 | Olfactomedin-like protein 2A | OLFML2A |
| Q68BL8 | Olfactomedin-like protein 2B | OLFML2B |
| Q68DV7 | E3 ubiquitin-protein ligase RNF43 | RNF43 |
| Q6B9Z1 | Insulin growth factor-like family member 4 | IGFL4 |
| Q6BAA4 | Fc receptor-like B | FCRLB |
| Q6E0U4 | Dermokine | DMKN |
| Q6EMK4 | Vasorin | VASN |
| Q6FHJ7 | Secreted frizzled-related protein 4 | SFRP4 |
| Q6GPI1 | Chymotrypsin B2 chain B | CTRB2 |
| Q6GTS8 | Probable carboxypeptidase PM20D1 | PM20D1 |
| Q6H9L7 | Isthmin-2 | ISM2 |
| Q6IE36 | Ovostatin homolog 2 | OVOS2 |
| Q6IE37 | Ovostatin homolog 1 | OVOS1 |
| Q6IE38 | Serine protease inhibitor Kazal-type 14 | SPINK14 |
| Q6ISS4 | Leukocyte-associated immunoglobulin-like | LAIR2 |
| | receptor 2 | |
| Q6JVE5 | Epididymal-specific lipocalin-12 | LCN12 |
| Q6JVE6 | Epididymal-specific lipocalin-10 | LCN10 |
| Q6JVE9 | Epididymal-specific lipocalin-8 | LCN8 |
| Q6KF10 | Growth/differentiation factor 6 | GDF6 |
| Q6MZW2 | Follistatin-related protein 4 | FSTL4 |
| Q6NSX1 | Coiled-coil domain-containing protein 70 | CCDC70 |
| Q6NT32 | Carboxylesterase 5A | CES5A |
| Q6NT52 | Choriogonadotropin subunit beta variant 2 | CGB2 |
| Q6NUI6 | Chondroadherin-like protein | CHADL |
| Q6NUJ1 | Saposin A-like | PSAPL1 |
| Q6P093 | Arylacetamide deacetylase-like 2 | AADACL2 |
| Q6P4A8 | Phospholipase B-like 1 | PLBD1 |
| Q6P5S2 | UPF0762 protein C6orf58 | C6orf58 |
| Q6P988 | Protein notum homolog | NOTUM |
| Q6PCB0 | von Willebrand factor A domain-containing | VWA1 |
| | protein 1 | |
| Q6PDA7 | Sperm-associated antigen 11A | SPAG11A |
| Q6PEW0 | Inactive serine protease 54 | PRSS54 |
| Q6PEZ8 | Podocan-like protein 1 | PODNL1 |
| Q6PKH6 | Dehydrogenase/reductase SDR family member 4- | DHRS4L2 |
| | like 2 | |
| Q6Q788 | Apolipoprotein A-V | APOA5 |
| Q6SPF0 | Atherin | SAMD1 |
| Q6UDR6 | Kunitz-type protease inhibitor 4 | SPINT4 |
| Q6URK8 | Testis, prostate and placenta-expressed protein | TEPP |
| Q6UW01 | Cerebellin-3 | CBLN3 |
| Q6UW10 | Surfactant-associated protein 2 | SFTA2 |
| Q6UW15 | Regenerating islet-derived protein 3-gamma | REG3G |
| Q6UW32 | Insulin growth factor-like family member 1 | IGFL1 |
| Q6UW78 | UPF0723 protein C11orf83 | C11orf83 |
| Q6UW88 | Epigen | EPGN |
| Q6UWE3 | Colipase-like protein 2 | CLPSL2 |
| Q6UWF7 | NXPE family member 4 | NXPE4 |
| Q6UWF9 | Protein FAM180A | FAM180A |
| Q6UWM5 | GLIPR1-like protein 1 | GLIPR1L1 |
| Q6UWN8 | Serine protease inhibitor Kazal-type 6 | SPINK6 |
| Q6UWP2 | Dehydrogenase/reductase SDR family member 11 | DHRS11 |
| Q6UWP8 | Suprabasin | SBSN |
| Q6UWQ5 | Lysozyme-like protein 1 | LYZL1 |
| Q6UWQ7 | Insulin growth factor-like family member 2 | IGFL2 |
| Q6UWR7 | Ectonucleotide pyrophosphatase/phosphodiesterase | ENPP6 |
| | family member 6 soluble form | |
| Q6UWT2 | Adropin | ENHO |
| Q6UWU2 | Beta-galactosidase-1-like protein | GLB1L |
| Q6UWW0 | Lipocalin-15 | LCN15 |
| Q6UWX4 | HHIP-like protein 2 | HHIPL2 |
| Q6UWY0 | Arylsulfatase K | ARSK |
| Q6UWY2 | Serine protease 57 | PRSS57 |
| Q6UWY5 | Olfactomedin-like protein 1 | OLFML1 |
| Q6UX06 | Olfactomedin-4 | OLFM4 |
| Q6UX07 | Dehydrogenase/reductase SDR family member 13 | DHRS13 |
| Q6UX39 | Amelotin | AMTN |
| Q6UX46 | Protein FAM150B | FAM150B |
| Q6UX73 | UPF0764 protein C16orf89 | C16orf89 |
| Q6UXB0 | Protein FAM131A | FAM131A |
| Q6UXB1 | Insulin growth factor-like family member 3 | IGFL3 |
| Q6UXB2 | VEGF co-regulated chemokine 1 | CXCL17 |
| Q6UXF7 | C-type lectin domain family 18 member B | CLEC18B |
| Q6UXH0 | Hepatocellular carcinoma-associated protein TD26 | C19orf80 |
| Q6UXH1 | Cysteine-rich with EGF-like domain protein 2 | CRELD2 |
| Q6UXH8 | Collagen and calcium-binding EGF domain- | CCBE1 |
| | containing protein 1 | |
| Q6UXH9 | Inactive serine protease PAMR1 | PAMR1 |
| Q6UXI7 | Vitrin | VIT |
| Q6UXI9 | Nephronectin | NPNT |
| Q6UXN2 | Trem-like transcript 4 protein | TREML4 |
| Q6UXS0 | C-type lectin domain family 19 member A | CLEC19A |
| Q6UXT8 | Protein FAM150A | FAM150A |
| Q6UXT9 | Abhydrolase domain-containing protein 15 | ABHD15 |
| Q6UXV4 | Apolipoprotein O-like | APOOL |
| Q6UXX5 | Inter-alpha-trypsin inhibitor heavy chain H6 | ITIH6 |
| Q6UXX9 | R-spondin-2 | RSPO2 |
| Q6UY14 | ADAMTS-like protein 4 | ADAMTSL4 |
| Q6UY27 | Prostate and testis expressed protein 2 | PATE2 |
| Q6W4X9 | Mucin-6 | MUC6 |
| Q6WN34 | Chordin-like protein 2 | CHRDL2 |
| Q6WRI0 | Immunoglobulin superfamily member 10 | IGSF10 |
| Q6X4U4 | Sclerostin domain-containing protein 1 | SOSTDC1 |
| Q6X784 | Zona pellucida-binding protein 2 | ZPBP2 |
| Q6XE38 | Secretoglobin family 1D member 4 | SCGB1D4 |
| Q6XPR3 | Repetin | RPTN |
| Q6XZB0 | Lipase member I | LIPI |
| Q6ZMM2 | ADAMTS-like protein 5 | ADAMTSL5 |
| Q6ZMP0 | Thrombospondin type-1 domain-containing | THSD4 |
| | protein 4 | |
| Q6ZNF0 | Iron/zinc purple acid phosphatase-like protein | PAPL |
| Q6ZRI0 | Otogelin | OTOG |
| Q6ZRP7 | Sulfhydryl oxidase 2 | QSOX2 |
| Q6ZWJ8 | Kielin/chordin-like protein | KCP |
| Q75N90 | Fibrillin-3 | FBN3 |
| Q765I0 | Urotensin-2B | UTS2D |
| Q76B58 | Protein FAM5C | FAM5C |
| Q76LX8 | A disintegrin and metalloproteinase with | ADAMTS13 |
| | thrombospondin motifs 13 | |
| Q76M96 | Coiled-coil domain-containing protein 80 | CCDC80 |
| Q7L1S5 | Carbohydrate sulfotransferase 9 | CHST9 |
| Q7L513 | Fc receptor-like A | FCRLA |
| Q7L8A9 | Vasohibin-1 | VASH1 |
| Q7RTM1 | Otopetrin-1 | OTOP1 |
| Q7RTW8 | Otoancorin | OTOA |
| Q7RTY5 | Serine protease 48 | PRSS48 |
| Q7RTY7 | Ovochymase-1 | OVCH1 |
| Q7RTZ1 | Ovochymase-2 | OVCH2 |
| Q7Z304 | MAM domain-containing protein 2 | MAMDC2 |
| Q7Z3S9 | Notch homolog 2 N-terminal-like protein | NOTCH2NL |
| Q7Z4H4 | Intermedin-short | ADM2 |
| Q7Z4P5 | Growth/differentiation factor 7 | GDF7 |
| Q7Z4R8 | UPF0669 protein C6orf120 | C6orf120 |
| Q7Z4W2 | Lysozyme-like protein 2 | LYZL2 |
| Q7Z5A4 | Serine protease 42 | PRSS42 |
| Q7Z5A7 | Protein FAM19A5 | FAM19A5 |
| Q7Z5A8 | Protein FAM19A3 | FAM19A3 |
| Q7Z5A9 | Protein FAM19A1 | FAM19A1 |
| Q7Z5J1 | Hydroxysteroid 11-beta-dehydrogenase 1-like | HSD11B1L |
| | protein | |
| Q7Z5L0 | Vitelline membrane outer layer protein 1 homolog | VMO1 |
| Q7Z5L3 | Complement C1q-like protein 2 | C1QL2 |
| Q7Z5L7 | Podocan | PODN |
| Q7Z5P4 | 17-beta-hydroxysteroid dehydrogenase 13 | HSD17B13 |
| Q7Z5P9 | Mucin-19 | MUC19 |
| Q7Z5Y6 | Bone morphogenetic protein 8A | BMP8A |
| Q7Z7B7 | Beta-defensin 132 | DEFB132 |
| Q7Z7B8 | Beta-defensin 128 | DEFB128 |
| Q7Z7C8 | Transcription initiation factor TFIID subunit 8 | TAF8 |
| Q7Z7H5 | Transmembrane emp24 domain-containing protein 4 | TMED4 |
| Q86SG7 | Lysozyme g-like protein 2 | LYG2 |
| Q86SI9 | Protein CEI | C5orf38 |
| Q86TE4 | Leucine zipper protein 2 | LUZP2 |
| Q86TH1 | ADAMTS-like protein 2 | ADAMTSL2 |
| Q86U17 | Serpin A11 | SERPINA11 |
| Q86UU9 | Endokinin-A | TAC4 |
| Q86UW8 | Hyaluronan and proteoglycan link protein 4 | HAPLN4 |
| Q86UX2 | Inter-alpha-trypsin inhibitor heavy chain H5 | ITIH5 |
| Q86V24 | Adiponectin receptor protein 2 | ADIPOR2 |
| Q86VB7 | Soluble CD163 | CD163 |
| Q86VR8 | Four-jointed box protein 1 | FJX1 |
| Q86WD7 | Serpin A9 | SERPINA9 |
| Q86WN2 | Interferon epsilon | IFNE |
| Q86WS3 | Placenta-specific 1-like protein | PLAC1L |
| Q86X52 | Chondroitin sulfate synthase 1 | CHSY1 |
| Q86XP6 | Gastrokine-2 | GKN2 |
| Q86XS5 | Angiopoietin-related protein 5 | ANGPTL5 |
| Q86Y27 | B melanoma antigen 5 | BAGE5 |
| Q86Y28 | B melanoma antigen 4 | BAGE4 |
| Q86Y29 | B melanoma antigen 3 | BAGE3 |
| Q86Y30 | B melanoma antigen 2 | BAGE2 |
| Q86Y38 | Xylosyltransferase 1 | XYLT1 |
| Q86Y78 | Ly6/PLAUR domain-containing protein 6 | LYPD6 |
| Q86YD3 | Transmembrane protein 25 | TMEM25 |
| Q86YJ6 | Threonine synthase-like 2 | THNSL2 |
| Q86YW7 | Glycoprotein hormone beta-5 | GPHB5 |
| Q86Z23 | Complement C1q-like protein 4 | C1QL4 |
| Q8IU57 | Interleukin-28 receptor subunit alpha | IL28RA |
| Q8IUA0 | WAP four-disulfide core domain protein 8 | WFDC8 |
| Q8IUB2 | WAP four-disulfide core domain protein 3 | WFDC3 |
| Q8IUB3 | Protein WFDC10B | WFDC10B |
| Q8IUB5 | WAP four-disulfide core domain protein 13 | WFDC13 |
| Q8IUH2 | Protein CREG2 | CREG2 |
| Q8IUK5 | Plexin domain-containing protein 1 | PLXDC1 |
| Q8IUL8 | Cartilage intermediate layer protein 2 C2 | CILP2 |
| Q8IUX7 | Adipocyte enhancer-binding protein 1 | AEBP1 |
| Q8IUX8 | Epidermal growth factor-like protein 6 | EGFL6 |
| Q8IVL8 | Carboxypeptidase O | CPO |
| Q8IVN8 | Somatomedin-B and thrombospondin type-1 | SBSPON |
| | domain-containing protein | |
| Q8IVW8 | Protein spinster homolog 2 | SPNS2 |
| Q8IW75 | Serpin A12 | SERPINA12 |
| Q8IW92 | Beta-galactosidase-1-like protein 2 | GLB1L2 |
| Q8IWL1 | Pulmonary surfactant-associated protein A2 | SFTPA2 |
| Q8IWL2 | Pulmonary surfactant-associated protein A1 | SFTPA1 |
| Q8IWV2 | Contactin-4 | CNTN4 |
| Q8IWY4 | Signal peptide, CUB and EGF-like domain- | SCUBE1 |
| | containing protein 1 | |
| Q8IX30 | Signal peptide, CUB and EGF-like domain- | SCUBE3 |
| | containing protein 3 | |
| Q8IXA5 | Sperm acrosome membrane-associated protein 3, | SPACA3 |
| | membrane form | |
| Q8IXB1 | DnaJ homolog subfamily C member 10 | DNAJC10 |
| Q8IXL6 | Extracellular serine/threonine protein kinase | FAM20C |
| | Fam20C | |
| Q8IYD9 | Lung adenoma susceptibility protein 2 | LAS2 |
| Q8IYP2 | Serine protease 58 | PRSS58 |
| Q8IYS5 | Osteoclast-associated immunoglobulin-like | OSCAR |
| | receptor | |
| Q8IZC6 | Collagen alpha-1(XXVII) chain | COL27A1 |
| Q8IZJ3 | C3 and PZP-like alpha-2-macroglobulin domain- | CPAMD8 |
| | containing protein 8 | |
| Q8IZN7 | Beta-defensin 107 | DEFB107B |
| Q8N0V4 | Leucine-rich repeat LGI family member 2 | LGI2 |
| Q8N104 | Beta-defensin 106 | DEFB106B |
| Q8N119 | Matrix metalloproteinase-21 | MMP21 |
| Q8N129 | Protein canopy homolog 4 | CNPY4 |
| Q8N135 | Leucine-rich repeat LGI family member 4 | LGI4 |
| Q8N145 | Leucine-rich repeat LGI family member 3 | LGI3 |
| Q8N158 | Glypican-2 | GPC2 |
| Q8N1E2 | Lysozyme g-like protein 1 | LYG1 |
| Q8N2E2 | von Willebrand factor D and EGF domain- | VWDE |
| | containing protein | |
| Q8N2E6 | Prosalusin | TOR2A |
| Q8N2S1 | Latent-transforming growth factor beta-binding | LTBP4 |
| | protein 4 | |
| Q8N302 | Angiogenic factor with G patch and FHA domains 1 | AGGF1 |
| Q8N307 | Mucin-20 | MUC20 |
| Q8N323 | NXPE family member 1 | NXPE1 |
| Q8N387 | Mucin-15 | MUC15 |
| Q8N3Z0 | Inactive serine protease 35 | PRSS35 |
| Q8N436 | Inactive carboxypeptidase-like protein X2 | CPXM2 |
| Q8N474 | Secreted frizzled-related protein 1 | SFRP1 |
| Q8N475 | Follistatin-related protein 5 | FSTL5 |
| Q8N4F0 | BPI fold-containing family B member 2 | BPIFB2 |
| Q8N4T0 | Carboxypeptidase A6 | CPA6 |
| Q8N5W8 | Protein FAM24B | FAM24B |
| Q8N687 | Beta-defensin 125 | DEFB125 |
| Q8N688 | Beta-defensin 123 | DEFB123 |
| Q8N690 | Beta-defensin 119 | DEFB119 |
| Q8N6C5 | Immunoglobulin superfamily member 1 | IGSF1 |
| Q8N6C8 | Leukocyte immunoglobulin-like receptor | LILRA3 |
| | subfamily A member 3 | |
| Q8N6G6 | ADAMTS-like protein 1 | ADAMTSL1 |
| Q8N6Y2 | Leucine-rich repeat-containing protein 17 | LRRC17 |
| Q8N729 | Neuropeptide W-23 | NPW |
| Q8N8U9 | BMP-binding endothelial regulator protein | BMPER |
| Q8N907 | DAN domain family member 5 | DAND5 |
| Q8NAT1 | Glycosyltransferase-like domain-containing | GTDC2 |
| | protein 2 | |
| Q8NAU1 | Fibronectin type III domain-containing protein 5 | FNDC5 |
| Q8NB37 | Parkinson disease 7 domain-containing protein 1 | PDDC1 |
| Q8NBI3 | Draxin | DRAXIN |
| Q8NBM8 | Prenylcysteine oxidase-like | PCYOX1L |
| Q8NBP7 | Proprotein convertase subtilisin/kexin type 9 | PCSK9 |
| Q8NBQ5 | Estradiol 17-beta-dehydrogenase 11 | HSD17B11 |
| Q8NBV8 | Synaptotagmin-8 | SYT8 |
| Q8NCC3 | Group XV phospholipase A2 | PLA2G15 |
| Q8NCF0 | C-type lectin domain family 18 member C | CLEC18C |
| Q8NCW5 | NAD(P)H-hydrate epimerase | APOA1BP |
| Q8NDA2 | Hemicentin-2 | HMCN2 |
| Q8NDX9 | Lymphocyte antigen 6 complex locus protein G5b | LY6G5B |
| Q8NDZ4 | Deleted in autism protein 1 | C3orf58 |
| Q8NEB7 | Acrosin-binding protein | ACRBP |
| Q8NES8 | Beta-defensin 124 | DEFB124 |
| Q8NET1 | Beta-defensin 108B | DEFB108B |
| Q8NEX5 | Protein WFDC9 | WFDC9 |
| Q8NEX6 | Protein WFDC11 | WFDC11 |
| Q8NF86 | Serine protease 33 | PRSS33 |
| Q8NFM7 | Interleukin-17 receptor D | IL17RD |
| Q8NFQ5 | BPI fold-containing family B member 6 | BPIFB6 |
| Q8NFQ6 | BPI fold-containing family C protein | BPIFC |
| Q8NFU4 | Follicular dendritic cell secreted peptide | FDCSP |
| Q8NFW1 | Collagen alpha-1(XXII) chain | COL22A1 |
| Q8NG35 | Beta-defensin 105 | DEFB105B |
| Q8NG41 | Neuropeptide B-23 | NPB |
| Q8NHW6 | Otospiralin | OTOS |
| Q8NI99 | Angiopoietin-related protein 6 | ANGPTL6 |
| Q8TAA1 | Probable ribonuclease 11 | RNASE11 |
| Q8TAG5 | V-set and transmembrane domain-containing | VSTM2A |
| | protein 2A | |
| Q8TAL6 | Fin bud initiation factor homolog | FIBIN |
| Q8TAT2 | Fibroblast growth factor-binding protein 3 | FGFBP3 |
| Q8TAX7 | Mucin-7 | MUC7 |
| Q8TB22 | Spermatogenesis-associated protein 20 | SPATA20 |
| Q8TB73 | Protein NDNF | NDNF |
| Q8TB96 | T-cell immunomodulatory protein | ITFG1 |
| Q8TC92 | Protein disulfide-thiol oxidoreductase | ENOX1 |
| Q8TCV5 | WAP four-disulfide core domain protein 5 | WFDC5 |
| Q8TD06 | Anterior gradient protein 3 homolog | AGR3 |
| Q8TD33 | Secretoglobin family 1C member 1 | SCGB1C1 |
| Q8TD46 | Cell surface glycoprotein CD200 receptor 1 | CD200R1 |
| Q8TDE3 | Ribonuclease 8 | RNASE8 |
| Q8TDF5 | Neuropilin and tolloid-like protein 1 | NETO1 |
| Q8TDL5 | BPI fold-containing family B member 1 | BPIFB1 |
| Q8TE56 | A disintegrin and metalloproteinase with | ADAMTS17 |
| | thrombospondin motifs 17 | |
| Q8TE57 | A disintegrin and metalloproteinase with | ADAMTS16 |
| | thrombospondin motifs 16 | |
| Q8TE58 | A disintegrin and metalloproteinase with | ADAMTS15 |
| | thrombospondin motifs 15 | |
| Q8TE59 | A disintegrin and metalloproteinase with | ADAMTS19 |
| | thrombospondin motifs 19 | |
| Q8TE60 | A disintegrin and metalloproteinase with | ADAMTS18 |
| | thrombospondin motifs 18 | |
| Q8TE99 | Acid phosphatase-like protein 2 | ACPL2 |
| Q8TER0 | Sushi, nidogen and EGF-like domain-containing | SNED1 |
| | protein 1 | |
| Q8TEU8 | WAP, kazal, immunoglobulin, kunitz and NTR | WFIKKN2 |
| | domain-containing protein 2 | |
| Q8WTQ1 | Beta-defensin 104 | DEFB104B |
| Q8WTR8 | Netrin-5 | NTN5 |
| Q8WTU2 | Scavenger receptor cysteine-rich domain- | SRCRB4D |
| | containing group B protein | |
| Q8WU66 | Protein TSPEAR | TSPEAR |
| Q8WUA8 | Tsukushin | TSKU |
| Q8WUF8 | Protein FAM172A | FAM172A |
| Q8WUJ1 | Neuferricin | CYB5D2 |
| Q8WUY1 | UPF0670 protein THEM6 | THEM6 |
| Q8WVN6 | Secreted and transmembrane protein 1 | SECTM1 |
| Q8WVQ1 | Soluble calcium-activated nucleotidase 1 | CANT1 |
| Q8WWA0 | Intelectin-1 | ITLN1 |
| Q8WWG1 | Neuregulin-4 | NRG4 |
| Q8WWQ2 | Inactive heparanase-2 | HPSE2 |
| Q8WWU7 | Intelectin-2 | ITLN2 |
| Q8WWY7 | WAP four-disulfide core domain protein 12 | WFDC12 |
| Q8WWY8 | Lipase member H | LIPH |
| Q8WWZ8 | Oncoprotein-induced transcript 3 protein | OIT3 |
| Q8WX39 | Epididymal-specific lipocalin-9 | LCN9 |
| Q8WXA2 | Prostate and testis expressed protein 1 | PATE1 |
| Q8WXD2 | Secretogranin-3 | SCG3 |
| Q8WXF3 | Relaxin-3 A chain | RLN3 |
| Q8WXI7 | Mucin-16 | MUC16 |
| Q8WXQ8 | Carboxypeptidase A5 | CPA5 |
| Q8WXS8 | A disintegrin and metalloproteinase with | ADAMTS14 |
| | thrombospondin motifs 14 | |
| Q92484 | Acid sphingomyelinase-like phosphodiesterase 3a | SMPDL3A |
| Q92485 | Acid sphingomyelinase-like phosphodiesterase 3b | SMPDL3B |
| Q92496 | Complement factor H-related protein 4 | CFHR4 |
| Q92520 | Protein FAM3C | FAM3C |
| Q92563 | Testican-2 | SPOCK2 |
| Q92583 | C-C motif chemokine 17 | CCL17 |
| Q92626 | Peroxidasin homolog | PXDN |
| Q92743 | Serine protease HTRA1 | HTRA1 |
| Q92752 | Tenascin-R | TNR |
| Q92765 | Secreted frizzled-related protein 3 | FRZB |
| Q92819 | Hyaluronan synthase 2 | HAS2 |
| Q92820 | Gamma-glutamyl hydrolase | GGH |
| Q92824 | Proprotein convertase subtilisin/kexin type 5 | PCSK5 |
| Q92832 | Protein kinase C-binding protein NELL1 | NELL1 |
| Q92838 | Ectodysplasin-A, membrane form | EDA |
| Q92874 | Deoxyribonuclease-1-like 2 | DNASE1L2 |
| Q92876 | Kallikrein-6 | KLK6 |
| Q92913 | Fibroblast growth factor 13 | FGF13 |
| Q92954 | Proteoglycan 4 C-terminal part | PRG4 |
| Q93038 | Tumor necrosis factor receptor superfamily | TNFRSF25 |
| | member 25 | |
| Q93091 | Ribonuclease K6 | RNASE6 |
| Q93097 | Protein Wnt-2b | WNT2B |
| Q93098 | Protein Wnt-8b | WNT8B |
| Q95460 | Major histocompatibility complex class I-related | MR1 |
| | gene protein | |
| Q969D9 | Thymic stromal lymphopoietin | TSLP |
| Q969E1 | Liver-expressed antimicrobial peptide 2 | LEAP2 |
| Q969H8 | UPF0556 protein C19orf10 | C19orf10 |
| Q969Y0 | NXPE family member 3 | NXPE3 |
| Q96A54 | Adiponectin receptor protein 1 | ADIPOR1 |
| Q96A83 | Collagen alpha-1(XXVI) chain | EMID2 |
| Q96A84 | EMI domain-containing protein 1 | EMID1 |
| Q96A98 | Tuberoinfundibular peptide of 39 residues | PTH2 |
| Q96A99 | Pentraxin-4 | PTX4 |
| Q96BH3 | Epididymal sperm-binding protein 1 | ELSPBP1 |
| Q96BQ1 | Protein FAM3D | FAM3D |
| Q96CG8 | Collagen triple helix repeat-containing protein 1 | CTHRC1 |
| Q96DA0 | Zymogen granule protein 16 homolog B | ZG16B |
| Q96DN2 | von Willebrand factor C and EGF domain- | VWCE |
| | containing protein | |
| Q96DR5 | BPI fold-containing family A member 2 | BPIFA2 |
| Q96DR8 | Mucin-like protein 1 | MUCL1 |
| Q96DX4 | RING finger and SPRY domain-containing protein 1 | RSPRY1 |
| Q96EE4 | Coiled-coil domain-containing protein 126 | CCDC126 |
| Q96GS6 | Abhydrolase domain-containing protein | FAM108A1 |
| | FAM108A1 | |
| Q96GW7 | Brevican core protein | BCAN |
| Q96HF1 | Secreted frizzled-related protein 2 | SFRP2 |
| Q96I82 | Kazal-type serine protease inhibitor domain- | KAZALD1 |
| | containing protein 1 | |
| Q96ID5 | Immunoglobulin superfamily member 21 | IGSF21 |
| Q96II8 | Leucine-rich repeat and calponin homology | LRCH3 |
| | domain-containing protein 3 | |
| Q96IY4 | Carboxypeptidase B2 | CPB2 |
| Q96JB6 | Lysyl oxidase homolog 4 | LOXL4 |
| Q96JK4 | HHIP-like protein 1 | HHIPL1 |
| Q96KN2 | Beta-Ala-His dipeptidase | CNDP1 |
| Q96KW9 | Protein SPACA7 | SPACA7 |
| Q96KX0 | Lysozyme-like protein 4 | LYZL4 |
| Q96L15 | Ecto-ADP-ribosyltransferase 5 | ART5 |
| Q96LB8 | Peptidoglycan recognition protein 4 | PGLYRP4 |
| Q96LB9 | Peptidoglycan recognition protein 3 | PGLYRP3 |
| Q96LC7 | Sialic acid-binding Ig-like lectin 10 | SIGLEC10 |
| Q96LR4 | Protein FAM19A4 | FAM19A4 |
| Q96MK3 | Protein FAM20A | FAM20A |
| Q96MS3 | Glycosyltransferase 1 domain-containing protein 1 | GLT1D1 |
| Q96NY8 | Processed poliovirus receptor-related protein 4 | PVRL4 |
| Q96NZ8 | WAP, kazal, immunoglobulin, kunitz and NTR | WFIKKN1 |
| | domain-containing protein 1 | |
| Q96NZ9 | Proline-rich acidic protein 1 | PRAP1 |
| Q96P44 | Collagen alpha-1(XXI) chain | COL21A1 |
| Q96PB7 | Noelin-3 | OLFM3 |
| Q96PC5 | Melanoma inhibitory activity protein 2 | MIA2 |
| Q96PD5 | N-acetylmuramoyl-L-alanine amidase | PGLYRP2 |
| Q96PH6 | Beta-defensin 118 | DEFB118 |
| Q96PL1 | Secretoglobin family 3A member 2 | SCGB3A2 |
| Q96PL2 | Beta-tectorin | TECTB |
| Q96QH8 | Sperm acrosome-associated protein 5 | SPACA5 |
| Q96QR1 | Secretoglobin family 3A member 1 | SCGB3A1 |
| Q96QU1 | Protocadherin-15 | PCDH15 |
| Q96QV1 | Hedgehog-interacting protein | HHIP |
| Q96RW7 | Hemicentin-1 | HMCN1 |
| Q96S42 | Nodal homolog | NODAL |
| Q96S86 | Hyaluronan and proteoglycan link protein 3 | HAPLN3 |
| Q96SL4 | Glutathione peroxidase 7 | GPX7 |
| Q96SM3 | Probable carboxypeptidase X1 | CPXM1 |
| Q96T91 | Glycoprotein hormone alpha-2 | GPHA2 |
| Q99062 | Granulocyte colony-stimulating factor receptor | CSF3R |
| Q99102 | Mucin-4 alpha chain | MUC4 |
| Q99217 | Amelogenin, X isoform | AMELX |
| Q99218 | Amelogenin, Y isoform | AMELY |
| Q99435 | Protein kinase C-binding protein NELL2 | NELL2 |
| Q99470 | Stromal cell-derived factor 2 | SDF2 |
| Q99542 | Matrix metalloproteinase-19 | MMP19 |
| Q99574 | Neuroserpin | SERPINI1 |
| Q99584 | Protein S100-A13 | S100A13 |
| Q99616 | C-C motif chemokine 13 | CCL13 |
| Q99645 | Epiphycan | EPYC |
| Q99674 | Cell growth regulator with EF hand domain | CGREF1 |
| | protein 1 | |
| Q99715 | Collagen alpha-1(XII) chain | COL12A1 |
| Q99727 | Metalloproteinase inhibitor 4 | TIMP4 |
| Q99731 | C-C motif chemokine 19 | CCL19 |
| Q99748 | Neurturin | NRTN |
| Q99935 | Proline-rich protein 1 | PROL1 |
| Q99942 | E3 ubiquitin-protein ligase RNF5 | RNF5 |
| Q99944 | Epidermal growth factor-like protein 8 | EGFL8 |
| Q99954 | Submaxillary gland androgen-regulated protein 3A | SMR3A |
| Q99969 | Retinoic acid receptor responder protein 2 | RARRES2 |
| Q99972 | Myocilin | MYOC |
| Q99983 | Osteomodulin | OMD |
| Q99985 | Semaphorin-3C | SEMA3C |
| Q99988 | Growth/differentiation factor 15 | GDF15 |
| Q9BPW4 | Apolipoprotein L4 | APOL4 |
| Q9BQ08 | Resistin-like beta | RETNLB |
| Q9BQ16 | Testican-3 | SPOCK3 |
| Q9BQ51 | Programmed cell death 1 ligand 2 | PDCD1LG2 |
| Q9BQB4 | Sclerostin | SOST |
| Q9BQI4 | Coiled-coil domain-containing protein 3 | CCDC3 |
| Q9BQP9 | BPI fold-containing family A member 3 | BPIFA3 |
| Q9BQR3 | Serine protease 27 | PRSS27 |
| Q9BQY6 | WAP four-disulfide core domain protein 6 | WFDC6 |
| Q9BRR6 | ADP-dependent glucokinase | ADPGK |
| Q9BS86 | Zona pellucida-binding protein 1 | ZPBP |
| Q9BSG0 | Protease-associated domain-containing protein 1 | PRADC1 |
| Q9BSG5 | Retbindin | RTBDN |
| Q9BT30 | Probable alpha-ketoglutarate-dependent | ALKBH7 |
| | dioxygenase ABH7 | |
| Q9BT56 | Spexin | C12orf39 |
| Q9BT67 | NEDD4 family-interacting protein 1 | NDFIP1 |
| Q9BTY2 | Plasma alpha-L-fucosidase | FUCA2 |
| Q9BU40 | Chordin-like protein 1 | CHRDL1 |
| Q9BUD6 | Spondin-2 | SPON2 |
| Q9BUN1 | Protein MENT | MENT |
| Q9BUR5 | Apolipoprotein O | APOO |
| Q9BV94 | ER degradation-enhancing alpha-mannosidase-like 2 | EDEM2 |
| Q9BWP8 | Collectin-11 | COLEC11 |
| Q9BWS9 | Chitinase domain-containing protein 1 | CHID1 |
| Q9BX67 | Junctional adhesion molecule C | JAM3 |
| Q9BX93 | Group XIIB secretory phospholipase A2-like | PLA2G12B |
| | protein | |
| Q9BXI9 | Complement C1q tumor necrosis factor-related | C1QTNF6 |
| | protein 6 | |
| Q9BXJ0 | Complement C1q tumor necrosis factor-related | C1QTNF5 |
| | protein 5 | |
| Q9BXJ1 | Complement C1q tumor necrosis factor-related | C1QTNF1 |
| | protein 1 | |
| Q9BXJ2 | Complement C1q tumor necrosis factor-related | C1QTNF7 |
| | protein 7 | |
| Q9BXJ3 | Complement C1q tumor necrosis factor-related | C1QTNF4 |
| | protein 4 | |
| Q9BXJ4 | Complement C1q tumor necrosis factor-related | C1QTNF3 |
| | protein 3 | |
| Q9BXJ5 | Complement C1q tumor necrosis factor-related | C1QTNF2 |
| | protein 2 | |
| Q9BXN1 | Asporin | ASPN |
| Q9BXP8 | Pappalysin-2 | PAPPA2 |
| Q9BXR6 | Complement factor H-related protein 5 | CFHR5 |
| Q9BXS0 | Collagen alpha-1(XXV) chain | COL25A1 |
| Q9BXX0 | EMILIN-2 | EMILIN2 |
| Q9BXY4 | R-spondin-3 | RSPO3 |
| Q9BY15 | EGF-like module-containing mucin-like hormone | EMR3 |
| | receptor-like 3 subunit beta | |
| Q9BY50 | Signal peptidase complex catalytic subunit | SEC11C |
| | SEC11C | |
| Q9BY76 | Angiopoietin-related protein 4 | ANGPTL4 |
| Q9BYF1 | Processed angiotensin-converting enzyme 2 | ACE2 |
| Q9BYJ0 | Fibroblast growth factor-binding protein 2 | FGFBP2 |
| Q9BYW3 | Beta-defensin 126 | DEFB126 |
| Q9BYX4 | Interferon-induced helicase C domain-containing | IFIH1 |
| | protein 1 | |
| Q9BYZ8 | Regenerating islet-derived protein 4 | REG4 |
| Q9BZ76 | Contactin-associated protein-like 3 | CNTNAP3 |
| Q9BZG9 | Ly-6/neurotoxin-like protein 1 | LYNX1 |
| Q9BZJ3 | Tryptase delta | TPSD1 |
| Q9BZM1 | Group XIIA secretory phospholipase A2 | PLA2G12A |
| Q9BZM2 | Group IIF secretory phospholipase A2 | PLA2G2F |
| Q9BZM5 | NKG2D ligand 2 | ULBP2 |
| Q9BZP6 | Acidic mammalian chitinase | CHIA |
| Q9BZZ2 | Sialoadhesin | SIGLEC1 |
| Q9C0B6 | Protein FAM5B | FAM5B |
| Q9GZM7 | Tubulointerstitial nephritis antigen-like | TINAGL1 |
| Q9GZN4 | Brain-specific serine protease 4 | PRSS22 |
| Q9GZP0 | Platelet-derived growth factor D, receptor- | PDGFD |
| | binding form | |
| Q9GZT5 | Protein Wnt-10a | WNT10A |
| Q9GZU5 | Nyctalopin | NYX |
| Q9GZV7 | Hyaluronan and proteoglycan link protein 2 | HAPLN2 |
| Q9GZV9 | Fibroblast growth factor 23 | FGF23 |
| Q9GZX9 | Twisted gastrulation protein homolog 1 | TWSG1 |
| Q9GZZ7 | GDNF family receptor alpha-4 | GFRA4 |
| Q9GZZ8 | Extracellular glycoprotein lacritin | LACRT |
| Q9H0B8 | Cysteine-rich secretory protein LCCL domain- | CRISPLD2 |
| | containing 2 | |
| Q9H106 | Signal-regulatory protein delta | SIRPD |
| Q9H114 | Cystatin-like 1 | CSTL1 |
| Q9H173 | Nucleotide exchange factor SIL1 | SIL1 |
| Q9H1E1 | Ribonuclease 7 | RNASE7 |
| Q9H1F0 | WAP four-disulfide core domain protein 10A | WFDC10A |
| Q9H1J5 | Protein Wnt-8a | WNT8A |
| Q9H1J7 | Protein Wnt-5b | WNT5B |
| Q9H1M3 | Beta-defensin 129 | DEFB129 |
| Q9H1M4 | Beta-defensin 127 | DEFB127 |
| Q9H1Z8 | Augurin | C2orf40 |
| Q9H239 | Matrix metalloproteinase-28 | MMP28 |
| Q9H2A7 | C-X-C motif chemokine 16 | CXCL16 |
| Q9H2A9 | Carbohydrate sulfotransferase 8 | CHST8 |
| Q9H2R5 | Kallikrein-15 | KLK15 |
| Q9H2X0 | Chordin | CHRD |
| Q9H2X3 | C-type lectin domain family 4 member M | CLEC4M |
| Q9H306 | Matrix metalloproteinase-27 | MMP27 |
| Q9H324 | A disintegrin and metalloproteinase with | ADAMTS10 |
| | thrombospondin motifs 10 | |
| Q9H336 | Cysteine-rich secretory protein LCCL domain- | CRISPLD1 |
| | containing 1 | |
| Q9H3E2 | Sorting nexin-25 | SNX25 |
| Q9H3R2 | Mucin-13 | MUC13 |
| Q9H3U7 | SPARC-related modular calcium-binding protein 2 | SMOC2 |
| Q9H3Y0 | Peptidase inhibitor R3HDML | R3HDML |
| Q9H4A4 | Aminopeptidase B | RNPEP |
| Q9H4F8 | SPARC-related modular calcium-binding protein 1 | SMOC1 |
| Q9H4G1 | Cystatin-9-like | CST9L |
| Q9H5V8 | CUB domain-containing protein 1 | CDCP1 |
| Q9H6B9 | Epoxide hydrolase 3 | EPHX3 |
| Q9H6E4 | Coiled-coil domain-containing protein 134 | CCDC134 |
| Q9H741 | UPF0454 protein C12orf49 | C12orf49 |
| Q9H772 | Gremlin-2 | GREM2 |
| Q9H7Y0 | Deleted in autism-related protein 1 | CXorf36 |
| Q9H8L6 | Multimerin-2 | MMRN2 |
| Q9H9S5 | Fukutin-related protein | FKRP |
| Q9HAT2 | Sialate O-acetylesterase | SIAE |
| Q9HB40 | Retinoid-inducible serine carboxypeptidase | SCPEP1 |
| Q9HB63 | Netrin-4 | NTN4 |
| Q9HBJ0 | Placenta-specific protein 1 | PLAC1 |
| Q9HC23 | Prokineticin-2 | PROK2 |
| Q9HC57 | WAP four-disulfide core domain protein 1 | WFDC1 |
| Q9HC73 | Cytokine receptor-like factor 2 | CRLF2 |
| Q9HC84 | Mucin-5B | MUC5B |
| Q9HCB6 | Spondin-1 | SPON1 |
| Q9HCQ7 | Neuropeptide NPSF | NPVF |
| Q9HCT0 | Fibroblast growth factor 22 | FGF22 |
| Q9HD89 | Resistin | RETN |
| Q9NNX1 | Tuftelin | TUFT1 |
| Q9NNX6 | CD209 antigen | CD209 |
| Q9NP55 | BPI fold-containing family A member 1 | BPIFA1 |
| Q9NP70 | Ameloblastin | AMBN |
| Q9NP95 | Fibroblast growth factor 20 | FGF20 |
| Q9NP99 | Triggering receptor expressed on myeloid cells 1 | TREM1 |
| Q9NPA2 | Matrix metalloproteinase-25 | MMP25 |
| Q9NPE2 | Neugrin | NGRN |
| Q9NPH0 | Lysophosphatidic acid phosphatase type 6 | ACP6 |
| Q9NPH6 | Odorant-binding protein 2b | OBP2B |
| Q9NQ30 | Endothelial cell-specific molecule 1 | ESM1 |
| Q9NQ36 | Signal peptide, CUB and EGF-like domain- | SCUBE2 |
| | containing protein 2 | |
| Q9NQ38 | Serine protease inhibitor Kazal-type 5 | SPINK5 |
| Q9NQ76 | Matrix extracellular phosphoglycoprotein | MEPE |
| Q9NQ79 | Cartilage acidic protein 1 | CRTAC1 |
| Q9NR16 | Scavenger receptor cysteine-rich type 1 | CD163L1 |
| | protein M160 | |
| Q9NR23 | Growth/differentiation factor 3 | GDF3 |
| Q9NR71 | Neutral ceramidase | ASAH2 |
| Q9NR99 | Matrix-remodeling-associated protein 5 | MXRA5 |
| Q9NRA1 | Platelet-derived growth factor C | PDGFC |
| Q9NRC9 | Otoraplin | OTOR |
| Q9NRE1 | Matrix metalloproteinase-26 | MMP26 |
| Q9NRJ3 | C-C motif chemokine 28 | CCL28 |
| Q9NRM1 | Enamelin | ENAM |
| Q9NRN5 | Olfactomedin-like protein 3 | OLFML3 |
| Q9NRR1 | Cytokine-like protein 1 | CYTL1 |
| Q9NS15 | Latent-transforming growth factor beta-binding | LTBP3 |
| | protein 3 | |
| Q9NS62 | Thrombospondin type-1 domain-containing | THSD1 |
| | protein 1 | |
| Q9NS71 | Gastrokine-1 | GKN1 |
| Q9NS98 | Semaphorin-3G | SEMA3G |
| Q9NSA1 | Fibroblast growth factor 21 | FGF21 |
| Q9NT22 | EMILIN-3 | EMILIN3 |
| Q9NTU7 | Cerebellin-4 | CBLN4 |
| Q9NVR0 | Kelch-like protein 11 | KLHL11 |
| Q9NWH7 | Spermatogenesis-associated protein 6 | SPATA6 |
| Q9NXC2 | Glucose-fructose oxidoreductase domain- | GFOD1 |
| | containing protein 1 | |
| Q9NY56 | Odorant-binding protein 2a | OBP2A |
| Q9NY84 | Vascular non-inflammatory molecule 3 | VNN3 |
| Q9NZ20 | Group 3 secretory phospholipase A2 | PLA2G3 |
| Q9NZC2 | Triggering receptor expressed on myeloid cells 2 | TREM2 |
| Q9NZK5 | Adenosine deaminase CECR1 | CECR1 |
| Q9NZK7 | Group IIE secretory phospholipase A2 | PLA2G2E |
| Q9NZP8 | Complement C1r subcomponent-like protein | C1RL |
| Q9NZV1 | Cysteine-rich motor neuron 1 protein | CRIM1 |
| Q9NZW4 | Dentin sialoprotein | DSPP |
| Q9P0G3 | Kallikrein-14 | KLK14 |
| Q9P0W0 | Interferon kappa | IFNK |
| Q9P218 | Collagen alpha-1(XX) chain | COL20A1 |
| Q9P2C4 | Transmembrane protein 181 | TMEM181 |
| Q9P2K2 | Thioredoxin domain-containing protein 16 | TXNDC16 |
| Q9P2N4 | A disintegrin and metalloproteinase with | ADAMTS9 |
| | thrombospondin motifs 9 | |
| Q9UBC7 | Galanin-like peptide | GALP |
| Q9UBD3 | Cytokine SCM-1 beta | XCL2 |
| Q9UBD9 | Cardiotrophin-like cytokine factor 1 | CLCF1 |
| Q9UBM4 | Opticin | OPTC |
| Q9UBP4 | Dickkopf-related protein 3 | DKK3 |
| Q9UBQ6 | Exostosin-like 2 | EXTL2 |
| Q9UBR5 | Chemokine-like factor | CKLF |
| Q9UBS5 | Gamma-aminobutyric acid type B receptor subunit 1 | GABBR1 |
| Q9UBT3 | Dickkopf-related protein 4 short form | DKK4 |
| Q9UBU2 | Dickkopf-related protein 2 | DKK2 |
| Q9UBU3 | Ghrelin-28 | GHRL |
| Q9UBV4 | Protein Wnt-16 | WNT16 |
| Q9UBX5 | Fibulin-5 | FBLN5 |
| Q9UBX7 | Kallikrein-11 | KLK11 |
| Q9UEF7 | Klotho | KL |
| Q9UFP1 | Protein FAM198A | FAM198A |
| Q9UGM3 | Deleted in malignant brain tumors 1 protein | DMBT1 |
| Q9UGM5 | Fetuin-B | FETUB |
| Q9UGP8 | Translocation protein SEC63 homolog | SEC63 |
| Q9UHF0 | Neurokinin-B | TAC3 |
| Q9UHF1 | Epidermal growth factor-like protein 7 | EGFL7 |
| Q9UHG2 | ProSAAS | PCSK1N |
| Q9UHI8 | A disintegrin and metalloproteinase with | ADAMTS1 |
| | thrombospondin motifs 1 | |
| Q9UHL4 | Dipeptidyl peptidase 2 | DPP7 |
| Q9UI42 | Carboxypeptidase A4 | CPA4 |
| Q9UIG4 | Psoriasis susceptibility 1 candidate gene 2 protein | PSORS1C2 |
| Q9UIK5 | Tomoregulin-2 | TMEFF2 |
| Q9UIQ6 | Leucyl-cystinyl aminopeptidase, pregnancy serum | LNPEP |
| | form | |
| Q9UJA9 | Ectonucleotide pyrophosphatase/phosphodiesterase | ENPP5 |
| | family member 5 | |
| Q9UJH8 | Meteorin | METRN |
| Q9UJJ9 | N-acetylglucosamine-1-phosphotransferase | GNPTG |
| | subunit gamma | |
| Q9UJW2 | Tubulointerstitial nephritis antigen | TINAG |
| Q9UK05 | Growth/differentiation factor 2 | GDF2 |
| Q9UK55 | Protein Z-dependent protease inhibitor | SERPINA10 |
| Q9UK85 | Dickkopf-like protein 1 | DKKL1 |
| Q9UKJ1 | Paired immunoglobulin-like type 2 receptor alpha | PILRA |
| Q9UKP4 | A disintegrin and metalloproteinase with | ADAMTS7 |
| | thrombospondin motifs 7 | |
| Q9UKP5 | A disintegrin and metalloproteinase with | ADAMTS6 |
| | thrombospondin motifs 6 | |
| Q9UKQ2 | Disintegrin and metalloproteinase domain- | ADAM28 |
| | containing protein 28 | |
| Q9UKQ9 | Kallikrein-9 | KLK9 |
| Q9UKR0 | Kallikrein-12 | KLK12 |
| Q9UKR3 | Kallikrein-13 | KLK13 |
| Q9UKU9 | Angiopoietin-related protein 2 | ANGPTL2 |
| Q9UKZ9 | Procollagen C-endopeptidase enhancer 2 | PCOLCE2 |
| Q9UL52 | Transmembrane protease serine 11E non- | TMPRSS11E |
| | catalytic chain | |
| Q9ULC0 | Endomucin | EMCN |
| Q9ULI3 | Protein HEG homolog 1 | HEG1 |
| Q9ULZ1 | Apelin-13 | APLN |
| Q9ULZ9 | Matrix metalloproteinase-17 | MMP17 |
| Q9UM21 | Alpha-1,3-mannosyl-glycoprotein 4-beta-N- | MGAT4A |
| | acetylglucosaminyltransferase A soluble form | |
| Q9UM22 | Mammalian ependymin-related protein 1 | EPDR1 |
| Q9UM73 | ALK tyrosine kinase receptor | ALK |
| Q9UMD9 | 97 kDa linear IgA disease antigen | COL17A1 |
| Q9UMX5 | Neudesin | NENF |
| Q9UN73 | Protocadherin alpha-6 | PCDHA6 |
| Q9UNA0 | A disintegrin and metalloproteinase with | ADAMTS5 |
| | thrombospondin motifs 5 | |
| Q9UNI1 | Chymotrypsin-like elastase family member 1 | CELA1 |
| Q9UNK4 | Group IID secretory phospholipase A2 | PLA2G2D |
| Q9UP79 | A disintegrin and metalloproteinase with | ADAMTS8 |
| | thrombospondin motifs 8 | |
| Q9UPZ6 | Thrombospondin type-1 domain-containing | THSD7A |
| | protein 7A | |
| Q9UQ72 | Pregnancy-specific beta-1-glycoprotein 11 | PSG11 |
| Q9UQ74 | Pregnancy-specific beta-1-glycoprotein 8 | PSG8 |
| Q9UQC9 | Calcium-activated chloride channel regulator 2 | CLCA2 |
| Q9UQE7 | Structural maintenance of chromosomes protein 3 | SMC3 |
| Q9UQP3 | Tenascin-N | TNN |
| Q9Y223 | UDP-N-acetylglucosamine 2-epimerase | GNE |
| Q9Y240 | C-type lectin domain family 11 member A | CLEC11A |
| Q9Y251 | Heparanase 8 kDa subunit | HPSE |
| Q9Y258 | C-C motif chemokine 26 | CCL26 |
| Q9Y264 | Angiopoietin-4 | ANGPT4 |
| Q9Y275 | Tumor necrosis factor ligand superfamily member | TNFSF13B |
| | 13b, membrane form | |
| Q9Y287 | BRI2 intracellular domain | ITM2B |
| Q9Y2E5 | Epididymis-specific alpha-mannosidase | MAN2B2 |
| Q9Y334 | von Willebrand factor A domain-containing | VWA7 |
| | protein 7 | |
| Q9Y337 | Kallikrein-5 | KLK5 |
| Q9Y3B3 | Transmembrane emp24 domain-containing protein 7 | TMED7 |
| Q9Y3E2 | BolA-like protein 1 | BOLA1 |
| Q9Y426 | C2 domain-containing protein 2 | C2CD2 |
| Q9Y4K0 | Lysyl oxidase homolog 2 | LOXL2 |
| Q9Y4X3 | C-C motif chemokine 27 | CCL27 |
| Q9Y5C1 | Angiopoietin-related protein 3 | ANGPTL3 |
| Q9Y5I2 | Protocadherin alpha-10 | PCDHA10 |
| Q9Y5I3 | Protocadherin alpha-1 | PCDHA1 |
| Q9Y5K2 | Kallikrein-4 | KLK4 |
| Q9Y5L2 | Hypoxia-inducible lipid droplet-associated protein | HILPDA |
| Q9Y5Q5 | Atrial natriuretic peptide-converting enzyme | CORIN |
| Q9Y5R2 | Matrix metalloproteinase-24 | MMP24 |
| Q9Y5U5 | Tumor necrosis factor receptor superfamily | TNFRSF18 |
| | member 18 | |
| Q9Y5W5 | Wnt inhibitory factor 1 | WIF1 |
| Q9Y5X9 | Endothelial lipase | LIPG |
| Q9Y625 | Secreted glypican-6 | GPC6 |
| Q9Y646 | Carboxypeptidase Q | CPQ |
| Q9Y6C2 | EMILIN-1 | EMILIN1 |
| Q9Y6F9 | Protein Wnt-6 | WNT6 |
| Q9Y6I9 | Testis-expressed sequence 264 protein | TEX264 |
| Q9Y6L7 | Tolloid-like protein 2 | TLL2 |
| Q9Y6N3 | Calcium-activated chloride channel regulator | CLCA3P |
| | family member 3 | |
| Q9Y6N6 | Laminin subunit gamma-3 | LAMC3 |
| Q9Y6R7 | IgGFc-binding protein | FCGBP |
| Q9Y6Y9 | Lymphocyte antigen 96 | LY96 |
| Q9Y6Z7 | Collectin-10 | COLEC10 |

In some embodiments, the compositions and methods of the invention provide for the delivery of one or more mRNAs encoding one or more additional exemplary proteins listed in Table 2; thus, compositions of the invention may comprise an mRNA encoding a protein listed in Table 2 (or a homolog thereof) along with other components set out herein, and methods of the invention may comprise preparing and/or administering a composition comprising an mRNA encoding a protein chosen from the proteins listed in Table 2 (or a homolog thereof) along with other components set out herein.

TABLE 2
Additional Exemplary Proteins

| Uniprot ID | Protein Name | Gene Name |
|---|---|---|
| A6NGW2 | Putative stereocilin-like protein | STRCP1 |
| A6NIE9 | Putative serine protease 29 | PRSS29P |
| A6NJ16 | Putative V-set and immunoglobulin | IGHV4OR15-8 |
| | domain-containing-like protein | |
| | IGHV4OR15-8 | |
| A6NJS3 | Putative V-set and immunoglobulin | IGHV1OR21-1 |
| | domain-containing-like protein | |
| | IGHV1OR21-1 | |
| A6NMY6 | Putative annexin A2-like protein | ANXA2P2 |
| A8MT79 | Putative zinc-alpha-2-glycoprotein-like 1 | |
| A8MWS1 | Putative killer cell immunoglobulin-like | KIR3DP1 |
| | receptor like protein KIR3DP1 | |
| A8MXU0 | Putative beta-defensin 108A | DEFB108P1 |
| C9JUS6 | Putative adrenomedullin-5-like protein | ADM5 |
| P0C7V7 | Putative signal peptidase complex | SEC11B |
| | catalytic subunit SEC11B | |
| P0C854 | Putative cat eye syndrome critical region | CECR9 |
| | protein 9 | |
| Q13046 | Putative pregnancy-specific beta-1- | PSG7 |
| | glycoprotein 7 | |
| Q16609 | Putative apolipoprotein(a)-like protein 2 | LPAL2 |
| Q2TV78 | Putative macrophage-stimulating protein | MST1P9 |
| | MSTP9 | |
| Q5JQD4 | Putative peptide YY-3 | PYY3 |
| Q5R387 | Putative inactive group IIC secretory | PLA2G2C |
| | phospholipase A2 | |
| Q5VSP4 | Putative lipocalin 1-like protein 1 | LCN1P1 |
| Q5W188 | Putative cystatin-9-like protein CST9LP1 | CST9LP1 |
| Q6UXR4 | Putative serpin A13 | SERPINA13P |
| Q86SH4 | Putative testis-specific prion protein | PRNT |
| Q86YQ2 | Putative latherin | LATH |
| Q8IVG9 | Putative humanin peptide | MT-RNR2 |
| Q8NHM4 | Putative trypsin-6 | TRY6 |
| Q8NHW4 | C—C motif chemokine 4-like | CCL4L2 |
| Q9H7L2 | Putative killer cell immunoglobulin-like | KIR3DX1 |
| | receptor-like protein KIR3DX1 | |
| Q9NRI6 | Putative peptide YY-2 | PYY2 |
| Q9UF72 | Putative TP73 antisense gene protein 1 | TP73-AS1 |
| Q9UKY3 | Putative inactive carboxylesterase 4 | CES1P1 |

The Uniprot IDs set forth in Table 1 and Table 2 refer to the human versions the listed proteins and the sequences of each are available from the Uniprot database. Sequences of the listed proteins are also generally available for various animals, including various mammals and animals of veterinary or industrial interest. Accordingly, in some embodiments, compositions and methods of the invention provide for the delivery of one or more mRNAs encoding one or more proteins chosen from mammalian homologs or homologs from an animal of veterinary or industrial interest of the secreted proteins listed in Table 1 or Table 2; thus, compositions of the invention may comprise an mRNA encoding a protein chosen from mammalian homologs or homologs from an animal of veterinary or industrial interest of a protein listed in Table 1 or Table 2 along with other components set out herein, and methods of the invention may comprise preparing and/or administering a composition comprising an mRNA encoding a protein chosen from mammalian homologs or homologs from an animal of veterinary or industrial interest of a protein listed in Table 1 or Table 2 along with other components set out herein. In some embodiments, mammalian homologs are chosen from mouse, rat, hamster, gerbil, horse, pig, cow, llama, alpaca, mink, dog, cat, ferret, sheep, goat, or camel homologs. In some embodiments, the animal of veterinary or industrial interest is chosen from the mammals listed above and/or chicken, duck, turkey, salmon, catfish, or tilapia.

In embodiments, the compositions and methods of the invention provide for the delivery of mRNA encoding a lysosomal protein chosen from Table 3. In some embodiments, the compositions and methods of the invention provide for the delivery of one or more mRNAs encoding one or more lysosomal and/or related proteins listed in Table 3; thus, compositions of the invention may comprise an mRNA encoding a protein listed in Table 3 (or a homolog thereof) along with other components set out herein, and methods of the invention may comprise preparing and/or administering a composition comprising an mRNA encoding a protein chosen from the proteins listed in Table 3 (or a homolog thereof) along with other components set out herein.

TABLE 3
Lysosomal and Related Proteins

α-fucosidase
α-galactosidase
α-glucosidase
α-Iduronidase
α-mannosidase
α-N-acetylgalactosaminidase (α-galactosidase B)
β-galactosidase
β-glucuronidase
β-hexosaminidase
β-mannosidase
3-hydroxy-3-methylglutaryl-CoA (HMG-CoA) lyase
3-methylcrotonyl-CoA carboxylase
3-O-sulfogalactosyl cerebroside sulfatase (arylsulfatase A)
acetyl-CoA transferase
acid alpha-glucosidase
acid ceramidase
acid lipase
acid phosphatase
acid sphingomyelinase
alpha-galactosidase A
arylsulfatase A
beta-galactosidase
beta-glucocerebrosidase
beta-hexosaminidase
biotinidase
cathepsin A
cathepsin K
CLN3
CLN5
CLN6
CLN8
CLN9
cystine transporter (cystinosin)
cytosolic protein beta3A subunit of the adaptor protein-3 complex, AP3
formyl-Glycine generating enzyme (FGE)
galactocerebrosidase
galactose-1-phosphate uridyltransferase (GALT)
galactose 6-sulfate sulfatase
(also known as N-acetylgalactosamine-6-sulfatase)
glucocerebrosidase
glucuronate sulfatase
glucuronidase
glycoprotein cleaving enzymes
glycosaminoglycan cleaving enzymes
glycosylasparaginase (aspartylglucosaminidase)
GM2-AP
Heparan-alpha-glucosaminide N-acetyltransferase (HGSNAT, TMEM76)
Heparan sulfatase
hexosaminidase A lysosomal proteases methylmalonyl-CoA mutase
hyaluronidase
Iduronate sulfatase
LAMP-2
lysosomal α-mannosidase
Lysosomal p40 (C2orf18)
Major facilitator superfamily domain containing 8 protein
(MFSD8 or CLN7)
N-acetylgalactosamine 4-sulfatase
N-acetyl glucosamine 6-sulfatase
N-acetyl glucosaminidase
N-acetylglucosamine-1-phosphate transferase
NPC1
NPC2
palmitoyl-protein thioesterase
palmitoyl-protein thioesterase (CLN1)
Saposin A (Sphingolipid activator protein A)
Saposin B (Sphingolipid activator protein B)
Saposin C (Sphingolipid activator protein C)
Saposin D (Sphingolipid activator protein D)
sialic acid transporter (sialin)
sialidase
Sialin
sulfatase
Transmembrane protein 74 (TMEM74)
tripeptidyl-peptidase
tripeptidyl-peptidase I (CLN2)
UDP-N-acetylglucosamine-phosphotransferase

Information regarding lysosomal proteins is available from Lubke et al., “Proteomics of the Lysosome,” Biochim Biophys Acta. (2009) 1793: 625-635. In some embodiments, the protein listed in Table 3 and encoded by mRNA in the compositions and methods of the invention is a human protein. Sequences of the listed proteins are also available for various animals, including various mammals and animals of veterinary or industrial interest as described above.

In some embodiments, the compositions and methods of the invention provide for the delivery of mRNA encoding a therapeutic protein (e.g., cytosolic, transmembrane or secreted) such as those listed in Table 4. In some embodiments, the compositions and methods of the invention provide for the delivery of an mRNA encoding a therapeutic protein useful in treating a disease or disorder (i.e., indication) listed in Table 4; thus, compositions of the invention may comprise an mRNA encoding a therapeutic protein listed or not listed in Table 4 (or a homolog thereof, as discussed below) along with other components set out herein for treating a disease or disorder (i.e., indication) listed in Table 4, and methods of the invention may comprise preparing and/or administering a composition comprising an mRNA encoding a such a protein (or a homolog thereof, as discussed below) along with other components set out herein for treatment of a disease or disorder listed in Table 4.

TABLE 4
Exemplary Indications and Related Proteins

| Indication | Therapeutic Protein |
|---|---|
| 3-Methylcrotonyl-CoA carboxylase deficiency | Methylcrotonoyl-CoA carboxylase |
| 3-Methylglutaconic aciduria | Methylglutaconyl-CoA hydratase |
| Actinic keratosis | |
| Acute intermittent porphyria | Porphobilinogen deaminase |
| Acute lymphocytic leukemia | |
| Acute myeloid leukemia | |
| Addison's disease | |
| Adenosine deaminase deficiency | Adenosine deaminase |
| Adrenoleukodystrophy | ABCD1 |
| Adrenomyeloneuropathy | |
| AIDS/HIV | |
| Alcohol use disorders | |
| Alkaptonuria | Homogentisate 1,2-dioxygenase |
| Allergic asthma | Anti-IgE mAb |
| Allergies (dermatitis, rhinitis) | |
| Alopecia areata | |
| Alpers' disease | POLG |
| Alpers-Huttenlocher syndrome | |
| Alpha 1-antitrypsin deficiency | Alpha 1 protease inhibitor |
| Alpha-mannosidosis | Alpha-D-mannosidase |
| Alport syndrome | |
| Alzheimer's disease | |
| Amyloid light-chain amyloidosis | |
| Amyotrophic lateral sclerosis (ALS) | |
| Anemia | Erythropoietin |
| Aortic valve stenosis | |
| Argininemia | Arginase |
| Argininosuccinic acidemia | Argininosuccinate lyase |
| Arrhythmogenic right ventricular dysplasia | |
| Autism | |
| Autosomal dominant and recessive progressive | |
| external ophthalmoplegia with mitochondrial DNA | |
| deletions | |
| Autosomal recessive polycystic kidney disease | ARPKD |
| Bacterial infections | |
| Basal cell carcinoma | |
| Batten disease | Battenin + others |
| B-cell chronic lymphocytic leukemia | |
| Becker muscular dystrophy | Dystrophin |
| Beta-thalassemia | Beta globin |
| Binge eating disorder | |
| Bipolar disorder | |
| Bladder cancer | |
| Blepharospasm, Cervical dystonia, Chronic migraine, | Botulinum toxin |
| more | |
| Bronchiolitis obliterans | |
| Brugada syndrome | |
| Buerger's disease | |
| CACNA1A | |
| CACNB4-related Episodic Ataxia Type 2 | |
| Cancer and depression | |
| Cancer and sexual dysfunction | |
| Cancer in pregnancy | |
| Carbamylphosphate synthetase deficiency | Carbamylphosphate synthetase |
| Carcinoma of the gallbladder | |
| Cardiomyopathy (diabetic) | |
| Cardiomyopathy (hypertrophic) | |
| Carnitine uptake defect | SLC22A5 |
| Catecholaminergic polymorphic ventricular | |
| tachycardia | |
| CDKL5-related Atypical Rett Syndrome | |
| Celiac disease | |
| Cellulitis | |
| Cerebrovascular disease | |
| Cervix uteri cancer | |
| Chronic fatigue syndrome | |
| Chronic graft versus host disease | |
| Chronic idiopathic urticaria | |
| Chronic immune thrombocytopenia | Thrombopoietin |
| Chronic kidney kisease | |
| Chronic liver disease | |
| Chronic lymphocytic leukemia | |
| Chronic myeloid leukemia | |
| Chronic pancreatitis | |
| Cirrhosis of the liver | |
| Citrullinemia, type I | Argininosuccinate synthase |
| Classic Rett Syndrome | |
| Classical galactosemia | Galactose-1-phosphate uridylyltransferase |
| Clostridium difficile associated diarrhea | |
| Clotting disorders | |
| COAD/COPD | |
| Cocaine addiction | |
| COL4A5-related disorders | |
| Cold contact urticaria | |
| Contraception, female | |
| Coronary artery diseases | |
| Corpus uteri cancer | |
| Corticobasal degeneration | |
| Crigler-Najjar syndrome | UDP-glucuronosyltransferase |
| Critical limb ischemia | |
| CTNS-related cystinosis | |
| Cutaneous lupus erythematosus | |
| Cutaneous neuroendocrine carcinoma (Merkel Cell) | |
| Cystic fibrosis | CFTR |
| Cystic fibrosis | Deoxyribonuclease I |
| Cystinosis | Cystinosin |
| Cystinuria | SLC7A9 |
| Dementia (Lewy body) | |
| Depression | |
| Diabetic foot infections | |
| Diabetic foot ulcer | |
| Diabetic peripheral neuropathy | |
| Diabetic ulcers | |
| Diarrhoeal diseases | |
| Diffuse large B-cell lymphoma | |
| DiGeorge syndrome | |
| Diverticulitis | |
| Drug use disorders | |
| Duchenne muscular dystrophy | Dystrophin |
| Dysarthria | |
| Dyskinesia (levodopa-induced) | |
| Early-onset autosomal dominant Alzheimer's disease | |
| Eczema | |
| Ehlers-Danlos syndrome, type 1 | |
| EIF2B1 | |
| EIF2B2 | |
| EIF2B3 | |
| EIF2B4 | |
| EIF2B5-related childhood ataxia with central nervous | |
| system hypomyelination/vanishing white matter | |
| Eosinophilic esophagitis | |
| Epilepsy | |
| Erectile dysfunction | |
| Erythropoietic protoporphyria | Ferrochelatase |
| Esophageal carcinoma | |
| Essential tremor | |
| Fabry disease | Alpha galactosidase |
| Familial adenomatous polyposis | APC |
| Familial chylomicronemia | Lipoprotein lipase |
| Familial dysbetalipoproteinemia | Apolipoprotein E |
| Familial isolated dilated cardiomyopathy | |
| Familial mediterranean fever | Pyrin (MEFV) |
| Familial melanoma | |
| Female infertility | Follicle stimulating hormone |
| Female sexual dysfunction | |
| Fibromyalgia | |
| FMR1-related disorders | |
| Fracture healing | |
| Fragile X Premature Ovarian Failure Syndrome | |
| Fragile X syndrome | FMRP |
| Fragile X-Associated Tremor/Ataxia Syndrome | |
| Friedreich's ataxia | |
| Frontotemporal dementia | |
| Fryns syndrome | |
| Galactocerebrosidase deficiencies | |
| GALE deficiency | Galactose epimerase |
| GALK deficiency | Galactokinase |
| GALT-related galactosemia | |
| Gastric cancer | |
| Gastroesophageal reflux disease | |
| Gaucher disease | Glucocerebrosidase |
| Gilbert syndrome | UDP-glucuronosyltransferase |
| Glioblastoma multiforme | |
| Glomerulonephritis | |
| Glutaric acidemia, type I | Glutaryl-CoA dehydrogenase |
| GM2 gangliosidosis | HEXA, HEXB |
| Gout | Urate oxidase |
| Graft versus host disease | |
| Growth hormone deficiency | Growth hormone 1/Growth hormone 2 |
| Head and neck cancer, Metastatic colorectal cancer | Anti-EGFr mAb |
| Hearing loss, adult onset | |
| Heart failure | |
| Hemachromatosis | HFE protein |
| Hemifacial spasm | |
| Hemolytic uremic syndrome | Anti-complement factor C5 mAb |
| Hemophilia A | Factor VIII |
| Hemophilia A, Hemophilia B | Factor VII |
| Hemophilia B | Factor IX |
| Hepatitis B, Hepatitis C | Interferon alpha |
| HER2+ breast cancer, gastric cancer | Anti-HER2 mAb |
| Hereditary angioedema | C1 esterase inhibitor |
| Hereditary hemorrhagic telangiectasia | |
| Hereditary hemorrhagic telangiectasia (AT) | |
| Hereditary spherocytosis | |
| Hidradenitis suppurativa | |
| Homocystinuria | Cystathionine beta-synthase |
| Homozygous familial hypercholesterolemia | LDL receptor |
| Hunter syndrome (MPS II) | Iduronate-2-sulfatase |
| Huntington disease | Huntingtin |
| Hurler syndrome (MPS I) | Alpha-L iduronidase |
| Hydrolethalus | |
| Hyperalgesia | |
| Hyperbilirubinemia | |
| Hyperhidrosis | |
| Hyperlipidemia | |
| Hypermethioninemia | Methionine adenosyltransferase |
| Hyperoxaluria, type I | Serine-pyruvate aminotransferase |
| Hypertension | |
| Hyperuricemia | |
| Hyponatremia | |
| Hypoparathyroidism | Parathyroid hormone |
| Hypophosphatasia | TNSALP |
| Idiopathic pulmonary fibrosis | |
| Iminoglycinuria | |
| Immunoglobulin deficiency | Immunoglobulin |
| Infection (adenovirus) | |
| Infection (anthrax prophylaxis) | |
| Infection (BK virus) | |
| Infection (Clostridium difficile prophylaxis) | |
| Infection (Dengue fever prophylaxis) | |
| Infection (Epstein-Barr virus) | |
| Infection (Hepatitis-D) | |
| Infection (Lyme disease prophylaxis) | |
| Infection (Smallpox virus) | |
| Infectious diseases vaccines | Infectious antigen |
| Inflammatory heart diseases | |
| Insomnia | |
| Interstitial cystitis | |
| Iron-deficiency anaemia | |
| Irritable bowel disease | |
| Ischaemic heart disease | |
| Isovaleric aciduria | Isovaleric acid CoA dehydrogenase deficiency |
| Jansky-Bielschowsky disease | |
| Juvenile Batten disease | |
| Juvenile Neuronal Ceroid Lipofuscinosis (JNCL) | |
| Juvenile rheumatoid arthritis | TNF-alpha inhibitors |
| Kennedy's disease (SBMA) | |
| Keratoconus | |
| Krabbe disease | Galactocerebrosidase |
| Leber's hereditary optic neuropathy | NADH dehydrogenase |
| Leiomyosarcoma | |
| Lennox-Gastaut syndrome | |
| Lesch-Nyhan syndrome | Hypoxanthine phosphoribosyltransferase 1 |
| Leukaemia | |
| Li-Fraumeni syndrome | TP53 |
| Lipoma | |
| Liposarcoma | |
| Liver cancer | |
| Long-chain 3-OH acyl-CoA dehydrogenase deficiency | Long-chain-3-hydroxyacyl-CoA dehydrogenase |
| Lower respiratory infections | |
| Lysosomal acid lipase deficiency | Lysosomal acid lipase |
| Macular degeneration | |
| Major depressive disorder | |
| Malignant fibrous histiocytoma | |
| Mantle cell lymphoma | |
| Maple syrup urine disease | 3-methyl-2-oxobutanoate dehydrogenase |
| Marfan syndrome | FBN1 |
| Maroteaux-Lamy syndrome (MPS VI) | N-acetylgalactosamine 4-sulfatase |
| Mastocytosis | |
| McArdle disease | Muscle glycogen phosphorylase |
| MECP2-related disorders | |
| MECP2-related Severe Neonatal Encephalopathy | |
| Medium-chain acyl-CoA dehydrogenase deficiency | Acyl-CoA dehydrogenase |
| Melanoma | Anti-CTLA4 mAb |
| Metachromatic leukodystrophy | Arylsulfatase A |
| Metastatic colorectal cancer, NSCLC, others | Anti-VEGF mAb |
| Methylmalonyl-CoA mutase deficiency | Methylmalonyl-CoA mutase |
| Migraine | |
| Mitochondrial oxidative phosphorylation disorders | |
| Morquio syndrome, type A (MPS IVA) | Galactose 6-sulfate sulfatase |
| Morquio syndrome, type B (MPS IVB) | Beta-galactosidase |
| Mouth and oropharynx cancers | |
| Multiple carboxylase deficiency | Biotin-methylcrotonoyl-CoA-carboxylase ligase |
| Multiple myeloma | |
| Multiple sclerosis | Anti-VLA-4 mAb |
| Multiple sclerosis | Interferon beta |
| Multiple system atrophy | |
| Myasthenia gravis | |
| Myelofibrosis | |
| Narcolepsy | |
| Neonatal bronchopulmonary dysplasia | |
| Neonatal infections | |
| Nephritis and nephrosis | |
| Neurofibromatosis, type 1 | NF-1 |
| Neuronal ceroid lipofuscinoses-related diseases | |
| Neutropenia | G-CSF |
| Niemann Pick disease, type A/B | SMPD1 |
| Niemann Pick disease, type C | NPC1 |
| Niemann-Pick disease Type C1 | |
| Nocturia | |
| Non-alcoholic fatty liver disease | |
| Non-Hodgkin lymphoma | Anti-CD20 mAb |
| Non-small cell lung cancer | |
| Notch-3 related cerebral autosomal dominant | |
| arteriopathy with subcortical infarcts and | |
| leukoencephalopathy (CADASIL) | |
| Obesity | |
| Ophthalmoparesis | |
| Opioid induced constipation | |
| Ornithine transcarbamylase deficiency | Ornithine transcarbamylase |
| Osteoarthritis | |
| Osteopetrosis | |
| Osteoporosis | Anti-RANKL mAb |
| Ovarian cancer | |
| Paget disease of bone | Sequestosome 1 |
| Pain | |
| Pancreatic carcinoma | |
| Panic disorder | |
| Parkinson disease | |
| Paroxysmal nocturnal hemoglobinuria | Anti-complement factor C5 Mab |
| Pediculosis capitis (head lice) | |
| Pelizaeus-Merzbacher disease | |
| Pemphigus vulgaris | |
| Peptic ulcer disease | |
| Peripheral neuropathy | |
| Peyronie's disease | |
| Phenylketonuria | Phenylalanine hydroxylase |
| Pneumococcal infection prophylaxis | |
| POLG-related sensory ataxic neuropathy | |
| Polycystic kidney disease | |
| Polycystic ovary syndrome | |
| Polycythaemia vera | |
| Polymerase G-related disorders | |
| Polymorphous light eruption | |
| Pompe disease | Alpha glucosidase |
| Porphyria cutanea tarda | Uroporphyrinogen decarboxylase |
| Post herpetic neuralgia | |
| Post-organ transplant | |
| Pouchitis | |
| PPM-X Syndrome | |
| Prader-Willi syndrome | |
| Preeclampsia | |
| Premature ejaculation | |
| Prematurity and low birth weight | |
| Primary ciliary dyskinesia | |
| Primary glomerular diseases | |
| Primary humoral immune deficiencies (e.g., CVID) | Immunoglobulin |
| Proctitis | |
| Progressive multifocal leukoencephalopathy | |
| Progressive supranuclear palsy | |
| Propionic acidemia | Propionyl-CoA carboxylase |
| Prostate cancer | |
| Psoriasis | Anti-IL-12 & IL-23 mAb |
| Psoriatic arthritis | TNF-alpha inhibitors |
| PTT-1 | |
| Pulmonary arterial hypertension | |
| Pulmonary arterial hypertension | |
| Raynaud's phenomenon | |
| Refractive errors | |
| Renal cell carcinoma | |
| Restless leg syndrome | |
| Retinitis pigmentosa | |
| Rheumatic heart disease | |
| Rheumatoid arthritis | Anti-interleukin-6 (IL-6) mAb |
| Rheumatoid arthritis | T-cell costimulation blocker |
| Rheumatoid arthritis | TNF-alpha inhibitor |
| Romano-Ward syndrome | |
| Rosacea | |
| Sanfilippo syndrome, type A (MPS IIIA) | Heparan N-sulfatase |
| Sanfilippo syndrome, type B (MPS IIIB) | N-acetyl-alpha-D-glucosaminidase |
| Santavuori-Haltia disease | |
| Schizophrenia | |
| Schnitzler syndrome | |
| Scleroderma | |
| SCN1A | |
| SCN1B-related seizure disorders | |
| Short-chain acyl-CoA dehydrogenase deficiency | Butyryl-CoA dehydrogenase |
| Sickle cell disease | Hemoglobin |
| SLC3A1-related disorders | |
| Small cell lung cancer | |
| SMN-1-related spinal muscular atrophy (SMA) | |
| Spinal muscular atrophy | Survival motor neuron protein |
| Squamous cell carcinoma of head and neck | |
| Stickler syndrome | |
| Stomach cancer | |
| Stroke prophylaxis | |
| Synovial sarcoma | |
| Systemic lupus erythematosus | Anti-BAFF |
| Systemic sclerosis | |
| Tetrahydrobiopterin-deficient hyperphenylalaninemia | Tetrahydrobiopterin |
| Thromboangiitis obliterans | |
| Thrombotic disorders | |
| Thyroid cancer | |
| TPP1 deficiencies | |
| Trachea, bronchus, lung cancers | |
| Tricuspid atresia | |
| TSC1 | |
| TSC2-related tuberous sclerosis | |
| Type 2 diabetes mellitus | Glucagon-like peptide 1 (GLP-1) agonist |
| Type 2 diabetes mellitus | Insulin |
| Tyrosinemia, type I | Fumarylacetoacetase |
| Ulcerative colitis | |
| Uterine fibroids | |
| Varicose veins | |
| Venous thromboembolism | |
| Very long-chain acyl-CoA dehydrogenase deficiency | Long-chain-acyl-CoA dehydrogenase |
| von Gierke's disease | Glucose-6-phosphatase |
| Von Hippel-Lindau disease | pVHL |
| Wegener granulomatosis | |
| Wilson disease | Wilson disease protein |
| X-Linked adrenal hypoplasia | |
| X-linked adrenoleukodystrophy | |
| X-linked agammaglobulinemia | Bruton's tyrosine kinase |

In some embodiments, the present invention is used to prevent, treat and/or cure a subject affected with a disease or disorder listed or associated with the proteins listed in Tables 1, 2, 3 or 4. In some embodiments, an mRNA encodes one or more of argininosuccinate synthetase (ASS1), Factor IX, survival motor neuron 1 (SMN1), or phenylalanine hydroxylase

Synthesis of mRNA

mRNAs according to the present invention may be synthesized according to any of a variety of known methods. For example, mRNAs according to the present invention may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7 or SP6 RNA polymerase), DNAse I, pyrophosphatase, and/or RNAse inhibitor. The exact conditions will vary according to the specific application.

In some embodiments, for the preparation of mRNA according to the invention, a DNA template is transcribed in vitro. A suitable DNA template typically has a promoter, for example a T3, T7 or SP6 promoter, for in vitro transcription, followed by desired nucleotide sequence for desired mRNA and a termination signal.

Desired mRNA sequence(s) according to the invention may be determined and incorporated into a DNA template using standard methods. For example, starting from a desired amino acid sequence (e.g., an enzyme sequence), a virtual reverse translation is carried out based on the degenerated genetic code. Optimization algorithms may then be used for selection of suitable codons. Typically, the G/C content can be optimized to achieve the highest possible G/C content on one hand, taking into the best possible account the frequency of the tRNAs according to codon usage on the other hand. The optimized RNA sequence can be established and displayed, for example, with the aid of an appropriate display device and compared with the original (wild-type) sequence. A secondary structure can also be analyzed to calculate stabilizing and destabilizing properties or, respectively, regions of the RNA.

Modified mRNA

In some embodiments, mRNA according to the present invention may be synthesized as unmodified or modified mRNA. Typically, mRNAs are modified to enhance stability. Modifications of mRNA can include, for example, modifications of the nucleotides of the RNA. An modified mRNA according to the invention can thus include, for example, backbone modifications, sugar modifications or base modifications. In some embodiments, mRNAs may be synthesized from naturally occurring nucleotides and/or nucleotide analogues (modified nucleotides) including, but not limited to, purines (adenine (A), guanine (G)) or pyrimidines (thymine (T), cytosine (C), uracil (U)), and as modified nucleotides analogues or derivatives of purines and pyrimidines, such as e.g. 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxyacetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, .beta.-D-mannosyl-queosine, wybutoxosine, and phosphoramidates, phosphorothioates, peptide nucleotides, methylphosphonates, 7-deazaguanosine, 5-methylcytosine and inosine. The preparation of such analogues is known to a person skilled in the art e.g. from the U.S. Pat. Nos. 4,373,071, 4,401,796, 4,415,732, 4,458,066, 4,500,707, 4,668,777, 4,973,679, 5,047,524, 5,132,418, 5,153,319, 5,262,530 and 5,700,642, the disclosures of which are incorporated by reference in their entirety.

In some embodiments, mRNAs (e.g., enzyme encoding mRNAs) may contain RNA backbone modifications. Typically, a backbone modification is a modification in which the phosphates of the backbone of the nucleotides contained in the RNA are modified chemically. Exemplary backbone modifications typically include, but are not limited to, modifications from the group consisting of methylphosphonates, methylphosphoramidates, phosphoramidates, phosphorothioates (e.g. cytidine 5′-O-(1-thiophosphate)), boranophosphates, positively charged guanidinium groups etc., which means by replacing the phosphodiester linkage by other anionic, cationic or neutral groups.

In some embodiments, mRNAs (e.g., enzyme encoding mRNAs) may contain sugar modifications. A typical sugar modification is a chemical modification of the sugar of the nucleotides it contains including, but not limited to, sugar modifications chosen from the group consisting of 2′-deoxy-2′-fluoro-oligoribonucleotide (2′-fluoro-2′-deoxycytidine 5′-triphosphate, 2′-fluoro-2′-deoxyuridine 5′-triphosphate), 2′-deoxy-2′-deamine-oligoribonucleotide (2′-amino-2′-deoxycytidine 5′-triphosphate, 2′-amino-2′-deoxyuridine 5′-triphosphate), 2′-O-alkyloligoribonucleotide, 2′-deoxy-2′-C-alkyloligoribonucleotide (2′-O-methylcytidine 5′-triphosphate, 2′-methyluridine 5′-triphosphate), 2′-C-alkyloligoribonucleotide, and isomers thereof (2′-aracytidine 5′-triphosphate, 2′-arauridine 5′-triphosphate), or azidotriphosphates (2′-azido-2′-deoxycyti dine 5′-triphosphate, 2′-azido-2′-deoxyuridine 5′-triphosphate).

In some embodiments, mRNAs (e.g., enzyme encoding mRNAs) may contain modifications of the bases of the nucleotides (base modifications). A modified nucleotide which contains a base modification is also called a base-modified nucleotide. Examples of such base-modified nucleotides include, but are not limited to, 2-amino-6-chloropurine riboside 5′-triphosphate, 2-aminoadenosine 5′-triphosphate, 2-thiocytidine 5′-triphosphate, 2-thiouridine 5′-triphosphate, 4-thiouridine 5′-triphosphate, 5-aminoallylcytidine 5′-triphosphate, 5-aminoallyluridine 5′-triphosphate, 5-bromocytidine 5′-triphosphate, 5-bromouridine 5′-triphosphate, 5-iodocytidine 5′-triphosphate, 5-iodouridine 5′-triphosphate, 5-methylcytidine 5′-triphosphate, 5-methyluridine 5′-triphosphate, 6-azacytidine 5′-triphosphate, 6-azauridine 5′-triphosphate, 6-chloropurine riboside 5′-triphosphate, 7-deazaadenosine 5′-triphosphate, 7-deazaguanosine 5′-triphosphate, 8-azaadenosine 5′-triphosphate, 8-azidoadenosine 5′-triphosphate, benzimidazole riboside 5′-triphosphate, N1-methyladenosine 5′-triphosphate, N1-methylguanosine 5′-triphosphate, N6-methyladenosine 5′-triphosphate, 06-methylguanosine 5′-triphosphate, pseudouridine 5′-triphosphate, puromycin 5′-triphosphate or xanthosine 5′-triphosphate.

### Cap Structure

Typically, mRNA synthesis includes the addition of a “cap” on the N-terminal (5′) end, and a “tail” on the C-terminal (3′) end. The presence of the cap is important in providing resistance to nucleases found in most eukaryotic cells. The presence of a “tail” serves to protect the mRNA from exonuclease degradation.

Thus, in some embodiments, mRNAs (e.g., enzyme encoding mRNAs) include a 5′ cap structure. A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp (5′(A,G(5′)ppp(5′)A and G(5′)ppp(5′)G.

In some embodiments, naturally occurring cap structures comprise a 7-methyl guanosine that is linked via a triphosphate bridge to the 5′-end of the first transcribed nucleotide, resulting in a dinucleotide cap of m⁷G(5′)ppp(5′)N, where N is any nucleoside. In vivo, the cap is added enzymatically. The cap is added in the nucleus and is catalyzed by the enzyme guanylyl transferase. The addition of the cap to the 5′ terminal end of RNA occurs immediately after initiation of transcription. The terminal nucleoside is typically a guanosine, and is in the reverse orientation to all the other nucleotides, i.e., G(5′)ppp(5′)GpNpNp.

A common cap for mRNA produced by in vitro transcription is m⁷G(5′)ppp(5′)G, which has been used as the dinucleotide cap in transcription with T7 or SP6 RNA polymerase in vitro to obtain RNAs having a cap structure in their 5′-termini. The prevailing method for the in vitro synthesis of capped mRNA employs a pre-formed dinucleotide of the form m⁷G(5′)ppp(5′)G (“m⁷GpppG”) as an initiator of transcription.

To date, a usual form of a synthetic dinucleotide cap used in in vitro translation experiments is the Anti-Reverse Cap Analog (“ARCA”) or modified ARCA, which is generally a modified cap analog in which the 2′ or 3′ OH group is replaced with —OCH₃.

Additional cap analogs include, but are not limited to, chemical structures selected from the group consisting of m⁷GpppG, m⁷GpppA, m⁷GpppC; unmethylated cap analogs (e.g., GpppG); dimethylated cap analog (e.g., m²′⁷GpppG), trimethylated cap analog (e.g., m^{2,2,7}GpppG), dimethylated symmetrical cap analogs (e.g., m⁷Gpppm⁷G), or anti reverse cap analogs (e.g., ARCA; m^{7,2′Ome}GpppG, m^{72′d}GpppG, m^{7,3′Ome}GpppG, m^{7,3′d}GpppG and their tetraphosphate derivatives) (see, e.g., Jemielity, J. et al., “Novel ‘anti-reverse’ cap analogs with superior translational properties”, RNA, 9: 1108-1122 (2003)).

In some embodiments, a suitable cap is a 7-methyl guanylate (“m⁷G”) linked via a triphosphate bridge to the 5′-end of the first transcribed nucleotide, resulting in m⁷G(5′)ppp(5′)N, where N is any nucleoside. A preferred embodiment of a m⁷G cap utilized in embodiments of the invention is m⁷G(5′)ppp(5′)G.

In some embodiments, the cap is a Cap0 structure. Cap0 structures lack a 2-O-methyl residue of the ribose attached to bases 1 and 2. In some embodiments, the cap is a Cap1 structure. Cap1 structures have a 2-O-methyl residue at base 2. In some embodiments, the cap is a Cap2 structure. Cap2 structures have a 2-O-methyl residue attached to both bases 2 and 3.

A variety of m⁷G cap analogs are known in the art, many of which are commercially available. These include the m⁷GpppG described above, as well as the ARCA 3′-OCH₃ and 2′-OCH₃ cap analogs (Jemielity, J. et al., RNA, 9: 1108-1122 (2003)). Additional cap analogs for use in embodiments of the invention include N7-benzylated dinucleoside tetraphosphate analogs (described in Grudzien, E. et al., RNA, 10: 1479-1487 (2004)), phosphorothioate cap analogs (described in Grudzien-Nogalska, E., et al., RNA, 13: 1745-1755 (2007)), and cap analogs (including biotinylated cap analogs) described in U.S. Pat. Nos. 8,093,367 and 8,304,529, incorporated by reference herein.

### Tail Structure

Typically, the presence of a “tail” serves to protect the mRNA from exonuclease degradation. The poly A tail is thought to stabilize natural messengers and synthetic sense RNA. Therefore, in certain embodiments a long poly A tail can be added to an mRNA molecule thus rendering the RNA more stable. Poly A tails can be added using a variety of art-recognized techniques. For example, long poly A tails can be added to synthetic or in vitro transcribed RNA using poly A polymerase (Yokoe, et al. Nature Biotechnology. 1996; 14: 1252-1256). A transcription vector can also encode long poly A tails. In addition, poly A tails can be added by transcription directly from PCR products. Poly A may also be ligated to the 3′ end of a sense RNA with RNA ligase (see, e.g., Molecular Cloning A Laboratory Manual, 2nd Ed., ed. by Sambrook, Fritsch and Maniatis (Cold Spring Harbor Laboratory Press: 1991 edition)).

In some embodiments, mRNAs (e.g., enzyme encoding mRNAs) include a 3′ poly(A) tail structure. Typically, the length of the poly A tail can be at least about 10, 50, 100, 200, 300, 400 at least 500 nucleotides (SEQ ID NO: 12). In some embodiments, a poly-A tail on the 3′ terminus of mRNA typically includes about 10 to 300 adenosine nucleotides (SEQ ID NO: 13) (e.g., about 10 to 200 adenosine nucleotides, about 10 to 150 adenosine nucleotides, about 10 to 100 adenosine nucleotides, about 20 to 70 adenosine nucleotides, or about 20 to 60 adenosine nucleotides). In some embodiments, mRNAs include a 3′ poly(C) tail structure. A suitable poly-C tail on the 3′ terminus of mRNA typically include about 10 to 200 cytosine nucleotides (SEQ ID NO: 14) (e.g., about 10 to 150 cytosine nucleotides, about 10 to 100 cytosine nucleotides, about 20 to 70 cytosine nucleotides, about 20 to 60 cytosine nucleotides, or about 10 to 40 cytosine nucleotides). The poly-C tail may be added to the poly-A tail or may substitute the poly-A tail.

In some embodiments, the length of the poly A or poly C tail is adjusted to control the stability of a modified sense mRNA molecule of the invention and, thus, the transcription of protein. For example, since the length of the poly A tail can influence the half-life of a sense mRNA molecule, the length of the poly A tail can be adjusted to modify the level of resistance of the mRNA to nucleases and thereby control the time course of polynucleotide expression and/or polypeptide production in a target cell.

### 5′ and 3′ Untranslated Region

In some embodiments, mRNAs include a 5′ and/or 3′ untranslated region. In some embodiments, a 5′ untranslated region includes one or more elements that affect an mRNA's stability or translation, for example, an iron responsive element. In some embodiments, a 5′ untranslated region may be between about 50 and 500 nucleotides in length.

In some embodiments, a 3′ untranslated region includes one or more of a polyadenylation signal, a binding site for proteins that affect an mRNA's stability of location in a cell, or one or more binding sites for miRNAs. In some embodiments, a 3′ untranslated region may be between 50 and 500 nucleotides in length or longer.

Exemplary 3′ and/or 5′ UTR sequences can be derived from mRNA molecules which are stable (e.g., globin, actin, GAPDH, tubulin, histone, or citric acid cycle enzymes) to increase the stability of the sense mRNA molecule. For example, a 5′ UTR sequence may include a partial sequence of a CMV immediate-early 1 (IE1) gene, or a fragment thereof to improve the nuclease resistance and/or improve the half-life of the polynucleotide. Also contemplated is the inclusion of a sequence encoding human growth hormone (hGH), or a fragment thereof to the 3′ end or untranslated region of the polynucleotide (e.g., mRNA) to further stabilize the polynucleotide. Generally, these modifications improve the stability and/or pharmacokinetic properties (e.g., half-life) of the polynucleotide relative to their unmodified counterparts, and include, for example modifications made to improve such polynucleotides' resistance to in vivo nuclease digestion.

According to various embodiments, any size mRNA may be encapsulated by provided liposomes. In some embodiments, the provided liposomes may encapsulate mRNA of greater than about 0.5 kb, 1 kb, 1.5 kb, 2 kb, 2.5 kb, 3 kb, 3.5 kb, 4 kb, 4.5 kb, or 5 kb in length.

### Formation of Liposomes

The liposomes for use in provided compositions can be prepared by various techniques which are presently known in the art. For example, multilamellar vesicles (MLV) may be prepared according to conventional techniques, such as by depositing a selected lipid on the inside wall of a suitable container or vessel by dissolving the lipid in an appropriate solvent, and then evaporating the solvent to leave a thin film on the inside of the vessel or by spray drying. An aqueous phase may then added to the vessel with a vortexing motion which results in the formation of ML Vs. Uni-lamellar vesicles (ULV) can then be formed by homogenization, sonication or extrusion of the multi-lamellar vesicles. In addition, unilamellar vesicles can be formed by detergent removal techniques.

In certain embodiments, provided compositions comprise a liposome wherein the mRNA is associated on both the surface of the liposome and encapsulated within the same liposome. For example, during preparation of the compositions of the present invention, cationic liposomes may associate with the mRNA through electrostatic interactions. For example, during preparation of the compositions of the present invention, cationic liposomes may associate with the mRNA through electrostatic interactions.

In some embodiments, the compositions and methods of the invention comprise mRNA encapsulated in a liposome. In some embodiments, the one or more mRNA species may be encapsulated in the same liposome. In some embodiments, the one or more mRNA species may be encapsulated in different liposomes. In some embodiments, the mRNA is encapsulated in one or more liposomes, which differ in their lipid composition, molar ratio of lipid components, size, charge (Zeta potential), targeting ligands and/or combinations thereof. In some embodiments, the one or more liposome may have a different composition of cationic lipids, neutral lipid, PEG-modified lipid and/or combinations thereof. In some embodiments the one or more lipisomes may have a different molar ratio of cationic lipid, neutral lipid, cholesterol and PEG-modified lipid used to create the liposome.

The process of incorporation of a desired mRNA into a liposome is often referred to as “loading”. Exemplary methods are described in Lasic, et al., FEBS Lett., 312: 255-258, 1992, which is incorporated herein by reference. The liposome-incorporated nucleic acids may be completely or partially located in the interior space of the liposome, within the bilayer membrane of the liposome, or associated with the exterior surface of the liposome membrane. The incorporation of a nucleic acid into liposomes is also referred to herein as “encapsulation” wherein the nucleic acid is entirely contained within the interior space of the liposome. The purpose of incorporating a mRNA into a transfer vehicle, such as a liposome, is often to protect the nucleic acid from an environment which may contain enzymes or chemicals that degrade nucleic acids and/or systems or receptors that cause the rapid excretion of the nucleic acids. Accordingly, in some embodiments, a suitable delivery vehicle is capable of enhancing the stability of the mRNA contained therein and/or facilitate the delivery of mRNA to the target cell or tissue.

### Liposome Size

Suitable liposomes in accordance with the present invention may be made in various sizes. In some embodiments, provided liposomes may be made smaller than previously known mRNA encapsulating liposomes. In some embodiments, decreased size of liposomes is associated with more efficient delivery of mRNA. Selection of an appropriate liposome size may take into consideration the site of the target cell or tissue and to some extent the application for which the liposome is being made.

In some embodiments, an appropriate size of liposome is selected to facilitate systemic distribution of antibody encoded by the mRNA. In some embodiments, it may be desirable to limit transfection of the mRNA to certain cells or tissues. For example, to target hepatocytes a liposome may be sized such that its dimensions are smaller than the fenestrations of the endothelial layer lining hepatic sinusoids in the liver; in such cases the liposome could readily penetrate such endothelial fenestrations to reach the target hepatocytes.

Alternatively or additionally, a liposome may be sized such that the dimensions of the liposome are of a sufficient diameter to limit or expressly avoid distribution into certain cells or tissues. For example, a liposome may be sized such that its dimensions are larger than the fenestrations of the endothelial layer lining hepatic sinusoids to thereby limit distribution of the liposomes to hepatocytes.

In some embodiments, the size of a liposome is determined by the length of the largest diameter of the lipososme particle. In some embodiments, a suitable liposome has a size of or less than about 500 nm, 450 nm, 400 nm, 350 nm, 300 nm, 250 nm, 200 nm, 150 nm, 125 nm, 110 nm, 100 nm, 95 nm, 90 nm, 85 nm, 80 nm, 75 nm, 70 nm, 65 nm, 60 nm, 55 nm, or 50 nm. In some embodiments, a suitable liposome has a size no greater than about 250 nm (e.g., no greater than about 225 nm, 200 nm, 175 nm, 150 nm, 125 nm, 100 nm, 75 nm, or 50 nm). In some embodiments, a suitable liposome has a size ranging from about 10-250 nm (e.g., ranging from about 10-225 nm, 10-200 nm, 10-175 nm, 10-150 nm, 10-125 nm, 10-100 nm, 10-75 nm, or 10-50 nm). In some embodiments, a suitable liposome has a size ranging from about 100-250 nm (e.g., ranging from about 100-225 nm, 100-200 nm, 100-175 nm, 100-150 nm). In some embodiments, a suitable liposome has a size ranging from about 10-100 nm (e.g., ranging from about 10-90 nm, 10-80 nm, 10-70 nm, 10-60 nm, or 10-50 nm).

A variety of alternative methods known in the art are available for sizing of a population of liposomes. One such sizing method is described in U.S. Pat. No. 4,737,323, incorporated herein by reference. Sonicating a liposome suspension either by bath or probe sonication produces a progressive size reduction down to small ULV less than about 0.05 microns in diameter. Homogenization is another method that relies on shearing energy to fragment large liposomes into smaller ones. In a typical homogenization procedure, MLV are recirculated through a standard emulsion homogenizer until selected liposome sizes, typically between about 0.1 and 0.5 microns, are observed. The size of the liposomes may be determined by quasi-electric light scattering (QELS) as described in Bloomfield, Ann. Rev. Biophys. Bioeng., 10:421-150 (1981), incorporated herein by reference. Average liposome diameter may be reduced by sonication of formed liposomes. Intermittent sonication cycles may be alternated with QELS assessment to guide efficient liposome synthesis.

### Pharmaceutical Compositions

To facilitate expression of mRNA in vivo, delivery vehicles such as liposomes can be formulated in combination with one or more additional nucleic acids, carriers, targeting ligands or stabilizing reagents, or in pharmacological compositions where it is mixed with suitable excipients. Techniques for formulation and administration of drugs may be found in “Remington's Pharmaceutical Sciences,” Mack Publishing Co., Easton, Pa., latest edition.

Provided liposomally-encapsulated or associated mRNAs, and compositions containing the same, may be administered and dosed in accordance with current medical practice, taking into account the clinical condition of the subject, the site and method of administration, the scheduling of administration, the subject's age, sex, body weight and other factors relevant to clinicians of ordinary skill in the art. The “effective amount” for the purposes herein may be determined by such relevant considerations as are known to those of ordinary skill in experimental clinical research, pharmacological, clinical and medical arts. In some embodiments, the amount administered is effective to achieve at least some stabilization, improvement or elimination of symptoms and other indicators as are selected as appropriate measures of disease progress, regression or improvement by those of skill in the art. For example, a suitable amount and dosing regimen is one that causes at least transient protein (e.g., enzyme) production.

Suitable routes of administration include, for example, oral, rectal, vaginal, transmucosal, pulmonary including intratracheal or inhaled, or intestinal administration; parenteral delivery, including intradermal, transdermal (topical), intramuscular, subcutaneous, intramedullary injections, as well as intrathecal, direct intraventricular, intravenous, intraperitoneal, and/or intranasal administration.

Alternately or additionally, liposomally encapsulated mRNAs and compositions of the invention may be administered in a local rather than systemic manner, for example, via injection of the pharmaceutical composition directly into a targeted tissue, preferably in a sustained release formulation. Local delivery can be affected in various ways, depending on the tissue to be targeted. For example, aerosols containing compositions of the present invention can be inhaled (for nasal, tracheal, or bronchial delivery); compositions of the present invention can be injected into the site of injury, disease manifestation, or pain, for example; compositions can be provided in lozenges for oral, tracheal, or esophageal application; can be supplied in liquid, tablet or capsule form for administration to the stomach or intestines, can be supplied in suppository form for rectal or vaginal application; or can even be delivered to the eye by use of creams, drops, or even injection. Formulations containing provided compositions complexed with therapeutic molecules or ligands can even be surgically administered, for example in association with a polymer or other structure or substance that can allow the compositions to diffuse from the site of implantation to surrounding cells. Alternatively, they can be applied surgically without the use of polymers or supports.

In some embodiments, provided liposomes and/or compositions are formulated such that they are suitable for extended-release of the mRNA contained therein. Such extended-release compositions may be conveniently administered to a subject at extended dosing intervals. For example, in one embodiment, the compositions of the present invention are administered to a subject twice day, daily or every other day. In a preferred embodiment, the compositions of the present invention are administered to a subject twice a week, once a week, every ten days, every two weeks, every three weeks, or more preferably every four weeks, once a month, every six weeks, every eight weeks, every other month, every three months, every four months, every six months, every eight months, every nine months or annually. Also contemplated are compositions and liposomes which are formulated for depot administration (e.g., intramuscularly, subcutaneously, intravitreally) to either deliver or release a mRNA over extended periods of time. Preferably, the extended-release means employed are combined with modifications made to the mRNA to enhance stability.

Also contemplated herein are lyophilized pharmaceutical compositions comprising one or more of the liposomes disclosed herein and related methods for the use of such compositions as disclosed for example, in United States Provisional Application No. 61/494,882, filed Jun. 8, 2011, the teachings of which are incorporated herein by reference in their entirety. For example, lyophilized pharmaceutical compositions according to the invention may be reconstituted prior to administration or can be reconstituted in vivo. For example, a lyophilized pharmaceutical composition can be formulated in an appropriate dosage form (e.g., an intradermal dosage form such as a disk, rod or membrane) and administered such that the dosage form is rehydrated over time in vivo by the individual's bodily fluids.

Provided liposomes and compositions may be administered to any desired tissue. In some embodiments, the mRNA delivered by provided liposomes or compositions is expressed in the tissue in which the liposomes and/or compositions were administered. In some embodiments, the mRNA delivered is expressed in a tissue different from the tissue in which the liposomes and/or compositions were administered Exemplary tissues in which delivered mRNA may be delivered and/or expressed include, but are not limited to the liver, kidney, heart, spleen, serum, brain, skeletal muscle, lymph nodes, skin, and/or cerebrospinal fluid.

According to various embodiments, the timing of expression of delivered mRNAs can be tuned to suit a particular medical need. In some embodiments, the expression of the protein encoded by delivered mRNA is detectable 1, 2, 3, 6, 12, 18, 24, 30, 36, 42, 48, 54, 60, 66, and/or 72 hours in serum or target tissues after a single administration of provided liposomes or compositions. In some embodiments, the expression of the protein encoded by the mRNA is detectable 1 day, 2 days, 3 days, 4 days, 5 days, 6 days, and/or 7 days in serum or target tissues after a single administration of provided liposomes or compositions. In some embodiments, the expression of the protein encoded by the mRNA is detectable 1 week, 2 weeks, 3 weeks, and/or 4 weeks in serum or target tissues after a single administration of provided liposomes or compositions. In some embodiments, the expression of the protein encoded by the mRNA is detectable after a month or longer after a single administration of provided liposomes or compositions.

The present invention can be used to deliver mRNA at various doses. In some embodiments, an mRNA is administered at a dose ranging from about 0.1-5.0 mg/kg body weight, for example about 0.1-4.5,0.1-4.0,0.1-3.5,0.1-3.0,0.1-2.5,0.1-2.0, 0.1-1.5, 0.1-1.0,0.1-0.5,0.1-0.3,0.3-5.0,0.3-4.5,0.3-4.0,0.3-3.5,0.3-3.0,0.3-2.5,0.3-2.0,0.3-1.5,0.3-1.0,0.3-0.5,0.5-5.0,0.5-4.5,0.5-4.0,0.5-3.5,0.5-3.0,0.5-2.5,0.5-2.0, 0.5-1.5, or 0.5-1.0 mg/kg body weight. In some embodiments, an mRNA is administered at a dose of or less than about 5.0, 4.5, 4.0, 3.5, 3.0, 2.5, 2.0, 1.5, 1.0, 0.8, 0.6, 0.5, 0.4, 0.3, 0.2, or 0.1 mg/kg body weight.

### EXAMPLES

While certain compounds, compositions and methods of the present invention have been described with specificity in accordance with certain embodiments, the following examples serve only to illustrate the compounds of the invention and are not intended to limit the same.

### Example 1. Exemplary Liposome Formulations for mRNA Delivery and Expression

This example provides exemplary liposome formulations incorporating the cationic lipids described in this application, for example, cKK-E12, for effective delivery and expression of mRNA encoding therapeutic proteins in vivo.

### Lipid Materials

In general, the formulations described herein are based on a multi-component lipid mixture of varying ratios employing one or more cationic lipids, one or more helper lipids (e.g., non-cationic lipids and/or cholesterol-based lipids), and one or more PEGylated lipids designed to encapsulate various nucleic acid-based materials. As a non-limiting example, cKK-E12 (3,6-bis(4-(bis(2-hydroxydodecyl)amino)butyl)piperazine-2,5-dione) is used in various formulations described herein. Exemplary helper lipids include one or more of DSPC (1,2-distearoyl-sn-glycero-3-phosphocholine), DPPC (1,2-dipalmitoyl-sn-glycero-3-phosphocholine), DOPE (1,2-dioleyl-sn-glycero-3-phosphoethanolamine), DOPC (1,2-dioleyl-sn-glycero-3-phosphotidylcholine) DPPE (1,2-dipalmitoyl-sn-glycero-3-phosphoethanolamine), DMPE (1,2-dimyristoyl-sn-glycero-3-phosphoethanolamine), DOPG (,2-dioleoyl-sn-glycero-3-phospho-(1′-rac-glycerol)), cholesterol, etc. Exemplary PEGylated lipids include a poly(ethylene) glycol chain of up to 5 kDa in length covalently attached to a lipid with alkyl chain(s) of C₆-C₂₀ length, for example, PEG-2K. As non-limiting examples, liposome formulations used in various examples described herein include cKK-E12, DOPE, cholesterol and DMG-PEG2K at various ratios. For example, in some cases, the ratio of cKK-E12: DOPE: cholesterol:DMG-PEG2K is approximately 40:30:20:10 by weight. In other cases, the ratio of cKK-E12:DOPE:cholesterol:DMG-PEG2K is approximately 40:32:25:3 by weight. Unless otherwise specified, the below Examples include a mixture in the ratio of cKK-E 12: DOPE: cholesterol: DMG-PEG2K of approximately 40:30:25:5 by weight.

### Messenger RNA Material

The formulations described herein may be used to deliver any mRNA, in particular, therapeutic mRNA. As used herein, a therapeutic mRNA refers to an mRNA that encodes a therapeutic protein. The formulations described herein can also be used to deliver any modified or unmodified mRNA, or mRNA with naturally occurring sequences or codon-optimized.

As non-limiting examples, human Factor IX (FIX), codon-optimized Firefly Luciferase (FFL), codon-optimized human argininosuccinate synthetase (ASS1) messenger RNA, codon-optimized human Survival of Motor Neuron 1(SMN) mRNA were synthesized by in vitro transcription from a plasmid DNA template encoding the gene, which was followed by the addition of a 5′ cap structure (Cap 1) (Fechter, P.; Brownlee, G. G. “Recognition of mRNA cap structures by viral and cellular proteins” J. Gen. Virology 2005, 86, 1239-1249) and a 3′ poly(A) tail of, e.g., approximately 250 nucleotides in length (SEQ ID NO: 15) as determined by gel electrophoresis. Typically, 5′ and 3′ untranslated regions (UTR) are present in each mRNA product and are represented as X and Y, respectively. Example 5′ and 3′ UTR sequences are described below. The exemplary sequences of FIX, ASS1, and FFL mRNA used in the examples herein are listed below. Also shown are the 5′ and 3′ UTR sequences.

Human Factor IX (FIX) mRNA:
(SEQ ID NO.: 1)
XAUGCAGCGCGUGAACAUGAUCAUGGCAGAAUCACCAGGCCUCAUCACCAUCUGC
CUUUUAGGAUAUCUACUCAGUGCUGAAUGUACAGUUUUUCUUGAUCAUGAAAAC
GCCAACAAAAUUCUGAGGCGGAGAAGGAGGUAUAAUUCAGGUAAAUUGGAAGAG
UUUGUUCAAGGGAACCUUGAGAGAGAAUGUAUGGAAGAAAAGUGUAGUUUUGAA
GAAGCACGAGAAGUUUUUGAAAACACUGAAAGAACAACUGAAUUUUGGAAGCAG
UAUGUUGAUGGAGAUCAGUGUGAGUCCAAUCCAUGUUUAAAUGGCGGCAGUUGC
AAGGAUGACAUUAAUUCCUAUGAAUGUUGGUGUCCCUUUGGAUUUGAAGGAAAG
AACUGUGAAUUAGAUGUAACAUGUAACAUUAAGAAUGGCAGAUGCGAGCAGUUU
UGUAAAAAUAGUGCUGAUAACAAGGUGGUUUGCUCCUGUACUGAGGGAUAUCGA
CUUGCAGAAAACCAGAAGUCCUGUGAACCAGCAGUGCCAUUUCCAUGUGGAAGA
GUUUCUGUUUCACAAACUUCUAAGCUCACCCGUGCUGAGGCUGUUUUUCCUGAUG
UGGACUAUGUAAAUUCUACUGAAGCUGAAACCAUUUUGGAUAACAUCACUCAAA
GCACCCAAUCAUUUAAUGACUUCACUCGGGUUGUUGGUGGAGAAGAUGCCAAAC
CAGGUCAAUUCCCUUGGCAGGUUGUUUUGAAUGGUAAAGUUGAUGCAUUCUGUG
GAGGCUCUAUCGUUAAUGAAAAAUGGAUUGUAACUGCUGCCCACUGUGUUGAAA
CUGGUGUUAAAAUUACAGUUGUCGCAGGUGAACAUAAUAUUGAGGAGACAGAAC
AUACAGAGCAAAAGCGAAAUGUGAUUCGAAUUAUUCCUCACCACAACUACAAUG
CAGCUAUUAAUAAGUACAACCAUGACAUUGCCCUUCUGGAACUGGACGAACCCUU
AGUGCUAAACAGCUACGUUACACCUAUUUGCAUUGCUGACAAGGAAUACACGAA
CAUCUUCCUCAAAUUUGGAUCUGGCUAUGUAAGUGGCUGGGGAAGAGUCUUCCA
CAAAGGGAGAUCAGCUUUAGUUCUUCAGUACCUUAGAGUUCCACUUGUUGACCG
AGCCACAUGUCUUCGAUCUACAAAGUUCACCAUCUAUAACAACAUGUUCUGUGCU
GGCUUCCAUGAAGGAGGUAGAGAUUCAUGUCAAGGAGAUAGUGGGGGACCCCAU
GUUACUGAAGUGGAAGGGACCAGUUUCUUAACUGGAAUUAUUAGCUGGGGUGAA
GAGUGUGCAAUGAAAGGCAAAUAUGGAAUAUAUACCAAGGUAUCCCGGUAUGUC
AACUGGAUUAAGGAAAAAACAAAGCUCACUUAAY
Codon-Optimized Human Argininosuccinate Synthetase
(ASS1) mRNA:
(SEQ ID NO.: 2)
XAUGAGCAGCAAGGGCAGCGUGGUGCUGGCCUACAGCGGCGGCCUGGACACCAGC
UGCAUCCUGGUGUGGCUGAAGGAGCAGGGCUACGACGUGAUCGCCUACCUGGCCA
ACAUCGGCCAGAAGGAGGACUUCGAGGAGGCCCGCAAGAAGGCCCUGAAGCUGGG
CGCCAAGAAGGUGUUCAUCGAGGACGUGAGCCGCGAGUUCGUGGAGGAGUUCAU
CUGGCCCGCCAUCCAGAGCAGCGCCCUGUACGAGGACCGCUACCUGCUGGGCACC
AGCCUGGCCCGCCCCUGCAUCGCCCGCAAGCAGGUGGAGAUCGCCCAGCGCGAGG
GCGCCAAGUACGUGAGCCACGGCGCCACCGGCAAGGGCAACGACCAGGUGCGCUU
CGAGCUGAGCUGCUACAGCCUGGCCCCCCAGAUCAAGGUGAUCGCCCCCUGGCGC
AUGCCCGAGUUCUACAACCGCUUCAAGGGCCGCAACGACCUGAUGGAGUACGCCA
AGCAGCACGGCAUCCCCAUCCCCGUGACCCCCAAGAACCCCUGGAGCAUGGACGA
GAACCUGAUGCACAUCAGCUACGAGGCCGGCAUCCUGGAGAACCCCAAGAACCAG
GCCCCCCCCGGCCUGUACACCAAGACCCAGGACCCCGCCAAGGCCCCCAACACCCC
CGACAUCCUGGAGAUCGAGUUCAAGAAGGGCGUGCCCGUGAAGGUGACCAACGU
GAAGGACGGCACCACCCACCAGACCAGCCUGGAGCUGUUCAUGUACCUGAACGAG
GUGGCCGGCAAGCACGGCGUGGGCCGCAUCGACAUCGUGGAGAACCGCUUCAUCG
GCAUGAAGAGCCGCGGCAUCUACGAGACCCCCGCCGGCACCAUCCUGUACCACGC
CCACCUGGACAUCGAGGCCUUCACCAUGGACCGCGAGGUGCGCAAGAUCAAGCAG
GGCCUGGGCCUGAAGUUCGCCGAGCUGGUGUACACCGGCUUCUGGCACAGCCCCG
AGUGCGAGUUCGUGCGCCACUGCAUCGCCAAGAGCCAGGAGCGCGUGGAGGGCAA
GGUGCAGGUGAGCGUGCUGAAGGGCCAGGUGUACAUCCUGGGCCGCGAGAGCCCC
CUGAGCCUGUACAACGAGGAGCUGGUGAGCAUGAACGUGCAGGGCGACUACGAG
CCCACCGACGCCACCGGCUUCAUCAACAUCAACAGCCUGCGCCUGAAGGAGUACC
ACCGCCUGCAGAGCAAGGUGACCGCCAAGUGAY
Codon-Optimized Firefly Luciferase (FFL) mRNA:
(SEQ ID NO.: 3)
XAUGGAAGAUGCCAAAAACAUUAAGAAGGGCCCAGCGCCAUUCUACCCACUCGAA
GACGGGACCGCCGGCGAGCAGCUGCACAAAGCCAUGAAGCGCUACGCCCUGGUGC
CCGGCACCAUCGCCUUUACCGACGCACAUAUCGAGGUGGACAUUACCUACGCCGA
GUACUUCGAGAUGAGCGUUCGGCUGGCAGAAGCUAUGAAGCGCUAUGGGCUGAA
UACAAACCAUCGGAUCGUGGUGUGCAGCGAGAAUAGCUUGCAGUUCUUCAUGCCC
GUGUUGGGUGCCCUGUUCAUCGGUGUGGCUGUGGCCCCAGCUAACGACAUCUACA
ACGAGCGCGAGCUGCUGAACAGCAUGGGCAUCAGCCAGCCCACCGUCGUAUUCGU
GAGCAAGAAAGGGCUGCAAAAGAUCCUCAACGUGCAAAAGAAGCUACCGAUCAU
ACAAAAGAUCAUCAUCAUGGAUAGCAAGACCGACUACCAGGGCUUCCAAAGCAUG
UACACCUUCGUGACUUCCCAUUUGCCACCCGGCUUCAACGAGUACGACUUCGUGC
CCGAGAGCUUCGACCGGGACAAAACCAUCGCCCUGAUCAUGAACAGUAGUGGCAG
UACCGGAUUGCCCAAGGGCGUAGCCCUACCGCACCGCACCGCUUGUGUCCGAUUC
AGUCAUGCCCGCGACCCCAUCUUCGGCAACCAGAUCAUCCCCGACACCGCUAUCC
UCAGCGUGGUGCCAUUUCACCACGGCUUCGGCAUGUUCACCACGCUGGGCUACUU
GAUCUGCGGCUUUCGGGUCGUGCUCAUGUACCGCUUCGAGGAGGAGCUAUUCUU
GCGCAGCUUGCAAGACUAUAAGAUUCAAUCUGCCCUGCUGGUGCCCACACUAUUU
AGCUUCUUCGCUAAGAGCACUCUCAUCGACAAGUACGACCUAAGCAACUUGCACG
AGAUCGCCAGCGGCGGGGCGCCGCUCAGCAAGGAGGUAGGUGAGGCCGUGGCCAA
ACGCUUCCACCUACCAGGCAUCCGCCAGGGCUACGGCCUGACAGAAACAACCAGC
GCCAUUCUGAUCACCCCCGAAGGGGACGACAAGCCUGGCGCAGUAGGCAAGGUGG
UGCCCUUCUUCGAGGCUAAGGUGGUGGACUUGGACACCGGUAAGACACUGGGUG
UGAACCAGCGCGGCGAGCUGUGCGUCCGUGGCCCCAUGAUCAUGAGCGGCUACGU
UAACAACCCCGAGGCUACAAACGCUCUCAUCGACAAGGACGGCUGGCUGCACAGC
GGCGACAUCGCCUACUGGGACGAGGACGAGCACUUCUUCAUCGUGGACCGGCUGA
AGAGCCUGAUCAAAUACAAGGGCUACCAGGUAGCCCCAGCCGAACUGGAGAGCAU
CCUGCUGCAACACCCCAACAUCUUCGACGCCGGGGUCGCCGGCCUGCCCGACGAC
GAUGCCGGCGAGCUGCCCGCCGCAGUCUCGUGCUGGAACACGGUAAAACCAUGA
CCGAGAAGGAGAUCGUGGACUAUGUGGCCAGCCAGGUUACAACCGCCAAGAAGCU
GCGCGGUGGUGUUGUGUUCGUGGACGAGGUGCCUAAAGGACUGACCGGCAAGUU
GGACGCCCGCAAGAUCCGCGAGAUUCUCAUUAAGGCCAAGAAGGGCGGCAAGAUC
GCCGUGUAAY
Codon-Optimized Human Survival of Motor Neuron 1 (SMN)
mRNA:
(SEQ ID NO: 4)
XAUGGCCAUGAGCAGCGGAGGCAGCGGCGGAGGAGUGCCCGAGCAGGAGGACAG
CGUGCUGUUCAGGAGAGGCACCGGCCAGAGCGAUGACAGCGAUAUCUGGGACGA
UACCGCUCUGAUCAAGGCCUACGACAAGGCCGUGGCCAGCUUCAAGCACGCCCUG
AAAAACGGCGACAUCUGCGAGACCAGCGGCAAGCCCAAGACAACCCCCAAGAGAA
AGCCCGCCAAGAAGAAUAAGAGCCAGAAAAAGAACACCGCCGCCAGCCUGCAGCA
GUGGAAGGUGGGCGACAAGUGCAGCGCCAUCUGGAGCGAGGACGGCUGCAUCUA
CCCCGCCACCAUCGCCAGCAUCGACUUCAAGAGAGAGACCUGCGUGGUCGUGUAC
ACCGGCUACGGCAACAGAGAGGAGCAGAACCUGAGCGACCUGCUGAGCCCCAUUU
GUGAGGUGGCCAAUAACAUCGAACAGAACGCCCAGGAGAACGAGAAUGAAAGCC
AGGUGAGCACCGACGAGAGCGAGAACAGCAGAUCUCCUGGCAACAAGAGCGACAA
CAUCAAGCCUAAGUCUGCCCCUUGGAACAGCUUCCUGCCCCCUCCUCCACCCAUG
CCCGGACCCAGACUGGGACCCGGAAAACCUGGCCUGAAGUUCAACGGACCACCUC
CCCCUCCACCUCCUCCCCCACCUCAUCUCCUGAGCUGCUGGCUGCCACCCUUCCCC
AGCGGACCCCCUAUCAUCCCACCACCCCCUCCCAUCUGCCCCGACAGCCUGGACGA
CGCCGAUGCCCUGGGCAGCAUGCUGAUCAGCUGGUACAUGAGCGGCUACCACACA
GGAUACUACAUGGGCUUCAGACAGAACCAGAAGGAGGGCAGAUGCUCCCACUCCC
UGAACUGAY
5′ and 3′ UTR Sequences
X (5′ UTR Sequence) =
(SEQ ID NO.: 5)
GGACAGAUCGCCUGGAGACGCCAUCCACGCUGUUUUGACCUCCAUAGAAGACACC
GGGACCGAUCCAGCCUCCGCGGCCGGGAACGGUGCAUUGGAACGCGGAUUCCCCG
UGCCAAGAGUGACUCACCGUCCUUGACACG
Y (3′ UTR Sequence) =
(SEQ ID NO.: 6)
CGGGUGGCAUCCCUGUGACCCCUCCCCAGUGCCUCUCCUGGCCCUGGAAGUUGCC
ACUCCAGUGCCCACCAGCCUUGUCCUAAUAAAAUUAAGUUGCAUCAAGCU
or
(SEQ ID NO.: 7)
GGGUGGCAUCCCUGUGACCCCUCCCCAGUGCCUCUCCUGGCCCUGGAAGUUGCCA
CUCCAGUGCCCACCAGCCUUGUCCUAAUAAAAUUAAGUUGCAUCAAAGCU
C-terminal His₁₀ Codon-Optimized Human CFTR mRNA
(“His₁₀” disclosed as SEQ ID NO: 11):
(SEQ ID NO.: 8)
XAUGCAGCGGUCCCCGCUCGAAAAGGCCAGUGUCGUGUCCAAACUCUUCUUCUCA
UGGACUCGGCCUAUCCUUAGAAAGGGGUAUCGGCAGAGGCUUGAGUUGUCUGAC
AUCUACCAGAUCCCCUCGGUAGAUUCGGCGGAUAACCUCUCGGAGAAGCUCGAAC
GGGAAUGGGACCGCGAACUCGCGUCUAAGAAAAACCCGAAGCUCAUCAACGCACU
GAGAAGGUGCUUCUUCUGGCGGUUCAUGUUCUACGGUAUCUUCUUGUAUCUCGG
GGAGGUCACAAAAGCAGUCCAACCCCUGUUGUUGGGUCGCAUUAUCGCCUCGUAC
GACCCCGAUAACAAAGAAGAACGGAGCAUCGCGAUCUACCUCGGGAUCGGACUGU
GUUUGCUUUUCAUCGUCAGAACACUUUUGUUGCAUCCAGCAAUCUUCGGCCUCCA
UCACAUCGGUAUGCAGAUGCGAAUCGCUAUGUUUAGCUUGAUCUACAAAAAGAC
ACUGAAACUCUCGUCGCGGGUGUUGGAUAAGAUUUCCAUCGGUCAGUUGGUGUC
CCUGCUUAGUAAUAACCUCAACAAAUUCGAUGAGGGACUGGCGCUGGCACAUUUC
GUGUGGAUUGCCCCGUUGCAAGUCGCCCUUUUGAUGGGCCUUAUUUGGGAGCUG
UUGCAGGCAUCUGCCUUUUGUGGCCUGGGAUUUCUGAUUGUGUUGGCAUUGUUU
CAGGCUGGGCUUGGGCGGAUGAUGAUGAAGUAUCGCGACCAGAGAGCGGGUAAA
AUCUCGGAAAGACUCGUCAUCACUUCGGAAAUGAUCGAAAACAUCCAGUCGGUCA
AAGCCUAUUGCUGGGAAGAAGCUAUGGAGAAGAUGAUUGAAAACCUCCGCCAAA
CUGAGCUGAAACUGACCCGCAAGGCGGCGUAUGUCCGGUAUUUCAAUUCGUCAGC
GUUCUUCUUUUCCGGGUUCUUCGUUGUCUUUCUCUCGGUUUUGCCUUAUGCCUUG
AUUAAGGGGAUUAUCCUCCGCAAGAUUUUCACCACGAUUUCGUUCUGCAUUGUA
UUGCGCAUGGCAGUGACACGGCAAUUUCCGUGGGCCGUGCAGACAUGGUAUGAC
UCGCUUGGAGCGAUCAACAAAAUCCAAGACUUCUUGCAAAAGCAAGAGUACAAG
ACCCUGGAGUACAAUCUUACUACUACGGAGGUAGUAAUGGAGAAUGUGACGGCU
UUUUGGGAAGAGGGUUUUGGAGAACUGUUUGAGAAAGCAAAGCAGAAUAACAAC
AACCGCAAGACCUCAAAUGGGGACGAUUCCCUGUUUUUCUCGAACUUCUCCCUGC
UCGGAACACCCGUGUUGAAGGACAUCAAUUUCAAGAUUGAGAGGGGACAGCUUC
UCGCGGUAGCGGGAAGCACUGGUGCGGGAAAAACUAGCCUCUUGAUGGUGAUUA
UGGGGGAGCUUGAGCCCAGCGAGGGGAAGAUUAAACACUCCGGGCGUAUCUCAU
UCUGUAGCCAGUUUUCAUGGAUCAUGCCCGGAACCAUUAAAGAGAACAUCAUUU
UCGGAGUAUCCUAUGAUGAGUACCGAUACAGAUCGGUCAUUAAGGCGUGCCAGU
UGGAAGAGGACAUUUCUAAGUUCGCCGAGAAGGAUAACAUCGUCUUGGGAGAAG
GGGGUAUUACAUUGUCGGGAGGGCAGCGAGCGCGGAUCAGCCUCGCGAGAGCGG
UAUACAAAGAUGCAGAUUUGUAUCUGCUUGAUUCACCGUUUGGAUACCUCGACG
UAUUGACAGAAAAAGAAAUCUUCGAGUCGUGCGUGUGUAAACUUAUGGCUAAUA
AGACGAGAAUCCUGGUGACAUCAAAAAUGGAACACCUUAAGAAGGCGGACAAGA
UCCUGAUCCUCCACGAAGGAUCGUCCUACUUUUACGGCACUUUCUCAGAGUUGCA
AAACUUGCAGCCGGACUUCUCAAGCAAACUCAUGGGGUGUGACUCAUUCGACCAG
UUCAGCGCGGAACGGCGGAACUCGAUCUUGACGGAAACGCUGCACCGAUUCUCGC
UUGAGGGUGAUGCCCCGGUAUCGUGGACCGAGACAAAGAAGCAGUCGUUUAAGC
AGACAGGAGAAUUUGGUGAGAAAAGAAAGAACAGUAUCUUGAAUCCUAUUAACU
CAAUUCGCAAGUUCUCAAUCGUCCAGAAAACUCCACUGCAGAUGAAUGGAAUUG
AAGAGGAUUCGGACGAACCCCUGGAGCGCAGGCUUAGCCUCGUGCCGGAUUCAGA
GCAAGGGGAGGCCAUUCUUCCCCGGAUUUCGGUGAUUUCAACCGGACCUACACUU
CAGGCGAGGCGAAGGCAAUCCGUGCUCAACCUCAUGACGCAUUCGGUAAACCAGG
GGCAAAACAUUCACCGCAAAACGACGGCCUCAACGAGAAAAGUGUCACUUGCACC
CCAGGCGAAUUUGACUGAACUCGACAUCUACAGCCGUAGGCUUUCGCAAGAAACC
GGACUUGAGAUCAGCGAAGAAAUCAAUGAAGAAGAUUUGAAAGAGUGUUUCUUU
GAUGACAUGGAAUCAAUCCCAGCGGUGACAACGUGGAACACAUACUUGCGUUAC
AUCACGGUGCACAAGUCCUUGAUUUUCGUCCUCAUCUGGUGUCUCGUGAUCUUUC
UCGCUGAGGUCGCAGCGUCACUUGUGGUCCUCUGGCUGCUUGGUAAUACGCCCUU
GCAAGACAAAGGCAAUUCUACACACUCAAGAAACAAUUCCUAUGCCGUGAUUAUC
ACUUCUACAAGCUCGUAUUACGUGUUUUACAUCUACGUAGGAGUGGCCGACACUC
UGCUCGCGAUGGGUUUCUUCCGAGGACUCCCACUCGUUCACACGCUUAUCACUGU
CUCCAAGAUUCUCCACCAUAAGAUGCUUCAUAGCGUACUGCAGGCUCCCAUGUCC
ACCUUGAAUACGCUCAAGGCGGGAGGUAUUUUGAAUCGCUUCUCAAAAGAUAUU
GCAAUUUUGGAUGACCUUCUGCCCCUGACGAUCUUCGACUUCAUCCAGUUGUUGC
UGAUCGUGAUUGGGGCUAUUGCAGUAGUCGCUGUCCUCCAGCCUUACAUUUUUG
UCGCGACCGUUCCGGUGAUCGUGGCGUUUAUCAUGCUGCGGGCCUAUUUCUUGCA
GACGUCACAGCAGCUUAAGCAACUGGAGUCUGAAGGGAGGUCGCCUAUCUUUAC
GCAUCUUGUGACCAGUUUGAAGGGAUUGUGGACGUUGCGCGCCUUUGGCAGGCA
GCCCUACUUUGAAACACUGUUCCACAAAGCGCUGAAUCUCCAUACGGCAAAUUGG
UUUUUGUAUUUGAGUACCCUCCGAUGGUUUCAGAUGCGCAUUGAGAUGAUUUUU
GUGAUCUUCUUUAUCGCGGUGACUUUUAUCUCCAUCUUGACCACGGGAGAGGGC
GAGGGACGGGUCGGUAUUAUCCUGACACUCGCCAUGAACAUUAUGAGCACUUUG
CAGUGGGCAGUGAACAGCUCGAUUGAUGUGGAUAGCCUGAUGAGGUCCGUUUCG
AGGGUCUUUAAGUUCAUCGACAUGCCGACGGAGGGAAAGCCCACAAAAAGUACG
AAACCCUAUAAGAAUGGGCAAUUGAGUAAGGUAAUGAUCAUCGAGAACAGUCAC
GUGAAGAAGGAUGACAUCUGGCCUAGCGGGGGUCAGAUGACCGUGAAGGACCUG
ACGGCAAAAUACACCGAGGGAGGGAACGCAAUCCUUGAAAACAUCUCGUUCAGCA
UUAGCCCCGGUCAGCGUGUGGGGUUGCUCGGGAGGACCGGGUCAGGAAAAUCGA
CGUUGCUGUCGGCCUUCUUGAGACUUCUGAAUACAGAGGGUGAGAUCCAGAUCG
ACGGCGUUUCGUGGGAUAGCAUCACCUUGCAGCAGUGGCGGAAAGCGUUUGGAG
UAAUCCCCCAAAAGGUCUUUAUCUUUAGCGGAACCUUCCGAAAGAAUCUCGAUCC
UUAUGAACAGUGGUCAGAUCAAGAGAUUUGGAAAGUCGCGGACGAGGUUGGCCU
UCGGAGUGUAAUCGAGCAGUUUCCGGGAAAACUCGACUUUGUCCUUGUAGAUGG
GGGAUGCGUCCUGUCGCAUGGGCACAAGCAGCUCAUGUGCCUGGCGCGAUCCGUC
CUCUCUAAAGCGAAAAUUCUUCUCUUGGAUGAACCUUCGGCCCAUCUGGACCCGG
UAACGUAUCAGAUCAUCAGAAGGACACUUAAGCAGGCGUUUGCCGACUGCACGG
UGAUUCUCUGUGAGCAUCGUAUCGAGGCCAUGCUCGAAUGCCAGCAAUUUCUUG
UCAUCGAAGAGAAUAAGGUCCGCCAGUACGACUCCAUCCAGAAGCUGCUUAAUGA
GAGAUCAUUGUUCCGGCAGGCGAUUUCACCAUCCGAUAGGGUGAAACUUUUUCC
ACACAGAAAUUCGUCGAAGUGCAAGUCCAAACCGCAGAUCGCGGCCUUGAAAGAA
GAGACUGAAGAAGAAGUUCAAGACACGCGUCUUCACCAUCACCAUCACCAUCACC
AUCACCAUUAAY
Codon-Optimized Human CFTR mRNA:
(SEQ ID NO.: 9)
XAUGCAGCGGUCCCCGCUCGAAAAGGCCAGUGUCGUGUCCAAACUCUUCUUCUCA
UGGACUCGGCCUAUCCUUAGAAAGGGGUAUCGGCAGAGGCUUGAGUUGUCUGAC
AUCUACCAGAUCCCCUCGGUAGAUUCGGCGGAUAACCUCUCGGAGAAGCUCGAAC
GGGAAUGGGACCGCGAACUCGCGUCUAAGAAAAACCCGAAGCUCAUCAACGCACU
GAGAAGGUGCUUCUUCUGGCGGUUCAUGUUCUACGGUAUCUUCUUGUAUCUCGG
GGAGGUCACAAAAGCAGUCCAACCCCUGUUGUUGGGUCGCAUUAUCGCCUCGUAC
GACCCCGAUAACAAAGAAGAACGGAGCAUCGCGAUCUACCUCGGGAUCGGACUGU
GUUUGCUUUUCAUCGUCAGAACACUUUUGUUGCAUCCAGCAAUCUUCGGCCUCCA
UCACAUCGGUAUGCAGAUGCGAAUCGCUAUGUUUAGCUUGAUCUACAAAAAGAC
ACUGAAACUCUCGUCGCGGGUGUUGGAUAAGAUUUCCAUCGGUCAGUUGGUGUC
CCUGCUUAGUAAUAACCUCAACAAAUUCGAUGAGGGACUGGCGCUGGCACAUUUC
GUGUGGAUUGCCCCGUUGCAAGUCGCCCUUUUGAUGGGCCUUAUUUGGGAGCUG
UUGCAGGCAUCUGCCUUUUGUGGCCUGGGAUUUCUGAUUGUGUUGGCAUUGUUU
CAGGCUGGGCUUGGGCGGAUGAUGAUGAAGUAUCGCGACCAGAGAGCGGGUAAA
AUCUCGGAAAGACUCGUCAUCACUUCGGAAAUGAUCGAAAACAUCCAGUCGGUCA
AAGCCUAUUGCUGGGAAGAAGCUAUGGAGAAGAUGAUUGAAAACCUCCGCCAAA
CUGAGCUGAAACUGACCCGCAAGGCGGCGUAUGUCCGGUAUUUCAAUUCGUCAGC
GUUCUUCUUUUCCGGGUUCUUCGUUGUCUUUCUCUCGGUUUUGCCUUAUGCCUUG
AUUAAGGGGAUUAUCCUCCGCAAGAUUUUCACCACGAUUUCGUUCUGCAUUGUA
UUGCGCAUGGCAGUGACACGGCAAUUUCCGUGGGCCGUGCAGACAUGGUAUGAC
UCGCUUGGAGCGAUCAACAAAAUCCAAGACUUCUUGCAAAAGCAAGAGUACAAG
ACCCUGGAGUACAAUCUUACUACUACGGAGGUAGUAAUGGAGAAUGUGACGGCU
UUUUGGGAAGAGGGUUUUGGAGAACUGUUUGAGAAAGCAAAGCAGAAUAACAAC
AACCGCAAGACCUCAAAUGGGGACGAUUCCCUGUUUUUCUCGAACUUCUCCCUGC
UCGGAACACCCGUGUUGAAGGACAUCAAUUUCAAGAUUGAGAGGGGACAGCUUC
UCGCGGUAGCGGGAAGCACUGGUGCGGGAAAAACUAGCCUCUUGAUGGUGAUUA
UGGGGGAGCUUGAGCCCAGCGAGGGGAAGAUUAAACACUCCGGGCGUAUCUCAU
UCUGUAGCCAGUUUUCAUGGAUCAUGCCCGGAACCAUUAAAGAGAACAUCAUUU
UCGGAGUAUCCUAUGAUGAGUACCGAUACAGAUCGGUCAUUAAGGCGUGCCAGU
UGGAAGAGGACAUUUCUAAGUUCGCCGAGAAGGAUAACAUCGUCUUGGGAGAAG
GGGGUAUUACAUUGUCGGGAGGGCAGCGAGCGCGGAUCAGCCUCGCGAGAGCGG
UAUACAAAGAUGCAGAUUUGUAUCUGCUUGAUUCACCGUUUGGAUACCUCGACG
UAUUGACAGAAAAAGAAAUCUUCGAGUCGUGCGUGUGUAAACUUAUGGCUAAUA
AGACGAGAAUCCUGGUGACAUCAAAAAUGGAACACCUUAAGAAGGCGGACAAGA
UCCUGAUCCUCCACGAAGGAUCGUCCUACUUUUACGGCACUUUCUCAGAGUUGCA
AAACUUGCAGCCGGACUUCUCAAGCAAACUCAUGGGGUGUGACUCAUUCGACCAG
UUCAGCGCGGAACGGCGGAACUCGAUCUUGACGGAAACGCUGCACCGAUUCUCGC
UUGAGGGUGAUGCCCCGGUAUCGUGGACCGAGACAAAGAAGCAGUCGUUUAAGC
AGACAGGAGAAUUUGGUGAGAAAAGAAAGAACAGUAUCUUGAAUCCUAUUAACU
CAAUUCGCAAGUUCUCAAUCGUCCAGAAAACUCCACUGCAGAUGAAUGGAAUUG
AAGAGGAUUCGGACGAACCCCUGGAGCGCAGGCUUAGCCUCGUGCCGGAUUCAGA
GCAAGGGGAGGCCAUUCUUCCCCGGAUUUCGGUGAUUUCAACCGGACCUACACUU
CAGGCGAGGCGAAGGCAAUCCGUGCUCAACCUCAUGACGCAUUCGGUAAACCAGG
GGCAAAACAUUCACCGCAAAACGACGGCCUCAACGAGAAAAGUGUCACUUGCACC
CCAGGCGAAUUUGACUGAACUCGACAUCUACAGCCGUAGGCUUUCGCAAGAAACC
GGACUUGAGAUCAGCGAAGAAAUCAAUGAAGAAGAUUUGAAAGAGUGUUUCUUU
GAUGACAUGGAAUCAAUCCCAGCGGUGACAACGUGGAACACAUACUUGCGUUAC
AUCACGGUGCACAAGUCCUUGAUUUUCGUCCUCAUCUGGUGUCUCGUGAUCUUUC
UCGCUGAGGUCGCAGCGUCACUUGUGGUCCUCUGGCUGCUUGGUAAUACGCCCUU
GCAAGACAAAGGCAAUUCUACACACUCAAGAAACAAUUCCUAUGCCGUGAUUAUC
ACUUCUACAAGCUCGUAUUACGUGUUUUACAUCUACGUAGGAGUGGCCGACACUC
UGCUCGCGAUGGGUUUCUUCCGAGGACUCCCACUCGUUCACACGCUUAUCACUGU
CUCCAAGAUUCUCCACCAUAAGAUGCUUCAUAGCGUACUGCAGGCUCCCAUGUCC
ACCUUGAAUACGCUCAAGGCGGGAGGUAUUUUGAAUCGCUUCUCAAAAGAUAUU
GCAAUUUUGGAUGACCUUCUGCCCCUGACGAUCUUCGACUUCAUCCAGUUGUUGC
UGAUCGUGAUUGGGGCUAUUGCAGUAGUCGCUGUCCUCCAGCCUUACAUUUUUG
UCGCGACCGUUCCGGUGAUCGUGGCGUUUAUCAUGCUGCGGGCCUAUUUCUUGCA
GACGUCACAGCAGCUUAAGCAACUGGAGUCUGAAGGGAGGUCGCCUAUCUUUAC
GCAUCUUGUGACCAGUUUGAAGGGAUUGUGGACGUUGCGCGCCUUUGGCAGGCA
GCCCUACUUUGAAACACUGUUCCACAAAGCGCUGAAUCUCCAUACGGCAAAUUGG
UUUUUGUAUUUGAGUACCCUCCGAUGGUUUCAGAUGCGCAUUGAGAUGAUUUUU
GUGAUCUUCUUUAUCGCGGUGACUUUUAUCUCCAUCUUGACCACGGGAGAGGGC
GAGGGACGGGUCGGUAUUAUCCUGACACUCGCCAUGAACAUUAUGAGCACUUUG
CAGUGGGCAGUGAACAGCUCGAUUGAUGUGGAUAGCCUGAUGAGGUCCGUUUCG
AGGGUCUUUAAGUUCAUCGACAUGCCGACGGAGGGAAAGCCCACAAAAAGUACG
AAACCCUAUAAGAAUGGGCAAUUGAGUAAGGUAAUGAUCAUCGAGAACAGUCAC
GUGAAGAAGGAUGACAUCUGGCCUAGCGGGGGUCAGAUGACCGUGAAGGACCUG
ACGGCAAAAUACACCGAGGGAGGGAACGCAAUCCUUGAAAACAUCUCGUUCAGCA
UUAGCCCCGGUCAGCGUGUGGGGUUGCUCGGGAGGACCGGGUCAGGAAAAUCGA
CGUUGCUGUCGGCCUUCUUGAGACUUCUGAAUACAGAGGGUGAGAUCCAGAUCG
ACGGCGUUUCGUGGGAUAGCAUCACCUUGCAGCAGUGGCGGAAAGCGUUUGGAG
UAAUCCCCCAAAAGGUCUUUAUCUUUAGCGGAACCUUCCGAAAGAAUCUCGAUCC
UUAUGAACAGUGGUCAGAUCAAGAGAUUUGGAAAGUCGCGGACGAGGUUGGCCU
UCGGAGUGUAAUCGAGCAGUUUCCGGGAAAACUCGACUUUGUCCUUGUAGAUGG
GGGAUGCGUCCUGUCGCAUGGGCACAAGCAGCUCAUGUGCCUGGCGCGAUCCGUC
CUCUCUAAAGCGAAAAUUCUUCUCUUGGAUGAACCUUCGGCCCAUCUGGACCCGG
UAACGUAUCAGAUCAUCAGAAGGACACUUAAGCAGGCGUUUGCCGACUGCACGG
UGAUUCUCUGUGAGCAUCGUAUCGAGGCCAUGCUCGAAUGCCAGCAAUUUCUUG
UCAUCGAAGAGAAUAAGGUCCGCCAGUACGACUCCAUCCAGAAGCUGCUUAAUGA
GAGAUCAUUGUUCCGGCAGGCGAUUUCACCAUCCGAUAGGGUGAAACUUUUUCC
ACACAGAAAUUCGUCGAAGUGCAAGUCCAAACCGCAGAUCGCGGCCUUGAAAGAA
GAGACUGAAGAAGAAGUUCAAGACACGCGUCUUUAAY
Codon Optimized Human CFTR mRNA coding sequence with a
Growth Hormone Leader Sequence (italisized and underlined)
(SEQ ID NO: 10)
AUGGCCACUGGAUCAAGAACCUCACUGCUGCUCGCUUUUGGACUGCUUUGCCUGCC
CUGGUUGCAAGAAGGAUCGGCUUUCCCGACCAUCCCACUCUCCAUGCAGCGGUCCC
CGCUCGAAAAGGCCAGUGUCGUGUCCAAACUCUUCUUCUCAUGGACUCGGCCUAU
CCUUAGAAAGGGGUAUCGGCAGAGGCUUGAGUUGUCUGACAUCUACCAGAUCCCC
UCGGUAGAUUCGGCGGAUAACCUCUCGGAGAAGCUCGAACGGGAAUGGGACCGC
GAACUCGCGUCUAAGAAAAACCCGAAGCUCAUCAACGCACUGAGAAGGUGCUUCU
UCUGGCGGUUCAUGUUCUACGGUAUCUUCUUGUAUCUCGGGGAGGUCACAAAAG
CAGUCCAACCCCUGUUGUUGGGUCGCAUUAUCGCCUCGUACGACCCCGAUAACAA
AGAAGAACGGAGCAUCGCGAUCUACCUCGGGAUCGGACUGUGUUUGCUUUUCAU
CGUCAGAACACUUUUGUUGCAUCCAGCAAUCUUCGGCCUCCAUCACAUCGGUAUG
CAGAUGCGAAUCGCUAUGUUUAGCUUGAUCUACAAAAAGACACUGAAACUCUCG
UCGCGGGUGUUGGAUAAGAUUUCCAUCGGUCAGUUGGUGUCCCUGCUUAGUAAU
AACCUCAACAAAUUCGAUGAGGGACUGGCGCUGGCACAUUUCGUGUGGAUUGCCC
CGUUGCAAGUCGCCCUUUUGAUGGGCCUUAUUUGGGAGCUGUUGCAGGCAUCUG
CCUUUUGUGGCCUGGGAUUUCUGAUUGUGUUGGCAUUGUUUCAGGCUGGGCUUG
GGCGGAUGAUGAUGAAGUAUCGCGACCAGAGAGCGGGUAAAAUCUCGGAAAGAC
UCGUCAUCACUUCGGAAAUGAUCGAAAACAUCCAGUCGGUCAAAGCCUAUUGCUG
GGAAGAAGCUAUGGAGAAGAUGAUUGAAAACCUCCGCCAAACUGAGCUGAAACU
GACCCGCAAGGCGGCGUAUGUCCGGUAUUUCAAUUCGUCAGCGUUCUUCUUUUCC
GGGUUCUUCGUUGUCUUUCUCUCGGUUUUGCCUUAUGCCUUGAUUAAGGGGAUU
AUCCUCCGCAAGAUUUUCACCACGAUUUCGUUCUGCAUUGUAUUGCGCAUGGCAG
UGACACGGCAAUUUCCGUGGGCCGUGCAGACAUGGUAUGACUCGCUUGGAGCGA
UCAACAAAAUCCAAGACUUCUUGCAAAAGCAAGAGUACAAGACCCUGGAGUACA
AUCUUACUACUACGGAGGUAGUAAUGGAGAAUGUGACGGCUUUUUGGGAAGAGG
GUUUUGGAGAACUGUUUGAGAAAGCAAAGCAGAAUAACAACAACCGCAAGACCU
CAAAUGGGGACGAUUCCCUGUUUUUCUCGAACUUCUCCCUGCUCGGAACACCCGU
GUUGAAGGACAUCAAUUUCAAGAUUGAGAGGGGACAGCUUCUCGCGGUAGCGGG
AAGCACUGGUGCGGGAAAAACUAGCCUCUUGAUGGUGAUUAUGGGGGAGCUUGA
GCCCAGCGAGGGGAAGAUUAAACACUCCGGGCGUAUCUCAUUCUGUAGCCAGUUU
UCAUGGAUCAUGCCCGGAACCAUUAAAGAGAACAUCAUUUUCGGAGUAUCCUAU
GAUGAGUACCGAUACAGAUCGGUCAUUAAGGCGUGCCAGUUGGAAGAGGACAUU
UCUAAGUUCGCCGAGAAGGAUAACAUCGUCUUGGGAGAAGGGGGUAUUACAUUG
UCGGGAGGGCAGCGAGCGCGGAUCAGCCUCGCGAGAGCGGUAUACAAAGAUGCA
GAUUUGUAUCUGCUUGAUUCACCGUUUGGAUACCUCGACGUAUUGACAGAAAAA
GAAAUCUUCGAGUCGUGCGUGUGUAAACUUAUGGCUAAUAAGACGAGAAUCCUG
GUGACAUCAAAAAUGGAACACCUUAAGAAGGCGGACAAGAUCCUGAUCCUCCACG
AAGGAUCGUCCUACUUUUACGGCACUUUCUCAGAGUUGCAAAACUUGCAGCCGGA
CUUCUCAAGCAAACUCAUGGGGUGUGACUCAUUCGACCAGUUCAGCGCGGAACGG
CGGAACUCGAUCUUGACGGAAACGCUGCACCGAUUCUCGCUUGAGGGUGAUGCCC
CGGUAUCGUGGACCGAGACAAAGAAGCAGUCGUUUAAGCAGACAGGAGAAUUUG
GUGAGAAAAGAAAGAACAGUAUCUUGAAUCCUAUUAACUCAAUUCGCAAGUUCU
CAAUCGUCCAGAAAACUCCACUGCAGAUGAAUGGAAUUGAAGAGGAUUCGGACG
AACCCCUGGAGCGCAGGCUUAGCCUCGUGCCGGAUUCAGAGCAAGGGGAGGCCAU
UCUUCCCCGGAUUUCGGUGAUUUCAACCGGACCUACACUUCAGGCGAGGCGAAGG
CAAUCCGUGCUCAACCUCAUGACGCAUUCGGUAAACCAGGGGCAAAACAUUCACC
GCAAAACGACGGCCUCAACGAGAAAAGUGUCACUUGCACCCCAGGCGAAUUUGAC
UGAACUCGACAUCUACAGCCGUAGGCUUUCGCAAGAAACCGGACUUGAGAUCAGC
GAAGAAAUCAAUGAAGAAGAUUUGAAAGAGUGUUUCUUUGAUGACAUGGAAUCA
AUCCCAGCGGUGACAACGUGGAACACAUACUUGCGUUACAUCACGGUGCACAAGU
CCUUGAUUUUCGUCCUCAUCUGGUGUCUCGUGAUCUUUCUCGCUGAGGUCGCAGC
GUCACUUGUGGUCCUCUGGCUGCUUGGUAAUACGCCCUUGCAAGACAAAGGCAAU
UCUACACACUCAAGAAACAAUUCCUAUGCCGUGAUUAUCACUUCUACAAGCUCGU
AUUACGUGUUUUACAUCUACGUAGGAGUGGCCGACACUCUGCUCGCGAUGGGUU
UCUUCCGAGGACUCCCACUCGUUCACACGCUUAUCACUGUCUCCAAGAUUCUCCA
CCAUAAGAUGCUUCAUAGCGUACUGCAGGCUCCCAUGUCCACCUUGAAUACGCUC
AAGGCGGGAGGUAUUUUGAAUCGCUUCUCAAAAGAUAUUGCAAUUUUGGAUGAC
CUUCUGCCCCUGACGAUCUUCGACUUCAUCCAGUUGUUGCUGAUCGUGAUUGGGG
CUAUUGCAGUAGUCGCUGUCCUCCAGCCUUACAUUUUUGUCGCGACCGUUCCGGU
GAUCGUGGCGUUUAUCAUGCUGCGGGCCUAUUUCUUGCAGACGUCACAGCAGCUU
AAGCAACUGGAGUCUGAAGGGAGGUCGCCUAUCUUUACGCAUCUUGUGACCAGU
UUGAAGGGAUUGUGGACGUUGCGCGCCUUUGGCAGGCAGCCCUACUUUGAAACA
CUGUUCCACAAAGCGCUGAAUCUCCAUACGGCAAAUUGGUUUUUGUAUUUGAGU
ACCCUCCGAUGGUUUCAGAUGCGCAUUGAGAUGAUUUUUGUGAUCUUCUUUAUC
GCGGUGACUUUUAUCUCCAUCUUGACCACGGGAGAGGGCGAGGGACGGGUCGGU
AUUAUCCUGACACUCGCCAUGAACAUUAUGAGCACUUUGCAGUGGGCAGUGAAC
AGCUCGAUUGAUGUGGAUAGCCUGAUGAGGUCCGUUUCGAGGGUCUUUAAGUUC
AUCGACAUGCCGACGGAGGGAAAGCCCACAAAAAGUACGAAACCCUAUAAGAAU
GGGCAAUUGAGUAAGGUAAUGAUCAUCGAGAACAGUCACGUGAAGAAGGAUGAC
AUCUGGCCUAGCGGGGGUCAGAUGACCGUGAAGGACCUGACGGCAAAAUACACCG
AGGGAGGGAACGCAAUCCUUGAAAACAUCUCGUUCAGCAUUAGCCCCGGUCAGCG
UGUGGGGUUGCUCGGGAGGACCGGGUCAGGAAAAUCGACGUUGCUGUCGGCCUU
CUUGAGACUUCUGAAUACAGAGGGUGAGAUCCAGAUCGACGGCGUUUCGUGGGA
UAGCAUCACCUUGCAGCAGUGGCGGAAAGCGUUUGGAGUAAUCCCCCAAAAGGUC
UUUAUCUUUAGCGGAACCUUCCGAAAGAAUCUCGAUCCUUAUGAACAGUGGUCA
GAUCAAGAGAUUUGGAAAGUCGCGGACGAGGUUGGCCUUCGGAGUGUAAUCGAG
CAGUUUCCGGGAAAACUCGACUUUGUCCUUGUAGAUGGGGGAUGCGUCCUGUCG
CAUGGGCACAAGCAGCUCAUGUGCCUGGCGCGAUCCGUCCUCUCUAAAGCGAAAA
UUCUUCUCUUGGAUGAACCUUCGGCCCAUCUGGACCCGGUAACGUAUCAGAUCAU
CAGAAGGACACUUAAGCAGGCGUUUGCCGACUGCACGGUGAUUCUCUGUGAGCA
UCGUAUCGAGGCCAUGCUCGAAUGCCAGCAAUUUCUUGUCAUCGAAGAGAAUAA
GGUCCGCCAGUACGACUCCAUCCAGAAGCUGCUUAAUGAGAGAUCAUUGUUCCGG
CAGGCGAUUUCACCAUCCGAUAGGGUGAAACUUUUUCCACACAGAAAUUCGUCGA
AGUGCAAGUCCAAACCGCAGAUCGCGGCCUUGAAAGAAGAGACUGAAGAAGAAG
UUCAAGACACGCGUCUUUAA

Aliquots of 50 mg/mL ethanolic solutions of cKK-E12, DOPE, Chol and DMG-PEG2K were mixed in a molar ratio of 40:30:25:5 and diluted with ethanol to 3 mL final volume. Separately, an aqueous buffered solution (10 mM citrate/150 mM NaCl, pH 4.5) of FIX, ASS1, or FFL mRNA was prepared from a 1 mg/mL stock. The lipid solution was injected rapidly into the aqueous mRNA solution and shaken to yield a final suspension in 20% ethanol. The resulting nanoparticle suspension was filtered, diafiltrated with 1×PBS (pH 7.4), concentrated and stored at 2-8° C. The final concentration of FIX mRNA was approximately 0.77 mg/mL FIX mRNA (encapsulated), Zₐᵥₑ=76 nm, PDI=0.08. The final concentration of ASS1 mRNA was approximately 0.64 mg/mL ASS1 mRNA (encapsulated), Zₐᵥₑ=78 nm (Dv(50)=46 nm; Dv(90)=96 nm). The final concentration of FFL mRNA was approximately 1.31 mg/mL FFL mRNA (encapsulated), Zₐᵥₑ=75 nm, PDI—0.11. The final concentration of SMN mRNA was approximately 1.85 mg/mL SMN mRNA (encapsulated). Average particle size (Zₐᵥₑ)=71 nm, (particle size for 50% of particles was 44 nm or less (Dv(50))=44 nm; and the particle size for 90% of the particles was 93n or less (Dv(90)=93 nm)).

### Example 2. Administration of mRNA-Loaded Liposome Nanoparticles

This example illustrates exemplary methods of administering mRNA-loaded liposome nanoparticles and methods for analyzing delivered mRNA and subsequently expressed protein in various target tissues in vivo.

All studies were performed using male CD-1 mice of approximately 6-8 weeks of age at the beginning of each experiment. Samples were introduced by a single bolus tail-vein injection of an equivalent total dose of 1.0 mg/kg (or otherwise specified) of encapsulated FIX, FFL or ASS1 mRNA. Mice were sacrificed and perfused with saline at the designated time points.

Various organ tissues such as the liver, spleen, kidney and heart of each mouse was harvested, apportioned into separate parts, and stored in either 10% neutral buffered formalin or snap-frozen and stored at −80° C.; for analysis.

All animals were euthanized by CO₂ asphyxiation at designated time points post dose administration (±5%) followed by thoracotomy and terminal cardiac blood collection. Whole blood (maximal obtainable volume) was collected via cardiac puncture on euthanized animals into serum separator tubes, allowed to clot at room temperature for at least 30 minutes, centrifuged at 22° C.±5° C.; at 9300 g for 10 minutes, and the serum extracted. For interim blood collections, approximately 40-50 μL of whole blood was collected via facial vein puncture or tail snip. Samples collected from non-treatment animals were used as a baseline ASS1 levels for comparison to study animals.

### Enzyme-Linked Immunosorbent Assay (ELISA) Analysis

### A. Human FIX ELISA

Quantification of FIX protein was performed following procedures reported for human FIX ELISA kit (AssayMax, Assay Pro, Catalog #EF1009-1).

### B. Human ASS1 ELISA

Standard ELISA procedures were followed employing mouse anti-ASS1 2D1-2E12 IgG as the capture antibody with rabbit anti-ASS1 #3285 IgG as the secondary (detection) antibody (Shire Human Genetic Therapies). Horseradish peroxidase (HRP)-conjugated goat anti-rabbit IgG was used for activation of the 3,3′,5,5′-tetramethylbenzidine (TMB) substrate solution. The reaction was quenched using 2N H2SO4 after 20 minutes. Detection was monitored via absorption (450 nm) on a Molecular Device SpectraMax instrument. Untreated mouse serum and organs and human ASS1 protein were used as negative and positive controls, respectively.

### IVIS Bioluminometer Measurements

To visual luminescence in treated mice, several steps were followed. Anesthesia using isoflurane vaporizer at 1-3% (usually @2.5%) was initially employed. Using a microsprayer, 50 μL/animal of luciferin in PBS was administered at 60 mg/mL via intratracheal/intranasal. Luciferin was allowed to distribute for 5-10 minutes. Animals were placed in an isoflurane chamber until anesthetized. Anesthetized animals were placed into the IVIS imaging chamber at dorsal recumbency and positioned into the manifold. Pictures of mice were taken. In these Examples, the acquisition settings providing highest sensitivity were: camera height at D level, F/Stop at f1, binning at high resolution, and exposure time at 5 minutes. Exposures were repeated up to 3 times (5, 10 and 15 minutes post Luciferin Injection).

### In Situ Hybridization (ISH) Analysis

In situ hybridization was performed using “ZZ” probe technology. Probes were generated based on codon-optimized sequence of human messenger RNA. Tissues were fixed for 24-48 hours in 10% neutral buffered formalin and embedded in paraffin. Positive detection of desired mRNA was achieved through 6 consecutive amplification steps followed by chromagenic visualization using 3,3′-diaminobenzidine (DAB). Positive signal was compared to that of untreated mouse.

### Example 3. Highly Effective In Vivo Production of Therapeutic Proteins

This example demonstrates highly efficient and sustained production of proteins encoded by mRNA delivered by liposomes incorporating the cationic lipids described herein (e.g., cKK-E12) in serum and various organ tissues.

### In Vivo Human FIX Protein Production Results

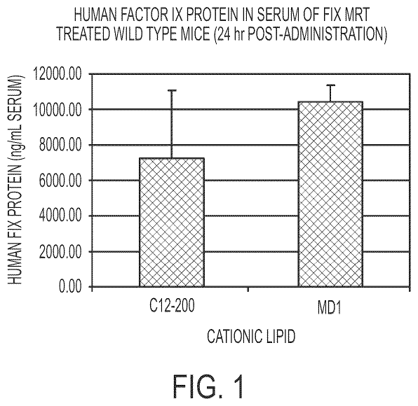

The production of human FIX protein via hFIX mRNA-loaded cKK-E12-based lipid nanoparticles was tested in CD-1 mice as a single, bolus intravenous injection. FIG. 1 represents the amount of human FIX protein detected via ELISA when treating mice with human FIX mRNA-loaded cKK-E12-based lipid nanoparticles as compared to a C12-200-based lipid nanoparticle encapsulating hFIX mRNA. The mice were sacrificed twenty-four hours post-injection and organs were harvested (as described above).

C12-200-based lipid nanoparticles have been shown to be an effective vehicle to deliver and express mRNA in vivo (see, PCT Application Publication NO. WO2012170930, the disclosure of which is hereby incorporated by reference). Surprisingly, as represented in FIG. 1, cKK-E12 based lipid nanoparticles are even more effective in delivering human FIX mRNA in vivo, resulting in close to 50% higher protein expression detected in the plasma of the treated mice, as compared to C12-200-based lipid nanoparticles.

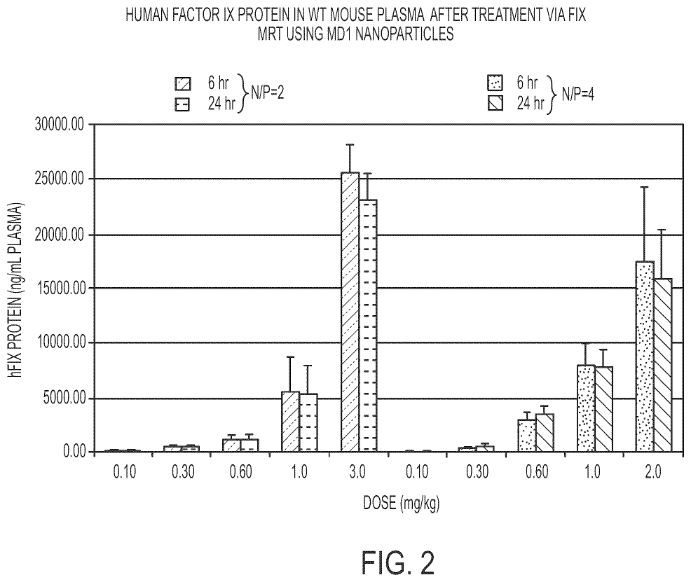

FIG. 2 shows the results of a dose-response experiment as represented by the amount of human FIX protein detected via ELISA when treating mice with human FIX mRNA-loaded cKK-E12-based lipid nanoparticles at various doses. The mice were bled at 6 hours and sacrificed twenty-four hours post-injection and organs were harvested (as described above).

A clear dose response was achieved when measuring liver levels of human FIX protein. The dosing range was from 0.10-3.0 mg/kg of encapsulated human FIX mRNA. These data demonstrate the ability of the lipid nanoparticles to efficiently deliver messenger RNA, release the payload and process this exogenous mRNA via translation to produce human FIX protein, which is then subsequently secreted into the bloodstream. Levels of human FIX protein are well above therapeutic levels (>100 ng/mL plasma) and surpass normal physiological levels (˜5 ug/mL plasma) when dosing at 1.0 mg/kg or greater. Further, the plasma residence time of this human protein is sustained through at least 24 hours post administration.

### In Vivo Human ASS1 Protein Production Results

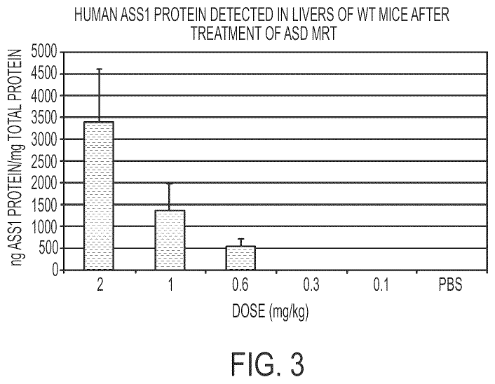

The production of human ASS1 protein via codon-optimized hASS1 mRNA-loaded cKK-E12-based lipid nanoparticles was tested in CD-1 mice as a single, bolus intravenous injection. FIG. 3 represents the amount of human ASS1 protein detected via ELISA when treating mice with human ASS1 mRNA-loaded cKK-E12-based lipid nanoparticles at various doses. The mice were sacrificed twenty-four hours post-injection and organs were harvested (as described above).

A clear dose response was achieved when measuring liver levels of human ASS1 protein. As shown in Table 5, the dosing range was from 0.10-2.0 mg/kg of encapsulated human ASS1 mRNA in cKK-E12 lipid nanoparticles. These data demonstrate the ability of the lipid nanoparticles to accumulate in the liver and release the mRNA payload and the liver to process this exogenous mRNA via translation to produce human ASS1 protein.

TABLE 5
Raw values of human ASS1 protein as measured via ELISA analysis
(as depicted in FIG. 1). Codon-optimized human ASS1 mRNA was
delivered via cKK-E12-based lipid nanoparticles. Doses are based
on encapsulated ASS1 mRNA. Values are depicted as nanogram of
human ASS1 protein per milligram total protein in liver.

| Dose Encapsulated | Human |
|---|---|
| ASS1 mRNA (mg/kg) | ASS1 Protein (ng/mg total protein) |
| 0.10 | BLD |
| 0.30 | BLD |
| 0.60 | 546 |
| 1.0 | 1388 |
| 2.0 | 3371 |

BLD = Below Limit of Detection for ELISA.

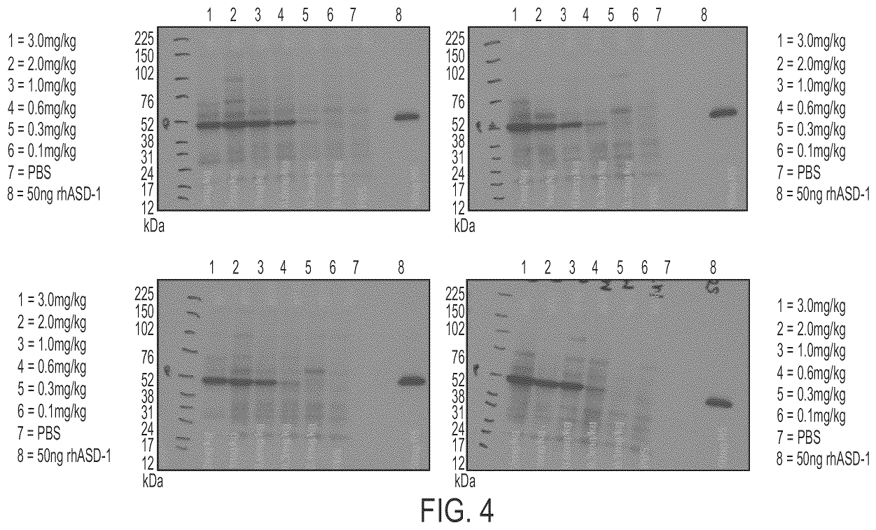

While the sensitivity of the ELISA has limitations at lower values, western blot analysis allows for clear visualization of the human ASS1 protein at lower doses (0.3-3.0 mg/kg) (see FIG. 4). FIG. 4 depicts a comparison of human ASS1 protein levels in liver as a function of dose via western blot analysis upon a single intravenous dose of human ASS1 mRNA-encapsulated cKK-E12 lipid nanoparticles. CD1 mice were sacrificed at 24 hours post-administration and livers were harvested and analyzed as described above.

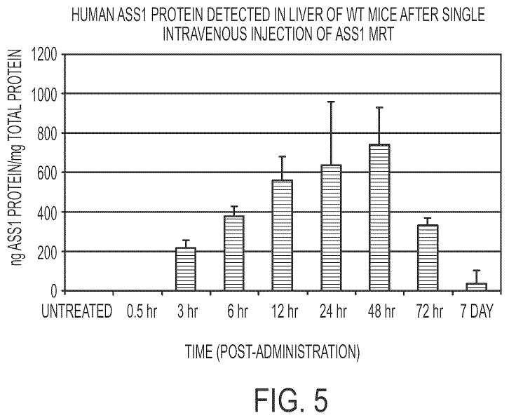

To further understand the ability of ASS1 mRNA-encapsulated lipid nanoparticles to facilitate the delivery of mRNA to selected organs (liver), a pharmacokinetic analysis was performed, monitoring human ASS1 protein levels in the liver over a one week time period. FIG. 5 depicts the quantity of human ASS1 protein detected in the liver at various time points up to 7 days after administration of human ASS1-loaded lipid nanoparticles (cKK-E12). This was accomplished as a single dose (1.0 mg/kg encapsulated mRNA) given intravenously.

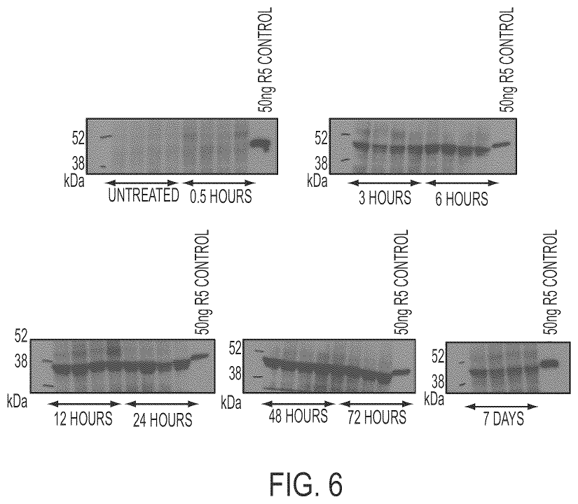

In this case, we observed a maximum serum level of human ASS1 protein at approximately 24-48 hours post-administration. Measurable levels of protein were still observed 1 week post-administration as determined by both ELISA and western blot (FIGS. 5 and 6, respectively). FIG. 6 depicts a comparison of human ASS1 protein levels in liver over time via western blot analysis upon a single intravenous dose of human ASS1 mRNA-encapsulated lipid nanoparticles (1.0 mg/kg dose).

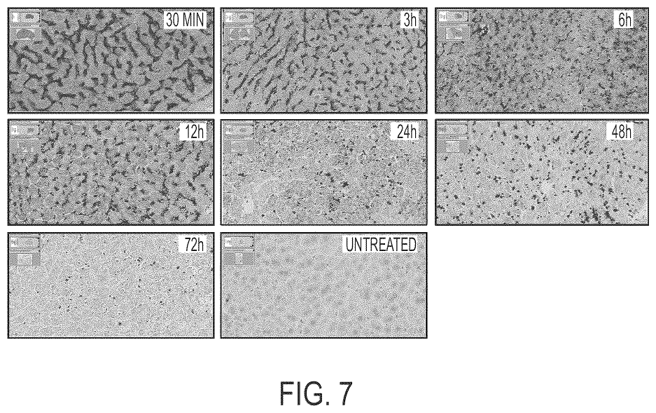

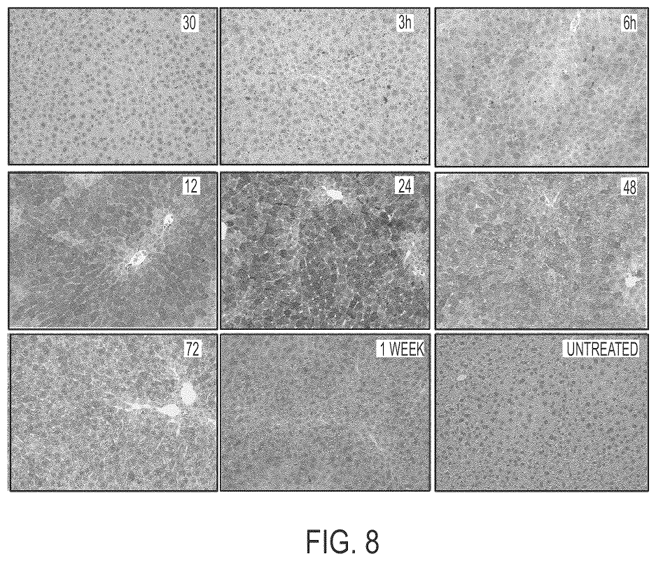

Direct detection of the active pharmaceutical ingredient (ASS1 mRNA) in the livers of the treated mice was achieved using in situ hybridization (ISH) based methods. As demonstrated in FIGS. 7 & 8, the exogenous human ASS1 messenger RNA could be detected in high levels at the earliest time point tested (30 minutes) and the signal remained strong for 48 hours after dosing. Further, human ASS1 mRNA was still detectable 72 hours post-administration.

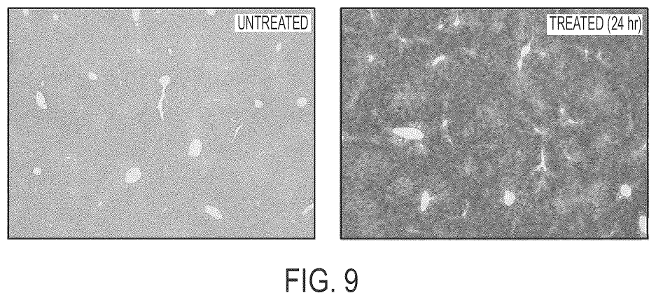

In addition to ISH, detection of the resulting human ASS1 protein was achieved using immunohistochemical (IHC) means. Using a mouse monoclonal antibody (02D2-2E12) for specific binding, the presence of target human ASS1 protein in the cytoplasm of hepatocytes of treated livers can be readily observed. FIG. 9 shows the immunohistochemical staining of human ASS1 protein in treated mouse livers 24 hours after administration.

In Vivo Delivery of FFL mRNA Via Nebulization

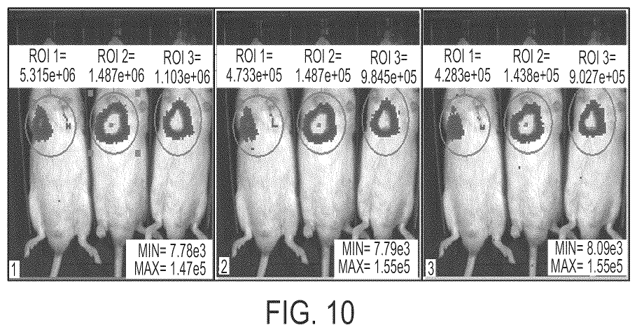

To assess whether additional routes of delivery were feasible, FFL mRNA was encapsulated in cKK-E12 liposomes and those liposomes were nebulized. As shown in FIG. 10, it is possible to efficiently nebulize cKK-E12 based lipid nanoparticles encapsulating mRNA. FIG. 10 represents mice treated with luciferin 24 hours after exposure to nebulized FFL mRNA loaded cKK-E12 lipid nanoparticles.

### Example 4. CNS Delivery of hSMN-1 mRNA

This example provides an exemplary cKK-E12 liposome formulations for effective delivery and expression of mRNA in the CNS. Specifically, the example demonstrates that delivery of human survival of motor neuron-1 (hSMN-1) mRNA into various tissues of the brain and spinal cord.

### Messenger RNA Material

Codon-optimized human Survival of Motor Neuron-1(hSMN-1) messenger RNA (see SEQ ID NO: 4) was synthesized by in vitro transcription from a plasmid DNA template encoding the gene, which was followed by the addition of a 5′ cap structure (Cap 1) (Fechter, P.; Brownlee, G. G. “Recognition of mRNA cap structures by viral and cellular proteins” J. Gen. Virology 2005, 86, 1239-1249) and a 3′ poly(A) tail of approximately 250 nucleotides in length (SEQ ID NO: 15) as determined by gel electrophoresis. The 5′ and 3′ untranslated regions present in each mRNA product are represented as X and Y, respectively and defined as stated in Example 1.

### Formulation Protocol

Lipid nanoparticles (LNP) were formed via standard ethanol injection methods (Ponsa, M.; Foradada, M.; Estelrich, J. “Liposomes obtained by the ethanol injection method” Int. J. Pharm. 1993, 95, 51-56). For the various lipid components, a 50 mg/ml ethanolic stock solutions was prepared and stored at −20° C. In preparation of the cKK-E12 lipid nanoparticle formulation listed in Table 6, each indicated lipid component was added to an ethanol solution to achieve a predetermined final concentration and molar ratio, and scaled to a 3 ml final volume of ethanol. Separately, an aqueous buffered solution (10 mM citrate/150 mM NaCl, pH 4.5) of hSMN-1 mRNA was prepared from a 1 mg/ml stock. The lipid solution was injected rapidly into the aqueous mRNA solution and shaken to yield a final suspension in 20% ethanol. The resulting nanoparticle suspension was filtered and dialysed against 1×PBS (pH 7.4), concentrated and stored between 2-8° C. SMN-1 mRNA concentration was determined via the Ribogreen assay (Invitrogen). Encapsulation of mRNA was calculated by performing the Ribogreen assay with and without the presence of 0.1% Triton-X 100. Particle sizes (dynamic light scattering (DLS)) and zeta potentials were determined using a Malvern Zetasizer instrument in 1×PBS and 1 mM KCl solutions, respectively.

TABLE 6
Exemplary cKK-E12 Lipid Nanoparticle formulation

| | | | Final | |
|---|---|---|---|---|
| | | Molar | mRNA | |
| Formu- | | Ratio | Concen- | Zeta |
| lations | Components | of lipids | tration | Parameters |
| 1 | cKK-E12 | 40:30:25:5 | 1.8 mg/ml | Zₐᵥₑ = 72 nm; |
| | DOPE | | | Dv₍₅₀₎ = 49 nm; |
| | Cholesterol | | | Dv₍₉₀₎ = 90 nm |
| | DMG-PEG-2K | | | |
| | hSMN-1 mRNA | | | |

Intrathecal Administration of mRNA Loaded Liposome Nanoparticles

All in vivo studies were performed using either rats or mice of approximately 6-8 weeks of age at the beginning of each experiment. At the start of the experiment, each animal was anesthetized with isoflurane (1-3%, to effect) by inhalation. Once anesthetized, each animal was shaved at the exact injection site (L4-L5 or L5-L6). Following insertion of the needle, reflexive flick of the tail was used to indicate puncture of the dura and confirm intrathecal placement. Each animal received a single bolus intrathecal injection of the test formulation listed in Table 6. All animals were sacrificed 24 hours post injection and perfused with saline.

### Isolation of Organ Tissues for Analysis

All animals had the whole brain and spinal cord harvested. The brain was cut longitudinally and placed in one histology cassette per animal. The whole spinal cord was stored ambient in a 15 ml tube containing 10% neutral buffered formalin (NBF) for at least 24 hours and no more than 72 hours before transfer into 70% histology grade alcohol solution. Each spinal cord sample was cut into cervical, thoracic and lumbar sections. Each spinal cord section cut in half and both halves were placed in individual cassettes per section (cervical, thoracic and lumbar) for processing. All three cassettes were embedded into one paraffin block per animal. When applicable, portions of brain and spinal cord were snap frozen and stored at −80° C.

hSMN-1 Western Blot Analysis

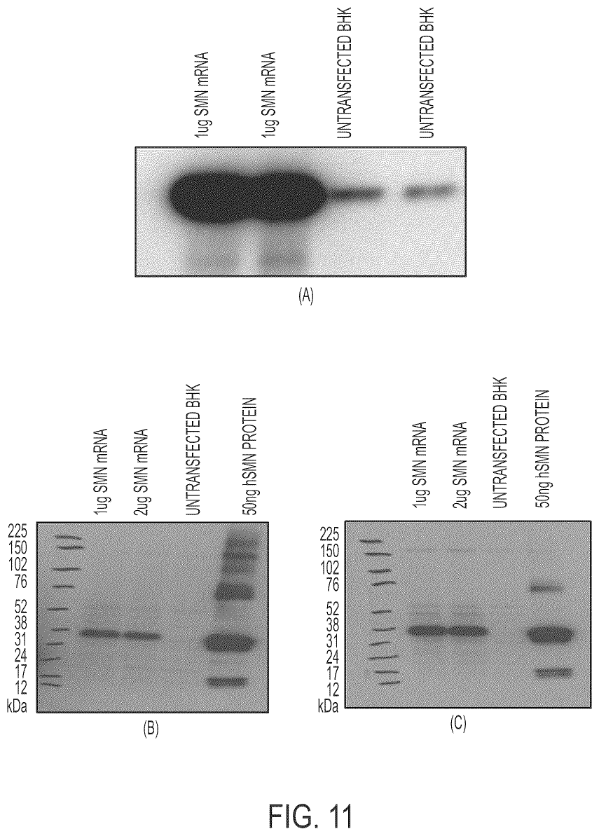

Standard western blot procedures were followed employing various antibodies that recognizes hSMN protein, such as: (A) anti-SMN 4F11 antibody at 1:1,000 dilution; (B) Pierce PA5-27309 a-SMN antibody at 1:1,000 dilution; and (C) LSBio C138149 a-SMN antibody at 1:1,000 dilution. For each experiment one microgram of hSMN mRNA was transfected into ˜1×10⁶ BHK-21 cells using Lipofectamine 2000. Cells were treated with OptiMem and harvested 16-18 hours post-transfection. Cell lysates were harvested, processed and loaded on to an 8-16% Tris Glycine gel. The gel was transferred using a PVDF membrane and treated with the respective primary antibody. Goat anti-mouse HRP antibody was used as the secondary antibody at 1:10,000 dilution for 45 minutes at room temperature followed by washing and development. The data demonstrates that each antibody tested showed a strong signal for hSMN-1 and was specific for human SMN, as indicated by an absence in a cross-reactive signal for untreated BHK cells (FIG. 11).

### In Situ Hybridzation (ISH) Analysis

Tissue from each representative sample, was assayed for hSMN-1 mRNA using a manual in situ hybridization analysis, performed using RNAscope® (Advanced Cell Diagnostic) “ZZ” probe technology. Probes were generated based on the codon-optimized sequence of human SMN messenger RNA (SEQ ID NO: 4). Briefly, the RNAscope® assay is an in situ hybridication assay designed to visualize single RNA molecules per cell in formalin-fixed, paraffin-embedded (FFPE) tissue mounted on slides. Each embedded tissue sample was pretreated according to the manufacturers protocol and incubated with a target specific hSMN-1 RNA probe. The hSMN-1 probe was shown to be specific for human SMN-1 and had little to no cross reactivity with mouse or rat SMN-1. Once bound, the hSMN-1 probe is hybridized to a cascade of signal amplification molecules, through a series of 6 consecutive rounds of amplification. The sample was then treated with an HRP-labeled probe specific to the signal amplification cassette and assayed by chromatic visualization using 3,3′-diaminobenzidine (DAB). A probe specific for Ubiquitin C was used as the positive control. Positive SMN signal was compared to that of untreated and vehicle control treated rat or mouse tissue. Stained samples were visualized under a standard bright field microscope.

### Immunohistochemical Analysis

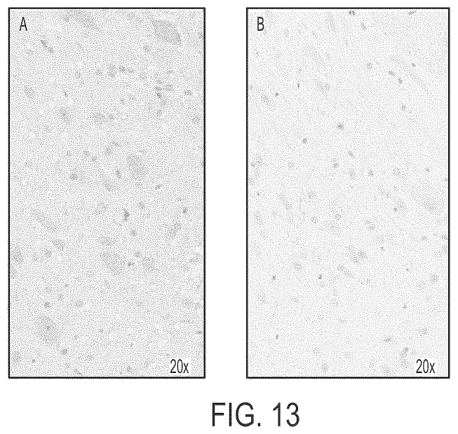

Human SMN-1 mRNA-loaded lipid nanoparticles were administered to rats via intrathecal injection, and tissue samples collected and processed 24 hours post administration in accordance with the methods described above. Rat spinal tissue samples were then assayed for hSMN-1 protein expression. Briefly, fixed tissue embedded in paraffin was processed and placed on slides. The slides were dewaxed, rehydrated and antigen retrieval was performed using a pressure cooker with citrate buffer. Several blocking buffers were employed followed by primary antibody incubation overnight at 4° C., using the 4F11 antibody at a 1:2500 dilution. The resulting slides were washed and incubated at ambient temperature with the secondary antibody polymer followed by washing and subsequent chromagen development. The data demonstrates that in as little as 24 hours post intrathecal adminiatration of hSMN-1 mRNA, staining is observed for human SMN-1 protein when compared to no-treatment control (FIG. 13). This supports the previous findings which demonstrate delivery of hSMN-1 mRNA to the spinal tissue. Furthermore, the data demonstrates that once delivered to the cell hSMN-1 mRNA is effectively expressed to generate hSMN-1 protein.

### Results

The data presented in this example demonstrates that intrathecal administration of hSMN-1 mRNA loaded liposomes (e.g., lipid or polymer-based nanoparticles) results in successful intracellular delivery of mRNA in neurons in the brain and spinal cord, including those difficult to treat cells, such as anterior horn cells and dorsal root ganglia.

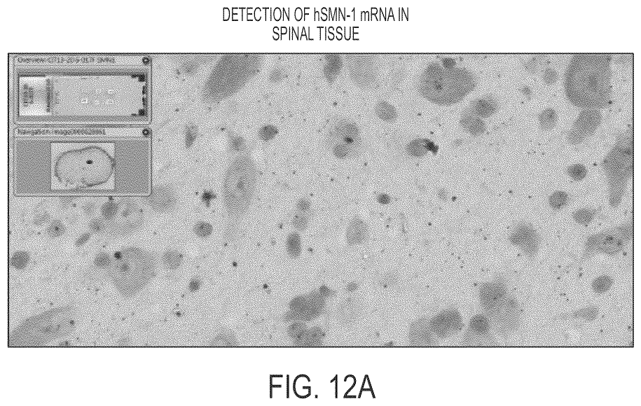

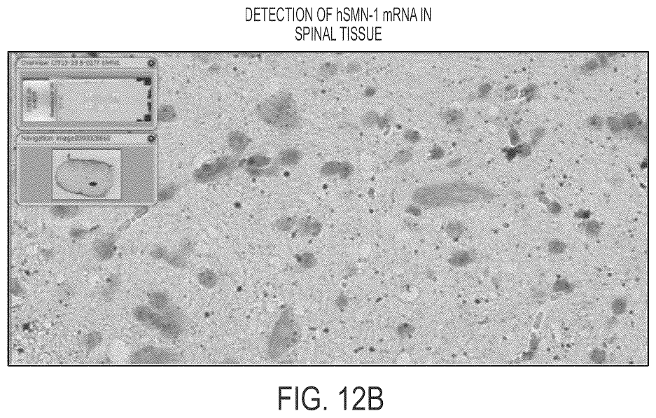

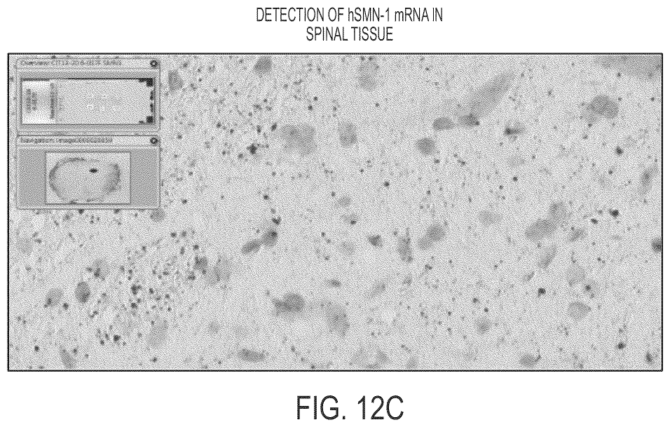

The results have shown that mRNA encapsulated within a lipid nanoparticle (e.g., lipid nanoparticle comprising cKK-E12) can be effectively delivered to various tissues of the CNS following intrathecal administrations. Using the exemplary formulation disclosed in Table 6, mRNA was effectively delivered and internalized within various neurons of the spinal cord (FIGS. 12A-12C), as verified by in situ hybridization assay. Surprisingly, intracellular mRNA delivery was demonstrated in the difficult to reach neuronal cells of the anterior horn, located deep within the tissues of the spinal column (FIGS. 12A-12C). Little to no background was observed with mouse or rat SMN-1, indicating specificity for the human SMN-1 probe. Positive SMN signal was compared to that of untreated and vehicle control treated rat or mouse tissue. Stained samples were visualized under a standard bright field microscope.

These data demonstrates that the lipid or polymer nanoparticle based mRNA delivery approach described herein was able to successfully permeate the complex and dense cell membrane of the spinal cord neurons and deliver the mRNA payload for the production of encoded proteins inside neurons. It was particularly surprising that the mRNA delivery approach described herein was equally successful in permeating difficult to treat neurons such as anterior horn cell and dorsal root ganglia. Thus, the data presented herein demonstrates that lipid or polymer nanoparticles, such as those comprising cKK-E12, may serve as a promising option for delivering mRNA to neuronal cells in the treatment of a CNS disease. In particular, the present example demonstrates that hSMN mRNA loaded nanoparticles can be effectively delivered to neurons, including those difficult to treat motor neurons in the spinal cord, and can be used for the production of SMN protein and treatment of spinal muscular atrophy.

### Example 5. In Vivo CO-CFTR-C-His₁₀ mRNA Delivery to CFTR Knockout Mice

Messenger RNA Synthesis. For the experiment, C-terminal His₁₀ tagged codon-optimized human cystic fibrosis transmembrane conductance regulator (CO-CFTR-C-His₁₀) (SEQ ID NO:8) (“His₁₀” disclosed as SEQ ID NO: 11) and non-tagged codon-optimized human CFTR (CO-CFTR) (SEQ ID NO:9) mRNA were synthesized by in vitro transcription from a plasmid DNA template using standard method. mRNAs used in this example and Example 6 were produced by IVT in which 25% of U residues were 2-thio-uridine and 25% of C residues were 5-methylcytidine.

Analysis of human CFTR protein produced via intratracheal administered mRNA-loaded nanoparticles. For the study, CFTR knockout mice were used. CFTR mRNA formulation or vehicle control was introduced using a PART Boy jet nebulizer. Mice were sacrificed and perfused with saline, after a predetermined period of time, to allow for protein expression from the mRNA.

PEI Formulation. PEI formulation has been used to deliver CFTR mRNA to the lung and was used as a control in this experiment. Polymeric nanoparticle formulations with 25 kDa branched PEI were prepared as follows. The required amount of mRNA was diluted just before application in water for injection (Braun, Melsungen) to a total volume of 4 ml and added quickly to 4 ml of an aqueous solution of branched PEI 25 kDa using a pipette at an N/P ratio of 10. The solution was mixed by pipetting up and down ten times and nebulized as two separate 4.0 ml fractions one after another to the mouse lungs using the indicated nebulizer.

cKK-E12 Formulation. For the lipid-based nanoparticle experiment, a lipid formulation was created using CO-CFTR-C-His₁₀ RNA in a formulation of cKK-E12:DOPE:Chol:PEGDMG2K (relative amounts 50:25:20:5 (mg:mg:mg:mg)). The solution was nebulized to the mouse lungs using the indicated nebulizer.

Nebulization (Aerosol) Administration of Human CO-CFTR-C His₁₀ mRNA. CFTR test materials were administered by a single aerosol inhalation via PART Boy jet nebulizer (nominal dose volume of up to 8 mL/group). The test material was delivered to a box containing the whole group of animals (n=4) and connected to oxygen flow and scavenger system.

Administration of Human CO-CFTR-C His₁₀ mRNA. CFTR mRNA was prepared in the manner described above. Four CFTR knockout mice were placed in an aerosol chamber box and exposed to 2 mg total codon optimized unmodified human CFTR mRNA (comprising the coding sequence of SEQ ID NO: 8) via nebulization (Para Boy jet nebulizer) over the course of approximately one hour. Mice were sacrificed 24 hours post-exposure.

Euthanasia. Animals were euthanized by CO₂ asphyxiation at representative times post-dose administration (±5%) followed by thoracotomy and exsanguinations. Whole blood (maximal obtainable volume) was collected via cardiac puncture and discarded.

Perfusion. Following exsanguination, all animals underwent cardiac perfusion with saline. In brief, whole body intracardiac perfusion was performed by inserting 23/21 gauge needle attached to 10 mL syringe containing saline set into the lumen of the left ventricle for perfusion. The right atrium was incised to provide a drainage outlet for perfusate. Gentle and steady pressure was applied to the plunger to perfuse the animal after the needle had been positioned in the heart. Adequate flow of the flushing solution was ensured when the exiting perfusate flows clear (free of visible blood) indicating that the flushing solution has saturated the body and the procedure was complete.

Tissue Collection. Following perfusion, all animals had their lungs (right and left) harvested. Both (right and left) lungs were snap frozen in liquid nitrogen and stored separately at nominally −70° C.

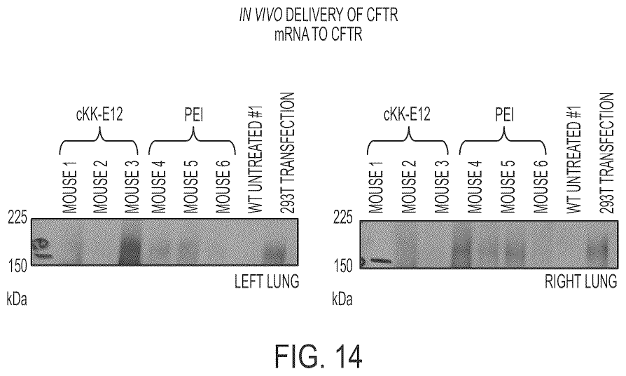

Expression of human CFTR in CO-CFTR-C-His₁₀ in CFTR knockout mice. CFTR expression was detected by Western blot analysis of tissue lysate collected from CFTR mRNA-treated mouse lungs. Mature “C” band was detected in left and right lungs of all treated mice, for both the cKK-E12-based and PEI-based formulations (FIG. 14). Expression of the mature “C” band was verified by comparison with lysate collected from HEK 293T human CO-CFTR—C-Hisao positive cells. In contrast, no detectable signal was observed in lysate collected from wild type untreated control mice (FIG. 14). Taken together, these data suggest that cKK-E12 may be used to deliver mRNA (e.g., CFTR mRNA) to the lung via, e.g., inhalation, as effectively as or even better than PEI based formulations.

### Example 6: In Vivo Expression in the Lung

This example further demonstrates successful in vivo expression in the lung following aerosol delivery of mRNA-loaded ckk-E12 based nanoparticles. All studies were performed using pigs of the German Landrace, obtained from Technical University Munich, Weihenstephan, Germany. The pigs had a body weight ranging from 35-90 kg. FFL/CO-CFTR-C-His10 mRNA formulation or vehicle control was introduced using a Pari jet nebulizer. Pigs were sacrificed and perfused with saline, after a predetermined period of time, to allow for protein expression from the mRNA.

Messenger RNA Synthesis. In the example, codon optimized fire fly luciferase (CO-FFL) mRNA was synthesized by in vitro transcription from plasmid DNA templates.

cKK-E12 Formulation. For the lipid-based nanoparticle experiment, a lipid formulation was created using 1 mg FFL+9 mg of CO-CFTR—C-His₁₀ mRNA encapsulated in a formulation of cKK-E12: DOPE: Chol:PEGDMG2K (relative amounts 40:30:25:5 (mol ratio). The solution was nebulized to the Pig lungs using the indicated nebulizer.

Aerosol Application. The aerosol (Saline or CO-FFL cKK-E12 formulation) was nebulized and inhaled into the anaesthetized pig. Sedation in pigs was initiated by premedication with azaperone 2 mg/kg body weight, ketamine 15 mg/kg body weight, atropine 0.1 mg/kg body weight and followed by insertion of an intravenous line to the lateral auricular vein. Pigs were anesthetized by intravenous injection of propofol 3-5 mg/kg body weight as required. Anesthesia was maintained by isoflurane (2-3%) with 1% propofol bolus injection at 4 to 8 mg/kg body weight to enhance anesthesia as required. Duration of the anesthesia was approximately 1-3 hrs. Pigs were killed with bolus injection of pentobarbital (100 mg/kg body weight) and potassium chloride via the lateral ear vein. Lungs were excised and tissue specimens were collected from various lung regions followed by incubation in cell culture medium overnight. The stored samples were subjected to bioluminescence detection.

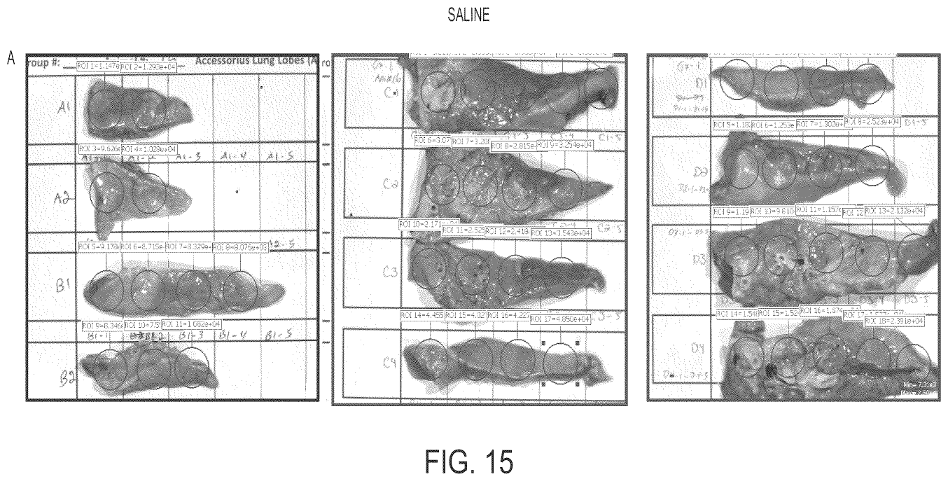

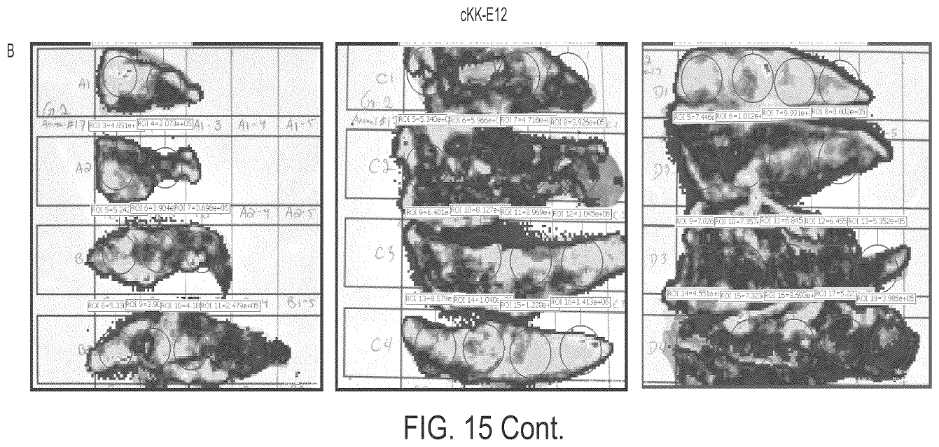

Bioluminescence Analysis. For measurement of luciferase activity, tissue specimens were either homogenized and analyzed in a tube luminometer or incubated in a medium bath comprising D-Luciferin substrate and subjected to ex vivo luciferase BLI. The data illustrate that a strong bioluminescence signal was observed for each of the (A)CO-FFL/CO-CFTR-C-His₁₀ mRNA treated pigs, when compared to (B) control lung tissue samples from control pigs (Saline vehicle control) (FIG. 15 A&B).

These data illustrate that FFL/CFTR mRNA were successfully delivered to and expressed in the lung by aerosol administration of a cKK-E12 based lipid formulation.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1-56. (canceled)

57. A method of delivery of messenger RNA (mRNA) in vivo, comprising administering to a subject in need of delivery a composition comprising an mRNA encapsulated within a liposome,
wherein administration of the composition results in the expression of a protein encoded by the mRNA in vivo;
wherein the liposome comprises a cationic lipid of formula I-g: 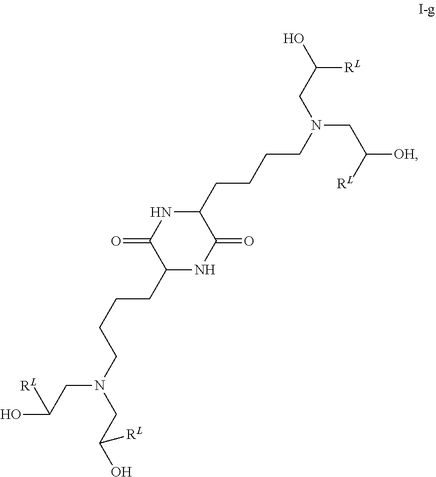
or a pharmaceutically acceptable salt thereof,
wherein R^{L} is linear C₈₋₁₂ alkyl and the liposome further comprises a non-cationic lipid, a cholesterol-based lipid, and a PEG-modified lipid, and
wherein the mRNA has a length of or greater than about 0.5 kb.

58. The method of claim 57, wherein R^{L} is linear —C₈H₁₇, —C₉H₁₉, —C₁₀H₂₁, —C₁₁H₂₃, or —C₁₂H₂₅.

59. The method of claim 58, wherein R^{L} is linear —C₈H₁₇.

60. The method of claim 57, wherein the non-cationic lipid is selected from DSPC (1,2-distearoyl-sn-glycero-3-phosphocholine), DPPC (1,2-dipalmitoyl-sn-glycero-3-phosphocholine), DOPE (1,2-dioleyl-sn-glycero-3-phosphoethanolamine), DOPC (1,2-dioleyl-sn-glycero-3-phosphotidylcholine) DPPE (1,2-dipalmitoyl-sn-glycero-3-phosphoethanolamine), DMPE (1,2-dimyristoyl-sn-glycero-3-phosphoethanolamine), and DOPG (1,2-dioleoyl-sn-glycero-3-phospho-(1′-rac-glycerol)).

61. The method of claim 57, wherein the cholesterol-based lipid is cholesterol or PEGylated cholesterol.

62. The method of claim 57, wherein the PEG-modified lipid comprises a poly(ethylene) glycol chain of up to 5 kDa in length covalently attached to a lipid with alkyl chain(s) of C₆-C₂₀ length.

63. The method of claim 59, wherein the liposome comprises the cationic lipid, DOPE, cholesterol, and DMG-PEG2K.

64. The method of claim 57, wherein the cationic lipid constitutes about 30-50% of the liposome by molar ratio.

65. The method of claim 64, wherein the cationic lipid constitutes about 40% of the liposome by molar ratio.

66. The method of claim 63, wherein the ratio of cationic lipid: DOPE: cholesterol:DMG-PEG2K is approximately 40:30:20:10, 40:30:25:5, or 40:32:25:3 by molar ratio.

67. The method of claim 57, wherein the liposome has a size less than about 250 nm, 200 nm, 150 nm, 100 nm, 75 nm, or 50 nm.

68. The method of claim 57, wherein the composition is to be administered intravenously, intrathecally, or via pulmonary delivery.

69. The method of claim 68, wherein the pulmonary delivery is by aerosolization, inhalation, nebulization or instillation.

70. The method of claim 57, wherein the expression of the protein encoded by the mRNA is detectable in liver, kidney, heart, spleen, serum, brain, skeletal muscle, lymph nodes, skin, and cerebrospinal fluid.

71. The method of claim 57, wherein the expression of the protein encoded by the mRNA is detectable 3 hours, 6 hours, 12 hours, 24 hours, or 1 week after the administration.

72. The method of claim 57, wherein the mRNA has a length of or greater than about 1 kb, 1.5 kb, 2 kb, 2.5 kb, 3 kb, 3.5 kb, 4 kb, 4.5 kb, or 5 kb.

73. The method of claim 57, wherein the protein encoded by the mRNA is a cytosolic protein, a secreted protein, or an enzyme.

74. The method of claim 57, wherein the protein encoded by the mRNA is cystic fibrosis transmembrane conductance regulator (CFTR), argininosuccinate synthetase (ASS1), factor IX (FIX), survival of motor neuron 1 (SMN-1), or phenylalanine hydroxylase.

75. The method of claim 57, wherein the mRNA is administered at a dose ranging from about 0.1-2.0 mg/kg body weight.

76. The method of claim 57, wherein the mRNA is administered at a dose of or less than about 1.0 mg/kg, 0.5 mg/kg, or 0.3 mg/kg body weight.

77. The method of claim 57, wherein the mRNA is unmodified or comprises one or more modified nucleotides.

78. The method of claim 77, wherein the one or more modified nucleotides comprise pseudouridine, N-1-methyl-pseudouridine, 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and/or 2-thiocytidine.
